(19) United States
(12) Patent Application Publication
(10) Pub. No.: US 2024/0299328 A1
(43) Pub. Date: Sep. 12, 2024

(54) COMPOSITIONS COMPRISING 15-HEPE AND METHODS OF TREATING AND/OR PREVENTING HEMATOLOGIC DISORDERS AND RELATED DISEASES

(71) Applicant: Afimmune Limited, Dublin (IE)

(72) Inventors: John Climax, Dublin (IE); Moayed Hamza, Dublin (IE)

(21) Appl. No.: 18/596,817

(22) Filed: Mar. 6, 2024

Related U.S. Application Data
(60) Provisional application No. 63/488,589, filed on Mar. 6, 2023.

(51) Int. Cl. A61K 31/202; A61K 9/00; A61K 31/232; A61P 7/06

(52) U.S. Cl. CPC A61K 31/202; A61K 9/0053; A61K 31/232; A61P 7/06

(57) ABSTRACT

Compositions comprising 15-hydroxyeicosapentaenoic acid (15-HEPE) in free acid form or a pharmaceutically acceptable ester, conjugate, or salt thereof; and therapeutic methods of treating a subject comprising administering a composition comprising 15-HEPE to treat diseases including red blood cell disorders, hemoglobin disorders, bleeding disorders, and blood cancer in a subject in need thereof.

### CROSS-REFERENCE TO RELATED APPLICATION(S)

The present application claims priority to U.S. Provisional Patent Application No. 63/488,589, filed on Mar. 6, 2023, the disclosure of which is incorporated by reference as if set out in full.

### TECHNICAL FIELD

The present application relates generally to compositions comprising 15-hydroxyeicosapentaenoic acid (15-HEPE), and to methods of using the same, including in the treatment of hematologic disorders.

### SEQUENCE LISTING

This application contains an ST.26 compliant Sequence Listing, which is submitted concurrently in xml format via Patent Center and is hereby incorporated by reference in its entirety. The .xml copy, created on Feb. 5, 2024, is named 1243188019US01.xml and is 13,954 bytes in size.

### BACKGROUND

Blood is the life-maintaining fluid that circulates through the body and is made up of liquid and solids. The liquid part, called plasma, is made of water, salts, and protein. The solid part contains red blood cells, white blood cells, and platelets. Hematologic disorders are diseases of the blood (e.g., red blood cells) and blood-forming organs. Common hematologic disorders include anemias, bleeding disorders, and blood cancers. A subclass of hematologic disorders, referred to as red blood cell disorders, are conditions that affect the production, lifespan, and shape of red blood cells (RBCs) and its oxygen transport molecule, hemoglobin. These conditions inhibit the transport of oxygen from the lungs to the rest of the body. One the other hand, bleeding disorders include hemophilia, a condition characterized by insufficient clotting factor and excessive bleeding, and thrombophilia, a condition characterized by increased risks of developing blood clots in veins and arteries. These clots can break loose from the blood vessel and travel through the blood stream to an organ, thereby preventing blood flow to that organ resulting in ischemia. Blood clots can lead to an increased risk of stroke and/or pulmonary embolism.

### SUMMARY

The application relates to compositions comprising 15-hydroxyeicosapentaenoic acid (15-HEPE) and to methods of using such compositions in the treatment of a variety of diseases and disorders, including hematologic diseases and disorders.

In one aspect, provided is a method of treating and/or preventing a hematologic disorder in a subject in need thereof, comprising administering to the subject a composition comprising 15-hydroxyeicosapentaenoic acid (15-HEPE) in free acid form or a pharmaceutically acceptable ester, conjugate, or salt thereof. In some embodiments, the hematologic disorder is selected from the group consisting of a red blood cell disorder, a hemoglobin disorder, a bleeding disorder, and a blood cancer.

In some embodiments, the red blood cell disorder is selected from the group consisting of anemia, hemolytic anemia, iron deficiency anemia, hereditary spherocytosis, chronic hemolysis, pyruvate kinase deficiency, and glucose-6-phosphate dehydrogenase (G6PD) deficiency.

In some embodiments, the hemoglobin disorder is selected from the group consisting of sickle cell disease, alpha thalassemia, and beta thalassemia.

In some embodiments, the bleeding disorder is selected from the group consisting of hemophilia, thrombophilia, thrombocytopenia, idiopathic thrombocytopenic purpura (ITP), heparin-induced thrombocytopenia (HIT), venous thromboembolism, arterial thrombosis, and embolism.

In some embodiments, the blood cancer is selected from the group consisting of myelodysplastic syndrome (MDS), lymphoma, leukemia, and myeloma.

In one aspect, provided is a method of treating and/or preventing sickle cell disease and/or symptoms associated thereof in a subject in need thereof, comprising administering to the subject a composition comprising 15-hydroxyeicosapentaenoic acid (15-HEPE) in free acid form or a pharmaceutically acceptable ester, conjugate, or salt thereof. In some embodiments, the symptoms associated thereof comprise inflammatory vasculopathy, vasoconstriction, bronchoconstriction, iron overload, hemoglobinopathy, chromic hemolysis, sickling, anemia, inflammation, pain, acute and/or chronic damage to an organ (e.g., lung, kidney, liver, spleen), and/or oxidative stress.

In one aspect, provided is a method of treating and/or preventing heparin-induced thrombocytopenia (HIT) in a subject in need thereof, comprising administering to the subject a composition comprising 15-hydroxyeicosapentaenoic acid (15-HEPE) in free acid form or a pharmaceutically acceptable ester, conjugate, or salt thereof.

In some embodiments, the subject has no change or a reduction in serum creatine levels after receiving the composition comprising 15-HEPE relative to baseline or to a control subject who is not administered the composition comprising 15-HEPE or who is administered placebo.

In some embodiments, the subject has no change or a reduction in C-reactive protein (CRP) levels after receiving the composition comprising 15-HEPE relative to baseline or to a control subject who is not administered the composition comprising 15-HEPE or who is administered placebo.

In some embodiments, the subject has no change or a reduction in pentraxin-2 levels after receiving the composition comprising 15-HEPE relative to baseline or to a control subject who is not administered the composition comprising 15-HEPE or who is administered placebo.

In some embodiments, the subject has no change or a reduction in monocyte chemoattractant protein-1 (MCP-1/CCL2) levels after receiving the composition comprising 15-HEPE relative to baseline or to a control subject who is not administered the composition comprising 15-HEPE or who is administered placebo.

In some embodiments, the subject has no change or a reduction in circulating neutrophils after receiving the composition comprising 15-HEPE relative to baseline or to a control subject who is not administered the composition comprising 15-HEPE or who is administered placebo.

In some embodiments, the subject has no change or a reduction in vascular cell adhesion molecule 1 (VCAM-1) levels after receiving the composition comprising 15-HEPE relative to baseline or to a control subject who is not administered the composition comprising 15-HEPE or who is administered placebo.

In some embodiments, the subject has no change or a reduction in intercellular adhesion molecule 1 (ICAM-1) levels after receiving the composition comprising 15-HEPE relative to baseline or to a control subject who is not administered the composition comprising 15-HEPE or who is administered placebo.

In some embodiments, the subject has no change or a reduction in endothelin-1 (ET-1) levels after receiving the composition comprising 15-HEPE relative to baseline or to a control subject who is not administered the composition comprising 15-HEPE or who is administered placebo.

In some embodiments, the subject has no change or an increase in hematocrit levels after receiving the composition comprising 15-HEPE relative to baseline or to a control subject who is not administered the composition comprising 15-HEPE or who is administered placebo.

In some embodiments, the subject has no change or an increase in hemoglobin levels after receiving the composition comprising 15-HEPE relative to baseline or to a control subject who is not administered the composition comprising 15-HEPE or who is administered placebo.

In some embodiments, the subject has no change or a reduction in sickled red blood cell numbers after receiving the composition comprising 15-HEPE relative to baseline or to a control subject who is not administered the composition comprising 15-HEPE or who is administered placebo.

In some embodiments, the subject has no change or a reduction in CD68 levels after receiving the composition comprising 15-HEPE relative to baseline or to a control subject who is not administered the composition comprising 15-HEPE or who is administered placebo.

In some embodiments, the subject has no change or a reduction in CD80 levels after receiving the composition comprising 15-HEPE relative to baseline or to a control subject who is not administered the composition comprising 15-HEPE or who is administered placebo.

In some embodiments, the subject has no change or a reduction in NF-kB p65 activation after receiving the composition comprising 15-HEPE relative to baseline or to a control subject who is not administered the composition comprising 15-HEPE or who is administered placebo.

In some embodiments, the subject has no change or a reduction in NLRP3 inflammasome activation after receiving the composition comprising 15-HEPE relative to baseline or to a control subject who is not administered the composition comprising 15-HEPE or who is administered placebo.

In some embodiments, the subject has no change or a reduction in vascular endothelial activation after receiving the composition comprising 15-HEPE relative to baseline or to a control subject who is not administered the composition comprising 15-HEPE or who is administered placebo.

In some embodiments, the subject has no change or a reduction in in neutrophil vascular recruitment after receiving the composition comprising 15-HEPE relative to baseline or to a control subject who is not administered the composition comprising 15-HEPE or who is administered placebo.

In some embodiments, the subject has no change or a reduction in inflammatory cell infiltration after receiving the composition comprising 15-HEPE relative to baseline or to a control subject who is not administered the composition comprising 15-HEPE or who is administered placebo.

In some embodiments, the subject has no change or a reduction in thrombi formation after receiving the composition comprising 15-HEPE relative to baseline or to a control subject who is not administered the composition comprising 15-HEPE or who is administered placebo.

In some embodiments, the subject has no change or a reduction in iron overload and/or liver iron accumulation after receiving the composition comprising 15-HEPE relative to baseline or to a control subject who is not administered the composition comprising 15-HEPE or who is administered placebo.

In some embodiments, the subject has no change or a reduction in oxidative stress after receiving the composition comprising 15-HEPE relative to baseline or to a control subject who is not administered the composition comprising 15-HEPE or who is administered placebo.

In some embodiments, the subject has no change or a reduction in nuclear factor erythroid 2-related factor 2 (Nrf2) levels after receiving the composition comprising 15-HEPE relative to baseline or to a control subject who is not administered the composition comprising 15-HEPE or who is administered placebo.

In some embodiments, the subject has no change or a reduction in oxidation and/or heme oxygenase-1 (HO-1) levels after receiving the composition comprising 15-HEPE relative to baseline or to a control subject who is not administered the composition comprising 15-HEPE or who is administered placebo.

In some embodiments, the subject has no change or a reduction in E-selectin levels after receiving the composition comprising 15-HEPE relative to baseline or to a control subject who is not administered the composition comprising 15-HEPE or who is administered placebo.

In some embodiments, the subject has no change or a reduction in red blood cell adhesion after receiving the composition comprising 15-HEPE relative to baseline or to a control subject who is not administered the composition comprising 15-HEPE or who is administered placebo.

In some embodiments, the 15-HEPE is in free acid form, salt form, or esterified form, for example, a C1-C5 alkyl ester (e.g., an ethyl ester) or a triglyceride.

In some embodiments, the 15-HEPE is in 15(S)-HEPE enantiomeric form and/or 15(R)-HEPE enantiomeric form.

In some embodiments, the composition comprises one or more additional active agent(s) selected from the group consisting of eicosapentaenoic acid (EPA), 5-hydroxy-eicosapentaenoic acid (5-HEPE), 12-hydroxy-eicosapentaenoic acid (12-HEPE), 18-hydroxy-eicosapentaenoic acid (18-HEPE), 15-hydroxy-eicosatrienoic acid (15-HETrE), 15-hydroxy-eicosatetraenoic acid (15-HETE), 14-hydroxy-docosahexaenoic acid (14-HDoHE), 17-hydroxy-docosahexaenoic acid (17-HDoHE), resolvin D1 (RvD1 or 17(S)-RvD1), and aspirin-triggered resolvin D1 (AT-RvD1 or 17(R)-RvD1).

In some embodiments, 15-HEPE represents at least 80% wt. of the composition, or at least 80% wt. of all fatty acids present in the composition. In some embodiments, the composition comprises no more than 10% wt. of omega-3 fatty acids other than 15-HEPE. In some embodiments, omega-3 fatty acids other than 15-HEPE represents no more than 10% wt. of all fatty acids present in the composition.

In some embodiments, the composition is formulated in one or more orally deliverable dosage units, for example, one or more capsules. In some embodiments, about 500 mg to about 1000 mg of 15-HEPE is present in each capsule. In some embodiments, the composition further comprises a pharmaceutically acceptable excipient.

In some embodiments, the composition is administered to the subject to provide a daily dose of about 1 g to about 8 g, or about 8 g, of 15-HEPE, for example, in 1 to 8 capsules per day.

### BRIEF DESCRIPTION OF THE DRAWINGS

FIG. 1 is a schematic diagram of the study described in Example 1 and its duration.

FIG. 2 shows the body weight changes of the animals according to the study described in Example 1.

FIG. 3 shows the body weight on the day of sacrifice of the animals according to the study described in Example 1.

FIGS. 4A-4D show the kidney weight and kidney-to-body weight ratio of the animals on the day of sacrifice according to the study described in Example 1.

FIG. 5 shows the kidney hydroxyproline content for the animals according to the study described in Example 1.

FIGS. 6A-6G show the Sirius red staining of the animals according to the study described in Example 1.

FIG. 7 shows a plot depicting the Sirius red-positive area (%) of the animals according to the study described in Example 1.

FIGS. 8A-8D show the gene expression analyses for α-SMA, TIMP-1, TGF-β, and Collagen Type 1 of the animals according to the study described in Example 1, respectively.

FIG. 9 is a schematic diagram of the study described in Example 2 and its duration.

FIG. 10 shows the body weight changes of the animals according to the study described in Example 2.

FIG. 11 shows the body weight on the day of sacrifice of the animals according to the study described in Example 2.

FIGS. 12A and 12B show the liver weight and liver-to-body weight ratio of the animals on the day of sacrifice according to the study described in Example 2, respectively.

FIG. 13 shows the changes of serum ALT levels of the animals according to the study described in Example 2.

FIG. 14 shows the changes of serum total bilirubin levels of the animals according to the study described in Example 2.

FIGS. 15A-15E show the Sirius red staining and the fibrosis area of the animals according to the study described in Example 2.

FIG. 16 shows a plot depicting the Sirius red-positive area (%) of the animals according to the study described in Example 2.

FIG. 17A-17D show the gene expression analyses for α-SMA, TIMP-1, TGF-β, and Collagen Type 1 of the animals according to the study described in Example 2, respectively.

FIGS. 18A and 18B show DS102 (15-HEPE) induced inhibition of TGF-β according the study described in Example 3.

FIGS. 18C-18G show DS102 effects on membrane translocation and degradation of Type I TGF-β receptor, Type II TGF-β receptor, Type III TGF-β receptor, EGFR and Caveolin-1 according to the study described in Example 3, respectively.

FIG. 19 is a schematic diagram of the study described in Example 4 and its duration.

FIG. 20 shows the baseline lipidomic profile of patients according to the study described in Example 4.

FIGS. 21A-21C are plots depicting the changes in insulin, glucose, and free fatty acid levels in patients administered Epeleuton (15-HEPE) and placebo, respectively.

FIGS. 22A and 22B are plots depicting the changes in HOMA-IR and apido-IR levels in patients administered Epeleuton and placebo, respectively.

FIGS. 23A and 23B are plots depicting the changes in mean HbA1C levels in patients and proportion of patients having HbA1C levels≥6.5% at week 16 administered Epeleuton and placebo, respectively.

FIGS. 24A and 24B are plots depicting the mean change and median (%) change in the lipid profile of patients administered Epeleuton and placebo, respectively.

FIGS. 25A-25C are plots depicting the changes in cholesterol, triglyceride, and VLDL-C levels in patients administered Epeleuton and placebo, respectively.

FIG. 26 is a chart depicting the changes in hepatoxic lipid profile of patients administered DS102.

FIG. 27 are plots that validate that administration DS102 resolves NASH using the OWL liver care test.

FIG. 28 are plots depicting the changes in hepatic fat content by CAP in patients administered DS102 and placebo.

FIGS. 29A-29C is a chart depicting changes in inflammatory and pro-fibrotic protein levels in patients administered DS102 and placebo.

FIG. 30 are plots depicting the changes in the protein expression including of NASH development targets in patients administered DS102 and placebo.

FIG. 31 is a volcano plot depicting a reduction in inflammatory and pro-fibrotic proteins in patients administered DS102 and placebo.

FIG. 32 are plots depicting the changes in vascular adhesion molecules in patients administered DS102 and placebo.

FIG. 33 are plots depicting the changes in cardiovascular risk proteins in patients administered DS102 and placebo.

FIG. 34 are plots depicting the changes in chemokines in patients administered DS102 and placebo.

FIG. 35 are plots depicting the changes in tumor necrosis factor receptor superfamily members in patients administered DS102 and placebo.

FIGS. 36A and 36B are plots depicting the reduction in ALP levels in patients administered DS102 and placebo.

FIG. 37 is a boxplot of 15-HEPE ethyl ester trough plasma relative concentrations.

FIG. 38A-38C are bar graphs depicting mean change and standard deviation of red blood cell counts, red blood cell distribution width, and reticulocyte counts in rats administered 15(S)-HEPE EE and placebo, respectively.

FIG. 39A-39C are bar graphs depicting mean change and standard deviation of prothrombin time, activated partial thromboplastin time, and fibrinogen concentration in rats administered 15(S)-HEPE EE and placebo, respectively.

FIGS. 40A-40H. In SCD mice, Epeleuton reduces circulating neutrophils and modulates inflammatory response with downregulation of markers of inflammatory vasculopathy. FIG. 40A. Circulating neutrophils identified by flow cytometric analysis as CD45+Ly6G+ cells in AA and SS mice treated with either vehicle or Epeleuton (1000 mg/Kg/d for 6 weeks). Data are presented as mean±SEM (n=4-9); * p<0.05 compared to healthy mice; P<0.05 compared to vehicle treated SS animals by one-way ANOVA. FIGS. 40B-40D. Immunoblot analysis using specific antibodies against phosphorylated (p-)NF-κB p65 and NF-κB p65 in lung (FIG. 40B), kidney (FIG. 40C) and liver (FIG. 40D) from AA and SS mice treated as in FIG. 40A. 75 μgr of protein loaded on an 8% T, 2.5% C polyacrylamide gel. GAPDH serves as protein loading control. One representative gel from 4 with similar results is shown. Densitometric analysis of immunoblots is shown in FIG. 49A. FIGS. 40E and 40F. Immunoblot analysis using specific antibodies against VCAM-1, ICAM-1 and ET-1 in lung (FIG. 40E), kidney (FIG. 40F) from AA and SS mice treated as in FIG. 40A. 75 μgr of protein loaded on an 11% T, 2.5% C polyacrylamide gel. GAPDH serves as protein loading control. One representative gel from 4 with similar results is shown. Densitometric analysis of immunoblots is shown in FIGS. 49B and 50A, respectively. FIG. 40G. Upper panel. Immunoblot analysis using specific antibodies against VCAM-1 and ET-1 in liver from AA and SS mice treated as in FIG. 40A. 50 μgr/μl of protein loaded on an 8% T, 2.5% C polyacrylamide gel. GAPDH serves as protein loading control. One representative gel from 4 with similar results is shown. Lower panel. Densitometric analysis of immunoblots is shown in the lower panel. Data are presented as means±SEM (n=4); * p<0.05 compared to AA mice; ºp<0.05 compared to vehicle by one-way ANOVA (DU: Densitometric Unit). FIG. 40H. OxyBlot analysis of the soluble fractions of liver from AA and SS mice treated as in FIG. 40B. The carbonylated proteins (1 mg) were detected by treating with 2,4-dinitrophenylhydrazine and blotted with anti-DNP antibody. GAPDH serves as protein loading control. Quantification of band area is shown in FIG. 50B.

FIGS. 41A-41E. In SCD mice exposed to H/R stress, Epeleuton reduces the H/R induced hemolysis and the inflammatory response. FIG. 41A. Hematocrit (Hct, left panel) and hemoglobin (Hb, right panel) in AA and SS mice exposed to H/R: hypoxia (8% oxygen; 10 hours), followed by reoxygenation (21% oxygen; 3 hours) treated with either vehicle or Epeleuton (1000 mg/Kg/day for 6 weeks). Data are presented as mean±SEM (n=4-9). * p<0.05 compared to AA mice; ºp<0.05 compared to vehicle treated mice by upaired t-test with Bonferroni correction. FIG. 41B. Erythrocyte morphology from blood smear of SS mice treated as in FIG. 41A. One representative image is shown. Original magnification 100×. Data are mean±SEM (n=6). \hat{ }p<0.05 compared to normoxia,° p<0.05 compared to vehicle treated mice by upaired t-test with Bonferroni correction. FIG. 41C. Immunoblot analysis using specific anti Phospho-Tyrosine antibody of red cell membrane proteins from mice treated as in FIG. 41A. 75 μg of proteins loaded on an 8% T, 2.5% C polyacrylamide gel. Coomassie staining was used as loading control. Densitometric analysis of the phosphorylation of Band-3 is shown on the right. Data are presented as means±SEM (n=3), * p<0.05 compared to AA mice; ºp<0.05 compared to vehicle treated mice by upaired t-test with Bonferroni correction. FIG. 41D. Lactate dehydrogenase (LDH) plasma values in AA and SS mice under normoxia condition or exposed to H/R and treated as in FIG. 41A. Data are mean±SEM (n=5), * p<0.05 compared to AA mice;° p<0.05 compared to vehicle treated mice one-way ANOVA. FIG. 41E. Circulating neutrophils identified by flow cytometric analysis as CD45+Ly6G+ cells in mice treated as in FIG. 41A. Data are mean±SEM (n=4-7), * p<0.05 compared to AA mice; ºp<0.05 compared to vehicle treated mice by one-way ANOVA.

FIGS. 42A-42C. Epeleuton reprograms spleen macrophages towards a pro-resolving pattern. FIGS. 42A and 42B. Flow cytometry gating strategy (FIG. 42A) and representative plots of spleen macrophages from AA or SS mice treated with vehicle or Epeleuton (FIG. 42B). FIG. 42C. M1 marker expression on spleen macrophages and RBC clearance from AA or SS mice fed with Epeleuton determined with flow cytometry in F4/80⁺ cells. Phagocytosis of erythrocytes was assessed as the percentage of F4/80⁺/Ter-119⁺ double positive cells. MFI, Mean fluorescence intensities. Results are means±SD (n=4 mice/group); * p<0.05 (one-way ANOVA).

FIGS. 43A-43C. In SCD mice, Epeleuton reduces lung injury, preventing the overactivation of NF-kB and the hypoxia induced lung inflammatory vasculopathy. FIG. 43A. Representative micro picture of Hematoxylin and eosin-stained (H&E) sections of lung at 200× magnification from SS mice under normoxia and exposed to H/R: hypoxia (8% oxygen; 10 hours), followed by reoxygenation (21% oxygen; 3 hours) treated with either vehicle or Epeleuton (1000 mg/Kg/d for 6 weeks), (scale bar: 50 μm) (see also Table 38). FIG. 43B. Immunoblot analysis using specific antibodies against phosphorylated (p-)NF-κB p65 and NF-κB p65 in lung from AA and SS mice under normoxia condition or exposed to H/R: hypoxia (8% oxygen; 10 hours), followed by reoxygenation (21% oxygen; 3 hours), treated with either vehicle or Epeleuton (1000 mg/Kg/d for 6 weeks). 75 μgr of protein loaded on an 8% T, 2.5% C polyacrylamide gel. GAPDH serves as protein loading control. One representative gel from 4 with similar results is shown. Right panel. Densitometric analysis of immunoblots is shown on the right. Data are presented as means±SEM (n=4); * p<0.05 compared to AA mice; \hat{ }p<0.05 compared to normoxia; °p<0.05 compared to vehicle by one-way ANOVA (DU: Densitometric Unit). FIG. 43C. Immunoblot analysis using specific antibodies against NLRP3, VCAM-1, E-selectin and TBXS (thromboxane synthase) in lung from AA and SS mice treated as in FIG. 43B. 75 μg of protein loaded on an 8% T, 2.5% C polyacrylamide gel. GAPDH serves as protein loading control. One representative gel from 4 with similar results is shown. Densitometric analysis of immunoblots is shown on the right. Data are presented as means±SEM (n=4); * p<0.05 compared to AA mice; \hat{ }p<0.05 compared to normoxia;° p<0.05 compared to vehicle by one-way ANOVA (DU: Densitometric Unit).

FIGS. 44A-44E. In SCD mice, Epeleuton diminishes the H/R induced kidney damage and the markers of vascular dysfunction. FIG. 44A. Representative micro-picture of Hematoxylin and eosin-stained (H&E) sections of kidney at 200× magnification from SS mice exposed to H/R: hypoxia (8% oxygen; 10 hours), followed by reoxygenation (21% oxygen; 3 hours) treated with either vehicle or Epeleuton (1000 mg/Kg/d for 6 weeks) (scale bar: 50 μm) (see also Table 38). FIG. 44B. Kidney leukocytes infiltrates determined by flow cytometry gating analysis (gating strategy is shown in FIGS. 54A and 54B). Data are presented as means±SEM (n=4-6), \hat{ }p<0.05 compared to normoxia; ºp<0.05 compared to vehicle by unpaired t-test with Bonferroni correction. FIG. 44C. Blood urea nitrogen (BUN) (upper panel) and plasma creatinine (lower panel) values in AA and SS mice under normoxia conditions or exposed to H/R: hypoxia (8% oxygen; 10 hours), followed by reoxygenation (21% oxygen; 3 hours), treated with either vehicle or Epeleuton (1000 mg/Kg/d for 6 weeks). Data are means±SEM (n=6). * p<0.05 compared to AA mice, \hat{ }p <0.05 compared to normoxia; ° p<0.05 compared to vehicle by one-way ANOVA. FIG. 44D. Immunoblot analysis using specific antibodies against phosphorylated (p-) NF-κB p65 and NF-κB p65 in kidney from AA and SS mice treated as in FIG. 44B. 75 μg of protein loaded on an 8% T, 2.5% C polyacrylamide gel. GAPDH serves as protein loading control. One representative gel from 4 with similar results is shown. Densitometric analysis of immunoblots is shown in lower panel. Data are presented as means±SEM (n=4). * p<0.05 compared to AA mice; \hat{ }p<0.05 compared to normoxia; ºp<0.05 compared to vehicle by one-way ANOVA (DU: Densitometric Unit). FIG. 44E. Immunoblot analysis, using specific antibodies against NLRP3, VCAM-1, ET-1 and TBXS (thromboxane synthase) in kidney from AA and SS mice treated as in FIG. 44B. 75 μg of protein loaded on an 11% T, 2.5% C polyacrylamide gel. GAPDH serves as protein loading control. One representative gel from 4 with similar results is shown. Densitometric analysis of immunoblots is shown on the right. Data are presented as means±SEM (n=4); * p<0.05 compared to AA mice; \hat{ }p<0.05 compared to normoxia; ºp<0.05 compared to vehicle by one-way ANOVA (DU: Densitometric Unit).

FIGS. 45A-45C. In SCD mice exposed to H/R stress, Epeleuton reduces liver injury and prevents the overactivation of inflammatory and redox related pathways. FIG. 45A. Representative micro picture of Hematoxylin and eosin-stained (H&E) and Perls' stained sections of liver at 200× magnification from SS mice exposed to H/R: hypoxia (8% oxygen; 10 hours), followed by reoxygenation (21% oxygen; 3 hours) treated with either vehicle or Epeleuton (1000 mg/Kg/d for 6 weeks) (scale bar: 50 μm) (see also Table 38). FIG. 45B. Immunoblot analysis using specific antibodies against phosphorylated (p-)NF-κB p65, NF-κB p65, p-Nrf2, and Nrf2 in liver from AA and SS mice under normoxia conditions or exposed to H/R: hypoxia (8% oxygen; 10 hours), followed by reoxygenation (21% oxygen; 3 hours), treated with either vehicle or Epeleuton (1000 mg/Kg/d for 6 weeks). 75 μg of protein loaded on an 8% T, 2.5% C polyacrylamide gel. GAPDH serves as protein loading control. One representative gel from 4 with similar results is shown. Densitometric analysis of immunoblots is shown in right panel. Data are presented as means±SEM (n=4); * p<0.05 compared to AA mice; \hat{ }p<0.05 compared to normoxia; ºp<0.05 compared to vehicle by one-way ANOVA (DU: Densitometric Unit). FIG. 45C. Immunoblot analysis, using specific antibodies against NLRP3, NQO1, VCAM-1 and ET-1 in liver from AA and SS mice treated as in FIG. 45B. 75 μg of protein loaded on an 11% T, 2.5% C polyacrylamide gel. GAPDH serves as protein loading control. One representative gel from ⅘ with similar results is shown. Densitometric analysis of immunoblots is shown on the right. Data are presented as means±SEM (n=⅘); * p<0.05 compared to AA mice; \hat{ }p<0.05 compared to normoxia; ºp<0.05 compared to vehicle by one-way ANOVA (DU: Densitometric Unit).

FIGS. 46A and 46B. Epeleuton protects against progression of inflammatory vasculopathy related to acute Hypoxia/Reoxygenation stress in SCD mice. FIG. 46A. Immunoblot analysis, using specific antibodies against VCAM-1 (40 μgr of protein loaded on an 8% T, 2.5% C polyacrylamide gel) in isolated aorta from AA and SS mice under normoxia or exposed to H/R treated with either vehicle or Epeleuton (1000 mg/Kg/d for 6 weeks). Actin serves as protein loading control. One representative gel from 5 with similar results is shown. Densitometric analysis of immunoblots is shown in lower panel. Data are presented as means±SEM (n=5); * p<0.05 compared to AA mice; \hat{ }p<0.05 compared to normoxia; º p<0.05 compared to vehicle by one-way ANOVA (DU: Densitometric Unit). FIG. 46B. Schematic diagram of the dual anti-inflammatory and pro-resolution effect of Epeleuton in the humanized mouse model for SCD (sickle cell disease). Epeleuton and its active moiety 15(S)-HEPE favor pro-resolving mechanisms targeting inflammation, ROS (reactive oxygen species) burst, NF-kB activation and NLRP3 inflammasome expression. This results in RBCs sickling prevention, inflammatory vasculopathy reduction and macrophages pro-resolving reprogramming (CD68 and CD80) in target organs for SCD. VCAM-1: Vascular Cell Adhesion Molecule 1; ICAM-1: Intercellular Adhesion Molecule 1; TBXs: thromboxane synthases.

FIGS. 47A-47C. FIG. 47A. Weekly body weight measurement of SS mice treated with Epeleuton (1000 mg/Kg/d) for 6 weeks. Data are presented as mean±SEM (n=10-17). FIG. 47B. Plasma blood urea nitrogen (BUN) (left panel) and creatinine (right panel) in AA and SS mice treated with either vehicle or Epeleuton (1000 mg/Kg/d for 6 weeks). Data are presented as mean±SEM (n=5) by one-way ANOVA. * p<0.05 compared to AA mice. FIG. 47C. Plasmatic alanine transaminase (ALT, left panel) and aspartate transaminase (AST, right panel) in AA and SS mice treated as in FIG. 47B. ºp<0.05 compared to vehicle by one-way ANOVA.

FIGS. 48A-48C. FIG. 48A. Plasmatic C-reactive protein (CRP, left panel), Pentraxin-2/SAP-1 (right panel). FIG. 48B. Plasmatic CCL-2 (left panel) and CXCL-2 (right panel) were evaluated in AA and SS mice treated with either vehicle or Epeleuton (1000 mg/Kg/day for 6 weeks). Data are presented as mean±SEM (n=4-5). * p<0.05 compared to AA mice. ºp<0.05 compared to vehicle by unpaired t-test with Bonferroni correction. FIG. 48C. Hematocrit level (Hct, left panel) and circulating reticulocytes count (retics, right panel) in AA and SS mice treated with either vehicle or Epeleuton (1000 mg/Kg/day for 6 weeks) under normoxia. Data are presented as mean±SEM (n=4-9) by unpaired t-test with Bonferroni correction.

FIGS. 49A-49C. FIG. 49A. Flow cytometric gating strategy for peripheral (upper panel) and spleen (lower panel) neutrophil identification. Neutrophils were recognized as CD45+Ly6G+ cells. FIGS. 49B and 49C. Spleen weight/mouse weight ratio (FIG. 49B) and splenic neutrophils (FIG. 49C) identified by flow cytometric analysis as CD45+Ly6G+ cells in AA and SS mice treated with either vehicle or Epeleuton (1000 mg/Kg/day for 6 weeks). Data are mean±SEM (n=4-8).

FIGS. 50A and 50B. FIG. 50A. Densitometric analysis of immunoblots shown in FIGS. 40B-40D. Data are presented as means±SEM (n=4); * p<0.05 compared to AA mice; ºp<0.05 compared to vehicle (DU: Densitometric Unit). FIG. 50B. Densitometric analysis of immunoblots shown in FIG. 40E. Data are presented as means ±SEM (n=4); * p<0.05 compared to AA mice; ºp<0.05 compared to vehicle by one-way ANOVA (DU: Densitometric Unit).

FIGS. 51A and 51B. FIG. 51A. Densitometric analysis of immunoblots shown in FIG. 40F. Data are presented as means±SEM (n=4); * p<0.05 compared to AA mice; ºp<0.05 compared to vehicle by one-way ANOVA (DU: Densitometric Unit). FIG. 51B. Quantification of band area of FIG. 40H. Data are presented as mean±SEM (n=5) (DU: Densitometric Unit). ºp<0.05 compared to vehicle.

FIGS. 52A-52C. FIG. 52A plasmatic C-reactive protein (CRP, left panel), Pentraxin-2/SAP-1 (right panel) and FIG. 52B spleen weight/mouse weight ratio (left panel) and splenic neutrophils (right panel) in AA and SS mice exposed to H/R: hypoxia (8% oxygen; 10 hours), followed by reoxygenation (21% oxygen; 3 hours), treated with either vehicle or Epeleuton (1000 mg/Kg/d for 6 weeks). Data are presented as means±SEM (n=3-9); * p<0.05 compared to AA mice;° p<0.05 compared to vehicle FIG. 52C. Liver iron score.

FIG. 53. Flow cytometric gating strategy for kidney leukocyte identification.

FIGS. 54A and 54B. FIG. 54A. OxyBlot analysis of the soluble fractions of liver from AA and SS mice under normoxia condition or exposed to H/R: hypoxia (8% oxygen; 10 hours), followed by reoxygenation (21% oxygen; 3 hours), treated with either vehicle or Epeleuton (1000 mg/Kg/d for 6 weeks). The carbonylated proteins (1 mg) were detected by treating with 2,4-dinitrophenylhydrazine and blotted with anti-DNP antibody. GAPDH serves as protein loading control. Quantification of band area is shown on the right. Data are presented as means±SEM (n=5); * p<0.05 compared to AA mice; \hat{ }p<0.05 compared to normoxia; ºp<0.05 compared to vehicle by one-way ANOVA. FIG. 54B. Immunoblot analysis using specific antibodies against HO-1 of liver from AA and SS mice under normoxia condition or exposed to H/R: hypoxia (8% oxygen; 10 hours), followed by reoxygenation (21% oxygen; 3 hours), treated with either vehicle or Epeleuton (1000 mg/Kg/d for 6 weeks). 50 μg of protein loaded on an 11% T, 2.5% C polyacrylamide gel. GAPDH serves as protein loading control. One representative gel from 4 with similar results is shown. Densitometric analysis of immunoblots is shown on the right. Data are presented as means±SEM (n=4); * p<0.05 compared to AA mice; \hat{ }p<0.05 compared to normoxia; º p<0.05 compared to vehicle by one-way ANOVA. (DU: Densitometric Unit).

FIG. 55. Immunoblot analysis using specific antibodies against ICAM1, ET-1 and VCAM1 in isolated aorta from AA and SS mice in normoxia treated with either vehicle or Epeleuton (1000 mg/Kg/d for 6 weeks). 75 μg of protein loaded on an 8% T, 2.5% C polyacrylamide gel. Actin serves as protein loading control. One representative gel from 4 with similar results is shown. Densitometric analysis of immunoblots is shown on the right. Data are presented as means±SEM (n=4); * p<0.05 compared to AA mice; ºp<0.05 compared to vehicle by one-way ANOVA (DU: Densitometric Unit).

FIGS. 56A-56D. 15(S)-HEPE decreased RBC adhesion to acutely heme-activated HUVECs. FIG. 56A. Pretreatment of RBCs with 15(S)-HEPE or vehicle control (n=7). FIG. 56B. Pretreatment of HUVECs with 15(S)-HEPE or vehicle control (n=7). Microscope 10× phase-contrast images of untreated HUVECs (FIG. 56C) and 15(S)-HEPE treated HUVECs (FIG. 56D) p-values were calculated using paired t-test. Error bars represent the standard error of the mean (SEM). Scale bare 50 μm.

### DETAILED DESCRIPTION

While the present technology is capable of being embodied in various forms, the description below of several embodiments is made with the understanding that the present disclosure is to be considered as an exemplification of the present technology, and is not intended to limit the present technology to the specific embodiments present technology. Headings are provided for convenience only and are not to be construed to limit the present technology in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations from a stated value can be used to achieve substantially the same results as the stated value. Also, the disclosure of ranges is intended as a continuous range including every value between the minimum and maximum values recited, as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein; and, in all instances, such ratios, ranges, and ranges of ratios represent various embodiments of the present technology.

Unless otherwise defined, all technical and scientific terms used herein have the same meaning as commonly understood by one of ordinary skill in the art to which this disclosure pertains. Although methods and materials similar or equivalent to those described herein can be used in the practice of the present disclosure, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

### Definitions

As used herein, “15-HEPE” refers to 15-hydroxy-eicosa-5Z,8Z,11Z,13E, 17Z-pentaenoic acid in its free acid form and/or a pharmaceutically acceptable ester, conjugate, or salt thereof, or mixtures of any of the foregoing. Similarly, as used herein, a fatty acid especially those written in short form (e.g., 15-HEPE) refers to the fatty acid in free acid form and/or a pharmaceutically acceptable ester, conjugate, or salt thereof, or mixtures of any of the foregoing, as long as the acyl group or the carbon chain portion of the molecule remains intact. The term “pharmaceutically acceptable” in the present context means that the substance in question does not produce unacceptable toxicity to the subject or interaction with other components of the composition. A “derivative” of 15-HEPE may be used instead in certain embodiments, which includes molecules where the acyl group or the carbon chain portion of the 15-HEPE molecule has one or more modifications, although it does not include any derivative compound missing the hydroxy group of 15-HEPE.

15-HEPE is a chiral molecule and may be used in the (S)-or (R)-enantiomeric form, or as a racemic mixture. As used herein, “15-HEPE” includes all such forms, with no limitation as to stereospecificity. In any of the embodiments disclosed herein, 15-HEPE may comprise the (S) form (i.e., 15(S)-hydroxy-(5Z,8Z,11Z,13E,17Z)-eicosapentaenoic acid), the (R) from (i.e., 15(R)-hydroxy-(5Z,8Z,11Z,13E,17Z)-eicosapentaenoic acid), or mixtures thereof. In some embodiments, the 15-HEPE EE comprises the (S) form, 15(S)-HEPE EE.

As used herein, “DS102” and “Epeleuton” refer to 15-HEPE EE or a composition comprising 15-HEPE EE.

As used herein, an “effective amount” refers to the amount of an active composition that is required to confer a therapeutic effect on the subject. A “therapeutically effective amount” refers to a sufficient amount of an agent or a compound being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the composition including a compound as disclosed herein required to provide a clinically significant decrease in disease symptoms without undue adverse side effects. In some embodiments, an appropriate “effective amount” in any individual case is determined using techniques, such as a dose escalation study. The term “therapeutically effective amount” includes, for example, a prophylactically effective amount. In other embodiments, an “effective amount” of a compound disclosed herein is an amount effective to achieve a desired pharmacologic effect or therapeutic improvement without undue adverse side effects. In other embodiments, it is understood that “an effect amount” or “a therapeutically effective amount” varies from subject to subject, due to variation in metabolism, age, weight, general condition of the subject, the condition being treated, the severity of the condition being treated, and the judgment of the prescribing physician.

As used herein, the term “treatment” or “treating” in relation to a given disease or disorder includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder. The term “prevention” or “preventing” in relation to a given disease or disorder includes preventing the onset of disease development if none had occurred; preventing the disease or disorder from occurring in a subject that may be predisposed to the disorder or disease but has not yet been diagnosed as having the disorder or disease; and/or preventing further disease/disorder development if already present.

### Compositions

In some embodiments, provided are compositions or pharmaceutical compositions comprising a therapeutically effective amount of 15-HEPE in its free acid form and/or a pharmaceutically acceptable ester, conjugate, or salt thereof, or mixtures of any of the foregoing, as an active ingredient. As use herein, the terms “composition” and “pharmaceutical composition” are used interchangeably. 15-HEPE, also occasionally referred to as 15-OHEPA, can be synthesized from eicosapentaenoic acid (EPA, eicosa-5,8,11,14,17-pentaenoic acid, or 20:5n-3), an omega-3 fatty acid, according to methods known in the art. For example, 15-HEPE can be synthesized by exposure of EPA to the enzyme 15-lipoxygenase. In some embodiments, the 15-HEPE is used in its free acid form. Alternatively, pharmaceutically acceptable esters, conjugates, and/or salts of 15-HEPE are used in the disclosure. In some embodiments, the 15-HEPE is in the form of a C1-C5 alkyl ester, such as methyl ester, ethyl ester, propyl ester, isopropyl ester, butyl ester, pentyl ester, or a combination of any of the foregoing. In some embodiments, the 15-HEPE is in a form of a glyceride (e.g., diglyceride or triglyceride).

In some embodiments, the 15-HEPE is in the form of an ethyl ester (also referred to herein as E-15-HEPE, 15-HEPE EE, or ethyl-15-HEPE). In some embodiments, the 15-HEPE comprises the optically active 15(S)-hydroxy-(5Z,8Z,11Z,13E,17Z)-eicosapentaenoic acid ethyl ester, or 15(S)-HEPE EE.

In some embodiments, the 15-HEPE comprises lithium 15-HEPE, or a mono-, di-or triglyceride of 15-HEPE.

In some embodiment, 15-HEPE (as the term “15-HEPE” is defined and exemplified herein) is present in the composition in an amount of about 1 mg to about 10,000 mg, about 25 mg to about 7500 mg, about 25 mg to about 5000 mg, about 50 mg to about 5000 mg, about 50 mg to about 3000 mg, about 75 mg to about 2500 mg, or about 100 mg to about 1000 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, or about 10,000 mg.

In some embodiments, the composition contains at least 50%, by weight of the composition, 15-HEPE (as the term “15-HEPE” is defined and exemplified herein), for example, at least 50%, at least 60%, at least 70%, at least 80%, or at least 90%15-HEPE. In some embodiments, 15-HEPE compositions can comprise even higher purity 15-HEPE, for example, at least 95%, at least 96%, at least 97%, at least 98%, or at least 99%15-HEPE. The purity of 15-HEPE can further be defined (e.g., impurity profile) by any of the descriptions of 15-HEPE provided herein. The nature of the essential fatty acids and their synthesis is such that the 15-HEPE composition may include moieties from other essential fatty acids in the essential fatty acid metabolic cascade.

In some embodiments, the composition contains no more than 20%, no more than 15%, no more than 10%, no more than 9%, no more than 8%, no more than 7%, no more than 6%, no more than 5%, no more than 4%, no more than 3%, no more than 2%, no more than 1%, no more than 0.6%, no more than 0.5%, no more than 0.4%, no more than 0.3%, no more than 0.2%, or no more than 0.1%, by weight of the composition, omega-3 fatty acids other than 15-HEPE. Illustrative examples of a “omega-3 fatty acids other than 15-HEPE” include alpha-linolenic acid (ALA), stearidonic acid (SDA), omega-3-eicosatetraenoic acid (ETA), eicosapentaenoic acid (EPA), docosapentaenoic acid (DPA), and docosahexaenoic acid (DHA). In some embodiments, the composition contains substantially no, or no such other omega-3 fatty acids.

In some embodiments, 15-HEPE (as the term “15-HEPE” is defined and exemplified herein) represents at least 50%, by weight, of total fatty acids present in the composition, for example, at least 50%, at least 60%, at least 66%, at least 70%, at least 75%, at least 80%, at least 90%, at least 95%, at least 96%, at least 97%, at least 98%, at least 99%, or 100%, by weight, of total fatty acids present in the composition.

In some embodiments, the composition contains no more than 20%, no more than 15%, no more than 10%, no more than 9%, no more than 8%, no more than 7%, no more than 6%, no more than 5%, no more than 4%, no more than 3%, no more than 2%, no more than 1%, no more than 0.6%, no more than 0.5%, no more than 0.4%, no more than 0.3%, no more than 0.2%, or no more than 0.1%, by weight of total fatty acids present in the composition, of any fatty acids other than 15-HEPE.

In some embodiments, there may be some residual EPA from the synthesis of the 15-HEPE present in the composition. In certain of these embodiments, the composition contains no more than 10%, no more than 9%, no more than 8%, no more than 7%, no more than 6%, no more than 5%, no more than 4%, no more than 3%, no more than 2%, no more than 1%, no more than 0.6%, no more than 0.5%, no more than 0.4%, no more than 0.3%, no more than 0.2%, or no more than 0.1%, by weight of the composition or of total fatty acids present in the composition, EPA in a form which has not been modified to have the hydroxyl-group. In other embodiments, the composition contains substantially no, or no EPA in a form which has not been modified to have the hydroxyl-group.

### Additional Active Agents

In some embodiments, the composition comprising 15-HEPE according to various embodiments of the present technology may further comprise one or more additional active agent(s). In some embodiments, the composition comprises an amount of the additional active agent that is less than the generally recognized therapeutically effective amount for that agent. In other embodiments, the composition comprises an amount of the additional active agent that is equal to or greater than the generally recognized therapeutically effective amount for that agent. If an additional active agent is to be used, the 15-HEPE can be co-formulated as a single dosage unit or can be formulated as two to a plurality of dosage units for coordinated, combination, or concomitant administration.

In some embodiments, the one or more additional active agent(s) comprise at least one selected from the group consisting of eicosapentaenoic acid (EPA), 5-hydroxy-eicosapentaenoic acid (5-HEPE), 12-hydroxy-eicosapentaenoic acid (12-HEPE), 18-hydroxy-eicosapentaenoic acid (18-HEPE), 15-hydroxy-eicosatrienoic acid (15-HETrE), 15-hydroxy-eicosatetraenoic acid (15-HETE), 14-hydroxy-docosahexaenoic acid (14-HDoHE), 17-hydroxy-docosahexaenoic acid (17-HDoHE), resolvin D1 (RvD1 or 17(S)-RvD1), and aspirin-triggered resolvin D1 (AT-RvD1 or 17(R)-RvD1).

As described herein, 15-HEPE can be synthesized from EPA, for example, by 15-lipoxygenase. EPA itself has beneficial properties in treating inflammatory and/or cardiovascular diseases, and thus it is possible to combine 15-HEPE with EPA in an alternative embodiment of the present composition.

As used herein, “5-HEPE” refers to 5-hydroxy-5Z,8Z,10E, 14Z, 17Z-eicosapentaenoic acid in its free acid form and/or a pharmaceutically acceptable ester, conjugate, or salt thereof, or mixtures of any of the foregoing. “12-HEPE” refers to 12-hydroxy-5Z,8Z, 10E, 14Z, 17Z-eicosapentaenoic acid in its free acid form and/or a pharmaceutically acceptable ester, conjugate, or salt thereof, or mixtures of any of the foregoing. “18-HEPE” refers to 8-hydroxy-5Z,8Z, 11Z, 14Z, 16E-eicosapentaenoic acid in its free acid form and/or a pharmaceutically acceptable ester, conjugate, or salt thereof, or mixtures of any of the foregoing. 5-HEPE, 12-HEPE, and 18-HEPE are derivatives of EPA and can be synthesized from EPA according to methods known in the art.

As used herein, “15-HETrE” refers to 15-hydroxy-eicosa-8Z, 11Z, 13E-trienoic acid in its free acid form and/or a pharmaceutically acceptable ester, conjugate, or salt thereof, or mixtures of any of the foregoing. 15-HETrE is an in vivo metabolite resulting from 15-lipoxygenation of dihomo-gamma-linoleic acid (DGLA) and can be synthesized from DGLA according to methods known in the art. For example, 15-HETrE can be synthesized by exposure of DGLA to the enzyme 15-lipoxygenase.

As used herein, “15-HETE” refers to 15-hydroxy-eicosa-5Z,8Z, 11Z, 13E-tetraenoic acid in its free acid form and/or a pharmaceutically acceptable ester, conjugate, or salt thereof, or mixtures of any of the foregoing. 15-HETE is a metabolite of arachidonic acid (AA) and is a key precursor to many biologically active pro-resolving mediators.

As used herein, “14-HDoHE” refers to 14-hydroxy-4Z,7Z,10Z, 12E, 16Z, 19Z-docosahexaenoic acid in its free acid form and/or a pharmaceutically acceptable ester, conjugate, or salt thereof, or mixtures of any of the foregoing. “17-HDoHE” refers to 17-hydroxy-4Z,7Z, 10Z,13Z, 15E, 19Z-docosahexaenoic acid in its free acid form and/or a pharmaceutically acceptable ester, conjugate, or salt thereof, or mixtures of any of the foregoing. 14-HDoHE and 17-HDoHE are derivatives of docosahexaenoic acid (DHA) and can be synthesized from DHA according to methods known in the art.

As used herein, “resolvin D1,” “RvD1,” or “17(S)-RvD1” refers to 7S,8R,17S-trihydroxy-4Z,9E, 11E, 13Z, 15E, 19Z-docosahexaenoic acid in its free acid form and/or a pharmaceutically acceptable ester, conjugate, or salt thereof, or mixtures of any of the foregoing. “Aspirin-triggered resolvin D1,” “AT-RvD1,” or “17(R)-RvD1” refers to 7S,8R, 17R-trihydroxy-4Z,9E,11E, 13Z, 15E, 19Z-docosahexaenoic acid in its free acid form and/or a pharmaceutically acceptable ester, conjugate, or salt thereof, or mixtures of any of the foregoing. RvD1 and AT-RvD1 are derivatives of DHA with potential anti-inflammatory and analgesic activities.

In any of the above embodiments, the fatty acid or derivative thereof as described may be in its free acid form. Alternatively, pharmaceutically acceptable esters, conjugates, and/or salts are used in the disclosure. In some embodiments, the ester may be a C1-C5 alkyl ester, such as methyl ester, ethyl ester, propyl ester, isopropyl ester, butyl ester, pentyl ester, or a combination of any of the foregoing. In some embodiments, the ester is in a form of a glyceride (e.g., diglyceride or triglyceride).

In some embodiments, the 15-HEPE and one or more additional active agent(s) are present in the composition, or are co-administered, in a weight ratio of 15-HEPE:additional active agent(s) of about 1:1000 to about 1000:1, about 1:500 to about 500:1, about 1:100 to about 100:1, about 1:50 to about 50:1, about 1:25 to about 25:1, about 1:10 to about 10:1, about 1:5 to about 5:1, about 1:4 to about 4:1 about 1:3 to about 3:1, about 1:2 to about 2:1, or about 1:1.

### Dosage Forms

In some embodiments, the composition comprising 15-HEPE according to various embodiments disclosed herein can be formulated as one or more dosage units. The term “dosage unit” refers to a portion of a pharmaceutical composition that contains an amount of a therapeutic agent suitable for a single administration to provide a therapeutic effect. Such dosage units may be administered one to a plurality (i.e., 2, 3, 4, 5, or more) of times per day, or as many times as needed to elicit a therapeutic response.

In some embodiments, the composition is orally deliverable or in a form suitable for oral administration. The term “orally deliverable” or “oral administration” includes any form of delivery of a pharmaceutical composition to a subject wherein the composition is placed in the mouth of the subject, whether or not the composition is swallowed. This therefore includes buccal and sublingual administration, as well as esophageal administration. Non-limiting examples of suitable orally deliverable dosage forms include tablets (e.g., suspension tablets, bite suspension tablets, rapid dispersion tablets, or chewable tablets), caplets, capsules (e.g., soft gelatin capsule, hard gelatin capsule, or HPMC capsule), lozenges, sachets, cachets, troches, pellets, suspension, elixirs, syrups, and any other solid dosage form reasonably adapted for oral administration. The composition can be in the form of liquid dosage forms or dose units to be imbibed directly or they can be mixed with food or beverage prior to ingestion. Non-limiting examples of suitable liquid dosage forms include solutions, suspensions, elixirs, syrups, liquid aerosol formulations, and the like.

In some embodiments, the dosage unit is a capsule, i.e., the composition is formulated in one or more capsules. The one or more capsules may be packaged in a blister pack or a receptacle (e.g., bottle).

In some embodiments, provided is a composition comprising 15-HEPE encapsulated in a capsule shell. In some embodiments, the composition is administered to a subject in need thereof in an amount sufficient to provide up to about 1 g, about 2 g, about 3 g, about 4 g, about 5 g, about 6 g, about 7 g, about 8 g, about 9 g, or about 10 g of 15-HEPE per day. In some embodiments, the composition is administered to the subject in an amount sufficient to provide about 4 g to about 8 g, about 1 g to about 2 g, about 2 g to about 4 g, about 3 g to about 8 g, about 4 g to about 6 g of 15-HEPE per day. In discussing the amount of 15-HEPE in the composition, it may be split over several dosage units. There is a limit as to the size for oral administration. For example, if a subject is to be administered about 4 g 15-HEPE a day, this may be split by up to 4 capsules, each providing about 1 g of 15-HEPE. In some embodiment, about 500 mg to about 1 g of 15-HEPE is encapsulated in the capsule shell.

In some embodiments, the capsule is a soft gelatin capsule. In some embodiments, the capsule is a hard gelatin capsule. In some embodiments, the capsule shell comprises gelatin (for example, Gelatin RXL or lime bone gelatin with a lower molecular weight) and/or plasticizers selected from one or more of D-Sorbitol and 1,4-sorbitans. In some embodiments, the capsule shell comprises Gelatin RXL that has been treated by proteolytic enzyme to cut the gelatin pattern and effectively decrease its molecular weight. In some embodiments, the composition comprises 15-HEPE esters of D-Sorbitol and 1,4-sorbitan. In some embodiments, the gelatin is as described in U.S. Pat. No. 7,485,323, which is incorporated by reference herein in its entirety.

In some embodiments, the plasticizer comprises 1,4-sorbitans in an amount from about 20% to about 30%, for example, about 24% to about 28%, 24%, or 28% (on a dry basis), and/or D-Sorbitol in an amount of about 30% to about 50%, for example, about 35% to about 45% (on a dry basis).

In some embodiments, the capsule shell comprises modified starch, carrageenan (e.g., extract of red seaweed), disodium phosphate, glycerol, and/or sorbitol. In some embodiments, the capsule shell further comprises water. In some embodiments, the capsule shell is stable up to a temperature of about 65° C. and/or pH of about 12.

In some embodiments, the capsule shell further comprises glycerol, purified water, titanium dioxide, medium chain triglycerides, and/or lecithin.

In some embodiments, the capsule shell is odorless and has a neutral color (e.g., colorless, white, or transparent).

Alternatively, compositions of the present disclosure can be formulated for rectal, topical, or parenteral (e.g., subcutaneous, intramuscular, intravenous, intradermal, or infusion) delivery.

In some embodiments, compositions of the present disclosure may comprise one or more pharmaceutically acceptable excipients. The term “pharmaceutically acceptable excipient” herein means any substance, not itself a therapeutic agent, used as a carrier or vehicle for delivery of a therapeutic agent to a subject or added to a composition to improve its handling or storage properties or to permit or facilitate formation of a unit dose of the composition, and that does not produce unacceptable toxicity or interaction with other components in the composition. By way of example only, a pharmaceutical composition according to the present disclosure may comprise one or more of antioxidants, surfactants, preservatives, flavoring agents, co-solvents, viscosity aids, suspension aids, and lipophilic phases.

In some embodiments, the composition comprises one or more antioxidants such as ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, niacinamide, and the like. In one embodiment, the composition comprises about 0.01 wt. % to about 2 wt. % of an antioxidant, for example about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. % of the one or more antioxidant.

### Therapeutic Methods

In some embodiments, provided are methods of treating and/or preventing a hematologic disorder and/or symptoms thereof in a subject in need thereof, the methods comprising administering to the subject a therapeutically effective amount of a composition comprising 15-HEPE according to various embodiments disclosed herein.

In some embodiments, hematologic disorders include, but are not limited to, the following diseases and conditions:
Red blood cell disorders
   Anemia
   Hemolytic anemia (inherited or acquired)
   Iron deficiency anemia
   Hereditary spherocytosis
   Chronic hemolysis
   Pyruvate kinase deficiency
   Glucose-6-phosphate dehydrogenase (G6PD) deficiency
Hemoglobin disorders
   Sickle cell disease
   Thalassemia (alpha and beta thalassemia)
Bleeding disorders
   Hemophilia
   Thrombophilia
   Thrombocytopenia
   Idiopathic thrombocytopenia purpura (ITP)
   Heparin-induced thrombocytopenia (HIT)
   Venous thromboembolism
   Arterial thrombosis
   Embolism
Blood cancer
   Myelodysplastic syndrome (MDS)
   Lymphoma
   Leukemia
   Myeloma

In some embodiments, the subject has diabetes, including, for example, type I diabetes or type II diabetes. In some embodiments, the method further comprises determining that the subject has diabetes before administering the composition comprising 15-HEPE. In some embodiments, the subject has elevated baseline fasting glucose levels of at least about 100 mg/dL, at least about 115 mg/dL, at least about 120 mg/dl, at least about 125 mg/dL, at least about 130 mg/dL, at least about 135 mg/dl, at least about 140 mg/dL, at least about 145 mg/dL, at least about 150 mg/dL, at least about 155 mg/dl, at least about 160 mg/dL, at least about 165 mg/dL, or at least about 170 mg/dL.

In some embodiments, the subject has a cardiovascular disease. The term “cardiovascular disease” refers to any disease or disorder of the heart (cardiac diseases or disorders) or blood vessels (vascular diseases or disorders) or any symptom thereof. Non-limiting examples of cardiovascular diseases include hypertriglyceridemia, hypercholesterolemia, mixed dyslipidemia, coronary heart disease, stroke, atherosclerosis, arrhythmia, hypertension, myocardial infarction, vasculitis, cardiomyopathy, pericarditis, congestive heart failure, myocardial necrosis, vascular ischemia, vascular disease, thrombotic disease, deep venous thrombosis, and other cardiovascular or related diseases. In some embodiments, the method further comprises determining that the subject has a cardiovascular disease before administering the composition comprising 15-HEPE.

In some embodiment, the subject has high blood pressure. In some embodiments, the method further comprises determining that the subject has high blood pressure before administering the composition comprising 15-HEPE. In some embodiments, the subject has a blood pressure of at least about 100 mmHg, at least about 115 mmHg, at least about 120 mmHg, at least about 125 mmHg, at least about 130 mmHg, at least about 135 mmHg, at least about 140 mmHg, at least about 145 mmHg, at least about 150 mmHg, at least about 155 mmHg, at least about 160 mmHg, at least about 165 mmHg, or at least about 170 mmHg.

In some embodiments, the method further comprises determining that the subject has at least one risk factor for hematologic disorders before administering the composition comprising 15-HEPE. Non-limiting examples of risk factors for hematologic disorders include reduced red blood cell counts, increased red blood cell distribution widths, and increased reticulocyte counts. In some embodiments, the method further comprises determining a baseline red blood cell count, a baseline red blood cell distribution width, and/or a baseline reticulocyte count of the subject before administering the composition comprising 15-HEPE.

In some embodiments, the subject has a reduced red blood cell count of at least 10% below normal, at least 15% below normal, at least 20% below normal, at least 25% below normal, or at least 30% below normal.

In some embodiments, the subject has an increased red blood cell distribution width, where the red blood cells vary in size by about 15-20%, by about 20-25%, by about 25-30%, by about 30-35%, or by about 35-40%.

In some embodiments, the subject has an increased reticulocyte count of at least about 5% of the total amount of red blood cells, at least about 10% of the total amount of red blood cells, at least about 15% of the total amount of red blood cells, or at least about 20% of the total amount of red blood cells.

In some embodiments, the subject exhibits an increase in the red blood cell count, a decrease in the red blood cell distribution width, and/or a decrease in the reticulocyte count after receiving the composition comprising 15-HEPE compared to baseline or a second subject who is not administered the composition comprising 15-HEPE.

In some embodiments, the method further comprises determining that the subject has at least one risk factor for thrombophilia disorders before administering the composition comprising 15-HEPE. Thrombophilia disorders refer to a class of conditions characterized by abnormal blood coagulation that increases the risk of thrombosis. Non-limiting examples of risk factors for thrombophilia disorders include decreased prothrombin times, decreased activated partial thromboplastin times, and increased fibrinogen concentration. In some embodiments, the method further comprises determining a baseline prothrombin time, a baseline activated partial thromboplastin time, and/or a baseline fibrinogen concentration of the subject before administering the composition comprising 15-HEPE.

In some embodiments, the subject has a reduced prothrombin time of at least 10% below normal, at least 15% below normal, at least 20% below normal, at least 25% below normal, or at least 30% below normal.

In some embodiments, the subject has a reduced activated partial thromboplastin time of at least 10% below normal, at least 15% below normal, at least 20% below normal, at least 25% below normal, or at least 30% below normal.

In some embodiments, the subject has an increased fibrinogen concentration of at least 10% below normal, at least 15% below normal, at least 20% below normal, at least 25% below normal, or at least 30% below normal.

In some embodiments, the subject exhibits an increase in the prothrombin time, an increase in the activated partial thromboplastin time, and/or a decrease in the fibrinogen concentration after receiving the composition comprising 15-HEPE compared to baseline or a second subject who is not administered the composition comprising 15-HEPE.

In some embodiments, provided are methods of treating and/or preventing endothelial dysfunction in a subject in need thereof, the methods comprising administering to the subject a therapeutically effective amount of a composition comprising 15-HEPE according to various embodiments disclosed herein.

In some embodiments, provided are methods of treating, preventing, and/or reducing cell stress apoptosis in a subject in need thereof, the methods comprising administering to the subject a therapeutically effective amount of a composition comprising 15-HEPE according to various embodiments disclosed herein. In some embodiments, the subject exhibits a reduction in markers associated with apoptosis such as proteins from the Bcl-2 family, activated fragments of caspases, and/or cleaved PARP-1 after receiving the composition comprising 15-HEPE compared to baseline or a second subject who is not administered the composition comprising 15-HEPE.

In some embodiments, provided are methods of treating, preventing, and/or reducing inflammation in a subject in need thereof, the methods comprising administering to the subject a therapeutically effective amount of a composition comprising 15-HEPE according to various embodiments disclosed herein. In some embodiments the subject exhibits a reduction in T cell activation, B cell activation, and/or chemotaxis after receiving the composition comprising 15-HEPE compared to baseline or a second subject who is not administered the composition comprising 15-HEPE.

In some embodiments, provided are methods of treating and/or preventing sickle cell disease and/or symptoms thereof in a subject in need thereof, the methods comprising administering to the subject a therapeutically effective amount of a composition comprising 15-HEPE according to various embodiments disclosed herein.

In some embodiments, sickle cell disease and symptoms associated thereof include, but are not limited to, inflammatory vasculopathy, vasoconstriction, bronchoconstriction, iron overload, hemoglobinopathy, chromic hemolysis, sickling, anemia, inflammation, pain, acute and/or chronic damage to organs (e.g., lung, kidney, liver, spleen), and oxidative stress.

In some embodiments, provided are methods of treating and/or preventing heparin induced thrombocytopenia (HIT) and/or symptoms thereof in a subject in need thereof, the methods comprising administering to the subject a therapeutically effective amount of a composition comprising 15-HEPE according to various embodiments disclosed herein.

In some embodiments, upon treatment with a composition of the present technology, the subject exhibits one or more of the following outcomes:
(a) no increase or a reduction in serum aminotransferase (ALT) and/or aspartate aminotransferase (AST) levels relative to baseline, placebo control, and/or untreated patient;
(b) no increase or a reduction in bilirubin (BUN) levels relative to baseline, placebo control, and/or untreated patient;
(c) no increase or a reduction in fibrosis area relative to baseline, placebo control, and/or untreated patient;
(d) no increase or a reduction in fasting glucose levels relative to baseline, placebo control, and/or untreated patient;
(e) no increase or a reduction in insulin levels relative to baseline, placebo control, and/or untreated patient;
(f) no increase or a reduction in alkaline phosphate (ALP) levels relative to

(g) no increase or a reduction in hemoglobin A1C (HbA1C) levels relative to baseline, placebo control, and/or untreated patient;

(h) no increase or a reduction in homeostatic model assessment of insulin resistance (HOMA-IR) levels relative to baseline, placebo control, and/or untreated patient;

(i) no increase or a reduction in adipose tissue insulin resistance (adipo-IR) levels relative to baseline, placebo control, and/or untreated patient;

(j) no increase or a reduction in total cholesterol levels relative to baseline, placebo control, and/or untreated patient;

(k) no increase or a reduction in triglyceride levels relative to baseline, placebo control, and/or untreated patient;

(I) no increase or a reduction in diglyceride levels relative to baseline, placebo control, and/or untreated patient;

(m) no increase or a reduction in very low-density lipoprotein cholesterol (VLDL-C) levels relative to baseline, placebo control, and/or untreated patient;

(n) no increase or a reduction in remnant-like particle cholesterol (RLP-C) levels relative to baseline, placebo control, and/or untreated patient;

(o) no increase or a reduction in non-high-density lipoprotein cholesterol (non-HDL-C) levels relative to baseline, placebo control, and/or untreated patient;

(p) no reduction or an increase in high density lipoprotein cholesterol (HDL-C) levels relative to baseline, placebo control, and/or untreated patient;

(q) no increase or a reduction in low density lipoprotein cholesterol (LDL-C) levels relative to baseline, placebo control, and/or untreated patient;

(r) no increase or a reduction in kidney hydroxyproline levels relative to baseline, placebo control, and/or untreated patient;

(s) no increase or a reduction in interleukin-33 (IL-33) levels relative to baseline, placebo control, and/or untreated patient;

(t) no increase or a reduction in interleukin-17 (IL-17) levels relative to baseline, placebo control, and/or untreated patient;

(u) no increase or a reduction in aryl hydrocarbon receptor (AhR) levels relative to baseline, placebo control, and/or untreated patient;

(v) no increase or a reduction in TNF-like ligand 1A (TL1A) levels relative to baseline, placebo control, and/or untreated patient;

(w) no increase or a reduction in tumor necrosis factor (TNF-α) levels relative to baseline, placebo control, and/or untreated patient;

(x) no increase or a reduction in interleukin-13 (IL-13) levels relative to baseline, placebo control, and/or untreated patient;

(y) no increase or a reduction in interleukin-6 (IL-6) levels relative to baseline, placebo control, and/or untreated patient;

(z) no increase or a reduction in interleukin 1 beta-β (IL-1B) levels relative to baseline, placebo control, and/or untreated patient;

(aa) no increase or a reduction in Collagen Type 1 levels relative to baseline, placebo control, and/or untreated patient;

(bb) no increase or a reduction in transforming growth factor-β (TGF-β) levels relative to baseline, placebo control, and/or untreated patient;

(cc) no increase or a reduction in α-smooth muscle action (α-SMA) levels relative to baseline, placebo control, and/or untreated patient;

(dd) no increase or a reduction in tumor necrosis factor ligand superfamily member 11A (TNRSF11A) levels relative to baseline, placebo control, and/or untreated patient;

(ee) no increase or a reduction in serine protease 2 (PRSS2) levels relative to baseline, placebo control, and/or untreated patient;

(ff) no increase or a reduction in amine oxidase, copper containing 3 (AOC3) levels relative to baseline, placebo control, and/or untreated patient;

(gg) no increase or a reduction in leukocyte immunoglobulin like receptor B1 (LILBR1) levels relative to baseline, placebo control, and/or untreated patient;

(hh) no increase or a reduction in transferrin receptor protein 1 (TR) levels relative to baseline, placebo control, and/or untreated patient;

(ii) no increase or a reduction in elafin (PI3) levels relative to baseline, placebo control, and/or untreated patient;

(jj) no increase or a reduction in serum amyloid A4 (SAA4) levels relative to baseline, placebo control, and/or untreated patient;

(kk) no increase or a reduction in monocyte chemoattractant protein-1 (MCP-1) levels relative to baseline, placebo control, and/or untreated patient;

(II) no increase or a reduction in chemokine (C-C motif) ligand 16 (CCL16) levels relative to baseline, placebo control, and/or untreated patient;

(mm) no increase or a reduction in trem-like transcript 2 (TLT2) levels relative to baseline, placebo control, and/or untreated patient;

(nn) no increase or a reduction in dipeptidyl peptidase 4 (DPP4) levels relative to baseline, placebo control, and/or untreated patient; and

(00) no increase or a reduction in metalloproteinase inhibitor-1 (TIMP-1) levels relative to baseline, placebo control, and/or untreated patient;

(pp) no increase or a reduction in plasminogen activator inhibitor-1 (PAI-1) levels relative to baseline, placebo control, and/or untreated patient;

(qq) no increase or a reduction in a NAFLD score (NFS) relative to baseline, placebo control, and/or untreated patient; and

(rr) no increase or a reduction in fibrosis-4 (FIB-4) levels relative to baseline, placebo control, and/or untreated patient;

(ss) no increase or a reduction in liver stiffness levels relative to baseline, placebo control, and/or untreated patient;

(tt) no increase or a reduction in hepatic fat content relative to baseline, placebo control, and/or untreated patient;

(uu) no reduction or an increase in glycerophospholipid levels relative to baseline, placebo control, and/or untreated patient;

(vv) no increase or a reduction in blood pressure relative to baseline, placebo control, and/or untreated patient;

(ww) no increase or a reduction in an enhanced liver fibrosis (ELF) score relative to baseline, placebo control, and/or untreated patient;

(xx) no increase or a reduction in collagen production in lung and/or dermal fibroblasts relative to baseline, placebo control, and/or untreated patient;

(yy) no reduction or an increase in glucose uptake relative to baseline, placebo control, and/or untreated patient;

(zz) an increase in lung and/or dermal fibroblast viability relative to baseline, placebo control, and/or untreated patient;

(ab) no change or a reduction in interleukin-8 (IL-8) levels relative to baseline, placebo control, and/or untreated patient;

(ac) no change or a reduction in interleukin-23 (IL-23) levels relative to baseline, placebo control, and/or untreated patient;

(ad) no change or a reduction in interlekin-11 (IL-11) levels relative to baseline, placebo control, and/or untreated patient; and/or

(ae) no change or a reduction in interferon γ (IFNγ) levels relative to baseline, placebo control, and/or untreated patient;

(af) no change or a reduction in T and/or B cell activation relative to baseline, placebo control, and/or untreated patient;

(ag) no change or a reduction in chemotaxis relative to baseline, placebo control, and/or untreated patient;

(ah) no change or a reduction in phosphorylated B-cell lymphoma 2 (Bcl-2) family members relative to baseline, placebo control, and/or untreated patient;

(ai) no change or a reduction in activated fragments of caspases levels relative to baseline, placebo control, and/or untreated patient;

(aj) no change or a reduction in cleaved poly (ADP-ribose) polymerase-1 (PARP-1) levels relative to baseline, placebo control, and/or untreated patient;

(ak) no change or a reduction in waist circumference relative to baseline, placebo control, and/or untreated patient;

(al) no change or a reduction in an AST to platelet ration index (ARPI) relative to baseline, placebo control, and/or untreated patient;

(am) no change or a reduction in a liver inflammation and fibrosis (LIF) score relative to baseline, placebo control, and/or untreated patient;

(an) no change or a reduction in a Lok score relative to baseline, placebo control, and/or untreated patient;

(ao) no change or a reduction in a fibrosis score relative to baseline, placebo control, and/or untreated patient;

(ap) no change or a reduction in a King score relative to baseline, placebo control, and/or untreated patient;

(aq) no change or a reduction in a Bonacini score relative to baseline, placebo control, and/or untreated patient;

(ar) no change or a reduction in a transient elastography (TE) score relative to baseline, placebo control, and/or untreated patient.

(as) no increase or a reduction in vascular adhesion molecules relative to baseline, placebo control, and/or untreated patient;

(at) no increase or a reduction in cardiovascular risk proteins relative to baseline, placebo control, and/or untreated patient;

(au) no increase or a reduction in chemokines relative to baseline, placebo control, and/or untreated patient;

(av) no increase or a reduction in tumor necrosis factor receptor superfamily members relative to baseline, placebo control, and/or untreated patient;

(aw) no change or an increase in red blood cell count relative to baseline, placebo control, and/or untreated patient;

(ax) no change or an increase in white blood cell count relative to baseline, placebo control, and/or untreated patient;

(ay) no change or an increase in platelet count relative to baseline, placebo control, and/or untreated patient;

(az) no change or an increase in prothrombin time relative to baseline, placebo control, and/or untreated patient;

(ba) no change or an increase in activated partial thromboplastin time relative to baseline, placebo control, and/or untreated patient;

(bc) no change or an increase in hemoglobin production relative to baseline, placebo control, and/or untreated patient;

(bd) no change or an increase in the synthesis of the beta globin chains of the hemoglobin tetramer relative to baseline, placebo control, and/or untreated patient;

(be) no change or a reduction in red blood cell hemolysis relative to baseline, placebo control, and/or untreated patient;

(bf) no change or a reduction in shortness of breath relative to baseline, placebo control, and/or untreated patient;

(bg) no change or a reduction in heart rate relative to baseline, placebo control, and/or untreated patient;

(bh) no change or a reduction in spleen size relative to baseline, placebo control, and/or untreated patient;

(bi) no change or a reduction in liver size relative to baseline, placebo control, and/or untreated patient;

(bj) no change or a reduction in dactylitis relative to baseline, placebo control, and/or untreated patient;

(bk) no change or a reduction in pain crises relative to baseline, placebo control, and/or untreated patient;

(bl) no change or a reduction in abnormal blood clot development relative to baseline, placebo control, and/or untreated patient;

(bm) no change or a reduction in red cell distribution width relative to baseline, placebo control, and/or untreated patient;

(bn) no change or a reduction in reticulocyte count relative to baseline, placebo control, and/or untreated patient; and/or

(bo) no change or a reduction in fibrinogen concentration relative to baseline, placebo control, and/or untreated patient.

(bp) no change or a reduction in serum creatine levels relative to baseline, placebo control, and/or untreated patient.

(bq) no change or a reduction in C-reactive protein (CRP) levels relative to baseline, placebo control, and/or untreated patient.

(br) no change or a reduction in pentraxin-2 levels relative to baseline, placebo control, and/or untreated patient.

(bs) no change or a reduction in monocyte chemoattractant protein-1 (MCP-1/CCL2) levels relative to baseline, placebo control, and/or untreated patient.

(bt) no change or a reduction in circulating neutrophils relative to baseline, placebo control, and/or untreated patient.

(bu) no change or a reduction in vascular cell adhesion molecule 1 (VCAM-1) levels relative to baseline, placebo control, and/or untreated patient.

(bv) no change or a reduction in intercellular adhesion molecule 1 (ICAM-1) levels relative to baseline, placebo control, and/or untreated patient.

(bw) no change or a reduction in endothelin-1 (ET-1) levels relative to baseline, placebo control, and/or untreated patient.

(bx) no change or an increase in hematocrit levels relative to baseline, placebo control, and/or untreated patient.

(by) no change or an increase in hemoglobin levels relative to baseline, placebo control, and/or untreated patient.

(bz) no change or a reduction in sickled red blood cell numbers relative to baseline, placebo control, and/or untreated patient.

(ca) no change or a reduction in CD68 levels relative to baseline, placebo control, and/or untreated patient.

(cb) no change or a reduction in CD80 levels relative to baseline, placebo control, and/or untreated patient.

(cc) no change or a reduction in NF-kB p65 activation relative to baseline, placebo control, and/or untreated patient.

(cd) no change or a reduction in NLRP3 (NOD-, LRR- and pyrin domain-containing protein 3) inflammasome activation relative to baseline, placebo control, and/or untreated patient.

(ce) no change or a reduction in vascular endothelial activation relative to baseline, placebo control, and/or untreated patient.

(cf) no change or a reduction in neutrophil vascular recruitment relative to baseline, placebo control, and/or untreated patient.

(cg) no change or a reduction in inflammatory cell infiltration relative to baseline, placebo control, and/or untreated patient.

(ch) no change or a reduction in thrombi formation relative to baseline, placebo control, and/or untreated patient.

(ci) no change or a reduction in iron overload and/or liver iron accumulation relative to baseline, placebo control, and/or untreated patient.

(cj) no change or a reduction in oxidative stress relative to baseline, placebo control, and/or untreated patient.

(ck) no change or a reduction in nuclear factor erythroid 2-related factor 2 (Nrf2) levels relative to baseline, placebo control, and/or untreated patient.

(cl) no change or a reduction in oxidation and/or heme oxygenase-1 (HO-1) levels relative to baseline, placebo control, and/or untreated patient.

(cm) no change or a reduction in E-selectin levels relative to baseline, placebo control, and/or untreated patient.

(cn) no change or a reduction in red blood cell adhesion relative to baseline, placebo control, and/or untreated patient.

In some embodiments, the method further comprises measuring a baseline level of one or more markers or parameters set forth in (a)-(cn) above in the subject prior to administering the composition comprising 15-HEPE. In some embodiments, the method further comprises measuring a second level of the one or more markers or parameters set forth in (a)-(cn) above in the subject after administering the composition comprising 15-HEPE.

In some embodiments, upon treatment with the composition comprising 15-HEPE, for example, over a period of about 1 to about 12 weeks, about 1 to about 8 weeks, or about 1 to about 4 weeks, the subject exhibits one or more outcomes described in (a)-(cn) above. For example, the subject exhibits one or more of the following outcomes:
(a) no increase or a reduction in serum ALT and/or AST levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;
(b) no increase or a reduction in BUN levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;
(c) no increase or a reduction in fibrosis area of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;
(d) no increase or a reduction in fasting glucose levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;
(e) no increase or a reduction in insulin of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;
(f) no increase or a reduction in ALP levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to

(g) no increase or a reduction in HB1Ac levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(h) no increase or a reduction in HOMA-IR levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(i) no increase or a reduction in adipo-IR levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(j) no increase or a reduction in total cholesterol levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(k) no increase or a reduction in triglyceride levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to

(l) no increase or a reduction in diglyceride levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(m) no increase or a reduction in VLDL-C levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(n) no increase or a reduction in RLP-C levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(o) no increase or a reduction in non-HDL-C levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(p) no reduction or an increase in HDL-C levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to

(q) no increase or a reduction in LDL-C levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(r) no increase or a reduction in kidney hydroxyproline levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(s) no increase or a reduction in IL-33 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(t) no increase or a reduction in IL-17 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(u) no increase or an reduction in AhR levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to

(v) no increase or a reduction in TL1A levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%; relative to baseline, placebo control, and/or untreated patient;

(w) no increase or a reduction in TNF-α levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(x) no increase or a reduction in IL-13 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(y) no increase or a reduction in IL-6 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(z) no increase or a reduction IL-13 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to

(aa) no increase or a reduction in Collagen Type 1 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bb) no increase or a reduction in TGF-β levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(cc) no increase or a reduction in α-SMA of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(dd) no increase or a reduction in TNRSF11A of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ee) no increase or a reduction in PRSS2 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(ff) no increase or a reduction in AOC3 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(gg) no increase or a reduction in LILBR1 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(hh) no increase or a reduction in TR levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ii) no increase or a reduction in PI3 of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(jj) no increase or a reduction in SAA4 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to

(kk) no increase or a reduction in MCP-1 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(II) no increase or a reduction in CCL16 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(mm) no increase or a reduction in TLT2 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(nn) no increase or a reduction in DPP4 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(oo) no increase or a reduction in TIMP-1 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to

(pp) no increase or a reduction in PAI-1 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(qq) no increase or a reduction in a NFS of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(rr) no increase or a reduction in FIB-4 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ss) no increase or a reduction in liver stiffness of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(tt) no increase or a reduction in hepatic fat content of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to

(uu) no reduction or an increase in glycerophospholipid levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(vv) no increase or a reduction in blood pressure of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ww) no increase or a reduction in an ELF score of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(zz) no increase or a reduction in collagen production in lung and/or dermal fibroblasts score of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(yy) no reduction or an increase in glucose uptake of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(zz) an increase in lung and/or dermal fibroblast viability of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ab) no change or a reduction in IL-8 levels score of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ac) no change or a decrease in IL-23 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% levels relative to baseline, placebo control, and/or untreated patient; and/or

(ad) no change or a reduction in IL-11 levels score of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ae) no change or a reduction in IFNγ levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(af) no change or a reduction in T and/or B cell activation at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ag) no change or a reduction in chemotaxis of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ah) no change or a reduction in Bcl-2 family members of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ai) no change or a reduction in activated fragments of caspases levels relative of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% to baseline, placebo control, and/or untreated patient;

(aj) no change or a reduction in cleaved PARP-1 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ak) no change or a reduction in waist circumference of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(al) no change or a reduction in an ARPI of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(am) no change or a reduction in a LIF score of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(an) no change or a reduction in a Lok score of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to

(ao) no change or a reduction in a fibrosis score of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ap) no change or a reduction in a King score of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(aq) no change or a reduction in a Bonacini score of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient; and/or

(ar) no change or a reduction in a TE score of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(as) an increase in vascular adhesion molecules of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to

(at) an increase in cardiovascular risk proteins of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(au) an increase in chemokines of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient; and

(av) an increase in tumor necrosis factor receptor superfamily members of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(aw) no change or an increase in red blood cell count of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ax) no change or an increase in white blood cell count of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ay) no change or an increase in platelet count of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(az) no change or an increase in prothrombin time of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(ba) no change or an increase in activated partial thromboplastin time of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bc) no change or an increase in hemoglobin production of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bd) no change or an increase in the synthesis of the beta globin chains of the hemoglobin tetramer of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(be) no change or a reduction in red blood cell hemolysis of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bf) no change or a reduction in shortness of breath of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bg) no change or a reduction in heart rate of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bh) no change or a reduction in spleen size of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bi) no change or a reduction in liver size of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bj) no change or a reduction in dactylitis of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bk) no change or a reduction in pain crises of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bl) no change or a reduction in abnormal blood clot development of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bm) no change or a reduction in red blood cell distribution of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient;

(bn) no change or a reduction in reticulocyte count of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient; and/or

(bo) no change or a reduction in fibrinogen concentration of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(bp) no change or a reduction in serum creatine levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(bq) no change or a reduction in C-reactive protein (CRP) levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(br) no change or a reduction in pentraxin-2 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(bs) no change or a reduction in monocyte chemoattractant protein-1 (MCP-1/CCL2) levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(bt) no change or a reduction in circulating neutrophils of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(bu) no change or a reduction in vascular cell adhesion molecule 1 (VCAM-1) levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(bv) no change or a reduction in intercellular adhesion molecule 1 (ICAM-1) levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(bw) no change or a reduction in endothelin-1 (ET-1) levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least 124318.8019.US01\65342029.2 about 75%, at least about 80%, at least about 85%, at least about 90% or at least about 95% relative to baseline, placebo control, and/or untreated patient.

(bx) no change or an increase in hematocrit levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(by) no change or an increase in hemoglobin levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(bz) no change or a reduction in sickled red blood cell numbers of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(ca) no change or a reduction in CD68 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(cb) no change or a reduction in CD80 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(cc) no change or a reduction in NF-kB p65 activation of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(cd) no change or a reduction in NLRP3 (NOD-, LRR- and pyrin domain-containing protein 3) inflammasome activation of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(ce) no change or a reduction in vascular endothelial activation of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(cf) no change or a reduction in neutrophil vascular recruitment of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(cg) no change or a reduction in inflammatory cell infiltration of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(ch) no change or a reduction in thrombi formation of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(ci) no change or a reduction in iron overload and/or liver iron accumulation of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(cj) no change or a reduction in oxidative stress of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(ck) no change or a reduction in nuclear factor erythroid 2-related factor 2 (Nrf2) levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(cl) no change or a reduction in oxidation and/or heme oxygenase-1 (HO-1) levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(cm) no change or a reduction in E-selectin levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

(cn) no change or a reduction in red blood cell adhesion of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to baseline, placebo control, and/or untreated patient.

In any of the above embodiments disclosed herein, the therapeutically effective dose of the composition of the present technology is between 1 mg and 20 g of 15-HEPE (as the term “15-HEPE” is defined and exemplified herein) per day, for example, about 50 mg, about 100 mg, about 500 mg, about 750 mg, about 1 g, about 2 g, about 3 g, about 4 g, about 5 g, about 6 g, about 7 g, about 8 g, about 9 g, about 10 g, about 11 g, about 12 g, about 13 g, about 14 g, about 15 g, about 16 g, about 17 g, about 18 g, about 19 g, or about 20 g of 15-HEPE per day. In some embodiments, the therapeutically effective dose is between 4 g and 10 g of 15-HEPE per day. In another embodiment, the therapeutically effective dose is 4 g of 15-HEPE per day.

In some embodiments, the composition administered to the subject in an amount sufficient to provide a daily dose of 15-HEPE (as the term “15-HEPE” is defined and exemplified herein) about 1 mg to about 20,000 mg, about 25 mg to about 10,000 mg, about 50 mg to about 5000 mg, about 75 mg to about 2500 mg, or about 100 mg to about 1000 mg, for example, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg.

In some embodiments, the composition is administered to the subject for a period of time between about 3 days to about 1 year, for example, about 3 days, about 4 days, about 5 days, about 6 days, about 1 week, about 1.5 weeks, about 2 weeks, about 2.5 weeks, about 3 weeks, about 1 month, about 2 months, about 3 months, about 4 months, about 5 months, about 6 months, about 7 months, about 8 months, about 9 months, about 10 months, about 11 months, or about 1 year.

In some embodiments, the composition is administered to the subject once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years.

In some embodiments, the composition is administered to the subject once or more times (e.g., twice, three times, four times, or more) a day. In some embodiments, the composition is administered to the subject every day, every other day, every third day, weekly, biweekly (i.e., every other week), every third week, monthly, every other month, or every third month. In some embodiments, the composition can be administered continuously or intermittently, for example, in one or more cycles. In those embodiments, within each cycle, the composition can be administered at various lengths and/or frequencies as described above.

In some embodiments, the composition may be administered over a pre-determined time. Alternatively, the composition may be administered until a particular therapeutic benchmark is reached. In some embodiments, the methods provided herein include a step of evaluating one or more therapeutic benchmarks in a biological sample, such as, but not limited to, a lipid biomarker, a metabolic biomarker, an inflammatory biomarker, a cancer biomarker, to determine whether to continue administration of the composition.

In some embodiments, the composition may be administered in various routes as determined by one skilled in the art as suitable for an indication of interest. In some embodiments, the composition is administered by oral or enteric administration, intravenous injection, intraperitoneal injection, intertumoral injection, bone marrow injection, lymph node injection, subcutaneous injection, and/or cerebrospinal fluid injection.

In some embodiments, the composition is administered with or without food. In some embodiments, the composition is administered to a subject in a fasting state, for example, having not consumed any food in the preceding 12 hours, 8 hours, 4 hours, or 2 hours. In some embodiments, the composition is administered to a subject in a fed state, for example, within 2 hours, 1 hour, 45 minutes, or 30 minutes of having a meal.

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1: Unilateral Ureteral Obstruction (UUO)-Induced Renal Interstitial Fibrosis

The objective of this study was to examine the effects of DS109 (15-HETrE) and DS102 (15-HEPE) on UUO-induced renal interstitial fibrosis.

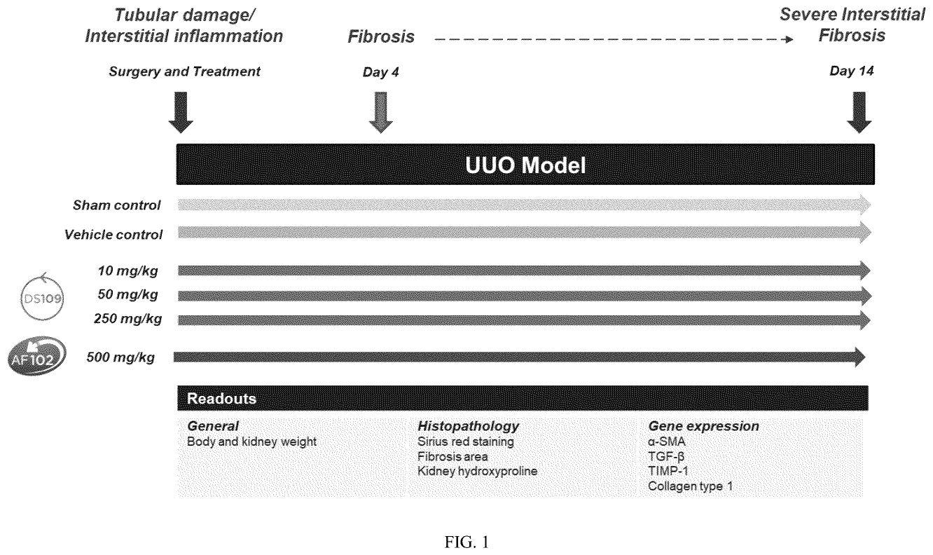

FIG. 1 depicts the study design from surgery and treatment to day 14 of the study.

### 1.1 Materials and Methods

Test Substances: The test substances for this study were DS109 (15-HETrE) and DS102 (15-HEPE). To prepare dosing solutions of each substance, DS109 was first weighed and then dissolved in a vehicle of 0.5% hydroxypropyl methyl cellulose (HPMC) and DS102 was diluted in a vehicle of 0.5% HPMC.

UUO Surgery: On day 0 of the study, UUO surgery was performed on mice under pentobarbital sodium anesthesia. The mouse's hair was first shaved and then abdomen cut open to exteriorize the mouse's left ureter. The ureter was ligated 4-0 nylon sutures at two points. The mouse's peritoneum and skin were then closed with sutures, and the mouse transferred to a clean cage until recovered from the anesthesia. Sham operated mice had their left ureter exposed but not ligated.

Drug Administration: DS109 and DS102 were administered to the mice orally in a volume of 10 milliliters (mL)/kilogram (Kg).

Treatment Doses: DS109 was administered at 3 dose levels of 5, 50, and 250 milligram (mg)/Kg once daily from Day 0 to Day 13 of the study. DS102 was administered at 2 doses levels of 50 and 500 mg/kg once daily from Day 0 to Day 13 of the study.

Animals: Seven-week-old female C57BL/6 mice (i.e., animals) were obtained and were housed and fed with a normal diet under controlled conditions. The animals were maintained in a specific-pathogen free (SPF) facility under controlled conditions of temperature (e.g., 23±2° C.), humidity (e.g., 45±10%), lighting (e.g., 12-hour artificial light and dark cycles; light from 8:00 to 20:00) and air exchange. A high pressure was maintained in the experimental room to prevent contamination of the facility. The animals were housed in TPX cages with a maximum of 4 mice per cage. Sterilized Paper-Clean was used for bedding and replaced once a week. Sterilized solid normal diet was provided ad libitum, being placed in a metal lid on the top of the cage. Pure water was also provided ad libitum from a water bottle equipped with a rubber stopper and a sipper tube. Water bottles were replaced once a week, cleaned, and sterilized in an autoclave and reused. Mice were identified by ear punch and each cage was labeled with a specific identification code.

Measurement of Kidney Biochemistry: To quantify kidney hydroxyproline content, frozen left kidney samples were processed by an alkaline-acid hydrolysis method as follows: kidney samples were dissolved in 2 normal (N) sodium hydroxide (NaOH) at 65° C. and autoclaved at 121° C. for 20 minutes. The lysed samples (400 μL) were acid-hydrolyzed with 400 L of 6N hydrochloride acid (HCl) at 121° C. for 20 minutes, and neutralized with 400 μL of 4N NaOH containing 10 mg/mL of activated carbon. An AC buffer (e.g., 2.2M acetic acid/0.48M citric acid, 400 μL) was added to the samples, followed by centrifugation to collect the supernatant. A standard curve of hydroxyproline was constructed with serial dilutions of trans-4-hydroxy-L-proline starting at 16 μg/mL. The prepared samples and standards (e.g., each 400 μL) were mixed with 400 μL chloramine T solution and incubated for 25 minutes at room temperature. The samples were then mixed with Ehrlich's solution (e.g., 400 μL) and heated at 65° C. for 20 minutes to develop the color. After samples were cooled on ice and centrifuged to remove precipitates, the optical density of each supernatant was measured at 560 nanometers (nm). The concentrations of hydroxyproline were calculated from the hydroxyproline standard curve. Protein concentrations of kidney samples were determined using a bicinchoninic (BCA) protein assay kit and used to normalize the calculated hydroxyproline values. Kidney hydroxyproline contents were expressed as microgram (μg) per mg protein.

Histopathological analysis: To visualize collagen deposition, kidney sections were stained using picro-Sirius red solution. For quantification of interstitial fibrosis area, bright field images in the corticomedullary region were captured using a digital camera at 200-fold magnification, and the positive areas in 5 fields/section were measured.

Quantitative RT-PCR: Total ribonucleic acid (RNA) was extracted from kidney samples using RNAiso. One μg of RNA was reverse-transcribed using a reaction mixture containing 4.4 micromolar (mM) of magnesium chloride (MgCl₂), 40 U RNase inhibitor, 0.5 mM dNTP, 6.28 UM random hexamer, 5× first strand buffer, 10 mM dithiothreitol and 200 U MMLV-RT in a final volume of 20 μL. The reaction was carried out for 1 hour at 37° C., followed by 5 minutes at 99° C. Real-time PCR was performed using real-time PCR DICE and TB Green™ Premix Ex Taq™ II. To calculate the relative microRNA (mRNA) expression level, the expression of each gene (e.g., α-SMA, TIMP-1, TGF-β and Collagen Type 1) was normalized to that of reference gene 36B4 (gene symbol: Rplp0). Information regarding the PCR-primer sets and the plate layout are described in Tables 1 and 2.

TABLE 1
PCR Primers for the Quantitative RT-PCR Measurements

| SEQ | | | | |
|---|---|---|---|---|
| ID | | | | |
| NO. | Gene | Set ID | | Sequence |
| 1 | 36B4 | MA057856 | forward | 5′-TTCCAGGCTTTGGGCATCA-3′ |
| 2 | | | reverse | 5′-ATGTTCAGCATGTTCAGCAGTGTG-3′ |
| 3 | Alpha- | MA057911 | forward | 5′-AAGAGCATCCGACACTGCTGAC-3′ |
| 4 | SMA | | reverse | 5′-AGCACAGCCTGAATAGCCACATAC-3′ |
| 5 | TIMP-1 | MA098519 | forward | 5′-TGAGCCCTGCTCAGCAAAGA-3′ |
| 6 | | | reverse | 5′-GAGGACCTGATCCGTCCACAA-3′ |
| 7 | TGF-β | MA030397 | forward | 5′-GTGTGGAGCAACATGTGGAACTCTA-3′ |
| 8 | | | reverse | 5′-TTGGTTCAGCCACTGCCGTA-3′ |
| 9 | Collagen | MA075477 | forward | 5′-CCAACAAGCATGTCTGGTTAGGAG-3′ |
| 10 | Type 1 | | reverse | 5′-GCAATGCTGTTCTTGCAGTGGTA-3′ |

36B4: Ribosomal protein, large, PO (Rplp0)
Alpha-SMA: Actin, alpha 2, smooth muscle, aorta (Acta2)
TIMP-1: Tissue inhibitor of metalloproteinase 1 (Timp1)
TGF-β: Transforming growth factor, beta 1 (Tgfb1)
Collagen Type 1: Collagen, type I, alpha 2 (Col1a2)

TABLE 2
Specifics Regarding the PCR Plates for
the Quantitative RT-PCR Measurements

Plate Mouse ID

| | 1 | 2 | 3 |
|---|---|---|---|
| | 101-308 | 401-608 | 701-708 |
| Alpha-SMA | Plate 1-2 | Plate 2-2 | Plate 3-2 |
| 36B4 | Plate 1-1 | Plate 2-1 | Plate 3-1 |
| TIMP-1 | Plate 1-3 | Plate 2-3 | Plate 3-2 |
| 36B4 | Plate 1-1 | Plate 2-1 | Plate 3-1 |
| TGF-β | Plate 1-4 | Plate 2-4 | Plate 3-3 |
| 36B4 | Plate 1-1 | Plate 2-1 | Plate 3-1 |
| Collagen Type 1 | Plate 1-5 | Plate 2-5 | Plate 3-3 |
| 36B4 | Plate 1-1 | Plate 2-1 | Plate 3-1 |

Sample Collection: For serum samples, non-fasting blood was collected in serum separate tubes without anticoagulant through direct cardiac puncture and centrifuged at 3,500×g for 4 minutes at 4° C. The supernatant was collected and stored at −80° C. for shipping. For kidney samples, the left kidney was collected and cut into 2 pieces horizontally. Superior part of left kidney was fixed in Bouin's solution and then embedded in paraffin. Paraffin blocks were stored at room temperature for histological analyses. The inferior part of left kidney was cut into 2 pieces coronally. The anterior part of left kidney was snap frozen in liquid nitrogen and stored at −80° C. for gene expression assay. The posterior part of left kidney was snap frozen in liquid nitrogen and stored at −80° C. for kidney biochemistry.

Statistical Tests: Statistical analyses were performed using Bonferroni Multiple Comparison Test. P values <0.05 were considered statistically significant. A trend or tendency was assumed when a one-tailed t-test returned P values <0.1. Results were expressed as mean±SD.

### 1.2 Experimental Design and Treatment

The study design included the following study groups:
Group 1 (Sham Control): Eight sham-operated mice kept without any treatment until sacrifice.
Group 2 (Vehicle): Eight UUO mice were orally administered vehicle [0.5% HPMC] in a volume of 10 mL/kg once daily from Day 0 to Day 13.
Group 3 (DS109 Low): Eight UUO mice were orally administered vehicle supplemented with DS109 at a dose of 5 mg/kg once daily from Day 0 to Day 13.
Group 4 (DS109 Middle): Eight UUO mice were orally administered vehicle supplemented with DS109 at a dose of 50 mg/kg once daily from Day 0 to Day 13.
Group 5 (DS109 High): Eight UUO mice were orally administered vehicle supplemented with DS109 at a dose of 250 mg/kg once daily from Day 0 to Day 13.
Group 6 (DS102 Low): Eight UUO mice were orally administered vehicle supplemented with DS102 at a dose of 50 mg/kg once daily from Day 0 to Day 13.
Group 7 (DS102 High): Eight UUO mice were orally administered vehicle supplemented with DS102 at a dose of 500 mg/kg once daily from Day 0 to Day 13.

Table 3 summarizes the treatment schedule for each of Groups 1-7 during the study.

TABLE 3
Summary of the Treatment Schedule

| | | | Test | Dose | Volume | | Sacrifice |
|---|---|---|---|---|---|---|---|
| Group | No. mice | Model | substance | (mg/kg) | (mL/kg) | Regimen | (Day) |
| 1 | 8 | Sham | — | — | — | Day 0-Day 13 | 14 |
| 2 | 8 | UUO | Vehicle | — | 10 | PO, QD, | 14 |
| | | | | | | Day 0-Day 13 | |
| 3 | 8 | UUO | DS109 | 5 | 10 | PO, QD, | 14 |
| | | | | | | Day 0-Day 13 | |
| 4 | 8 | UUO | DS109 | 50 | 10 | PO, QD, | 14 |
| | | | | | | Day 0-Day 13 | |
| 5 | 8 | UUO | DS109 | 250 | 10 | PO, QD, | 14 |
| | | | | | | Day 0-Day 13 | |
| 6 | 8 | UUO | DS102 | 50 | 10 | PO, QD, | 14 |
| | | | | | | Day 0-Day 13 | |
| 7 | 8 | UUO | DS102 | 500 | 10 | PO, QD, | 14 |
| | | | | | | Day 0-Day 13 | |

Animal Monitoring and Sacrifice: The viability, clinical signs and behavior for the mice were monitored daily. Individual body weight was measured daily before treatment during the treatment period. Mice were observed for significant clinical signs of toxicity, moribundity and mortality approximately 60 minutes after each administration. The animals were sacrificed by exsanguination through direct cardiac puncture under isoflurane anesthesia at Day 14.

### 1.3 Results

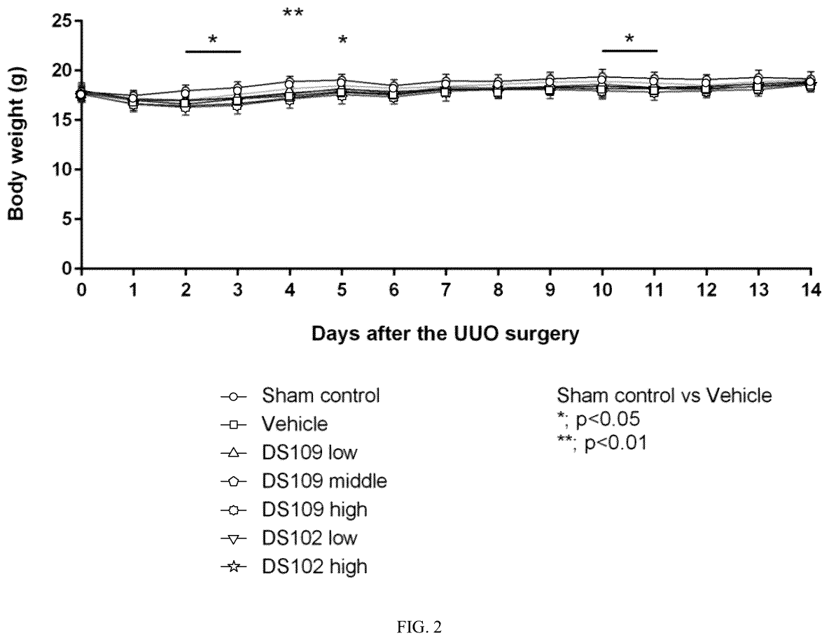

Body weight changes and general considerations: FIG. 2 shows the body weight changes for all animals. In all the animals, body weight decreased after surgery, and recovered gradually during the treatment period. Mean body weight of the Vehicle group was significantly lower than that of the Sham control group from Day 2 to Day 5 and from Day 10 to Day 11. There were no significant changes in mean body weight at any day during the treatment period between the Vehicle group and the treatment groups. There were no dead animals in all groups during the treatment period. In the present study, none of the animals showed deterioration in general condition.

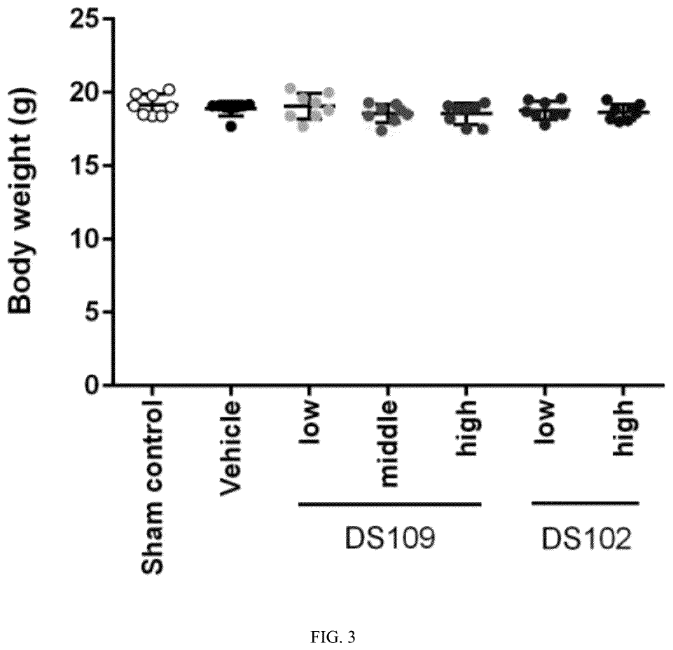

Body and kidney weight on the day of sacrifice: FIG. 3 and Table 4 show the body weight of the animals on the day of sacrifice. There was no significant difference in mean body weight on the day of sacrifice between the Sham control group and the Vehicle group. There were no significant differences in mean body weight on the day of sacrifice between the Vehicle group and the treatment groups.

TABLE 4
Body Weight on the Day of Sacrifice

| | | | Right Kidney- | | Left Kidney- |
|---|---|---|---|---|---|
| | | | to-Body | | to-Body |
| | Body Weight | Right Kidney | Weight Ratio | Left Kidney | Weight |
| Group | (g) | Weight (mg) | (%) | Weight (mg) | Ratio (%) |
| Sham control, | 19.2 ± 0.7 | 122 ± 8 | 0.64 ± 0.05 | 115 ± 14 | 0.60 ± 0.08 |
| (n = 8) | | | | | |
| Vehicle, (n = 8) | 18.9 ± 0.5 | 139 ± 15 | 0.73 ± 0.09 | 488 ± 150 | 2.56 ± 0.77 |
| DS109 low, | 19.1 ± 0.9 | 137 ± 7 | 0.71 ± 0.04 | 533 ± 138 | 2.79 ± 0.65 |
| (n = 8) | | | | | |
| DS109 middle, | 18.6 ± 0.6 | 135 ± 6 | 0.73 ± 0.05 | 523 ± 91 | 2.83 ± 0.50 |
| (n = 8) | | | | | |
| DS109 high, | 18.6 ± 0.7 | 130 ± 13 | 0.70 ± 0.05 | 425 ± 105 | 2.26 ± 0.53 |
| (n = 8) | | | | | |
| DS102 low, | 18.8 ± 0.6 | 133 ± 10 | 0.70 ± 0.05 | 469 ± 60 | 2.49 ± 0.30 |
| (n = 8) | | | | | |
| DS102 high, | 18.7 ± 0.5 | 136 ± 6 | 0.71 ± 0.04 | 487 ± 108 | 2.61 ± 0.57 |
| (n = 8) | | | | | |

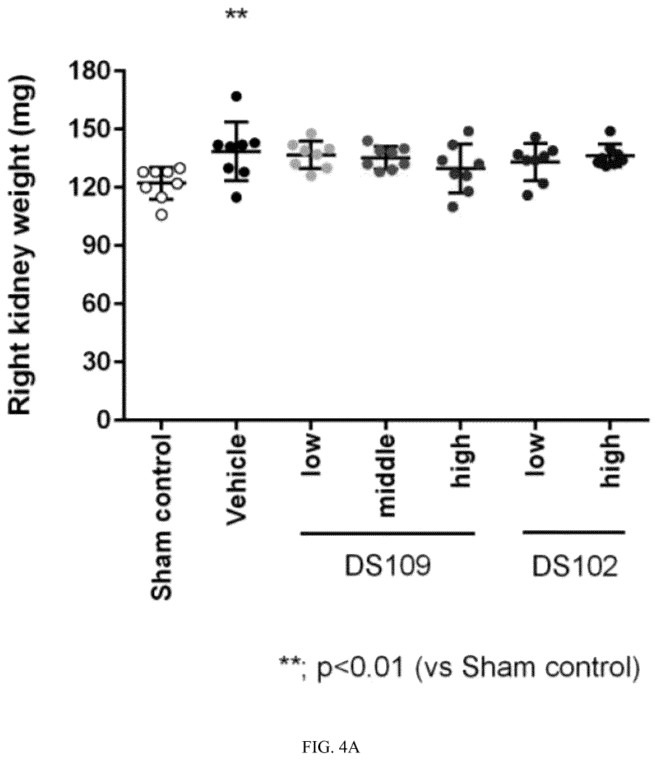

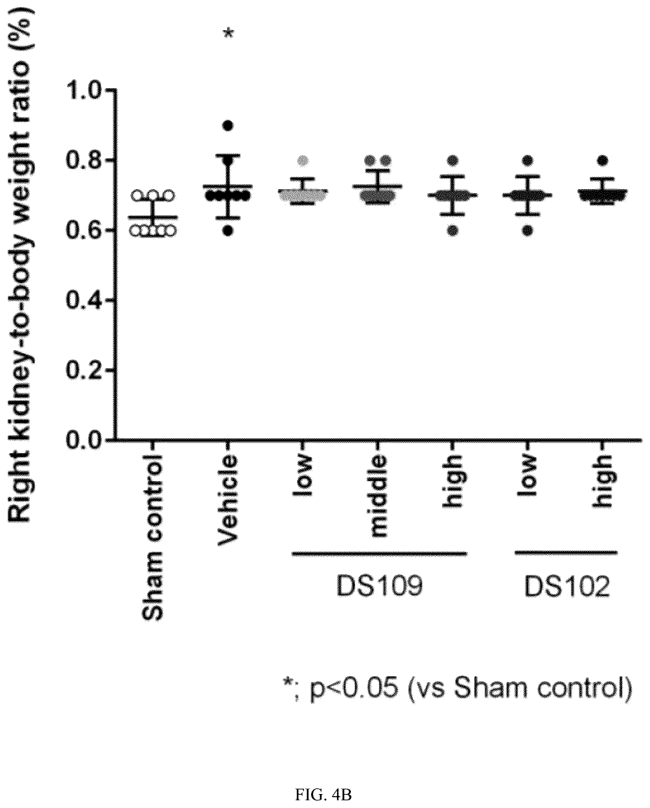

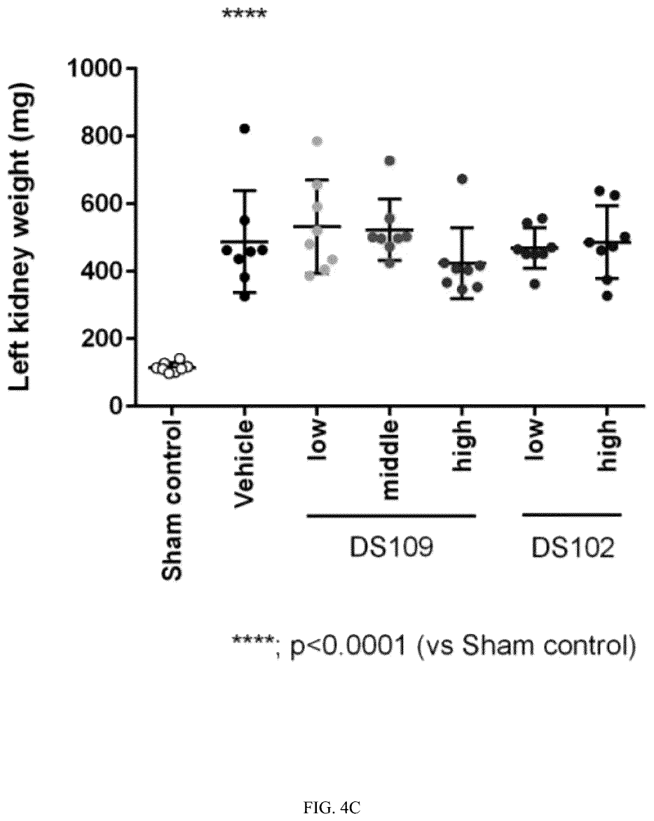

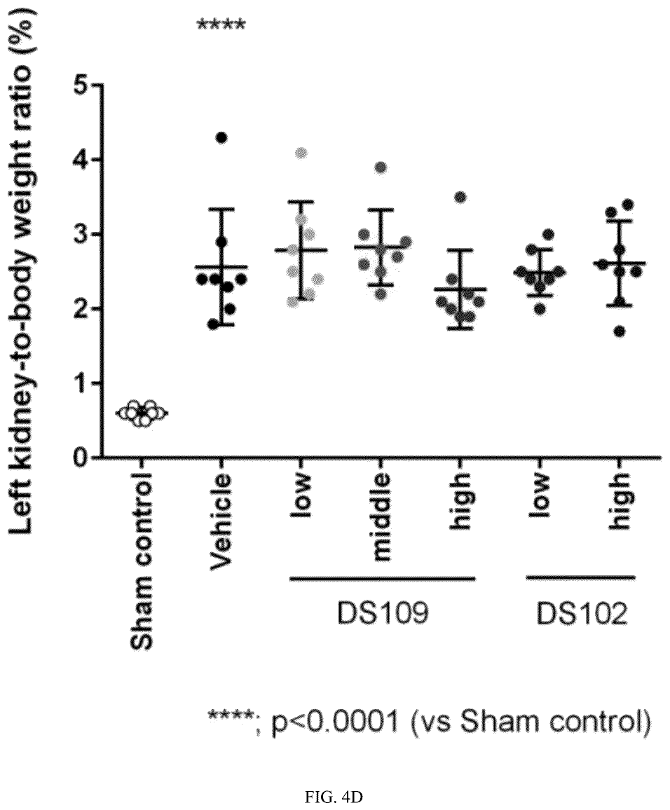

FIGS. 4A-4D and Table 4 show the kidney weight and kidney-to-body weight ratio of the animals on the day of sacrifice. The Vehicle group showed a significant increase in mean right kidney weight compared with the Sham control group. However, there were no significant differences in mean right kidney weight between the Vehicle group and the treatment groups. The Vehicle group also showed a significant increase in mean right kidney-to-body weight ratio compared with the Sham control group. There were no significant differences in mean right kidney-to-body weight ratio between the Vehicle group and the treatment groups. The Vehicle group also showed a significant increase in mean left kidney weight compared with the Sham control group. There were no significant differences in mean left kidney weight between the Vehicle group and the treatment groups. Lastly, the Vehicle group showed a significant increase in mean left kidney-to-body weight ratio compared with the Sham control group, but there were no significant differences in mean left kidney-to-body weight ratio between the Vehicle group and the treatment groups.

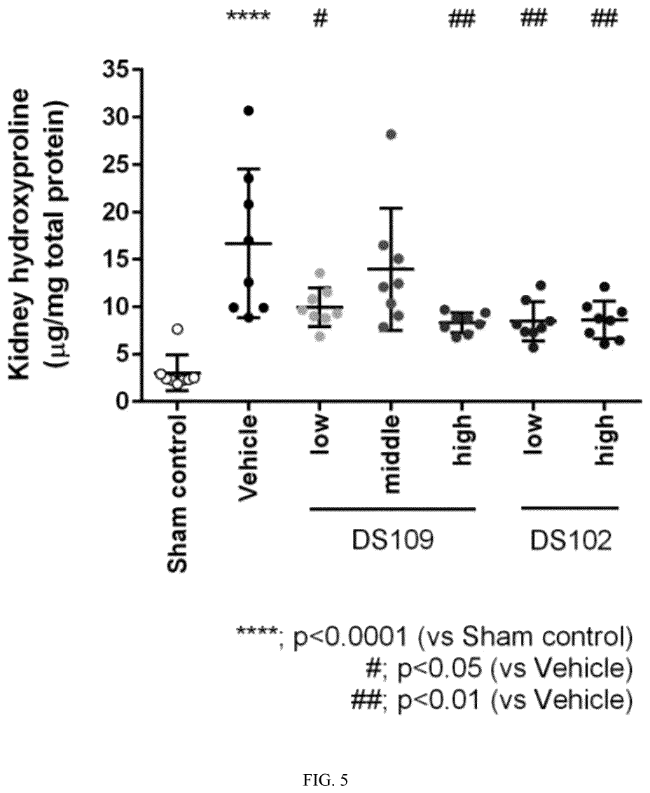

Kidney Chemistry: FIG. 5 and Table 5 show the kidney hydroxyproline content for the animals. The Vehicle group showed a significant increase in kidney hydroxyproline contents compared with the Sham control group. The DS109 low, DS109 high, DS102 low and DS102 high groups showed significant decreases in kidney hydroxyproline contents compared with the Vehicle group. There was no significant difference in kidney hydroxyproline contents between the Vehicle group and the DS109 middle group.

TABLE 5
The Kidney Hydroxyproline Content

| | Kidney hydroxyproline |
|---|---|
| Group | (μg/mg total protein) |
| Sham control, (n = 8) | 3.02 ± 1.89 |
| Vehicle, (n = 8) | 16.67 ± 7.83 |
| DS109 low, (n = 8) | 9.94 ± 2.02 |
| DS109 middle, (n = 8) | 13.94 ± 6.44 |
| DS109 high, (n = 8) | 8.30 ± 1.04 |
| DS102 low, (n = 8) | 8.47 ± 2.08 |
| DS102 high, (n = 8) | 8.59 ± 1.99 |

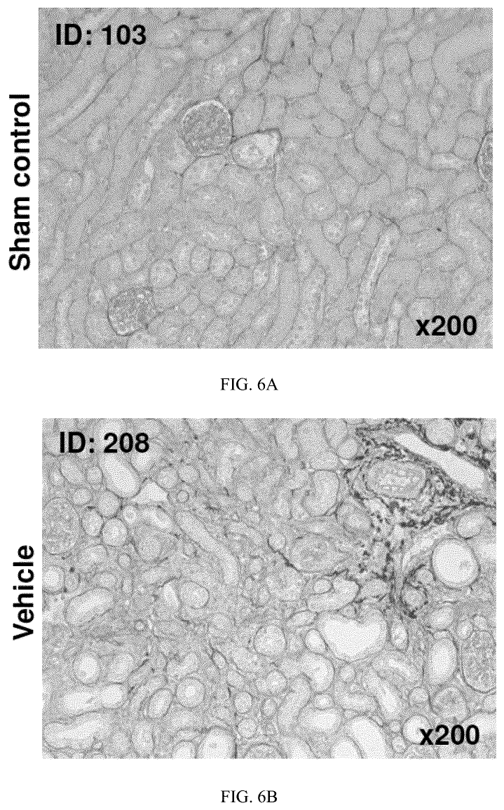

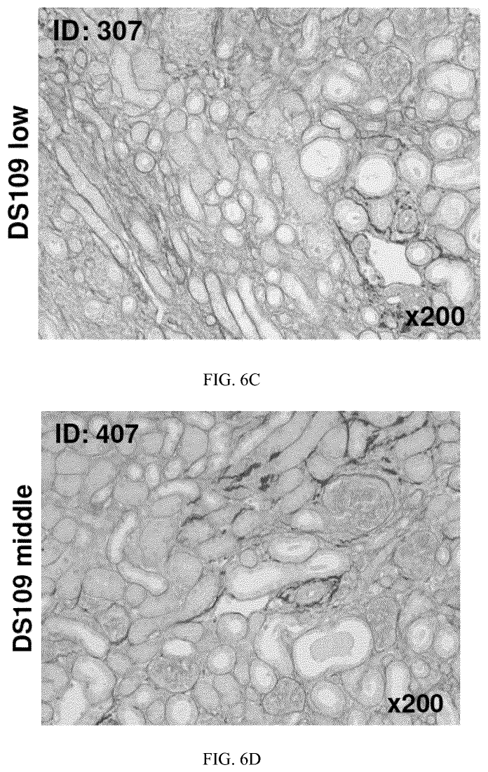

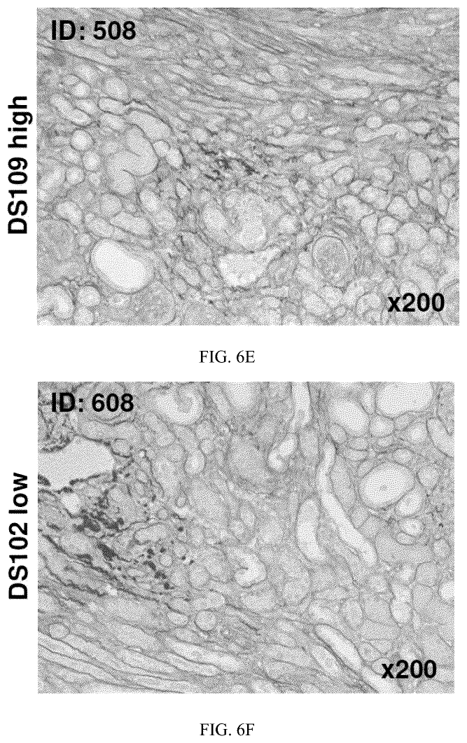

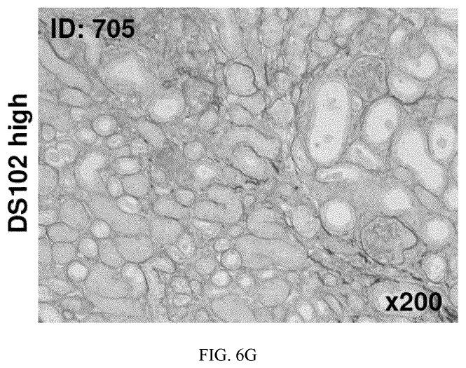

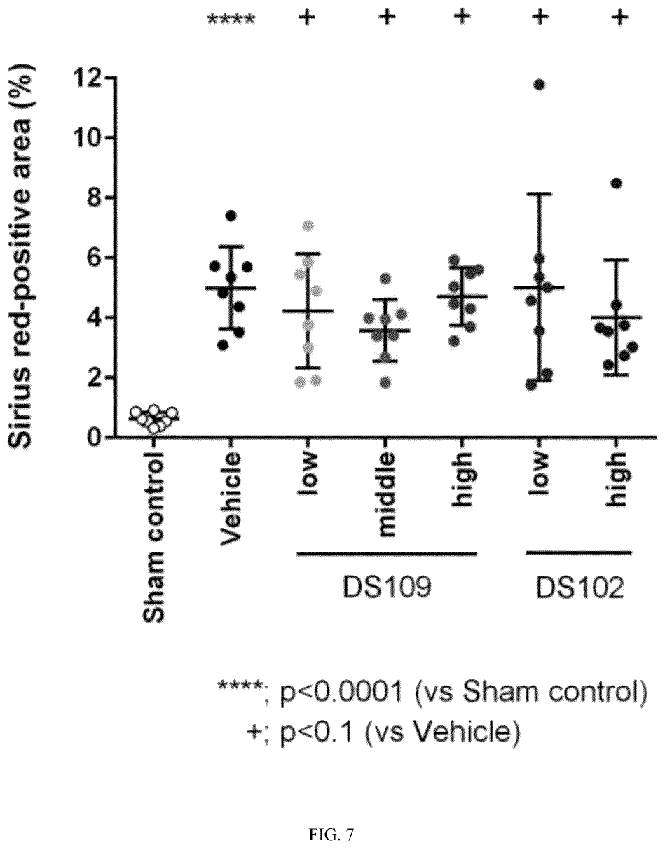

Histological Analysis: FIGS. 6A-6G and Table 6 show the Sirius red staining and FIG. 7, the fibrosis area of the animals. FIG. 6A displays representative photomicrographs of Sirius red-stained kidney sections. The Vehicle group showed a significant increase in the percentage of fibrosis area (Sirius red-positive area) compared with the Sham control group. Bonferroni Multiple Comparison revealed that the fibrosis area in the DS109 middle group tended to decrease compared with the Vehicle group. There were no significant differences in fibrosis area between the Vehicle group and the other treatment groups. Mann-Whitney U test was conducted due to the presence of notable outliers and revealed that the DS109 low, DS109 middle, DS109 high, DS102 low and DS102 high groups fibrosis area tended to decrease (p<0.1) compared with the Vehicle group.

TABLE 6
Fibrosis Area of the Animals

| | Sirius red- | Sirius red- |
|---|---|---|
| Group | positive area (%) | positive area (%)_Median |
| Sham control, (n = 8) | 0.63 ± 0.22 | 0.51 ± 0.15 |
| Vehicle, (n = 8) | 5.00 ± 1.37 | 4.55 ± 0.94 |
| DS109 low, (n = 8) | 4.23 ± 1.90 | 3.22 ± 1.47 |
| DS109 middle, (n = 8) | 3.58 ± 1.04 | 3.28 ± 1.41 |
| DS109 high, (n = 8) | 4.71 ± 0.96 | 3.76 ± 0.81 |
| DS102 low, (n = 8) | 5.01 ± 3.11 | 4.19 ± 3.33 |
| DS102 high, (n = 8) | 4.01 ± 1.92 | 3.08 ± 1.16 |

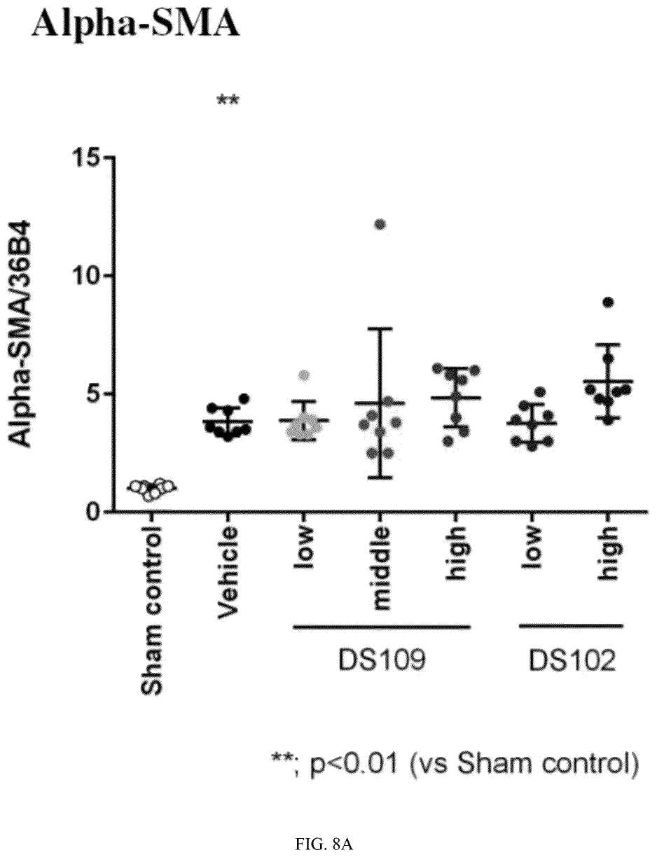

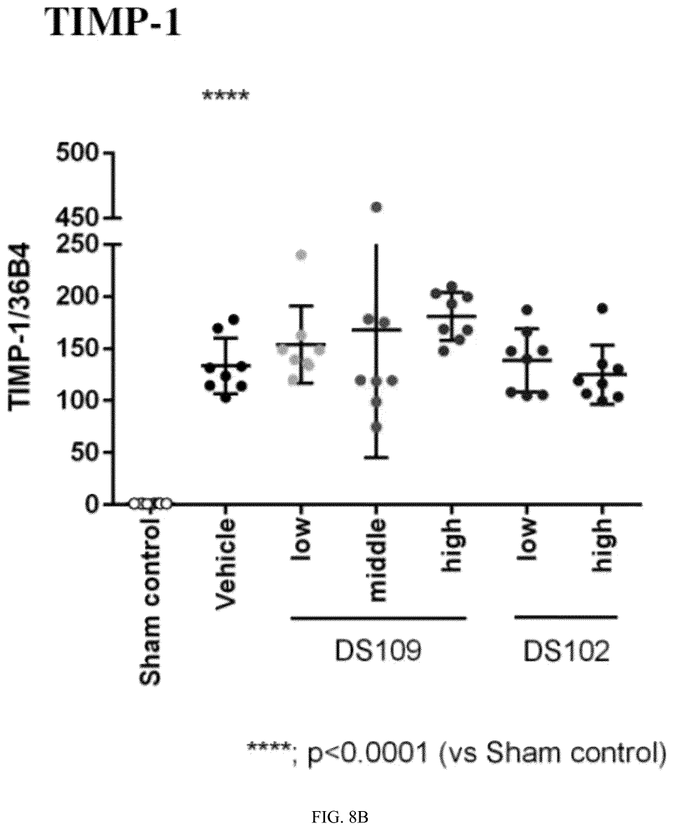

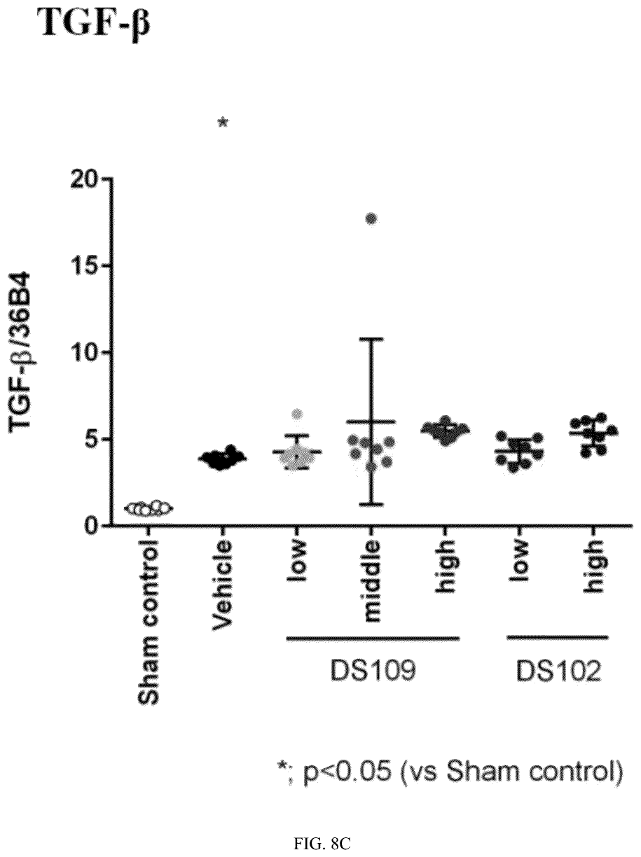

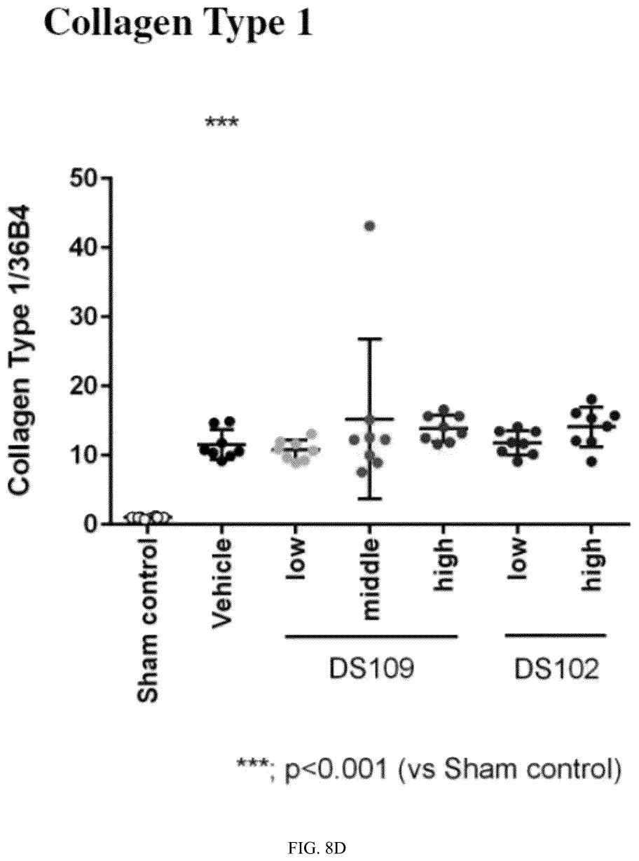

Gene Expression Analyses: Gene expression analyses for alpha-SMA, TIMP-1, TGF-β, Collagen Type 1 are shown in FIG. 8A-8D and Table 7.

TABLE 7
Gene Expression Analysis

| Group | Alpha-SMA | TIMP-1 | TGF-β | Collagen Type 1 |
|---|---|---|---|---|
| Sham control, (n = 8) | 1.00 ± 0.18 | 1.00 ± 0.14 | 1.00 ± 0.09 | 1.00 ± 0.13 |
| Vehicle, (n = 8) | 3.82 ± 0.58 | 133.20 ± 26.86 | 3.88 ± 0.30 | 11.52 ± 2.15 |
| DS109 low, (n = 8) | 3.87 ± 0.82 | 153.60 ± 37.12 | 4.29 ± 0.93 | 10.76 ± 1.45 |
| DS109 middle, (n = 8) | 4.61 ± 3.15 | 167.70 ± 122.60 | 6.01 ± 4.77 | 15.23 ± 11.52 |
| DS109 high, (n = 8) | 4.85 ± 1.22 | 180.90 ± 23.12 | 5.48 ± 0.36 | 13.88 ± 1.91 |
| DS102 low, (n = 8) | 3.74 ± 0.81 | 138.30 ± 30.39 | 4.31 ± 0.69 | 11.80 ± 1.78 |
| DS102 high, (n = 8) | 5.54 ± 1.54 | 124.80 ± 28.60 | 5.37 ± 0.76 | 14.09 ± 2.88 |

Alpha-SMA: The Vehicle group showed a significant increase in α-SMA mRNA expression level compared with the Sham control group. There were no significant differences in α-SMA mRNA expression level between the Vehicle group and the treatment groups.

TIMP-1: The Vehicle group showed a significant increase in TIMP-1 mRNA expression level compared with the Sham control group. There were no significant differences in TIMP-1 mRNA expression level between the Vehicle group and the treatment groups.

TGF-β: The Vehicle group showed a significant increase in TGF-β mRNA expression level compared with the Sham control group. There were no significant differences in TGF-β mRNA expression level between the Vehicle group and the treatment groups.

Collagen Type 1: The Vehicle group showed a significant increase in Collagen Type 1 mRNA expression level compared with the Sham control group. There were no significant differences in Collagen Type 1 mRNA expression level between the Vehicle group and the treatment groups.

### 1.4 Summary

As shown by Sirius-red staining and kidney hydroxyproline content, renal fibrosis was established in the Vehicle group in the present study.

DS109: Treatment with DS109 at low dose showed significant decreases (p<0.05) in kidney hydroxyproline contents and a reduction trend (p<0.1) in fibrosis area compared with the Vehicle group. Treatment with DS109 at middle dose showed reduction trend (p<0.1) in the fibrosis area compared with the Vehicle group. Lastly, treatment with DS109 at high dose showed significant decreases (p<0.05) in kidney hydroxyproline contents and a reduction trend (p<0.1) in fibrosis area compared with the Vehicle group.

DS102: Treatment with DS102 at low dose showed a significant decrease in kidney hydroxyproline contents (p<0.05), and a reduction trend (p<0.1) in fibrosis area compared with the Vehicle group. Treatment with DS102 at high dose showed significant decreases in kidney hydroxyproline contents (p<0.05), and a reduction trend (p<0.1) in fibrosis area compared with the Vehicle group.

In conclusion, the results from this study suggest that DS109 and DS102 have a suppressive effect on renal fibrogenesis and potential as therapeutic agents for chronic renal diseases that progress to fibrosis.

### Example 2: Cholestatic Liver Disease and/or Liver Fibrosis Bile Duct Ligation (BDL) Study

The objective of this study was to examine the effects of DS012 on cholestasis induced by BDL.

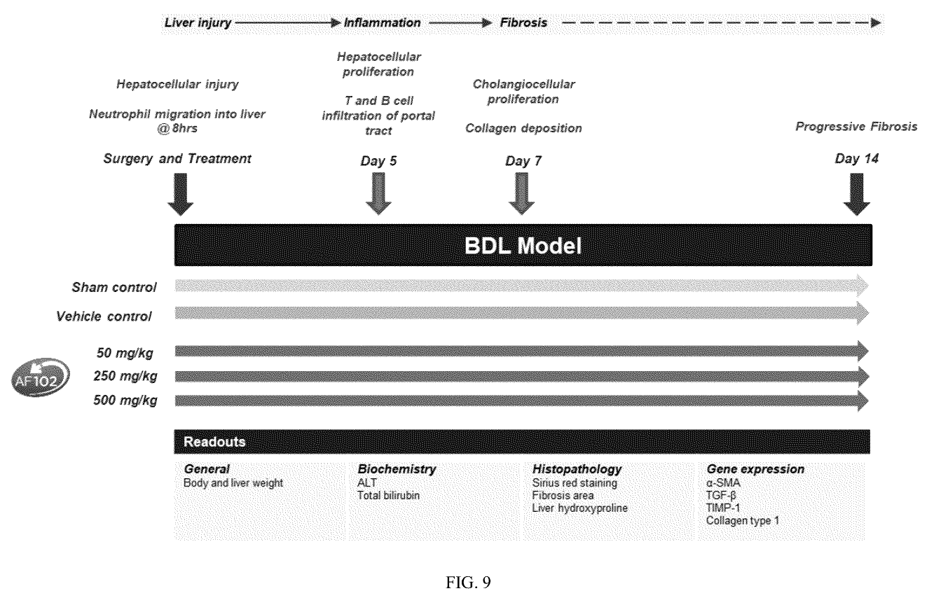

FIG. 9 depicts the study design from surgery and treatment to day 14 of the study.

### 2.1 Materials and Methods

Test Substance: The test substance for this study was DS102. To prepare dosing solutions of each substance, DS102 was diluted in a vehicle of 0.5% hydroxypropyl methyl cellulose (HPMC).

BDL Surgery: On Day 0 of the study, BDL surgery was performed under pentobarbital anesthesia. The mouse's hair was first shaved, the abdominal cavity cut open, and the common bile duct was ligated twice with 7-0 surgical silk. The mouse's peritoneum and the skin were closed with sutures, and the mice were transferred to a clean cage (e.g., resting cage) until recovered from anesthesia. Sham operated mice had their common bile duct exposed but not ligated.

Drug Administration: DS102 was administered to orally in a volume of 10 milliliters (mL)/kilogram (Kg).

Treatment Doses: DS102 was administered at 3 dose levels of 50, 250, and 500 milligram (mg)/Kg once daily from Day 0 to Day 13 of the study.

Animals: Pathogen-free 6 weeks of age male C57BL/6J mice were obtained. The animals were maintained in a specific-pathogen free (SPF) facility under controlled conditions of temperature (e.g., 23±2° C.), humidity (e.g., 45±10%), lighting (e.g., 12-hour artificial light and dark cycles; light from 8:00 to 20:00) and air exchange. A high pressure was maintained in the experimental room to prevent contamination of the facility. The animals were housed in TPX cages with a maximum of 4 mice per cage. Sterilized Paper-Clean was used for bedding and replaced once a week. Sterilized solid normal diet was provided ad libitum, being placed in a metal lid on the top of the cage. Pure water was also provided ad libitum from a water bottle equipped with a rubber stopper and a sipper tube. Water bottles were replaced once a week, cleaned, and sterilized in an autoclave and reused. Mice were identified by ear punch and each cage was labeled with a specific identification code.

Measurement of Serum Biochemistry: To visualize collagen deposition, Bouin's fixed liver sections were stained using picro-Sirius red solution. For quantitative analysis of fibrosis area, bright field images of Sirius red-stained sections were captured using a digital camera at 100-fold magnification, and the positive areas in 5 fields/section were measured.

Histological analysis: To visualize collagen deposition, kidney sections were stained using picro-Sirius red solution. For quantification of interstitial fibrosis area, bright field images in the corticomedullary region were captured using a digital camera at 200-fold magnification, and the positive areas in 5 fields/section were measured.

Quantitative RT-PCR: Total ribonucleic acid (RNA) was extracted from liver samples using RNAiso. One μg of RNA was reverse-transcribed using a reaction mixture containing 4.4 mM magnesium chloride (MgCl₂), 40 U RNase inhibitor, 0.5 mM dNTP, 6.28 μM random hexamer, 5× first strand buffer, 10 mM dithiothreitol and 200 U MMLV-RT in a final volume of 20 μL. The reaction was carried out for 1 hour at 37° C., followed by 5 minutes at 99° C. Real-time PCR was performed using real-time PCR DICE and TB Green™ Premix Ex Taq™ II. To calculate the relative mRNA expression level, the expression of each gene (α-SMA, TIMP-1, TGF-β and Collagen Type 1) was normalized to that of reference gene 36B4 (gene symbol: Rplp0). Information regarding the PCR-primer sets and the plate layout are described in Tables 8 and 9.

TABLE 8
PCR Primers for the Quantitative RT-PCR Measurements

| SEQ ID | | | | |
|---|---|---|---|---|
| NO. | Gene | Set ID | | Sequence |
| 1 | 36B4 | MA057856 | forward | 5′-TTCCAGGCTTTGGGCATCA-3′ |
| 2 | | | reverse | 5′-ATGTTCAGCATGTTCAGCAGTGTG-3′ |
| 3 | Alpha-SMA | MA057911 | forward | 5′-AAGAGCATCCGACACTGCTGAC-3′ |
| 4 | | | reverse | 5′-AGCACAGCCTGAATAGCCACATAC-3′ |
| 5 | TIMP-1 | MA098519 | forward | 5′-TGAGCCCTGCTCAGCAAAGA-3′ |
| 6 | | | reverse | 5′-GAGGACCTGATCCGTCCACAA-3′ |
| 7 | TGF-β | MA030397 | forward | 5′-GTGTGGAGCAACATGTGGAACTCTA- |
| | | | | 3′ |
| 8 | | | reverse | 5′-TTGGTTCAGCCACTGCCGTA-3′ |
| 9 | Collagen Type 1 | MA075477 | forward | 5′-CCAACAAGCATGTCTGGTTAGGAG-3′ |
| 10 | | | reverse | 5′-GCAATGCTGTTCTTGCAGTGGTA-3′ |

36B4: Ribosomal protein, large, PO (Rplp0)
Alpha-SMA: Actin, alpha 2, smooth muscle, aorta (Acta2)
TIMP-1: Tissue inhibitor of metalloproteinase 1 (Timp1)
TGF-β: Transforming growth factor, beta 1 (Tgfb1)
Collagen Type 1: Collagen, type I, alpha 2 (Col1a2)

TABLE 9
Specifics Regarding the PCR Plates for
the Quantitative RT-PCR Measurements

Plate Mouse ID

| | 1 | 2 | 3 |
|---|---|---|---|
| | 101-308 | 401-608 | 701-708 |
| Alpha-SMA | Plate 1-2 | Plate 2-2 | Plate 3-2 |
| 36B4 | Plate 1-1 | Plate 2-1 | Plate 3-1 |
| TIMP-1 | Plate 1-3 | Plate 2-3 | Plate 3-2 |
| 36B4 | Plate 1-1 | Plate 2-1 | Plate 3-1 |
| TGF-β | Plate 1-4 | Plate 2-4 | Plate 3-3 |
| 36B4 | Plate 1-1 | Plate 2-1 | Plate 3-1 |
| Collagen Type 1 | Plate 1-5 | Plate 2-5 | Plate 3-3 |
| 36B4 | Plate 1-1 | Plate 2-1 | Plate 3-1 |

Sample Collection: For serum samples, non-fasting blood was collected in serum separate tubes without anticoagulant through direct cardiac puncture and centrifuged at 3,500×g for 4 minutes at 4° C. The superatant was collected and stored at −80° C. for biochemistry (30 μL) and shipping (all the remaining). For liver samples, left lateral lobe was collected and cut into 6 pieces. Two pieces of left lateral lobe were fixed in Bouin's solution and then embedded in paraffin. Samples were stored at room temperature for histological analysis. The other 2 pieces of left lateral lobe were embedded in O.C.T. compound and quick frozen in liquid nitrogen. Samples were stored at −80° C. The remaining pieces of left lateral lobe was snap frozen in liquid nitrogen and stored at −80° C. for gene expression analyses. Right medial lobe, left medial lobe, right lobe and caudate lobe were snap frozen in liquid nitrogen and stored at −80° C. for shipping.

Statistical Tests: Statistical analyses were performed using Bonferroni Multiple Comparison Test. P values <0.05 were considered statistically significant. A trend or tendency was assumed when a one-tailed t-test returned P values <0.1. Results were expressed as mean±SD.

### 2.2 Experimental Design and Treatment

The study design included the following study groups:
Group 1 (Sham Control): Eight sham-operated mice kept without any treatment until sacrifice.
Group 2 (Vehicle): Fifteen BDL-operated mice were orally administered vehicle [0.5% HPMC] in a volume of 10 mL/kg once daily from Day 0 to Day 13.
Group 3 (DS102 Low): Fifteen BDL-operated mice were orally administered vehicle supplemented with DS102 at a dose of 50 mg/kg once daily from Day 0 to Day 13.
Group 4 (DS102 Middle): Fifteen BDL-operated mice were orally administered vehicle supplemented with DS102 at a dose of 250 mg/kg once daily from Day 0 to Day 13.
Group 5 (DS102 High): Fifteen BDL-operated mice were orally administered vehicle supplemented with DS102 at a dose of 500 mg/kg once daily from Day 0 to Day 13.

Table 10 summarizes the treatment schedule for each of Groups 1-5 during the study.

TABLE 10
Summary of the Treatment Schedule

| | | | Test | Dose | Volume | | |
|---|---|---|---|---|---|---|---|
| Group | No. mice | Mice | substance | (mg/kg) | (mL/kg) | Regimen | Sacrifice |
| 1 | 8 | Sham | — | — | — | — | Day 14 |
| 2 | 15 | BDL | Vehicle | — | 10 | PO, QD, | Day 14 |
| | | | | | | Day 0-13 | |
| 3 | 15 | BDL | DS102 | 50 | 10 | PO, QD, | Day 14 |
| | | | | | | Day 0-13 | |
| 4 | 15 | BDL | DS102 | 250 | 10 | PO, QD, | Day 14 |
| | | | | | | Day 0-13 | |
| 5 | 15 | BDL | DS102 | 500 | 10 | PO, QD, | Day 14 |
| | | | | | | Day 0-13 | |

Animal Monitoring and Sacrifice: The viability, clinical signs and behavior for the mice were monitored daily. Individual body weight was measured daily before treatment during the treatment period. Mice were observed for significant clinical signs of toxicity, moribundity and mortality approximately 60 minutes after each administration. The animals were sacrificed at Day 14 after BDL surgery by exsanguination through direct cardiac puncture under isoflurane anesthesia.

### 2.3 Results

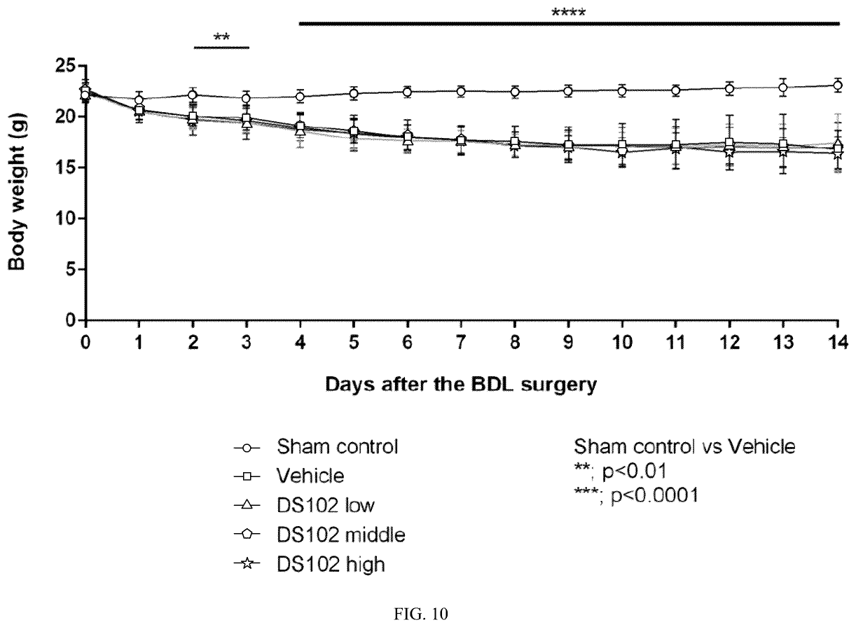

Body weight changes and general considerations: FIG. 10 shows the body weight changes for all animals. Mean body weight in all groups, except of the Sham control group, gradually decreased during the study period. Mean body weight of the Vehicle group was significantly lower than that of the Sham control group from Day 2 to Day 14. There were no significant changes in mean body weight at any day during the study period between the Vehicle group and the DS102 treatment groups.

During the treatment period, mice found dead before reaching Day 14 were as follows: three out of 15 mice were found dead in the Vehicle group; seven out of 15 mice were found dead in the DS102 low, DS102, middle and DS102 high groups. In this model, a percentage of deaths are expected simply due to disease induction and the observed mortality rate is consistent with historical data.

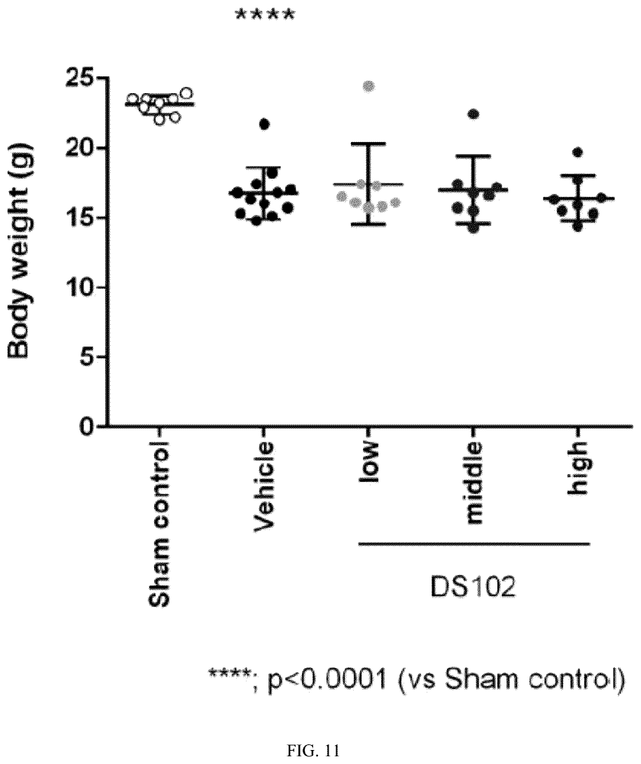

Body and liver weight on the day of sacrifice: FIG. 11 and Table 11 show the body weight of the animals on the day of sacrifice. The Vehicle group showed a significant decrease in mean body weight on the day of sacrifice compared with the Sham control group. There were no significant differences in mean body weight on the day of sacrifice between the Vehicle group and the DS102 treatment groups.

TABLE 11
Body Weight on the Day of Sacrifice

| | | | Liver-to- |
|---|---|---|---|
| | Body | Liver | Body Weight |
| Group | Weight (g) | Weight (mg) | Ratio (%) |
| Sham control, (n = 8) | 23.1 ± 0.7 | 1132 ± 90 | 4.9 ± 0.4 |
| Vehicle, (n = 12) | 16.8 ± 1.8 | 1438 ± 180 | 8.7 ± 1.4 |
| DS102 low, (n = 8) | 17.4 ± 2.9 | 1392 ± 328 | 8.3 ± 2.3 |
| DS102 middle, (n = 8) | 17.0 ± 2.4 | 1513 ± 182 | 9.0 ± 1.2 |
| DS102 high, (n = 8) | 16.4 ± 1.6 | 1231 ± 255 | 7.6 ± 1.5 |

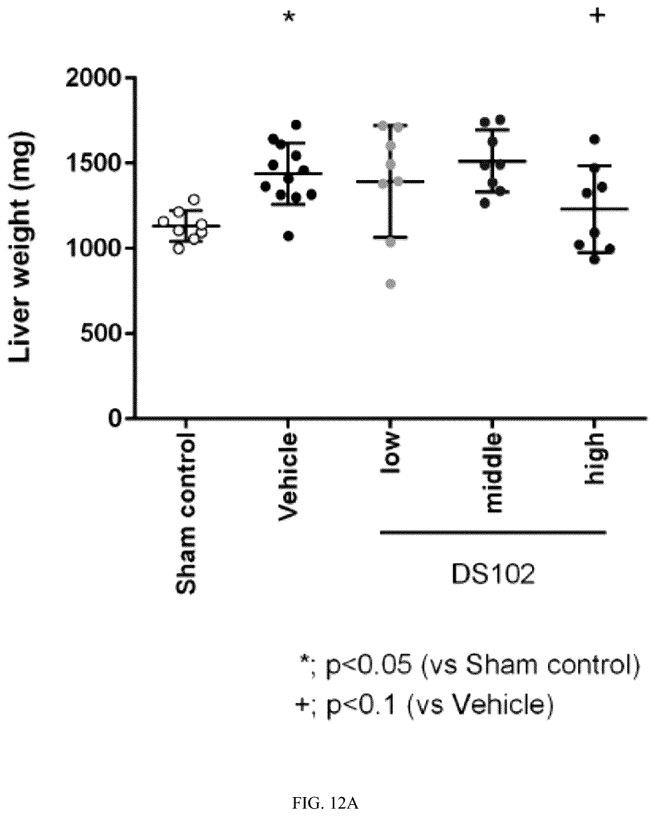

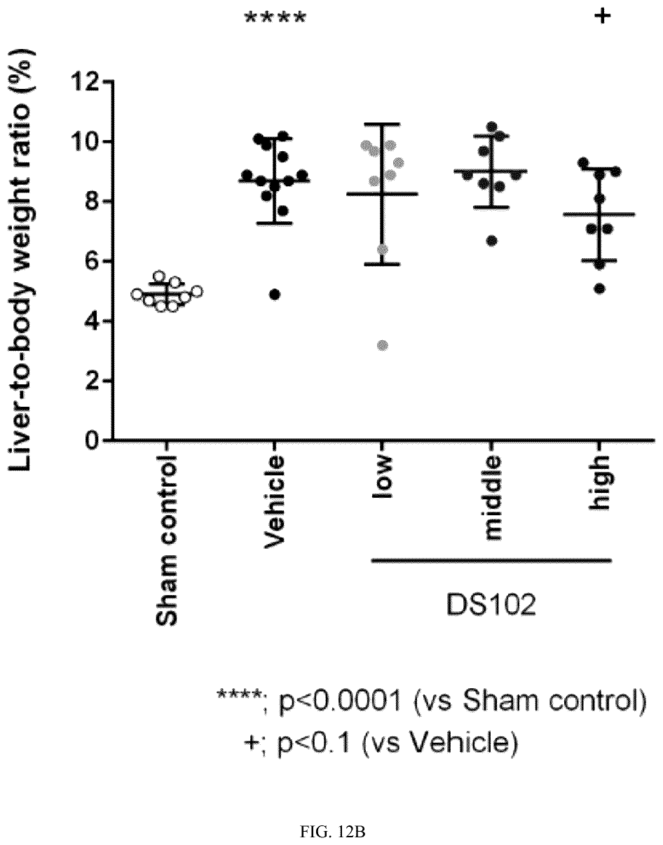

FIGS. 12A and 12B and Table 11 show the liver weight and liver-to-body weight ratio of the animals on the day of sacrifice. The Vehicle group showed a significant increase in mean liver weight compared with the Sham control group. Mean liver weight in the DS102 high group tended to decrease compared with the Vehicle group. There were no significant differences in mean liver weight between the Vehicle group and the other treatment groups. The Vehicle group showed a significant increase in mean liver-to-body weight ratio compared with the Sham control group. Mean liver-to-body weight ratio in the DS102 high group tended to decrease compared with the Vehicle group. There were no significant differences in mean liver-to-body weight ratio between the Vehicle group and the other treatment groups.

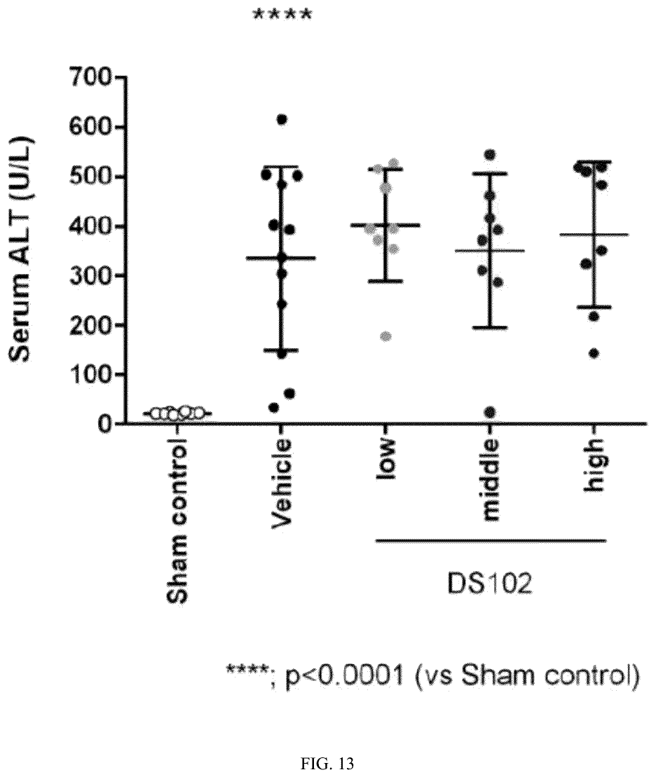

Biochemistry: FIG. 13 and Table 12 show the serum aminotransferase (ALT) for the animals. The Vehicle group showed a significant increase in serum ALT level compared with the Sham control group. There were no significant differences in serum ALT level between the Vehicle group and the DS102 treatment groups. However, from historical data for this model, ALT levels are known to decrease at Day 14 without treatment. As such, this may impact the ability to detect differences between the groups.

TABLE 12
Biochemistry

| | Serum | Serum Total |
|---|---|---|
| Group | ALT (U/L) | Bilirubin (mg/dL) |
| Sham control, (n = 8) | 22 ± 2 | 0.4 ± 0.1 |
| Vehicle, (n = 12) | 335 ± 185 | 24.9 ± 9.6 |
| DS102 low, (n = 8) | 402 ± 112 | 26.8 ± 6.9 |
| DS102 middle, (n = 8) | 351 ± 155 | 23.5 ± 9.8 |
| DS102 high, (n = 8) | 383 ± 147 | 26.2 ± 6.1 |

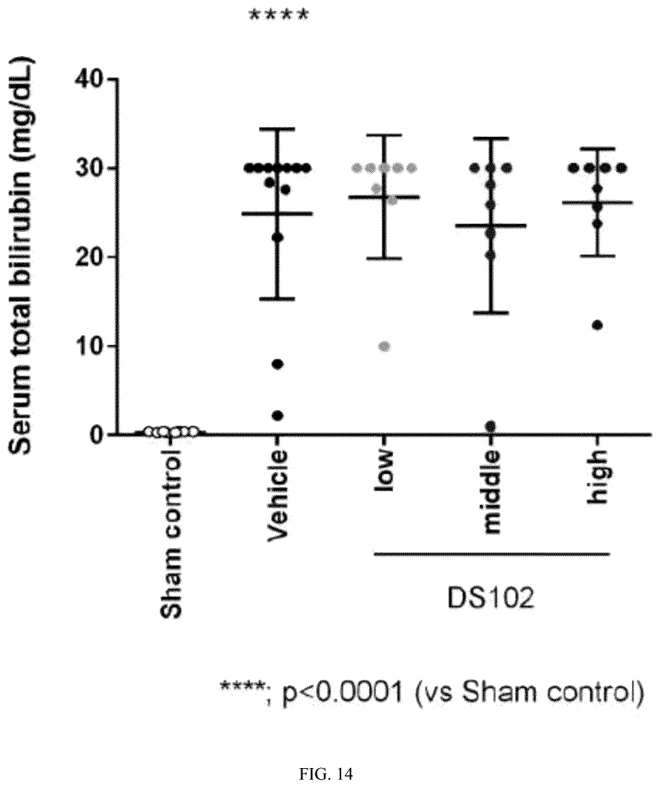

FIG. 14 and Table 12 show the serum total bilirubin for the animals. The Vehicle group showed a significant increase in serum total bilirubin level compared with the Sham control group. There were no significant differences in serum total bilirubin level between the Vehicle group and the DS102 treatment groups.

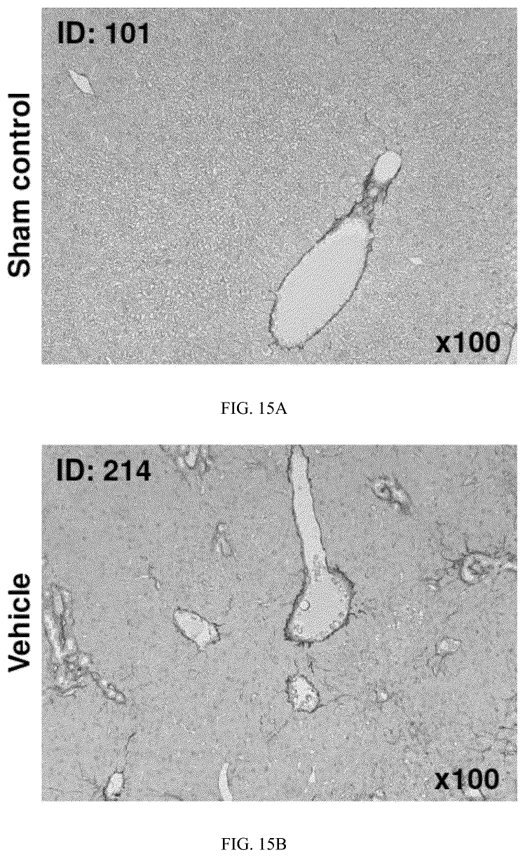

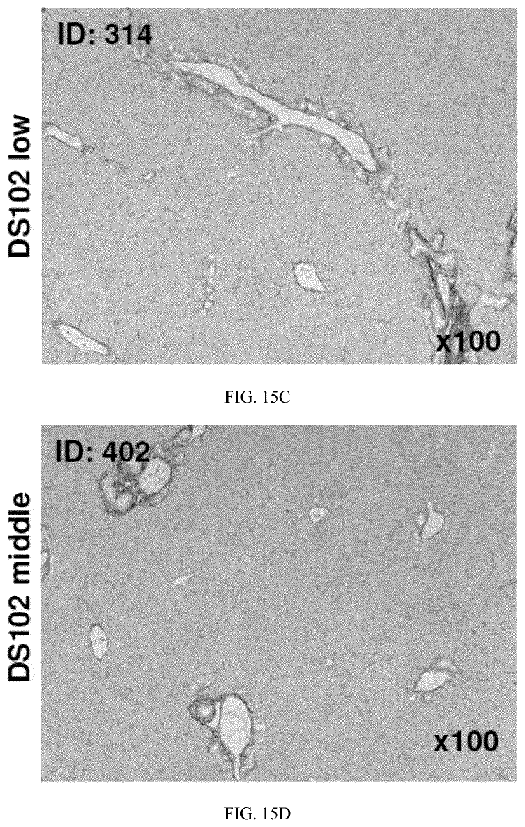

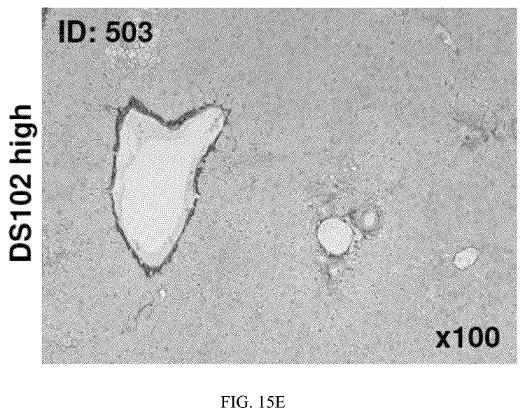

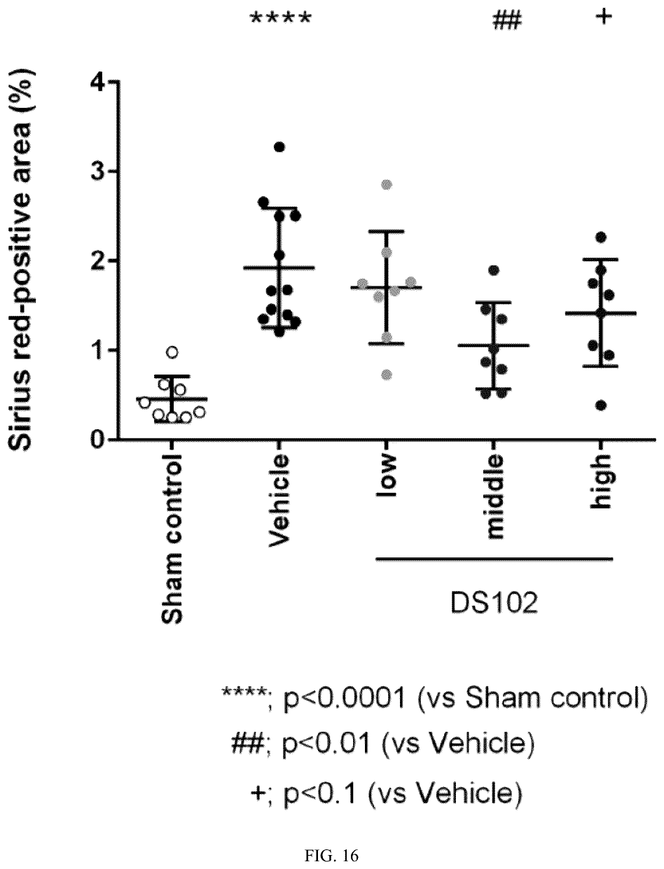

Histological Analysis: FIGS. 15A-15E and Table 13 show the Sirius red staining and FIG. 16, the fibrosis area of the animals. FIG. 14A displays representative of photomicrographs of Sirius red-stained liver sections. Liver sections from the Vehicle group showed increased collagen deposition in the portal region of liver lobule and PV—CV or PV-PV bridging fibrosis compared with the Sham control group. The Vehicle group showed a significant increase in the fibrosis area (Sirius red-positive area) compared with the Sham control group. The DS102 middle group showed a significant decrease in the fibrosis area compared with the Vehicle group. Fibrosis area in the DS102 high group tended to decrease compared with the Vehicle group. There was no significant difference in the fibrosis area between the Vehicle group and the DS102 low group.

TABLE 13
Histological Analysis

| | Sirius red- |
|---|---|
| Group | positive area (%) |
| Sham control, (n = 8) | 0.46 ± 0.25 |
| Vehicle, (n = 12) | 1.93 ± 0.67 |
| DS102 low, (n = 8) | 1.70 ± 0.63 |
| DS102 middle, (n = 8) | 1.06 ± 0.48 |
| DS102 high, (n = 8) | 1.42 ± 0.60 |

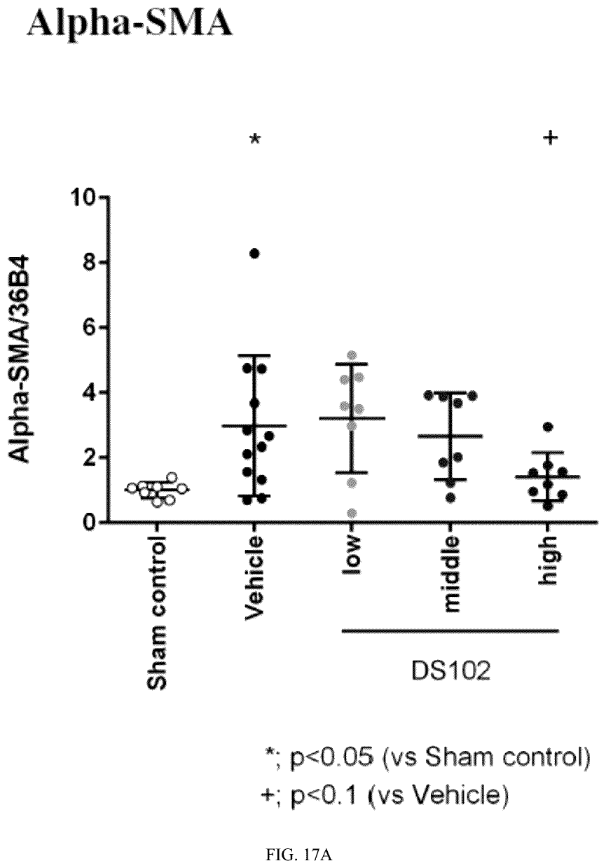

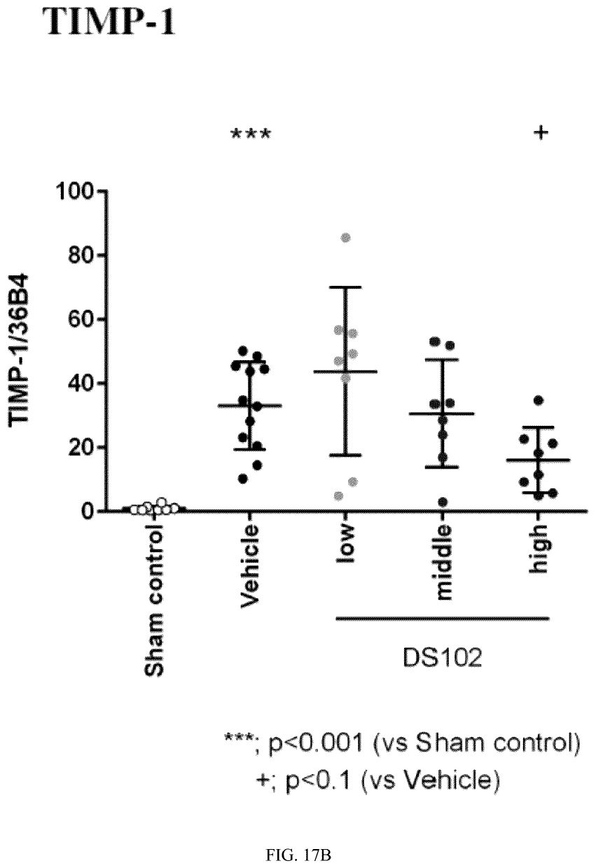

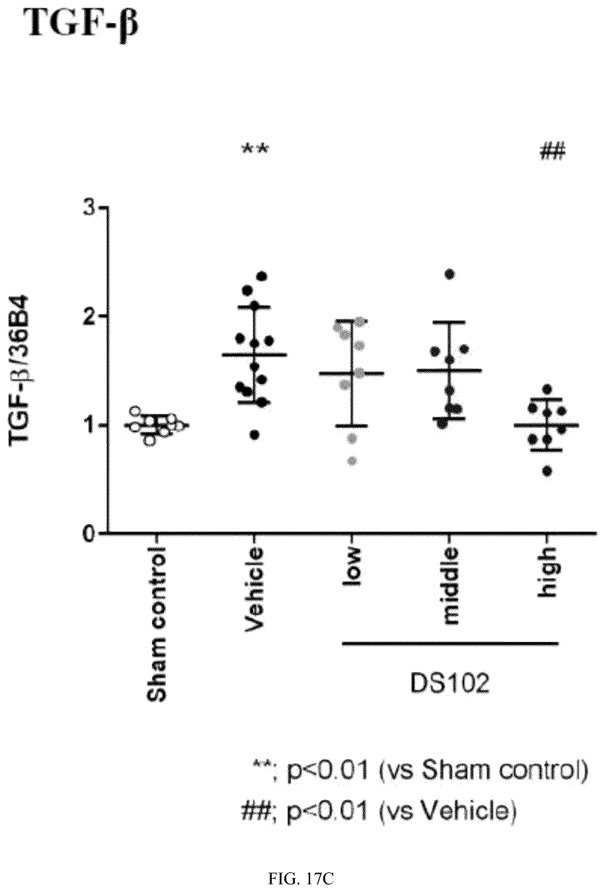

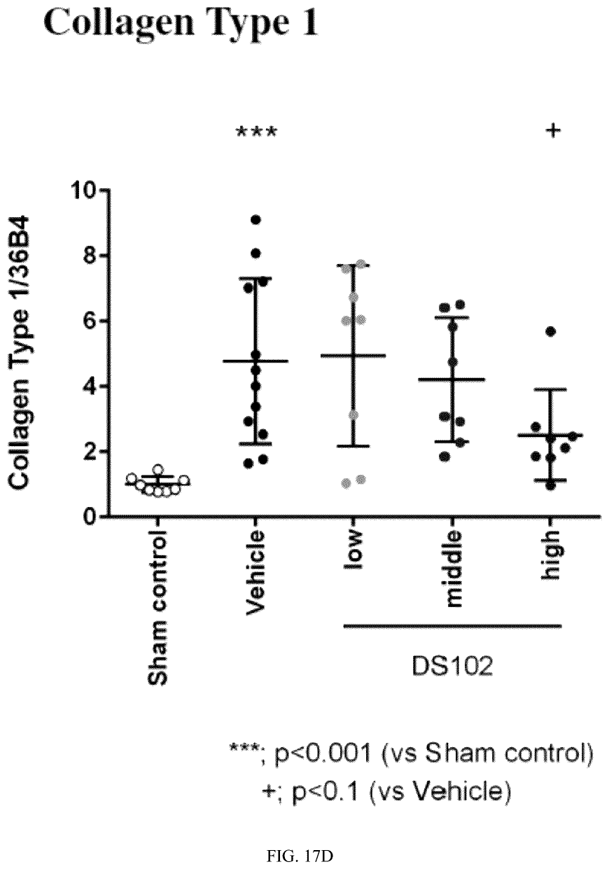

Gene Expression Analyses: Gene expression analyses for α-SMA, TIMP-1, TGF-β, and Collagen Type 1 are shown in FIGS. 17A-17D and Table 14.

TABLE 14
Gene Expression Analyses

| Group | Alpha-SMA | TIMP-1 | TGF-β | Collagen Type 1 |
|---|---|---|---|---|
| Sham control, (n = 8) | 1.00 ± 0.24 | 1.00 ± 0.82 | 1.00 ± 0.08 | 1.00 ± 0.24 |
| Vehicle, (n = 12) | 2.98 ± 2.16 | 33.09 ± 13.75 | 1.65 ± 0.44 | 4.77 ± 2.53 |
| DS102 low, (n = 8) | 3.21 ± 1.67 | 43.82 ± 26.19 | 1.48 ± 0.48 | 4.94 ± 2.76 |
| DS102 middle, (n = 8) | 2.66 ± 1.33 | 30.65 ± 16.80 | 1.50 ± 0.44 | 4.21 ± 1.90 |
| DS102 high, (n = 8) | 1.42 ± 0.75 | 16.09 ± 10.16 | 1.00 ± 0.23 | 2.51 ± 1.39 |

α-SMA: The Vehicle group showed a significant increase in the α-SMA mRNA expression level compared with the Sham control group. α-SMA mRNA expression level in the DS102 high group tended to decrease compared with the Vehicle group. There were no significant differences in α-SMA mRNA expression level between the Vehicle group and the DS102 treatment groups.

TIMP-1: The Vehicle group showed a significant increase in the TIMP-1 mRNA expression level compared with the Sham control group. TIMP-1 mRNA expression level in the DS102 high group tended to decrease compared with the Vehicle group. There were no significant differences in TIMP-1 mRNA expression level between the Vehicle group and the DS102 treatment groups.

TGF-β: The Vehicle group showed a significant increase in the TGF-β mRNA expression level compared with the Sham control group. The DS102 high group showed a significant decrease in the TGF-β mRNA expression level compared with the Vehicle group. There were no significant differences in TGF-β mRNA expression level between the Vehicle group and the DS102 treatment groups.

Collagen Type 1: The Vehicle group showed a significant increase in the Collagen Type 1 mRNA expression level compared with the Sham control group. Collagen Type 1 mRNA expression level in the DS102 high group tended to decrease compared with the Vehicle group. There were no significant differences in Collagen Type 1 mRNA expression level between the Vehicle group and the DS102 treatment groups.

### 2.4 Summary

In this study, biochemical parameters (e.g., ALT and total bilirubin), histological collagen deposition (e.g., fibrosis area) and gene expression levels (e.g., α-SMA, TIMP-1, TGF-β, Collagen Type 1) in the Vehicle group significantly increased compared with the Sham control group. These results suggested that cholestasis and liver fibrosis were established in the Vehicle group.

Treatment with DS102 at the middle dose showed a significant decrease (p<0.05) in fibrosis area compared with the Vehicle group. Treatment with DS102 at the high dose showed a significant decrease (p<0.05) in TGF-β mRNA expression level, and a trend approaching significance (p<0.01) for decrease in fibrosis area, liver weight, liver-to-body weight ratio, α-SMA, TIMP-1 and Collagen Type 1 mRNA expression levels compared with the Vehicle group. These results indicate that DS102 inhibits the production of multiple pro-fibrotic cytokines in the liver, has a suppressive effect on liver fibrosis and has potential as a therapeutic agent for cholestatic liver diseases.

### Example 3: Effects of DS102 on TGF-β Receptors, Signaling and Induced Fibrotic Proteins

The objective of this study was to examine the effects of 15-HEPE and 15-HEPE EE on the expression of TGF-β receptors, TGF-induced intracellular signaling and pro-fibrotic epithelial mesenchymal transition proteins.

### 3.1 Materials and Methods

Cytotoxicity testing: The cytotoxicity of 15-HEPE free acid and ethyl ester was tested in different liver (hepatoma) cell lines to understand the concentration range in the test system.

Transcriptional activity: A promoter (Luciferase) assay was conducted to measure TGFβ-induced transcriptional activation following administration of 15-HEPE.

Sucrose gradient ultracentrifugation and confocal microscopy were used to identify 15-HEPE induced microdomain translocation of TGF-β receptors by sucrose. Sucrose density gradient analysis of TGF-β receptors was conducted in the plasma membranes of Mv1Lu cells (mink lung epithelial cell) treated with 100 μM of 15-HEPE and an equal volume of DMSO (dimethyl sulfoxide) at 37° C. for 0, 1, 4, and 24 hours, and the cell lysates from these treated cells were subjected to sucrose density gradient ultracentrifugation. The sucrose gradient fractions were then analyzed by Western blot analysis using anti-TβR-I (type I TGF-β receptor), anti-TβR-II (type II TGF-β receptor), anti-TβR-III (type III TGF-β receptor, betaglycan), anti-EGFR (epidermal growth factor receptor), and anti-caveolin-1 antibodies. The lipid raft/caveolae, and non-lipid raft microdomain localization of TβR-I, TβR-II, TβR-III, EGFR and caveolin-1 in the plasma membrane of untreated cells or cells treated with 15-HEPE were assessed to determine the effects of 15-HEPE on the membrane microdomain localization of the TGF-β receptors.

### 3.2 Results

The effects of 15-HEPE on TGF-β-induced signaling and cellular responses were determined. To test the effect of 15-HEPE on TGF-β-induced signaling, the activities of 15-HEPE to regulate TGF-β-stimulated Smad2 phosphorylation and nuclear translocation, both of which are key signaling events leading to TGF-β-induced cellular responses, were tested. One important biological activity of TGF-β is transcriptional activation of genes responsible for epithelial-mesenchymal transition (EMT), which is a crucial event in wound healing, tissue fibrosis, and cancer progression. The effect of 15-HEPE on TGFβ-induced epithelial mesenchymal transition related proteins (such as fibronectin, PAI-1, and N-cadherin etc.) expression in HepG2 and LXR cells were assessed. HepG2 cells (human hepatoma cell line) were treated in increasing doses of 15-HEPE stage II in DMEM containing 0.1% of FBS for 1 hour and continually stimulated with or without 200 picomolar (pM) of TGF-β for 48 hours.

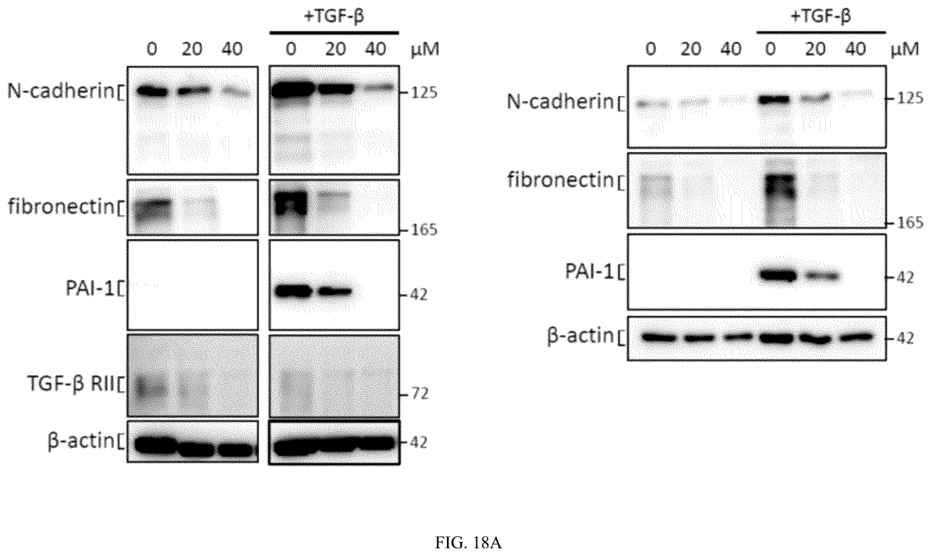

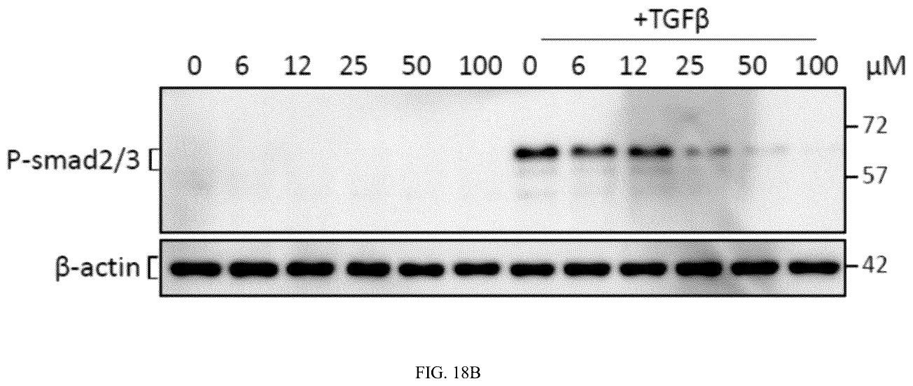

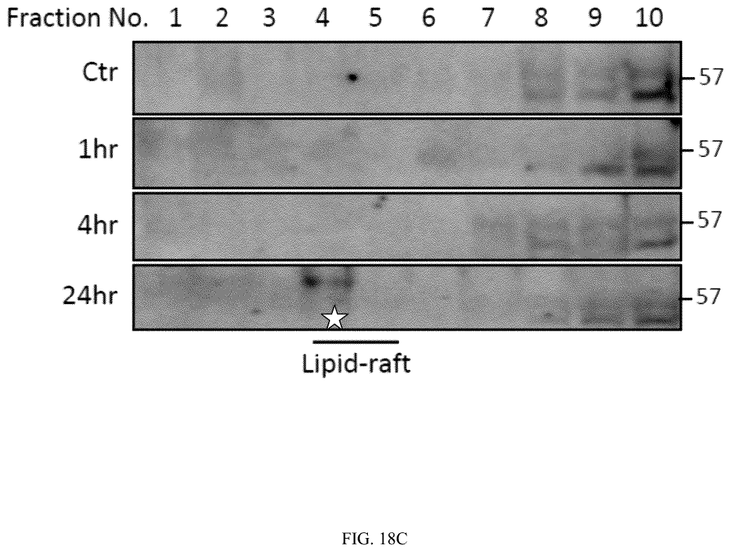

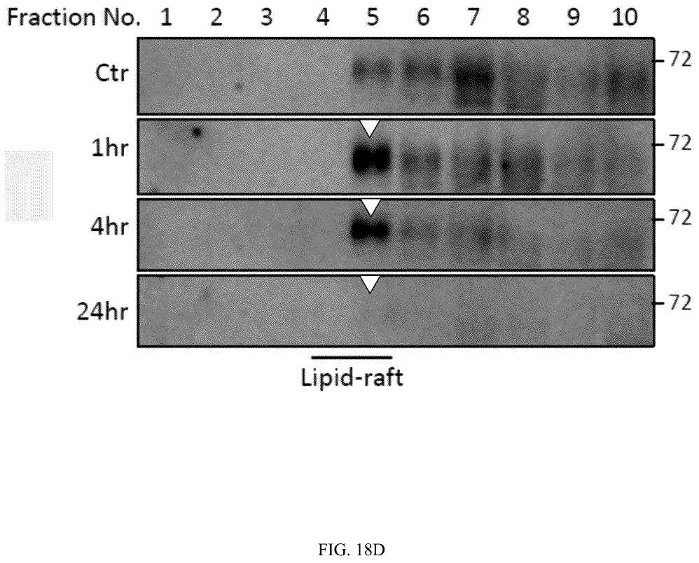

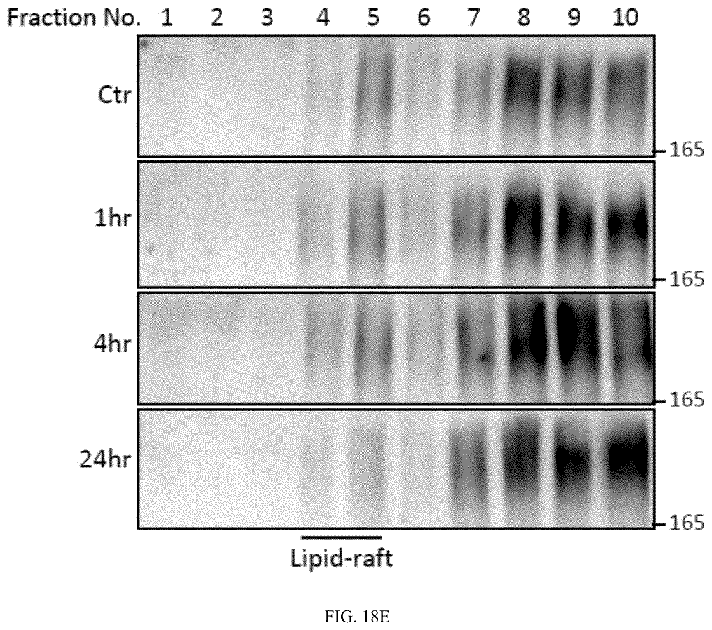

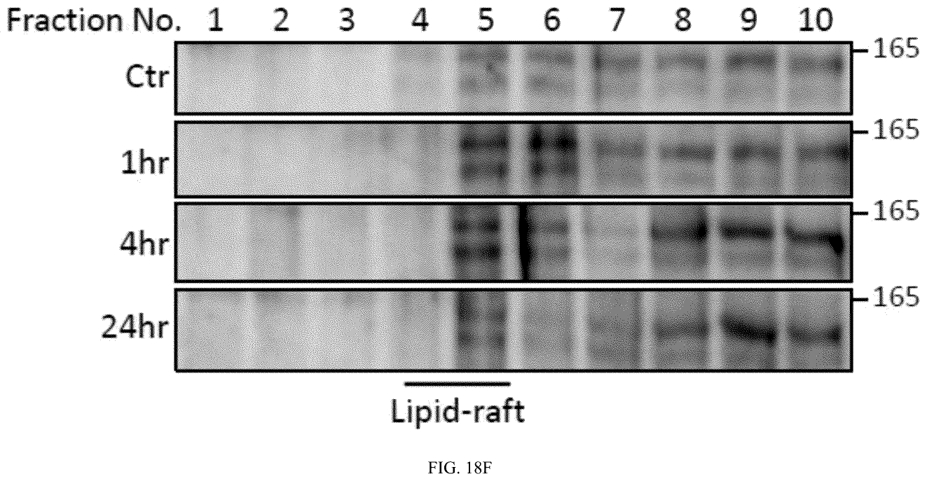

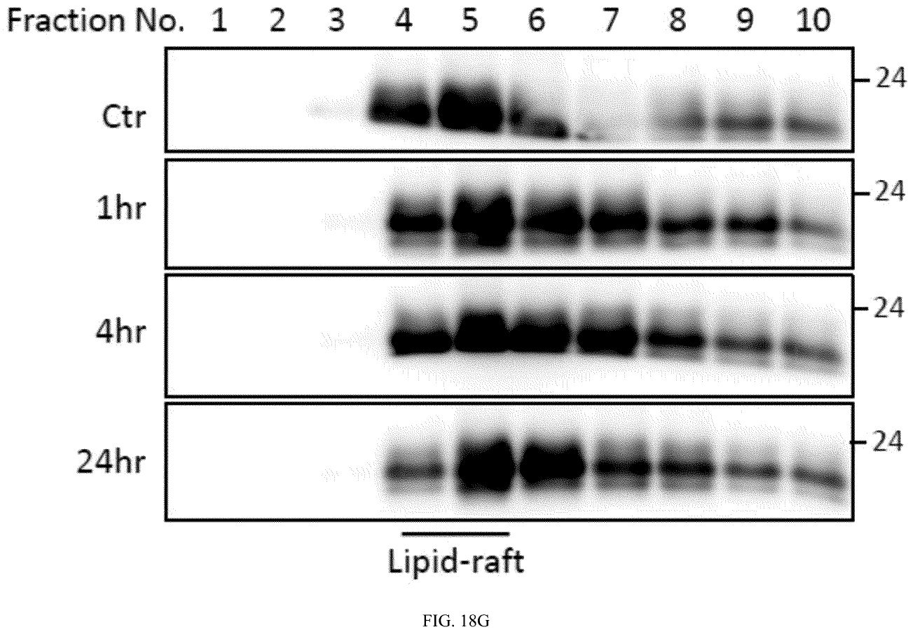

15-HEPE directly inhibited TGF-β signaling as shown in FIGS. 18A and 18B. Specifically, FIG. 18A shows that 15-HEPE induced degradation of type II TGF-β receptor and blocked TGF-βinduced epithelial mesenchymal transition (EMT) (i.e., pro-fibrotic) protein production. HepG2 cells (human hepatoma cell line) were treated in increasing doses of 15-HEPE stage II in DMEM containing 0.1% of FBS for 1 hour and continually stimulated with or without 200 picomolar (pM) of TGF-β for 48 hours. The two panels shown in FIG. 18A are two separate experiments run under the same experimental conditions. The results from these separate experiments indicate that 15-HEPE blocks TGF-β induced EMT protein production and induced degradation of type II TGF-β receptor. FIG. 18A also shows the effects of 15-HEPE on plasminogen activator inhibitor-1 (PA-1), a protein induced by TGF-β and associated with increased cardiovascular risk. FIG. 18B shows that 15-HEPE inhibits TGF-β-stimulated intracellular signaling (e.g., SMAD2/3 phosphorylation) in liver stellate cells. The experiment conducted in FIG. 18B included pretreating LX2 cells (human liver stellate cells) with increasing concentrations between 0 μM to 100 μM of DS102 for 24 hours followed by 30 min of TGF-β stimulation. The results of the experiment indicated that 15-HEPE inhibit TGF-β stimulation.

A 15-HEPE sucrose density gradient analysis of TGF-β receptors was conducted in the plasma membranes of mink lung epithelial cell (Mv1Lu) cells treated with 100 UM of 15-HEPE and an equal volume of dimethyl sulfoxide (DMSO) at 37° C. for 0, 1, 4, and 24 hours, and the cell lysates from these treated cells were subjected to sucrose density gradient ultracentrifugation. The sucrose gradient fractions were then analyzed by Western blot analysis using anti-TβR-I (type I TGF-β receptor-FIG. 18C), anti-TβR-II (type II TGF-β receptor-FIG. 18D), anti-TβR-III (type III TGF-β receptor, betaglycan-FIG. 18E), anti-EGFR (epidermal growth factor receptor-FIG. 18F), and anti-caveolin-1 antibodies (FIG. 18G). Fractions 4 to 5 contained lipid rafts/caveolae whereas fractions 7-10 were non-lipid raft fractions. Treatment with 15-HEPE did not affect the abundance of TβR-I proteins but induced translocation of TβR-I to lipid-raft at 24 hours treatment (FIG. 18C). Stars(*) indicate 15-HEPE increased abundance of TβR-I (24 hours) in the fraction in comparison with that of the control and shorter treatment durations (FIG. 18C). 15-HEPE induced TβR-II translocation from 1 to 4 hours and further induce degradation at 24 hours treatment (FIG. 18D). The triangles (V) indicate translocation and decreased abundance of TβR-II in the fraction in comparison with control cells (FIG. 18D). In FIGS. 18E, 18F and 18G, 15-HEPE did not change the localization and abundance of TβR-III, EGFR and caveolin-1.

### 3.3 Summary

Accordingly, taken together, these results demonstrate that 15-HEPE in both the free acid or ethyl ester form, induced translocation and degradation of type II TGF-β receptors, blocked TGF-β induced pro-fibrotic protein production, and inhibited TGF-β induced intracellular signaling (SMAD2/3) in liver stellate cells. As such, 15-HEPE and 15-HEPE EE can directly inhibit TGF-β signaling, results which support the therapeutic potential for 15-HEPE and 15-HEPE EE in treating multiple fibrotic diseases including non-alcoholic steatohepatitis (NASH), other fibrotic diseases, cardiometabolic diseases, as well as multiple indications for cancer.

### Example 4: The Efficacy of Orally Administered DS102 in Non-Alcoholic Fatty Liver Disease (NAFLD) Patients

The objective of the study was to assess the safety and efficacy of orally administered DS102 capsules versus placebo in the treatment of adult patients with NAFLD.

### 4.1 Study Endpoints

Primary Endpoints: The primary endpoints for this study included the efficacy as well as the safety for administering DS102. The efficacy was evaluated based on change in serum alanine aminotransferase (ALT) from baseline to Week 16 and change in liver stiffness measured by transient elastography from baseline to Week 16. The safety was evaluated on the number of treatment emergent adverse events (TEAEs) in each treatment group leading to treatment discontinuation.

Secondary Endpoints: The secondary endpoints for this study included a change in any one of the following: serum ALT from baseline to Weeks 2, 4, 8 and 12; aspartate aminotransferase (AST) from baseline to Weeks 2, 4, 8, 12 and 16; AST:ALT ratio from baseline to Weeks 2, 4, 8, 12 and 16; fibrosis-4 (FIB-4) index from baseline to Week 16; NAFLD fibrosis score (NFS) from baseline to week 16; change in hepatic fat measured by controlled attenuation parameter (CAP) from baseline to Week 16; enhanced liver fibrosis (ELF) score from baseline to Week 16; and homeostatic model assessment insulin resistance (HOMA-IR) and adipose tissue insulin resistance (adipo-IR) from baseline to Weeks 2, 4, 8, 12 and 16.

Exploratory analysis: The exploratory analysis included analysis of lipid and metabolic parameters including total cholesterol, triglycerides, very low-density lipoprotein cholesterol (VLDL-C), low density lipoprotein cholesterol (LDL-C), high density lipoprotein cholesterol (HDL-C), non-high-density lipoprotein cholesterol (non-HDL-C), remnant-like particle cholesterol (RLP-C), fasting glucose, insulin, free fatty acids and hemoglobin A1C (HbA1C). Additional exploratory analysis included high throughput lipidomics and proteomics.

### 4.2 Study Design

This was a randomized, placebo-controlled, double-blind, parallel group, multi-center exploratory phase IIa study to investigate the safety and efficacy of orally administered DS102 capsules and the dose-response relationship between two doses of DS102 and placebo in NAFLD patients aged 18 to 75 years. Three parallel groups of patients with confirmed NAFLD were investigated in this study to compare two different doses of DS102 with placebo over a 16-week treatment period. The study was planned to include 96 evaluable patients with 32 patients randomized per treatment group.

The study consisted of a screening period of 28 days, a 16-week treatment period and a 4 week follow up period. At the screening visit, patients were assessed using the screening examinations. Patients who meet the inclusion criteria and who do not meet the exclusion criteria were enrolled.

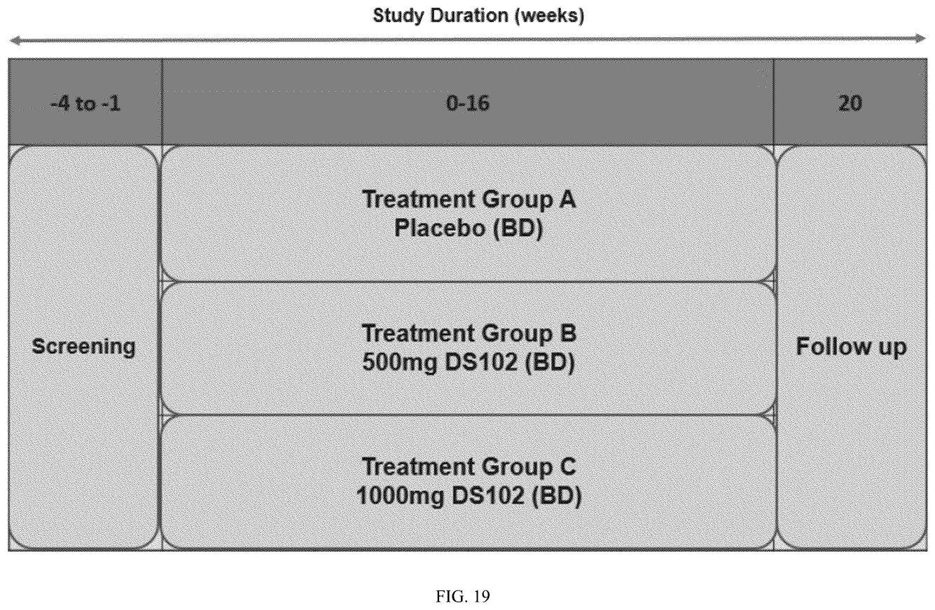

A schematic diagram of the overall timeframe of the study is provided in FIG. 19. Once patients were enrolled in the study they were restricted from using any other treatment for NAFLD. Any medication (e.g., prescription as well as over the counter (OTC) drugs) or therapeutic intervention deemed necessary for the patient, and which in the opinion of the Investigator do not interfere with the safety and efficacy evaluations, were continued unless they are included in the list of ‘Concomitant Medications’ provided below.

Before the comparative treatment period commenced, patients returned to the site for a baseline assessment of their disease and eligible patients were randomly allocated to one of the three parallel group treatment regimens in a 1:1:1 randomization:
Treatment group A: 2×Placebo 500 mg capsules orally administered twice a day (4 capsules daily) for 16 weeks.
Treatment group B: 1×DS102 500 mg capsule & 1×Placebo 500 mg capsule orally administered twice a day (4 capsules daily) for 16 weeks.
Treatment group C: 2×DS102 500 mg capsules orally administered twice a day (4 capsules daily) for 16 weeks.

To maintain the double-blind conditions, the DS102 capsule and placebo capsule were identical in appearance.

### 4.3 Patients and Screening

In order to participate in this study, the patients were required to meet all of the following inclusion criteria and must not have met any of the following exclusion criteria. The inclusion and exclusion criteria were verified at the screening visit (Visit 1) and at the start of treatment/baseline visit (Visit 2).

Inclusion Criteria for this study were as follows:
Patients diagnosed with NAFLD by the presence of hepatic steatosis on imaging or histology in the absence of any secondary causes.
Patients with an ALT≥1.5 ULN and <5 ULN on two occasions 7 or more days apart during screening.
Patients with historical liver biopsy showing NASH and/or ≥F1 fibrosis or NFS≥-1.455 or Fib-4≥1.3 or Fibroscan≥8 kPa within 3 months of screening.
Patients with a body mass index (BMI) between 25.0 and 40.0 kg/m². Patients with a history of controlled obesity or controlled diabetes were allowed in the study.
Patients whose pre-study clinical laboratory findings did not interfere with their participation in the study, in the opinion of the Investigator.
Patients aged between 18 and 75 years.
Female patients and male patients with female partners of child bearing potential were required to use adequate contraception or have a sterilized partner for the duration of the study. Adequate contraception is defined as: systemic hormonal contraceptives; intrauterine device or barrier method of contraception in conjunction with spermicide; or agree to sexual abstinence, defined as a patient refraining from heterosexual intercourse during the entire period of risk associated with the study treatments and in line with their preferred and usual lifestyle. Hormonal contraceptives were required to be on a stable dose for at least one month before baseline.
Patients who were able to communicate well with the Investigator, to understand and comply with the requirements of the study and understand and sign the written informed consent.

Exclusion Criteria for this study were as follows:
Patients with an unstable metabolic condition such as weight change>5% in the 3 months prior to inclusion.
Patients with medical/surgical history of gastric bypass surgery, orthotopic liver transplant (OLT) or listed for OLT.
Patients with uncontrolled diabetes mellitus type 2, i.e., HbA1c≥9% (75 mmol/mol) at the time of screening.
Patients with decompensated or severe liver disease as evidenced by one or more of the following: confirmed cirrhosis or suspicion of cirrhosis, esophageal varices, ascites, suspicion of portal hypertension, hospitalization for liver disease within 60 days of screening, bilirubin≥2×ULN, or ALT or AST≥5×ULN. Patients with Gilbert's syndrome were eligible if the conjugated bilirubin was ≤1.5×ULN.
Patients with inflammatory bowel disease that was either active or requiring medical therapy.
Patients with diagnosed or suspected autoimmune diseases such as systemic lupus erythematosus (SLE) and/or rheumatoid arthritis (RA).
Patients with a history of or active non-liver malignancies other than curatively treated skin cancer (basal cell or squamous cell carcinomas).
Patients with a significant systemic or major illness other than liver disease, including coronary artery disease, cerebrovascular disease, pulmonary disease, renal insufficiency, serious psychiatric disease, respiratory or hypertensive disease, as well as diabetes and arthritis that, in the opinion of the Investigator, precluded the patient from participating in and completing the study.
Patients requiring anti-diabetic treatment (including insulin sensitizing agents), and/or lipid lowering treatment, and who were not on a stable dose for at least 3 months prior to screening were excluded. If patients were insulin dependent this treatment should have commenced at least 3 months prior to screening, however changes in dose were permitted.
Patients with known hypersensitivity to any ingredients of the study treatment.
Patients with a positive test for human immunodeficiency virus (HIV) antibodies, Hepatitis B surface antigen or Hepatitis C antibodies at screening.
Patients with liver disease of other etiologies such as drug-induced, autoimmune hepatitis, primary biliary cholangitis (PBC), primary sclerosing cholangitis (PSC), haemochromatosis, alpha-1 antitrypsin (A1AT) deficiency or Wilson's disease.
Patients with a significant history of drug/solvent abuse, in the opinion of the Investigator.
Patients with a history of alcohol abuse in the opinion of the Investigator, or who drink in excess of 21 units per week (males) or 14 units per week (females), whereby a unit consists of 10 ml or 8 mg of pure alcohol.
Patients who used dietary supplements rich in omega-3 or omega-6 fatty acids in the 4 weeks prior to baseline.
Patients who have participated in any other clinical study with an investigational drug within 3 months before the first day of administration of study treatment.
Patients who are pregnant, planning pregnancy, breastfeeding and/or were unwilling to use adequate contraception during the trial.
Patients, in the opinion of the Investigator, not suitable to participate in the study.

### 4.4 Study Conduct

During the study, ten visits to the clinic were scheduled after the screening visit: one at the start of the comparative treatment period/baseline (Day 0/Visit 2) and eight in the comparative treatment period (Week 2/Visit 3, Week 4/Visit 4, Week 6/Visit 5, Week 8/Visit 6, Week 10/Visit 7, Week 12/Visit 8, Week 14/Visit 9, Week 16/Visit 10). A final safety follow-up visit (Visit 11) was conducted four weeks after Visit 10 or two weeks after the final visit attended if the patient did not complete the study. Table 15 describes the clinical visits for the study.

TABLE 15
Study Flow Chart of The Clinical Visits for The Study

| | | | | | | | | | | EOT/ | |
|---|---|---|---|---|---|---|---|---|---|---|---|
| | | | Visit | Visit | Visit | Visit | Visit | Visit | Visit | Visit | Follow |
| | | | 3/ | 4/ | 5/ | 6/ | 7/ | 8/ | 9/ | 10/ | Up/ |
| | | | Week | Week | Week | Week | Week | Week | Week | Week | Visit |
| | | | 2 | 4 | 6 | 8 | 10 | 12 | 14 | 16 | 11/Week |
| Study | Screening/ | | Day | Day | Day | Day | Day | Day | Day | Day | 20 |
| Procedure | Visit 1 | Baseline/ | 14 | 28 | 42 | 56 | 70 | 84 | 98 | 112 | Day 140 |
| Visit | Day −28 | Visit 2 | +/−2 | +/−2 | +/−2 | +/−2 | +/−2 | +/−2 | +/−2 | +/−2 | +/−4 |
| Window | to −1 | Day 0 | days | days | days | days | days | days | days | days | days |
| Informed consent | X | | | | | | | | | | |
| Inclusion Exclusion | X | X | | | | | | | | | |
| Demography | X | | | | | | | | | | |
| Medical history | X | X | | | | | | | | | |
| Physical | X | X | X | X | | X | | X | | X | X |
| examination | | | | | | | | | | | |
| 12-lead ECG | X | X | | | | | | | | X | |
| Plasma PK | | X | X | X | | X | | X | | X | X |
| sampling ¹ | | | | | | | | | | | |
| Vital signs | X | X | X | X | | X | | X | | X | X |
| Clinical laboratory | X | X | | X | | X | | X | | X | X |
| tests ² | | | | | | | | | | | |
| Lipid Profile ³ | | X | | | | X | | | | X | |
| Urinalysis | | X | | | | | | | | X | |
| Virology | X | | | | | | | | | | |
| Pregnancy test ⁴ | X | X | | X | | X | | X | | X | X |
| ALT, AST ⁵ | XX | X | X | X | | X | | X | | X | X |
| HOMA-IR/Adipo-IR | | X | X | X | | X | | X | | X | X |
| ELF | | X | | | | | | | | X | |
| Liver stiffness and | | X | | | | | | | | X | |
| CAP | | | | | | | | | | | |
| FIB-4 | | X | | | | | | | | X | |
| NFS [including | | X | | | | | | | | X | |
| BMI] | | | | | | | | | | | |
| Biomarkers Blood | | X | | | | X | | | | X | X |
| Sample | | | | | | | | | | | |
| Exploratory Blood | | X | | | | | | | | X | |
| Sample | | | | | | | | | | | |
| Patient | | X | | | | | | | | | |
| Randomisation | | | | | | | | | | | |
| IMP/Placebo | | X | X | X | X | X | X | X | X | | |
| Dispensing | | | | | | | | | | | |
| Study drug/placebo | | X---------------------------------------------------------------------------------------------------------- | | | | | | | | | |
| administration | | ----------X | | | | | | | | | |
| IMP | | X | X | X | X | X | X | X | X | X | |
| Accountability/Diary | | | | | | | | | | | |
| Card review | | | | | | | | | | | |

| AE assessment | | X-------------------------------------------------------------------------------------------------------------------------- | |
|---|---|---|---|
| | | -------------------------X | |
| Concomitant | X | | X--------------------------------------------------------------------- |
| medication | | | ----------------------------------------------------------------------- |
| assessment | | | ---------------------X |

¹ Pharmacokinetics Trough only. Sample was taken pre-dose.
² Includes biochemistry, haematology and coagulation tests. This was taken fasting (Minimum of 8 hours).
³ Lipid Profile was taken fasting (Minimum of 8 hours).
⁴ Female Patients of child bearing potential only.
⁵ ALT to be assessed on two occasions during screening 7 or more days apart.

Screening Visit (Visit 1): Once informed consent was obtained, patients were assigned a Patent Screen Number. Ideally the patient was fasted. The following screening assessments/sample collections were performed: verification of inclusion/exclusion criteria; demographic data; medical history; physical examination; 12-lead electrocardiogram (ECG); Vital signs (blood pressures, heart rate and body temperature); samples for clinical laboratory safety tests (hematology, serum biochemistry, and coagulation tests); virology; pregnancy test (for female patients of child-bearing potential); ALT, AST tests (ALT measured on two occasions during screening); and concomitant medication assessment. Unscheduled visits occurred when a patient needed to make a visit in between the scheduled visit dates due to an adverse event (AE), difficulty complying with the study protocol requirements, or a significant change in their disease state. All procedures that were medically necessary were followed. If qualified, before leaving the clinic the patient was instructed not to have any breakfast before the next visit to allow a minimum fasting period of 8 hours.

Treatment Period: Following completion of a successful screening visit, patients began the comparative treatment period (16 weeks). At the start of the comparative treatment period, after confirmation of continued eligibility, patients were randomly assigned to one of the three treatment regimens. Patients took the allocated investigation medicinal product (IMP) of a DS102 capsule or placebo capsule twice-daily throughout the comparative treatment period. Each self-administration of IMP was recorded in a patient diary card. Patients were instructed to take DS102 in the morning and in the evening with or after food (except on the mornings of clinic visits 3, 4, 6, 8 and 10 when patients were instructed to abstain from taking DS102 prior to the visit and to take DS102 as soon as possible after the clinic visit). At the start of the baseline (Visit 2), Week 2 (Visit 3), Week 4 (Visit 4), Week 8 (Visit 6), Week 12 (Visit 8), Week 16 (Visit 10), and Week 20 (Visit 11), the patient was asked if they had fasted for a minimum of 8 hours prior to the visit. If this was not the case, the duration of fasting period was documented, and the patient reinstructed about the duration of the fasting period. The patient was then provided with a light breakfast (e.g., tea or orange juice and toast). At baseline (Visit 2), Week 8 (Visit 8), and Week 16 (Visit 10), the blood sampling assessment was carried out prior to the patient receiving the light breakfast. Unscheduled visits occurred when a patient needed to make a visit in between the scheduled visit dates due to an adverse event (AE), difficulty complying with the study protocol requirements, or a significant change in their disease state. All procedures that were medically necessary were followed. Patients who discontinued the study early had study procedures scheduled for Visit 10 performed as soon as possible after patient withdrawal so that all study-related information could be recorded. At the discretion of Investigator, urine DOA and alcohol breath tests were performed at any time during the conduct of the trial.

Baseline (Visit 2): Patients attended the investigational site at Visit 2. Blood sampling was the first assessment carried out. After the blood sampling, the following assessments were performed: verification of inclusion/exclusion criteria; medical history; physical examination; 12-lead ECG; pharmacokinetic sampling; vital signs (blood pressures, heart rate and body temperature); samples for clinical laboratory safety tests (haematology, serum biochemistry, and coagulation tests); lipid profile; urinalysis; pregnancy test (for female patients of child-bearing potential); ALT, AST tests; HOMA-IR/Adipo-IR; ELF; liver stiffness and CAP; FIB-4; NFS (including BMI); biomarkers blood sample; exploratory blood sample; patient randomization; study drug/placebo administration; AE assessment; and concomitant medication assessment. If all study entry criteria were satisfied the Investigator randomized the patient and provided the patient with the designated IMP or placebo from one of the patient treatment packs. The first dose of IMP or placebo was administered at site once all baseline assessments had been completed. The patient took their second dose of IMP or placebo in the evening of Day 0. The capsules were then administered twice-daily. Patients did take IMP or placebo on the morning of their return site visit (Visit 3). Before leaving the clinic, the patient was instructed not to have any breakfast before the next visit to allow a minimum fasting period of 8 hours.

Week 2 (Visit 3): Patients returned to the investigational site at Visit 3. Patients did not take IMP or placebo on the morning of Visit 3. The following assessments were performed: physical examination; pharmacokinetic sampling; vital signs (e.g., blood pressures, heart rate and body temperature); ALT, AST tests; HOMA-IR/Adipo-IR; AE assessment; and concomitant medication assessment. The IMP or placebo was returned, and further IMP or placebo was supplied to the patient. The patient took their next dose of IMP or placebo as soon as all visit assessments had been completed. The capsules continued to be administered twice-daily. On completion of this visit, patients were advised that they were required to return to the investigational site in two weeks at Visit 4. Patients did not take IMP or placebo on the morning of their return site visit (Visit 4). Before leaving the clinic, the patient was instructed not to have any breakfast before the next visit to allow a minimum fasting period of 8 hours.

Week 4 (Visit 4): Patients returned to the investigational site at Visit 4. Patients did not take IMP or placebo on the morning of Visit 4. The following assessments were performed: physical examination; pharmacokinetic sampling; vital signs (blood pressures, heart rate and body temperature); samples for clinical laboratory safety tests (haematology, serum biochemistry and coagulation tests); pregnancy test (for female patients of child-bearing potential); ALT, AST tests; HOMA-IR/Adipo-IR; AE assessment; and concomitant medication assessment. The IMP or placebo was returned, and further IMP or placebo was supplied to the patient. The patient took their next dose of IMP or placebo as soon as all visit assessments had been completed. The capsule continued to be administered twice-daily. On completion of this visit, patients were advised that they were required to return to the investigational site in two weeks at Visit 5. Patients did not take IMP or placebo on the morning of their return site visit (Visit 5).

Week 6 (Visit 5): Patients returned to the investigational site at Visit 5. The following assessments were performed: AE assessment and Concomitant medication assessment. The IMP or placebo was returned and further IMP or was supplied to the patient. The patient took their next dose of IMP or placebo as soon as all visit assessments had been completed. The capsule continued to be administered twice-daily. On completion of this visit, patients were advised that they were required to return to the investigational site in two weeks at Visit 6. Patients did not take IMP or placebo on the morning of their return site visit (Visit 6). Before leaving the clinic, the patient was instructed not to have any breakfast before the next visit to allow a minimum fasting period of 8 hours.

Week 8 (Visit 6): Patients returned to the investigational site at Visit 6. Patients did not take IMP or placebo on the morning of Visit 6. Blood sampling was the first assessment carried out. After the blood sampling, the following assessments were performed: physical examination; pharmacokinetic sampling; vital signs (blood pressures, heart rate and body temperature); samples for clinical laboratory safety tests (haematology, serum biochemistry and coagulation tests); lipid profile; pregnancy test (for female patients of child-bearing potential); ALT, AST tests; HOMA-IR/Adipo-IR; biomarker blood samples; ae assessment; and concomitant medication assessment. The IMP or placebo were returned and further IMP or supplied to the patient. The patient took their next dose of IMP or placebo as soon as all visit assessments had been completed. The capsule continued to be administered twice-daily. On completion of this visit, patients were advised that they were required to return to the investigational site in two weeks at Visit 7. Patients did take IMP or placebo on the morning of their return site visit (Visit 7).

Week 10 (Visit 7): Patients returned to the investigational site at Visit 7. The following assessments were performed: AE assessment and concomitant medication assessment. The IMP or placebo were returned, and further IMP or placebo was supplied to the patient. The patient took their next dose of IMP or placebo as soon as all visit assessments had been completed. The capsule continued to be administered twice-daily. On completion of this visit, patients were advised that they were required to return to the investigational site in two weeks at Visit 8. Patients did not take IMP or placebo on the morning of their return site visit (Visit 8). Before leaving the clinic, the patient was instructed not to have any breakfast before the next visit to allow a minimum fasting period of 8 hours.

Week 12 (Visit 8): Patients returned to the investigational site at Visit 8. Patients did not take IMP or placebo on the morning of Visit 8. The following assessments were performed: physical examination; pharmacokinetic sampling; vital signs (blood pressures, heart rate and body temperature); samples for clinical laboratory safety tests (haematology, serum biochemistry, and coagulation tests); pregnancy test (for female patients of child-bearing potential); ALT, AST tests; HOMA-IR/Adipo-IR; AE assessment; and concomitant medication assessment. The IMP or placebo was returned, and further IMP or placebo supplied to the patient. The patient took their next dose of IMP or placebo as soon as all visit assessments had been completed. The capsule continued to be administered twice-daily. On completion of this visit, patients were advised that they were required to return to the investigational site in two weeks at Visit 9. Patients did not take IMP or placebo on the morning of their return site visit (Visit 9).

Week 14 (Visit 9): Patients returned to the investigational site at Visit 9. The following assessments were performed: AE assessment and concomitant medication assessment. The IMP or placebo were returned, and further IMP or placebo supplied to the patient. The patient took their next dose of IMP or placebo as soon as all visit assessments had been completed. The capsule continued to be administered twice-daily. On completion of this visit, patients were advised that they were required to return to the investigational site in two weeks at Visit 10 Patients should not take IMP or placebo on the morning of their return site visit (Visit 10). Before leaving the clinic, the patient was instructed not to have any breakfast before the next visit to allow a minimum fasting period of 8 hours.

Week 16 (Visit 10) or Early Withdrawal: Patients returned to the investigational site at Visit 10. Patients did not take IMP or placebo on the morning of Visit 10. Blood sampling was the first assessment carried out. After the blood sampling, the following assessments were performed: physical examination; 12-lead ECG; pharmacokinetic sampling; vital signs (blood pressures, heart rate and body temperature); samples for clinical laboratory safety tests (haematology, serum biochemistry, and coagulation tests); urinalysis; lipid profile; pregnancy test (for female patients of child-bearing potential); ALT, AST tests; HOMA-IR/Adipo-IR; ELF; liver stiffness and CAP; FIB-4; NFS [including BMI]; Biomarker blood samples; exploratory blood sample; AE assessment; and concomitant medication assessment. The IMP or placebo were returned. No further IMP or placebo blister packs or patient diary cards were issued. Following completion of the study assessments at this visit, there were continued study restrictions. On completion of this visit, patients were advised that they were required to return to the investigational site in four weeks at Visit 11 to assess any AEs since this visit and conduct safety and efficacy assessments. Before leaving the clinic, the patient was instructed not to have any breakfast before the next visit to allow a minimum fasting period of 8 hours.

Follow Up Visit (Week 20/Visit 11): Four weeks after Visit 10 (or 2 weeks after early withdrawal visit), patients returned to the investigational site. The following assessments were carried out: physical examination; pharmacokinetic sampling; vital signs (blood pressures, heart rate and body temperature); samples for clinical laboratory safety tests (haematology, serum biochemistry and coagulation tests); pregnancy test (for female patients of child-bearing potential); ALT, AST tests; HOMA-IR/Adipo-IR; biomarkers; AE assessment; and concomitant medication assessment.

### 4.5 Assessments

Efficacy assessments included: ALT, AST, ALT:AST ratio; HOMA-IR/Adipo-IR; ELF; liver stiffness and CAP; FIB-4 index; and NFS. A detailed description of each is provided below.

ALT, AST, ALT:AST ratio: Increased liver enzymes (ALT and AST) are a marker of liver injury and were assessed at Visit 1/Screening (on two occasions during screening 7 or more days apart), Visit 2/Baseline, Visit 3/Week 2, Visit 4/Week 4, Visit 6/Week 8, Visit 8/Week 12, Visit 10/Week 16 and Follow up Visit 11/Week 20.

HOMA-IR/Adipo-IR: HOMA-IR/Adipo-IR levels are a method of measuring insulin resistance. HOMA-IR is calculated by multiplying fasting plasma insulin (FPI) by fasting plasma glucose (FPG), then dividing by the constant 405. Adipo-IR is calculated by multiplying fasting non-esterified fatty acids (NEFA)×fasting insulin. Blood samples were taken to assess HOMA-IR and Adipo-IR at Visit 2/Baseline, Visit 3/Week 2, Visit 4/Week 4, Visit 6/Week 8, Visit 8/Week 12, Visit 10/Week 16 and Follow up Visit 11/Week 20. All subjects were required to have been fasted for a minimum of 8 hours prior to blood sampling. If subjects had not fasted for a minimum of 8 hours, the duration of fasting time was recorded, and subjects encouraged to fast appropriately for the next clinical visit.

ELF: An ELF score is an extracellular matrix marker set consisting of tissue inhibitor of metalloproteinases 1 (TIMP-1), amino-terminal propeptide of type III procollagen (PIIINP) and hyaluronic acid (HA). Blood samples were taken to perform this assessment at baseline (Visit 2) and Week 16 (Visit 10).

Liver stiffness and CAP: Liver stiffness and CAP were assessed using transient elastography (e.g., FibroScan® 502 Touch model or equivalent). Patients were fasted and scanned at the same time of the day, if possible, for baseline (Week 0) and Visit 10 (Week 16). For this assessment the following conditions were met: patients were lying in dorsal decubitus position with the right arm in maximal abduction behind the head, in a similar position to that used for liver biopsy (LB); the tip of the transducer was placed on the skin between the ribs over the right lobe of the liver (the physician took the measurements with the probe placed in the intercostal space); during the FibroScan® examination the choice of M+ or XL+ probe was determined by the Automatic Probe Selection tool (APS) (If, the APS tool advises to use the “XL+ probe” or “switch” continuously between “M+ and XL+ probe”, only the XL+ probe was used); and the operator, assisted by an ultrasonic time motion image, located a portion of the liver which is free of large vascular structures (the depth of measurement was between 35-75 mm for the XL+ probe and 25-65 mm for the M+ probe and the explored volume will be 3 cm³). For each patient, the operator performed an examination including at least 10 valid measurements or a maximum of 20 attempts, with the XL+ or M+ probe, at the same spot. The entire examination lasted no more than 10-15 minutes. The final stiffness and CAP values was recorded as median values of valid measurements.

FIB-4 Index: FIB-4 index is based on age, platelet count, ALT level, and AST level and was assessed at Baseline (Visit 2) and Week 16 (Visit 10). FIB-4 score is determined as shown by the equation below.

$$FIB - 4 = \frac{Age ( years ) \times AST ( U / L )}{Platelet count ( {10}^{9} / L ) \times √ ALT ( U / L )}$$

NFS: The NFS is based on age, hyperglycemia, BMI, platelet count, albumin level, and AST/ALT ratio. NAFLD fibrosis score=−1.675+0.037× age (years)+0.094× BMI (kg/m2)+1.13×IFG/diabetes (yes=1, no=0)+0.99×AST/ALT ratio-0.013× platelet (×109/1)-0.66× albumin (g/dl). NFS was assessed at Baseline (Visit 2) and Week 16 (Visit 10).

Safety Assessments included the following: medical history; physical examination; ECG; vital signs; clinical laboratory safety tests (e.g., hematology, serum biochemistry, coagulation, lipid profile, and urinalysis); virology; pregnancy test; blood sampling; pharmacokinetic sampling; exploratory blood collection; biomarker blood collection; urine DOA and alcohol breath test; adverse event assessment; concomitant medication; bioanalysis; sample, storage, handling; and shipping; and restrictions. A detailed description of each is provided below.

Medical History: A complete review of the patient's medical history was undertaken by the Investigator or designee at the Screening Visit (Visit 1) and Baseline (Visit 2) to ensure that no exclusion criteria had been met. Any concomitant disease, whether considered relevant for the study or not by the Investigator, was reported. The date of diagnosis or duration of the condition was noted where possible.

Physical Examination: A physical examination (including height and weight) was performed by the Investigator as per the study flow chart in Table 19 at Visit 1/Screening, Visit 2/Baseline, Visit 3/Week 2, Visit 4/Week 4, Visit 6/Week 8, Visit 8/Week 12, Visit 10/Week 16 and Follow up Visit 11/Week 20 in accordance with local practices. This examination was completed in full at baseline and symptom-directed thereafter (i.e., a standard panel of body systems was not assessed unless indicated by patient). For example, should the patient report to the Investigator the presence of ‘rash’ then the skin was evaluated. It was not required that additional body systems were assessed unless clinically warranted. Any abnormal results were recorded. Changes in findings of the physical examination compared with the baseline examination were recorded as an AE.

ECG: A 12-lead ECG 10 mm/1 mv, 25 mm/s with a 10 second lead II rhythm strip was recorded at each time point. ECGs were recorded using the GE Mac 1200 or equivalent model. Patients were rested quietly in a fully supine position for 5 minutes before the ECG was taken. Recordings were made on the days indicated in Study Flow Chart in Table 19 at Visit 1/Screening, Visit 2/Baseline and Visit 10/Week 16.

Vital Signs: Vital signs measurements were performed as per the Study Flow Chart in Table 19 at Visit 1/Screening, Visit 2/Baseline, Visit 3/Week 2, Visit 4/Week 4, Visit 6/Week 8, Visit 8/Week 12, Visit 10/Week 16 and Follow up Visit 11/Week 20. Vital signs measurements were performed before any blood samples were taken. All new findings or changes to previous findings considered clinically significant were recorded as an AE if the finding was made after the patient had signed. Vital sign measurements included: blood pressure performed as supine (e.g., after at least 5 minutes of rest) systolic and diastolic blood pressure (in mmHg); heart rate taken at rest in beats per minute (bpm); and temperature taken as per clinical practice.

Clinical Laboratory Safety Tests: Safety tests were performed for hematology, serum biochemistry, coagulation, lipid profile, and urinalysis: Blood and urine samples were taken as per the Study Flow Chart in Table 19 for routine hematology, serum biochemistry, coagulation and urinalysis tests, along with a Lipid Profile. All samples were analyzed in the central laboratory. All subjects were fasted for a minimum of 8 hours prior to blood sampling. If subjects had not fasted for a minimum of 8 hours, the duration of fasting time was recorded, and subjects encouraged to fast appropriately for the next clinical visit.
Hematology: Full blood count to include red cell count, hemoglobin, hematocrit, white cell count, differential white cell count, platelet count and reticulocyte count.
Serum biochemistry: Urea (blood urea nitrogen; BUN), creatinine, uric acid, total bilirubin, Indirect and Direct Bilirubin, sodium, bicarbonate potassium, phosphorus, calcium chloride, alkaline phosphatase (ALP), aspartate aminotransferase (AST), alanine aminotransferase (ALT), ALT/AST ratio, lactate dehydrogenase (LDH), creatine phosphokinase (CPK), albumin, total protein, cholesterol, triglycerides, glucose, C-reactive protein (CRP).
Coagulation: prothrombin time (PT), international normalized ratio (INR) and activated partial prothrombin time (APTT).
Lipid Profile: LDL, HDL, and VLDL-C.
Urinalysis: pH, protein, glucose, blood, ketones, leukocytes, leukocyte esterase, bilirubin, specific gravity, urobilinogen and nitrate. Reflex micro if blood, protein, leukocyte esterase or nitrate/nitrite are present.

Virology: A blood sample was taken to perform virology tests including HIV, Hep C and Hep B as detailed in the Study Flow Chart in Table 19.

Pregnancy Test: For female patients of childbearing potential, a pregnancy test was carried out as per the Study Flow Chart of Table 19 at Visit 1/Screening, Visit 2/Baseline, Visit 4/Week 4, Visit 6/Week 8, Visit 8/Week 12, Visit 10/Week 16 and Visit 11/Week 20.

Blood Sampling: Blood samples were obtained, and laboratory results reviewed for clinically significant values by each Investigator following sample analysis and verification. Additional blood may have been required for repeats of safety laboratory test.

Pharmacokinetic (PK) sampling: Blood samples for PK analysis were collected via direct venipuncture as per the Study Flow Chart in Table 19 at Visit 2/Baseline, Visit 3/Week 2, Visit 4/Week 4, Visit 6/Week 8, Visit 8/Week 12, Visit 10/Week 16 and Follow up Visit 11/Week 20. A 1 mL blood sample was taken at each timepoint. Following F centrifugation, plasma samples were split in two and a back-up sample kept at the central laboratory until bioanalytical assays had been completed.

Exploratory Blood Collection: Blood was collected as per the Study Flow Chart in Table 19 at baseline (Week 0) and Visit 10/Week 16 and stored for potential gene array analysis or additional exploratory testing at a later date.

Biomarker Blood Collection: Blood was collected as per the Study Flow Chart in Table 19 at baseline (Week 0), Visit 6/Week 8, Visit 10/Week 16 and Follow up Visit 11/Week 20 and was stored for potential biomarker analysis.

Urine DOA and Alcohol Breath Test: As clinically appropriate at the discretion of the Investigator, an alcohol breath test was performed, and a urine sample taken from patients at any time during the conduct of the trial and testing done to detect the following: amphetamine, barbiturate, benzodiazepine, cocaine, cannabinoids, and opiates.

Concomitant Medication: Patients were on a stable dose of any concomitant medications for at least 3 months prior to screening and that dose should have remained stable for the entire study duration. If patients were insulin dependent this treatment should have commenced at least 3 months prior to screening, however changes in dose were permitted.

Restrictions: The study include diet, alcohol, caffeine, and physical activity restrictions. For diet, patients avoided both during the study and for 4 weeks prior to baseline, ingesting food supplements rich in omega-3 or omega-6 fatty acids (e.g., cod liver oil capsules). For alcohol, patient avoided alcohol consumption in excess of 21 units per week (males) or 14 units per week (females), whereby a unit consists of 10 ml or 8 mg of pure alcohol. There were no caffeine restrictions either prior to or during the study. For physical activity, patients were to avoid exercise and strenuous physical activity for at least 3 to 4 hours before the safety laboratory test (e.g., biochemistry).

### 4.6 Investigational Drug

DS102 capsules were white, opaque hard-shelled capsules (size 0) containing 500 mg of 15-HEPE ethyl ester (EE) with 5% w/w of colloidal silicon dioxide as viscosity modifier.

DS102 placebo (paraffin oil) were white, opaque hard-shelled capsule (size 0) containing equivalent fill weight of liquid paraffin with 1% w/w of colloidal silicon dioxide as viscosity modifier.

DS102 and Placebo capsules were stored at 2-8° C. in a secure area (e.g., a locked cabinet or drug storage room), protected from unintended use. Labels were blinded to the dose and contained the randomization number.

Dosage and administration: This study involved a comparison of DS102 with placebo, administered orally twice daily for a total duration of 16 weeks. The last study drug administration occurred on the day preceding Week 16 visit/Early Termination (ET) visit. Patients were required to take the capsules with or after food. Medication(s) for other conditions that were permitted in the study were taken as usual. The walleted blister packs consisted of 7 days of 4 capsules and lastly, the patients took the assigned medication for 16 consecutive weeks.

### 4.7 Adverse Events and Serious Adverse Events

Adverse events (AEs), serious adverse events (SAEs), and Unexpected Adverse Event (UAE) for this study were defined as follows.

AE: Any undesirable experience occurring to a patient who has taken their first dose of the study drug, whether or not considered related to the investigational IMP(s). AEs were recorded in the case report form, defining relationship to IMP and severity.

SAE: If a patient experienced a serious adverse event after the first dose of the study drug, the event was recorded as a SAE. A SAE is characterized as an untoward medical occurrence at any dose to include any of the following: results in death, is life threatening, requires in-patient hospitalization or prolongation of existing hospitalization, or results in persistent or significant disability/incapacity. The term “life-threatening” refers to an event in which the patient was at risk of death at the time of the event. It does not refer to an event which hypothetically might have caused death if it were more severe. Examples of important, but not life threatening, events included intensive treatment in an emergency room, allergic bronchospasm, blood dyscrasias or convulsions that did not result in hospitalization, or the development of drug dependency or drug abuse.

UAE: An experience not previously reported in the Investigator's Brochure or similar product information sheet such as the Summary of Products Characteristics (SPC).

The intensity of an AE is an estimate of the relative severity of the event made by the Investigator based on his or her clinical experience. The following definitions were used to rate the severity of an AE:
Mild: The adverse event was transient and easily tolerated.
Moderate: The adverse event caused the patient discomfort and interrupted the patient's usual activities.
Severe: The adverse event caused considerable interference with the patient's usual activities and may have been incapacitating or life-threatening.

Causality of the AE to experimental treatment was also established by taking into account the patient's history, most recent physical examination findings, and concomitant medications. The following definitions were used to determine causality of an AE:
Not related: temporal relationship of the onset of the AE, relative to the experimental treatment was not reasonable or another cause could explain the occurrence of the AE.
Related: temporal relationship of the onset of the AE, relative to the experimental treatment was reasonable, followed a known response pattern to the treatment, and an alternative cause was unlikely.

All AEs were reported for the entire study duration up to and including the follow up period.

### 4.8 Drug-Induced Livery Injury

Severe drug-induced liver injury (DILI): Irrespective of perceived causation, in the event of severe DILI the investigational drug was discontinued until the episode was deemed resolved. In the event the investigational drug was deemed to be the cause of the liver injury then the patient was not rechallenged with the drug. Severe DILI stipulates evidence of hepatic impairment as demonstrated by a total bilirubin>2×ULN or INR>1.5.

Patients with abnormal baseline liver biochemistry: In determining abnormal baseline liver biochemistry, a fold increase was calculated against baseline levels instead of using the ULN. Thus, a figure of 3× baseline ALT or AST (or >200 IU/L) was followed by repeat testing within 72 hours to confirm/determine if the biochemical changes were improving or worsening. AE information was collected alongside a thorough physical examination. A liver etiology screen and/or other appropriate testing was undertaken. In the event of liver dysfunction, then the patient was managed as a severe DILI. Pausing of drug treatment was considered if any of the criteria under severe DILI occurred.

### 4.9 Serious Adverse Reactions and Unexpected Adverse Reactions

Adverse Reaction: All noxious and unintended responses to a medicinal product related to any dose were considered adverse drug reactions. The phrase “responses to a medicinal product” means that a causal relationship between a medicinal product and an AE was at least a reasonable possibility (i.e., the relationship cannot be ruled out). For marketed medicinal products, an adverse reaction is a response to a drug which is noxious and unintended, and which occurs at doses normally used in man for prophylaxis, diagnosis, or therapy of disease or for modification of physiological function.

Unexpected Adverse Reaction: An adverse reaction, the nature or severity of which was not consistent with the applicable product information.

Suspected Unexpected Serious Adverse Reaction (SUSAR): Any serious adverse reaction that might have been related to the IMP and was unexpected according to the definition above.

### 4.10 Statistical Methodology and Data Management

This clinical trial employed a randomized, double-blind, placebo-controlled parallel group design. Randomization was used to minimize assignment bias and to increase the likelihood that known and unknown patient attributes (e.g., demographic characteristics) were evenly balanced across the treatment groups. Blinding was used to reduce potential bias during data collection and evaluation of safety and efficacy. The use of placebo as comparator was justified as a reasonable design to assess safety and efficacy in patients based on the brevity of the study duration and the absence of any possible long-term irreversible damage that may have had the result of placebo treatment.

Estimation of Sample Size: Assuming a 20% delta in the percentage response between active drug and placebo arms, a standard deviation of 25% and a dropout rate of 20%, this resulted in a requirement of 32 patients per group for a statistical test with 5% level of significance, and 80% power. The sample size was re-estimated at an interim analysis based on recommendation from an unblinded. The sample size could have increased to a maximum of 150 patients in total to achieve a conditional power of 80% for the primary endpoint.

Blinding and Code Breaking Instructions: All study site personnel, as well as the personnel involved in the monitoring or conduct of the study, were blinded to the individual patient treatment assignments. Randomization details were kept strictly confidential, accessible only in an emergency to authorized persons, until the time of formal unblinding. The blinded code for the trial was broken only after all patient data has been recorded and verified and the database locked.

Interim Analysis and Data Monitoring: Interim analysis safety was carried out to estimate the conditional power when at least 50% of the patients had completed their Week 16 visit. The interim analysis was based on data collected for the primary and co-primary efficacy endpoints as well as the secondary endpoints and was used to estimate the conditional power to achieve the primary study objective, to potentially re-estimate the sample size and to potentially drop the less effective treatment arm.

Clinically Meaningful Response: A higher mean or median reduction of at least 20% of ALT or liver stiffness compared to placebo and higher mean or median reduction of at least 10% of both ALT and liver stiffness compared to placebo.

Analysis Sets included the enrolled set, the full analysis set (FAS), per-protocol set (PPS), safety analysis set (SAS), and the pharmacokinetic (PK) set. A detailed description of each analysis set is provided below.

Enrolled Set: Patients who signed the informed consent form. Screen failures were patients from the Enrolled Population who did not meet the eligibility requirements and were withdrawn from the study prior to randomization.

FAS: Randomized patients who received at least one administration of study treatment and had at least one post-baseline measurement. Patients were analyzed according to the treatment they were assigned to at randomization, irrespective of what treatment they actually received.

PPS: A subset of the FAS consisting of those patients of FAS who had no major protocol violations. All protocol deviations were assessed and documented on a case-by-case basis prior to the database lock, and major deviations considered as having a serious impact on the efficacy results lead to the relevant patient being excluded from the PPS.

SAS: Patients who took at least one administration of study treatment. Patients were analyzed according to the treatment actually taken.

PK Set: Patients in the SAS who had at least one DS102 PK concentration. Patients were analyzed according to the treatment actually received.

Safety Analysis: Demographic, medical history and physical examination data were listed for each patient and summarized descriptively. All AEs recorded during the study were coded to system organ class and preferred terms using the current version of the Medical Dictionary for Regulatory Activities (MedDRA). AEs were tabulated and summarized by treatment, relationship to treatment, seriousness and severity. Clinical laboratory values (e.g., hematology, biochemistry, and urinalysis) were listed for each patient by treatment and day. Values outside the laboratory normal ranges were listed separately with associated comments as to their clinical significance, with potentially clinically significant abnormalities highlighted and summarized by treatment. Clinical laboratory values obtained prior to dosing were defined as baseline values. Alcohol breath test and DOA test results were listed for each patient. Individual values of vital signs were listed and summarized descriptively for each treatment and day. 12 lead ECG assessments were listed for each patient with all associated comments and summarized by treatment and day. Concomitant medications (if any), categorized by medication group and subgroup according to the latest version of the World Health Organization drug dictionary, were listed and summarized by treatment. In general, appropriate descriptive statistics according to the nature of the variable were applied. Categorical variables were presented using counts and percentage, whilst continuous variables were presented using mean, standard deviation, median, minimum, maximum, coefficient of variation and number of patients.

Pharmacokinetic Analysis: Plasma concentrations of 15(S)-HEPE were tabulated and summarized descriptively. Individual and mean plasma concentration-time profiles of 15(S)-HEPE were presented graphically.

Primary Variables: The primary efficacy variable was the change from baseline in serum ALT at Week 16 (Visit 10). The active treatment groups were compared against placebo via an analysis of covariance (ANCOVA) model, including the corresponding baseline value as covariate. The comparisons against placebo were done according to Dunnett's multiple testing procedure. For missing Week 16 values, the last value available was carried forward (LOCF). Similar methods were applied for liver stiffness. For ALT, longitudinal modelling was considered in addition.

Secondary variables: The secondary efficacy variables and their changes from baseline to Week 16 (Visit 10) were summarized with descriptive statistics per treatment group and visit. This applied to the AST, AST:ALT ratio, hepatic fat measured by CAP, liver stiffness measurements by transient elastography, FIB-4, NFS, ELF and HOMA-IR/Adipo-IR. The change from baseline for the active treatment groups was compared against placebo via an ANOVA model, including a term for center effects. The 5% level of significance was used for all treatment comparisons.

Exploratory analysis: The exploratory analysis included analysis of lipid and metabolic parameters including total cholesterol, triglycerides, very low-density lipoprotein cholesterol (VLDL-C), low density lipoprotein cholesterol (LDL-C), high density lipoprotein cholesterol (HDL-C), non-high-density lipoprotein cholesterol (non-HDL-C), remnant-like particle cholesterol (RLP-C), fasting glucose, insulin, free fatty acids and hemoglobin A1C (HbA1C). Additional exploratory analysis included high throughput lipidomics and proteomics.

### 4.11 Results

This study demonstrated that patients diagnosed with NAFLD and administered DS102 at a dose of either 1 g or 2 g per day, exhibited a statistically significant reduction in markers of metabolic overload and an improvement in insulin sensitivity and glycemic control.

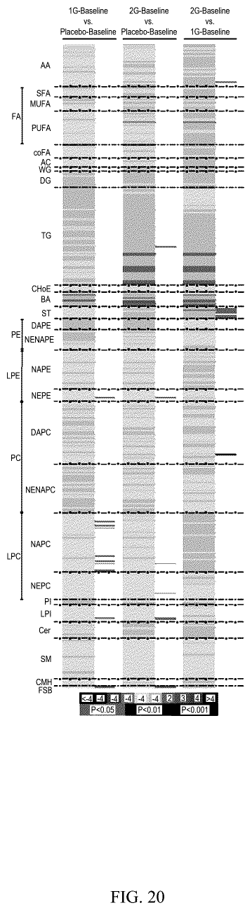

FIG. 20 shows that the three treatment groups were well balanced at baseline and had similar lipidomic and metabolomic profiles. The baseline characteristics of the patients are shown in Table 16 where 10 to 13% of the patients were on a statin therapy at baseline.

TABLE 16
Baseline Characteristics of the Subjects

| | 2 g DS102 | 1 g DS102 | Placebo |
|---|---|---|---|
| | (n = 31) | (n = 32) | (n = 30) |
| Age, median, years | 47.0 | 53.5 | 50.0 |
| Gender, % | | | |
| Female | 32.3 | 37.5 | 30.0 |
| Male | 67.7 | 62.5 | 70.0 |
| Race, % | | | |
| White | 100.00 | 93.75 | 90.00 |
| Other | 0.00 | 6.25 | 10.00 |
| BMI, median, kg/m² | 30.9 | 33.5 | 33.9 |
| Statin use, % | 9.7 | 12.5 | 13.3 |
| Fasting plasma glucose, | 119.1 | 110.9 | 120.3 |
| mean, mg/dL | | | |
| Hemoglobin A1C (%), | 6.3 | 6.2 | 6.2 |
| mean | | | |
| ALT, median, IU/L | 74.0 | 70.5 | 96.5 |
| Liver Stiffness, median, | 7.9 | 8.2 | 8.4 |
| kPa | | | |
| HOMA-IR, median | 4.4 | 5.1 | 5.0 |
| Triglycerides, median, | 242.0 | 146.0 | 150.0 |
| mg/dL | | | |

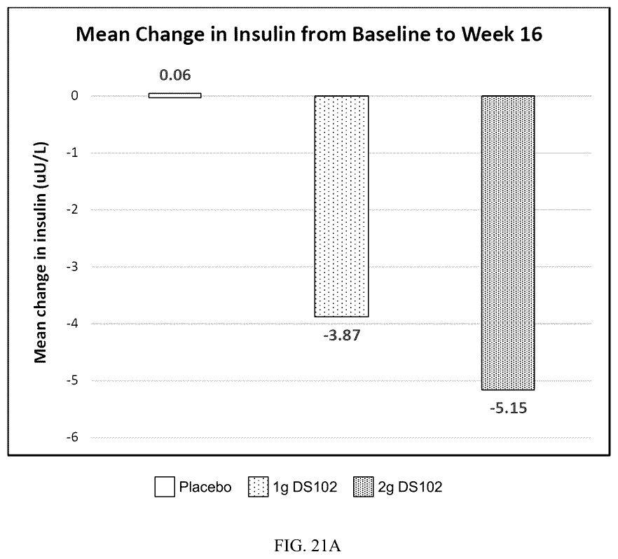

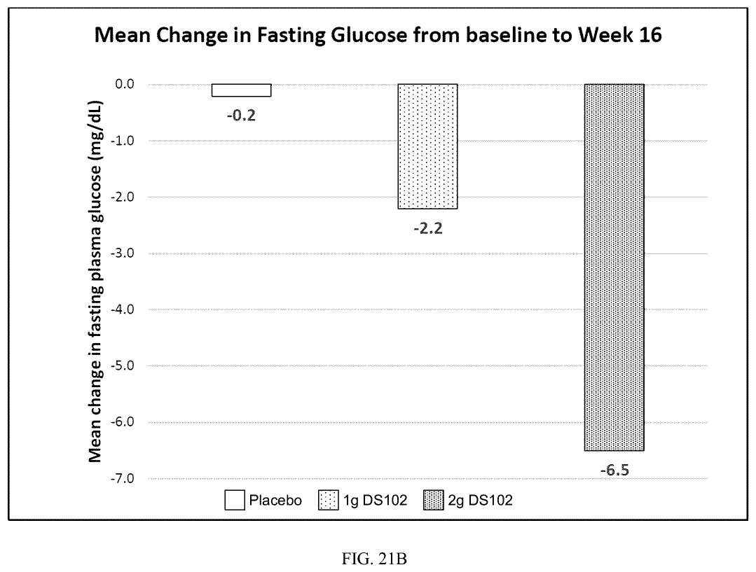

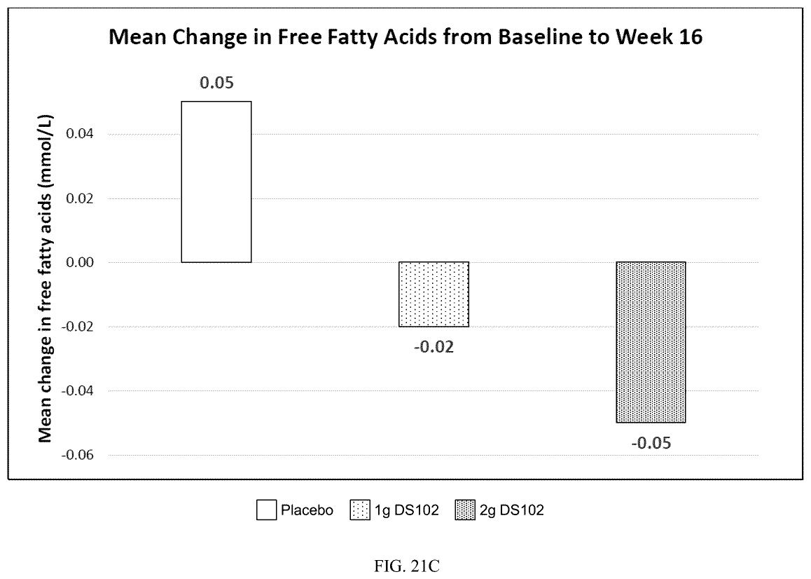

FIGS. 21A-21C depict the changes in insulin, glucose, and free fatty acid levels from baseline to Week 16 in patients administered DS102 either 1 g or 2 g per day as compared to a placebo. The reduction in insulin, glucose, and free fatty acid levels upon administration of DS102 is clinically significant as metabolic substrates including glucose, carbohydrates and free fatty acids drive the pathogenesis of NASH.

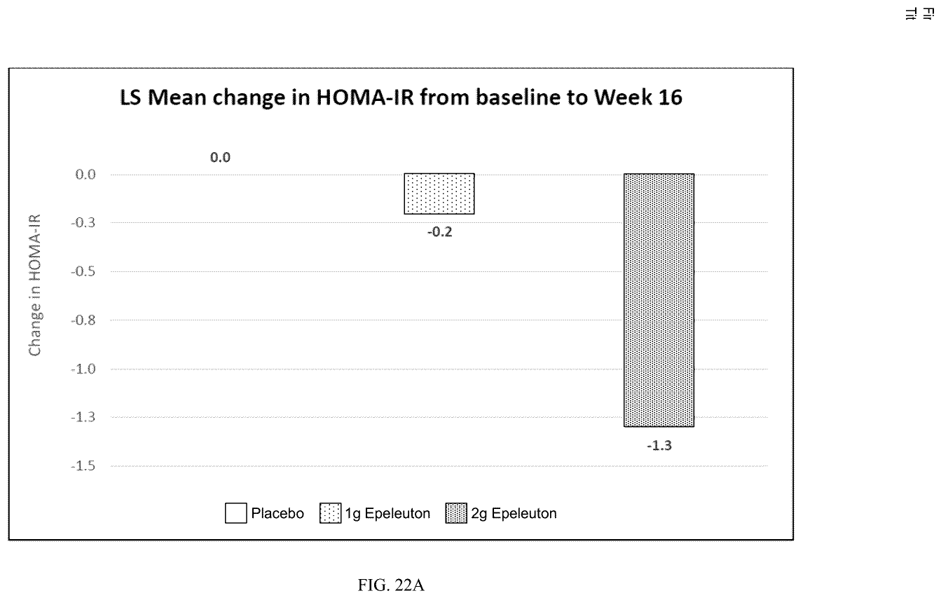

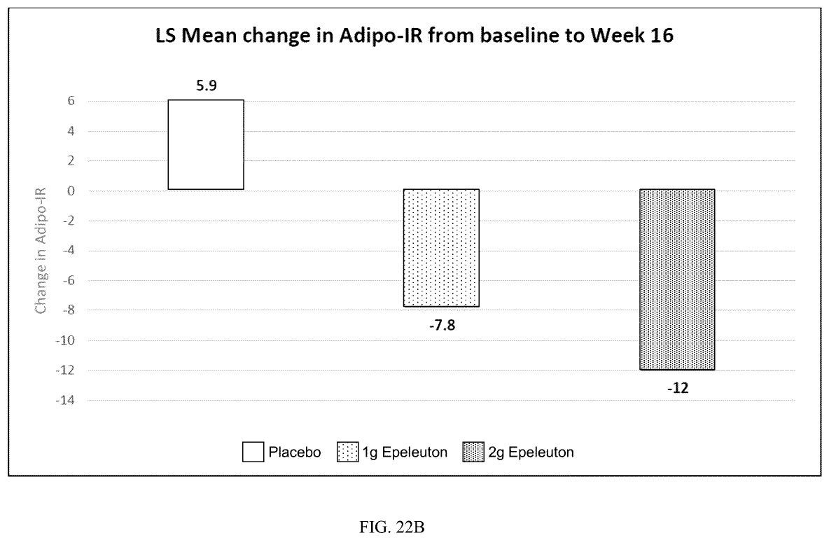

FIGS. 22A and 22B show the changes in HOMA-IR and adipo-IR levels from baseline to Week 16 in patients administered DS102 either 1 g or 2 g per day as compared to a placebo. The patients exhibited an improvement in both insulin resistance indices (e.g., a reduction in HOMA-IR and adipo-IR levels) at Week 16, with significant improvements in the Per Protocol Set (PPS) observed for those patients administered 2 g of DS102.

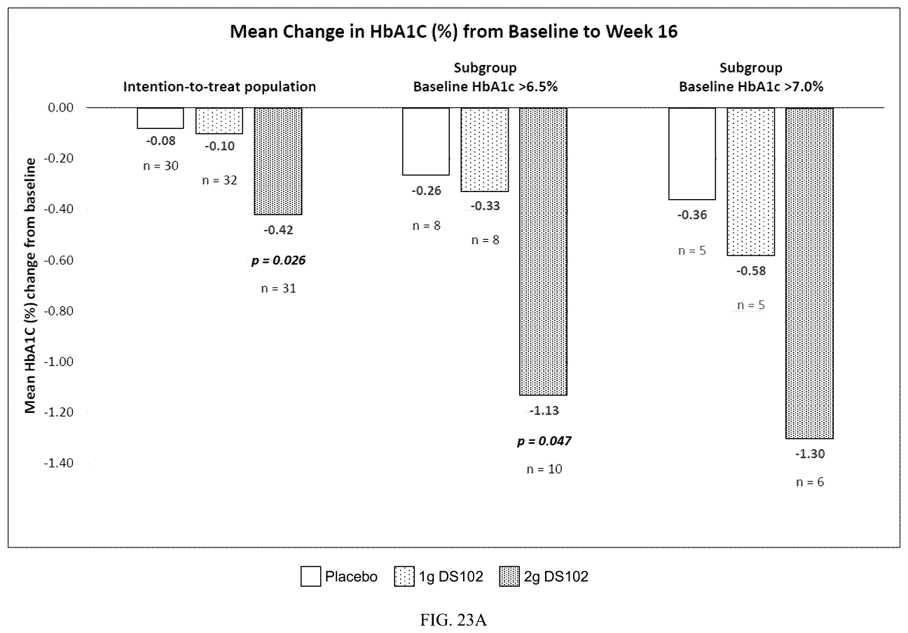

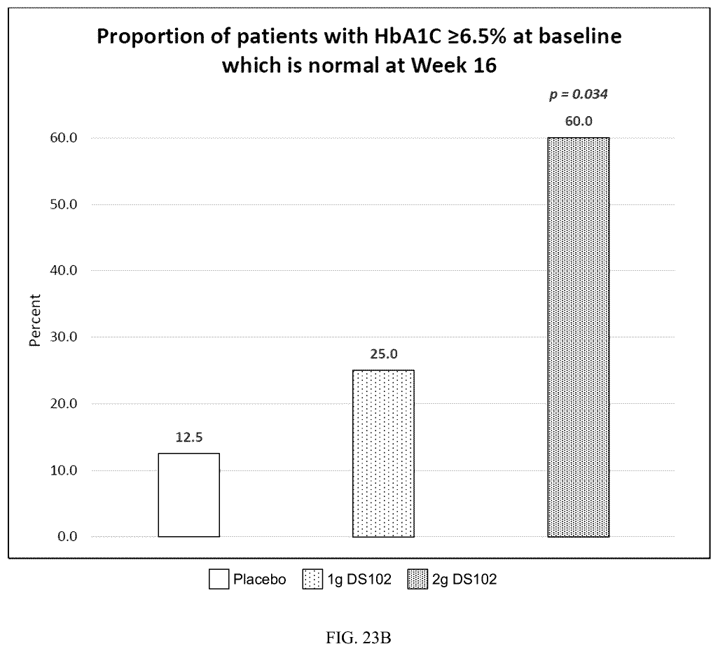

FIGS. 23A and 23B depict the changes in glycosylated hemoglobin (e.g., HbA1c) levels from baseline to Week 16 in patients administered DS012 either 1 g or 2 g per day as compared to a placebo. Specifically, FIG. 23A shows the change in HbA1c levels and FIG. 23B shows the change in HbA1c levels in the proportion of patients who had high HbA1c levels at baseline but achieved normal levels at Week 16. Since Hb1Ac is a measure of the amount of glucose attached to the body's red blood cells and a surrogate for long-term glycemic control, these results indicate the administration of DS102 provides clinically significant improvements and normalizes glycemic control in a dose-dependent manner.

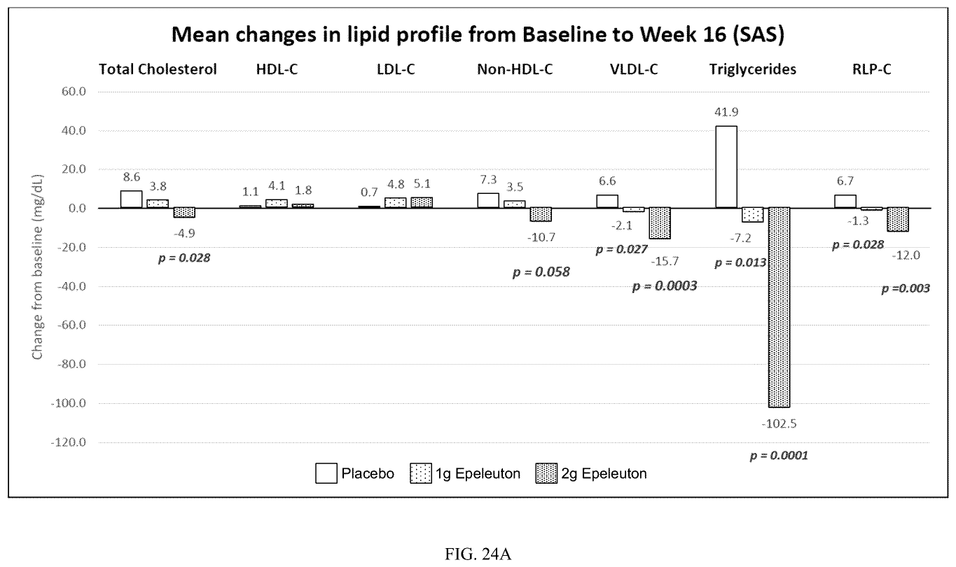

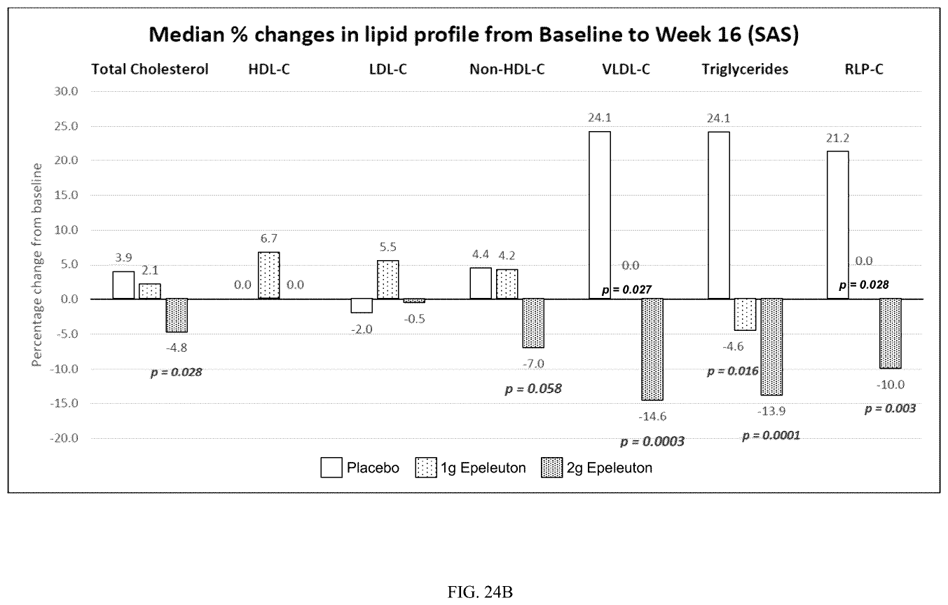

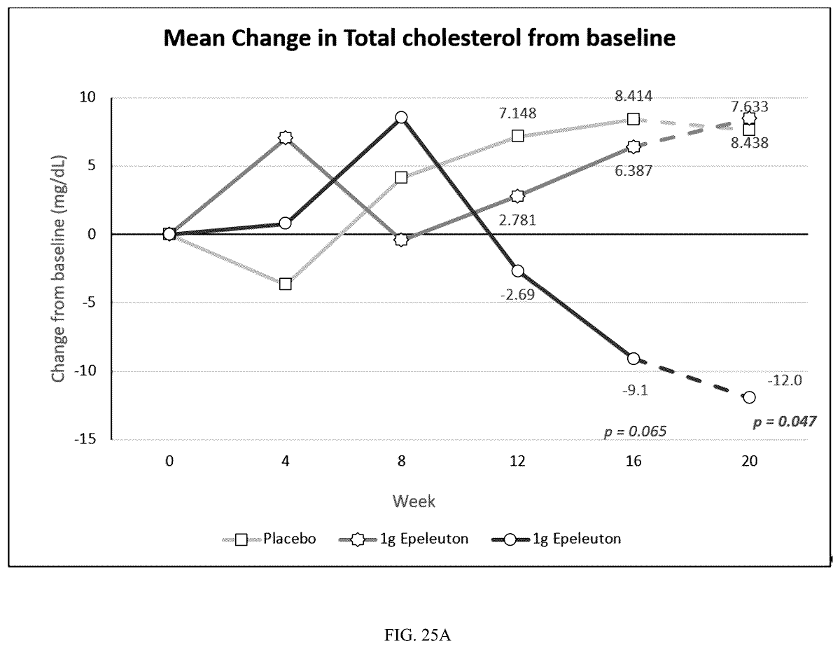

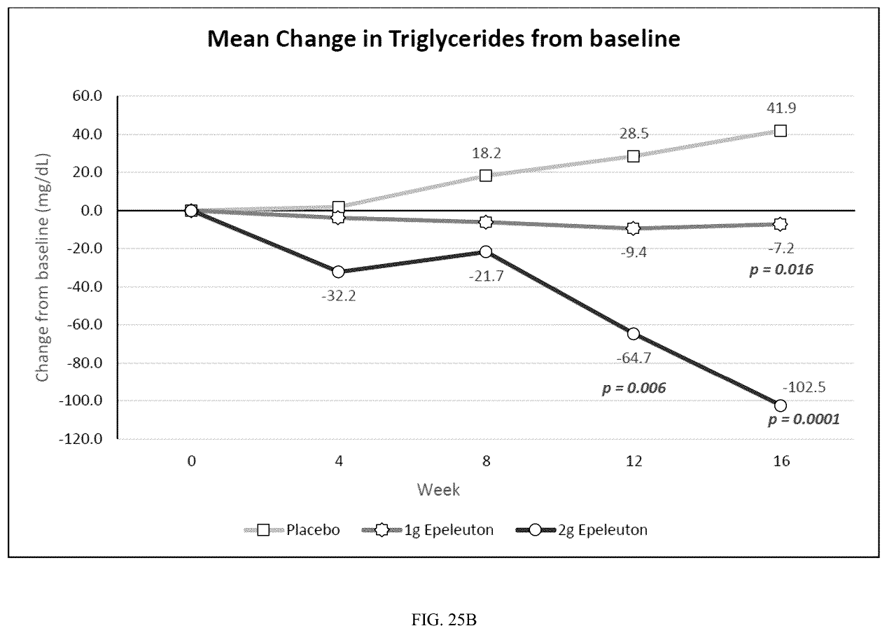

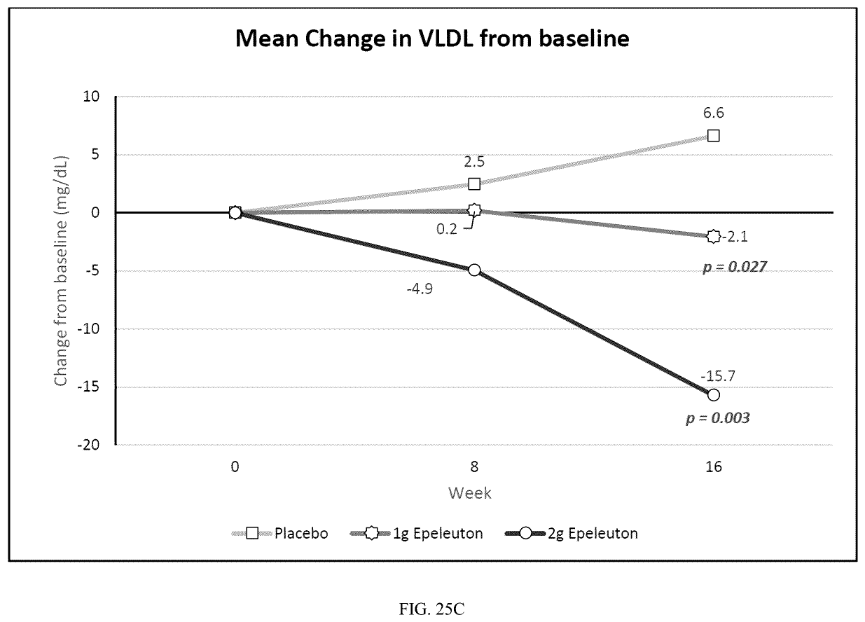

FIGS. 24A and 24B show the mean change and median (%) change in the patient's lipid profile at Week 16 in the safety analysis set (SAS). These results are further depicted in FIGS. 25A-25C and illustrate that the administration of DS102 significantly improved patient's lipid profile by either sustaining or reducing total cholesterol, VLDL-C, non-HDL-C, remnant-like particle (RLP) cholesterol and triglyceride levels in the patients. Significantly, the reductions did not plateau at Week 16, suggesting that the administration DS102 might induce even larger changes in studies of longer duration.

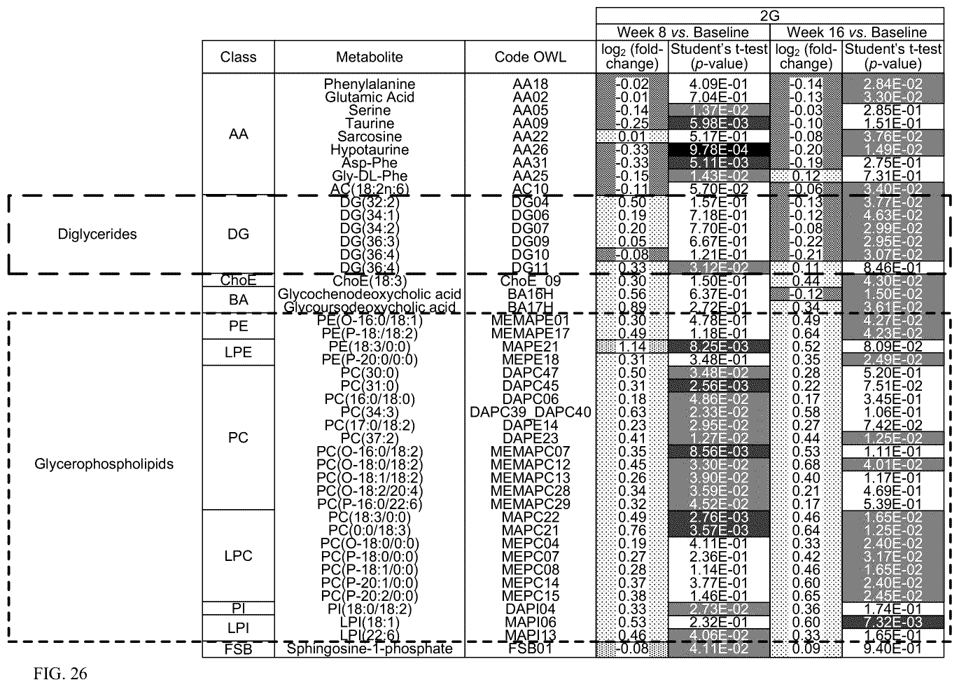

As shown in FIG. 26 the administration of DS102 also reverses the hepatotoxic lipid signature of NASH and improves multiple lipid classes that are altered in patients diagnosed with NASH. Specifically, the administration of 2 g of DS102 significantly decreased levels of multiple hepatotoxic diglycerides and significantly increased levels of multiple glycerophospholipid groups. This finding is important as patients with NASH have low levels of hepatic and plasma glycerophospholipids.

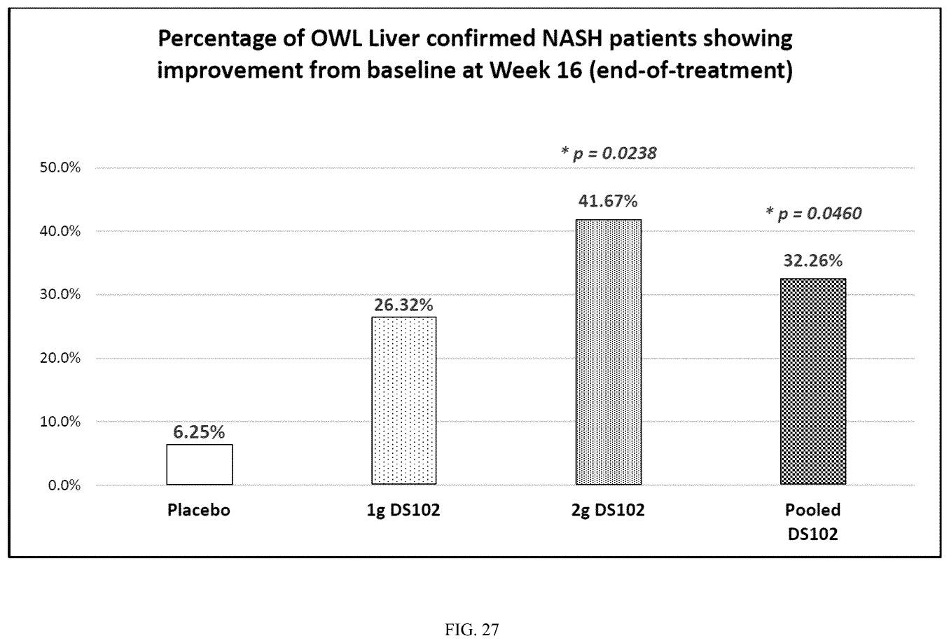

FIG. 27 shows that the administration of DS102 also resolved NASH based on validated diagnostic tests such as the OWL liver care non-invasive diagnostic test for NASH. OWL Liver Care is a test that was developed based on the plasma lipidomics in biopsy-confirmed NASH patients and provides high predictive values. OWL Liver Care has an area under the curve (AUC) of 0.88 for distinguishing NAFLD and normal liver patients and an AUC of 0.79 for distinguishing NAFLD without steatohepatitis and NASH patients. The administration of DS102 significantly improved and normalized OWL liver care diagnosed NASH in a dose dependent manner as compared to placebo at Week 16. Table 17 shows the test diagnoses at baseline for each treatment group and demonstrates that most patients were classified as NASH or NAFLD at baseline with a lower percentage of the patients in the 2 g DS102 group.

TABLE 17
OWL Test Diagnoses at Baseline

| | | | Change in Diagnosis | | |
|---|---|---|---|---|---|
| | | Base- | (Week 16 vs. baseline) | | |
| Arm | Diagnosis | line | Improvement | Stable | Worsening |
| Placebo | NASH | 16 | 1 | 15 | — |
| | Steatosis | 7 | 1 | 4 | 2 |
| | No NAFLD | 3 | — | 1** | 2 |
| 1 g DS102 | NASH | 19 | 5 | 14 | — |
| | Steatosis | 5 | — | 2 | 3 |
| | No NAFLD | 4 | — | 3 | 1 |
| 2 g DS102 | NASH | 12 | 5 | 7 | — |
| | Steatosis | 14 | 1 | 9 | 4 |
| | No NAFLD | 3* | — | 2*, ** | 1 |

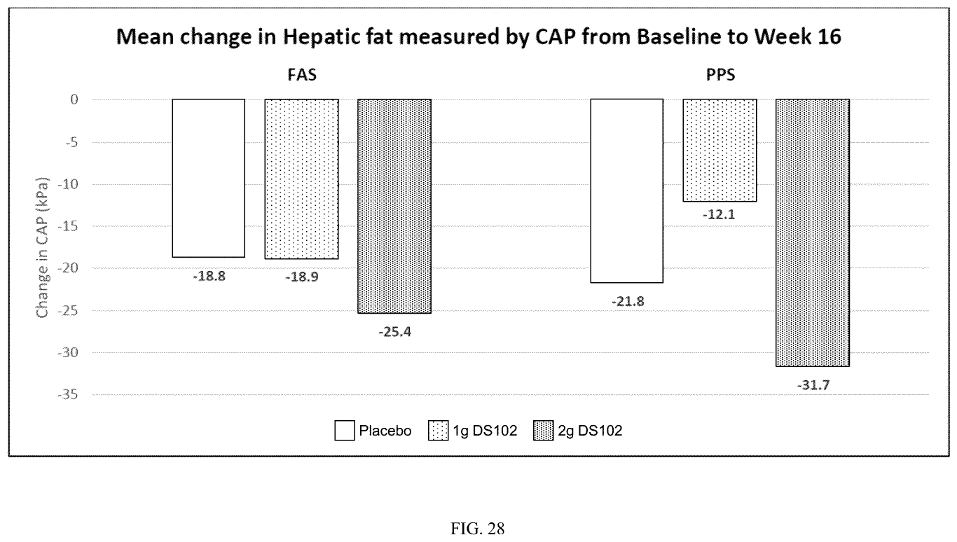

The administration of DS102 also reduced hepatic fat content as assessed by CAP in patients diagnosed with NAFLD as shown in FIG. 28. It is further contemplated that DS102 is expected to induce larger changes in hepatic content in studies of longer duration and when assessed by more sensitive methods.

The administration of DS102 also lowered triglyceride levels in patients as shown in Table 18 and it is thus contemplated that DS102 would also be effective at lowering cardiovascular risk.

TABLE 18
Changes Triglyceride Levels of Patients
Administered DS102 at Week 16

| | Triglycerides (median |
|---|---|
| | placebo-corrected % |
| DS102 (2 g) | change from baseline) |
| Full Analysis Set | −40.2 |
| Subgroup: Triglycerides > Upper Limit | −39.0 |
| Normal (>150 mg/dL) | |
| Subgroup: High Triglycerides (>200 | −20.4 |
| mg/dL) | |

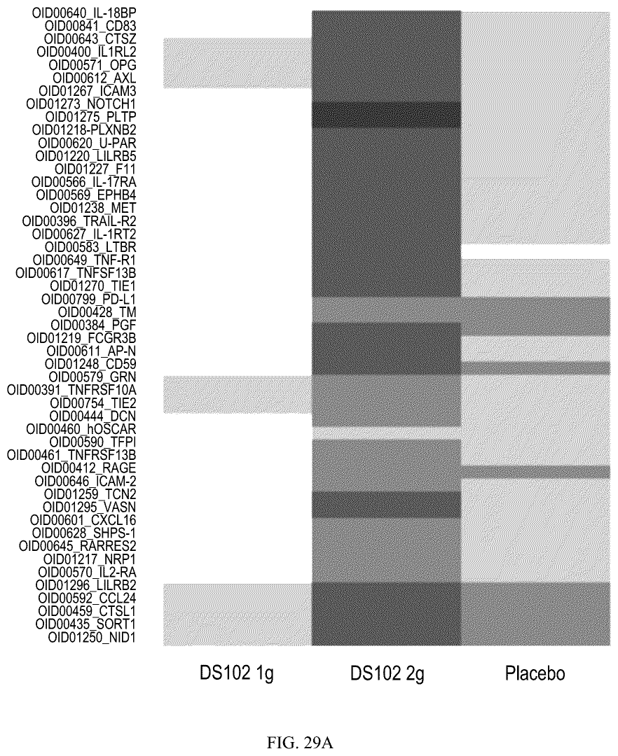

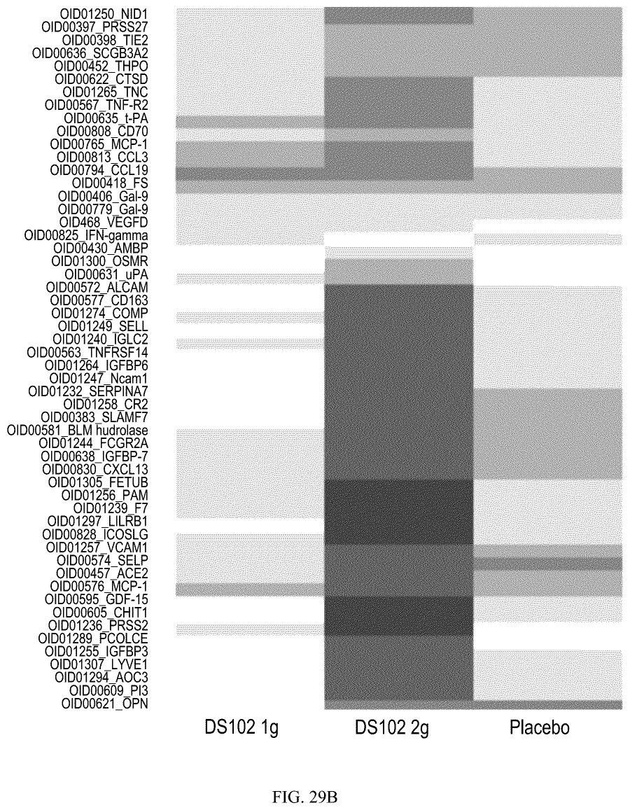

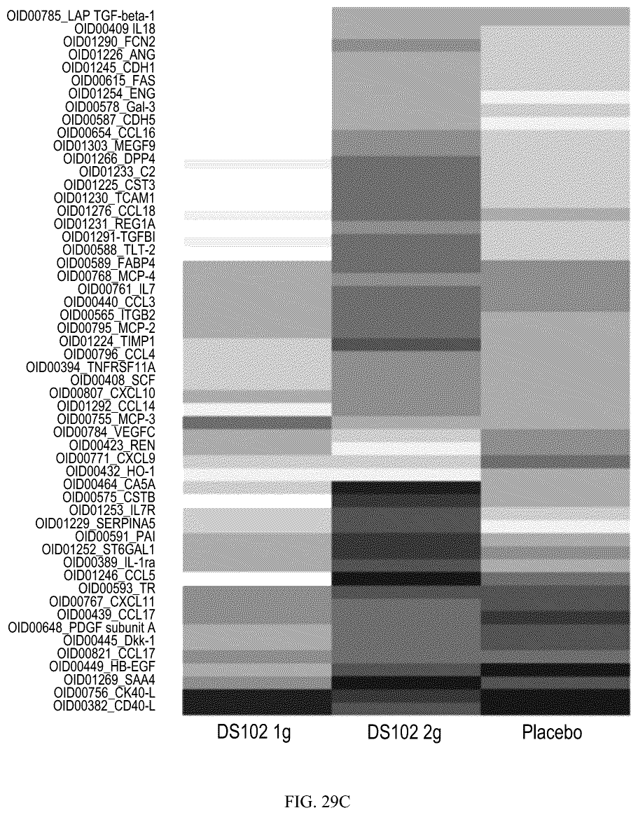

FIGS. 29A-29C shows that the administration of DS102 decreases inflammatory and pro-fibrotic proteins. For this assessment, blood samples before and after treatment with DS102 were analyzed for a panel of greater than 350 different protein biomarkers. Treatment with 2 g of DS102 significantly downregulated the expression of over 150 markers associated with inflammation, fibrosis, lipid metabolism, apoptosis, and chemotaxis. The resolution of metabolic overload and lipotoxicity was observed following treatment with DS102, which prevents subsequent cell stress, inflammation, and fibrosis. The reduction in the inflammatory and pro-fibrotic proteins suggests the potential of DS102 to provide resolution for NASH and the prevention of fibrosis.

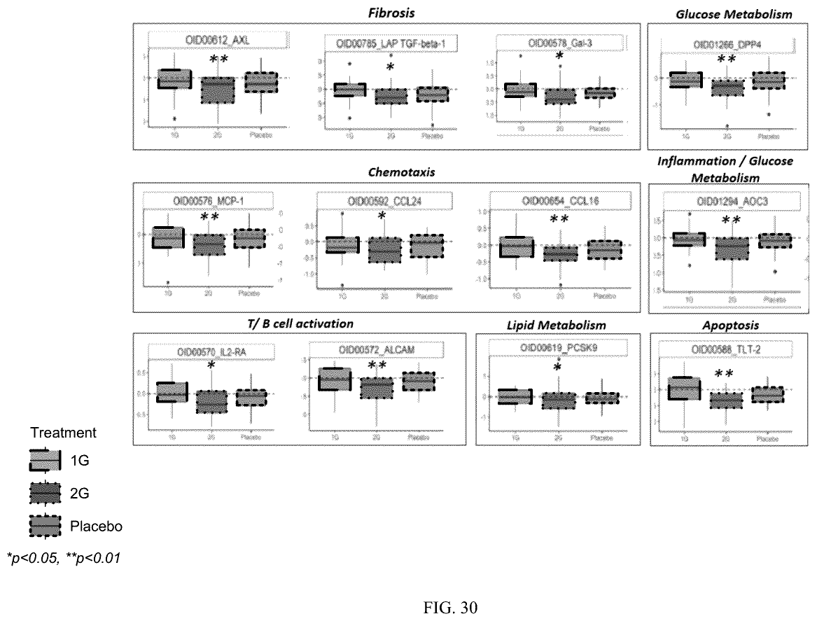

The administration of DS102 also decreased the expression of multiple NASH development targets as shown in FIG. 30. In particular, the administration of 2 g of DS102 decreased the NASH drug development targets to include CCR2/5 signaling (Cenicriviroc-Allergan), Galectin3 (GR-MD-02-Galectin), and AOC3 (Boehringer Ingelheim).

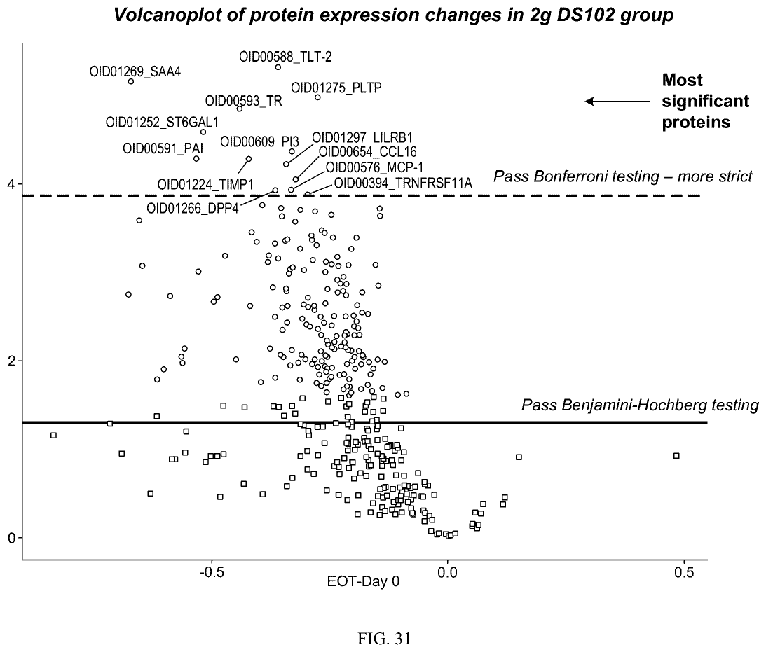

The volcano plot in FIG. 31 (data with Bonferroni and Benjamini-Hochberg testing) shows that the administration of 2 g of DS102 decreased inflammatory and pro-fibrotic proteins based on changes in protein expression. Table 19 shows the most significant inflammatory and pro-fibrotic protein reductions based on Bonferroni Testing.

TABLE 19
Significant Proteins based on Bonferroni Testing

| Protein | Description |
|---|---|
| PAI-1 | TGF-β induced protein, increased in metabolic |
| | syndrome, atherosclerosis, fibrosis |
| TIMP-1 | Pro-fibrotic |
| DPP4 | T-cell activation, DPP4 inhibition increases |
| | GLP-1 |
| TLT2 | Enhances T-cell activation, clearance of |
| | apoptotic cells |
| CCL16 | chemokine that binds to CCR 1, 2, 5, 8. |
| | Cenicriviroc (Allergan) is a CCR2/5 antagonist |
| MCP-1 (CCL2) | chemokine that binds to CCR 2, 4. Cenicriviroc |
| | (Allergan) is a CCR2/5 antagonist. Increased in |
| | NASH, atherosclerosis etc. |
| SAA4 | Apolipoprotein |
| PI3/Elafin | Increased in graft versus host disease |
| TR | Iron homeostasis |
| LILRB1 | Receptor for class I MHC antigens |
| TNRSF11A | T-cell/dendritic cell interaction |

Administration of 1 g of DS102 also showed significant reductions for TR. Table 20 shows the most significant inflammatory and pro-fibrotic protein reductions based on an analysis with a linear model.

TABLE 20
Significant Proteins based on the Linear Model

| Protein | Description |
|---|---|
| TLT2 | Enhances T-cell activation, clearance of apoptotic cells |
| AOC3 | Impairs glucose homeostasis. Increased in NASH, |
| | atherosclerosis. |
| PRSS2 | Expressed in pancreas, involved in cell adhesion |

This analysis also indicated that 1 protein in 1 g DS107 group and 0 proteins in Placebo group passed the 1st threshold based on the Benjamini-Hochberg testing.

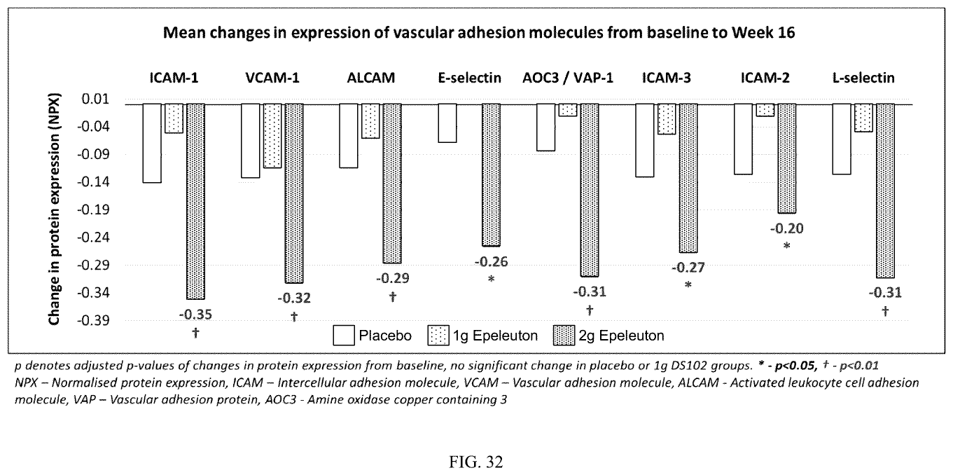

The administration of DS102 also decreased the expression of multiple vascular adhesion molecules as shown in FIG. 32. Vascular adhesion molecules are implicated in atherosclerosis and their circulating levels are associated with cardiovascular risk.

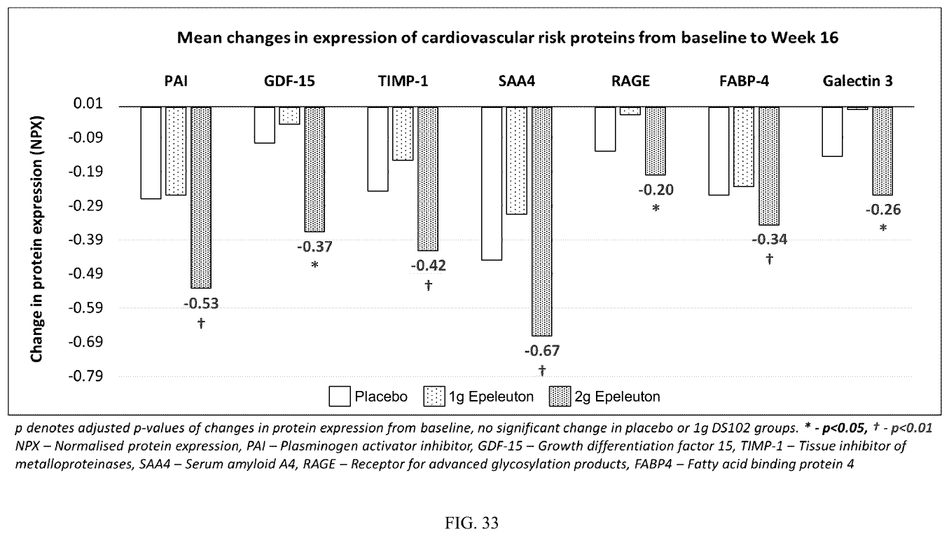

The administration of DS102 also decreased the expression of multiple proteins that are associated with increased cardiovascular risk as shown in FIG. 33.

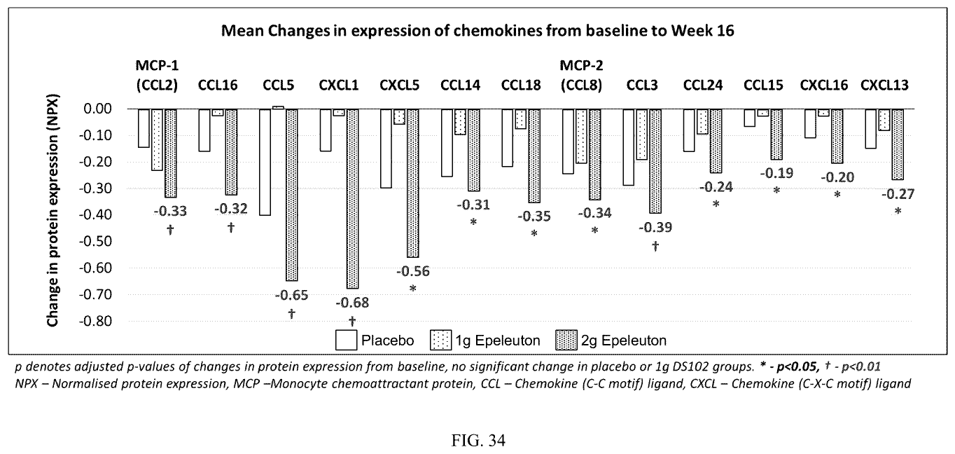

The administration of DS102 also decreased the expression of multiple circulating chemokines as shown in FIG. 34. Chemokines are important drivers of the chronic inflammation of atherosclerosis.

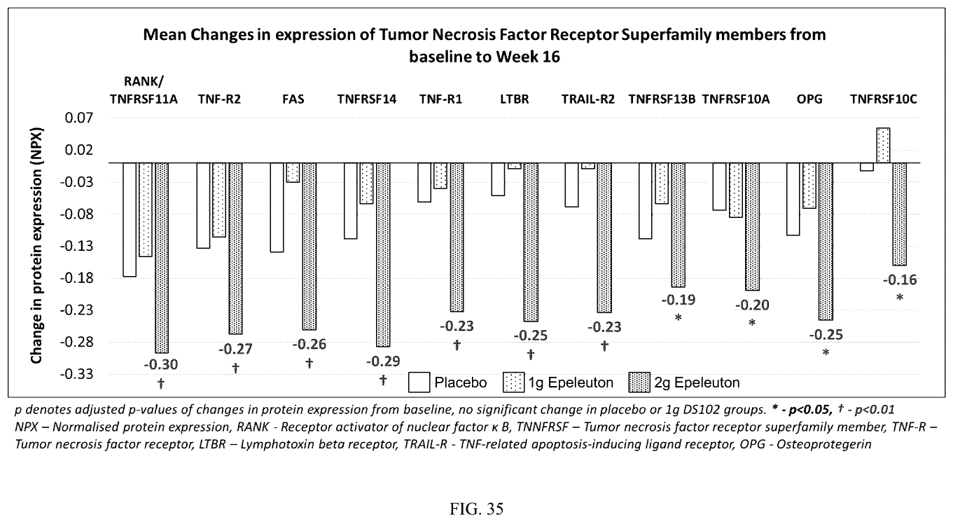

The administration of DS102 also decreased the expression of multiple tumor necrosis factor receptor superfamily members as shown in FIG. 35. Tumor necrosis factor receptor superfamily members are implicated in inflammation and atherosclerosis.

DS102 was also proven to be safe and well tolerated, with no observed safety and tolerance differences as compared to placebo. There was no drug related serious adverse events (SAEs) and a low incidence in adverse events (AEs), which was consistent across all treatment groups. Most AEs were mild to moderate as well as transient and no patients experienced an AEs that lead to treatment discontinuation. The safety profile across each treatment group is shown in Table 21.

TABLE 21
Safety Profile for Study Groups

| | 2 g DS102 | 1 g DS102 | Placebo |
|---|---|---|---|
| Patients, n (%) | (n = 30) | (n = 32) | (n = 31) |
| ≥1 AE | 15 (50.0) | 19 (59.4) | 17 (54.8) |
| ≥1 AE related | 3 (10.0) | 2 (6.3) | 4 (12.9) |
| to study drug | | | |
| ≥1 SAE | 1 (3.3) | 0 | 1 (3.2) |
| AEs leading to | 0 | 0 | 1 (3.2) |
| treatment | | | |
| discontinuation | | | |
| SAEs related to | 0 | 0 | 0 |
| drug study | | | |
| Death | 0 | 0 | 0 |

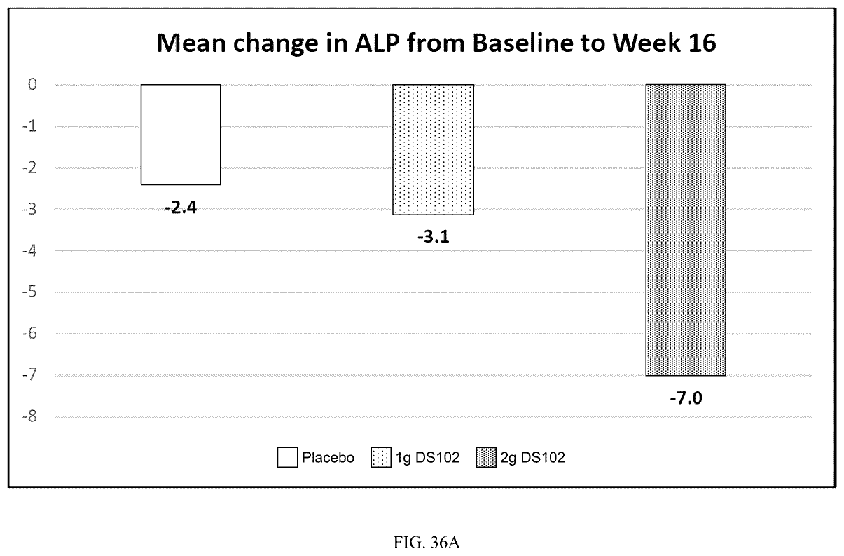

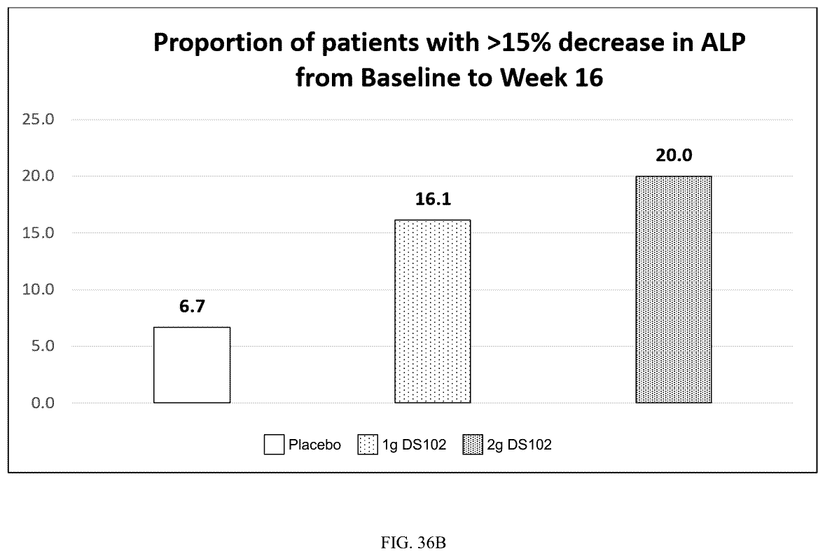

DS102 was also evaluated for potential efficacy in improving the related indications, primary biliary cirrhosis (PBC) and primary sclerosing cholangitis (PSC) as shown in FIGS. 36A and 36B. Importantly, administration of 2 g of DS102 reduced alkaline phosphate (ALP) as well as multiple markers for liver fibrosis suggesting that DS102 will also be effective in treating PBC and PSC.

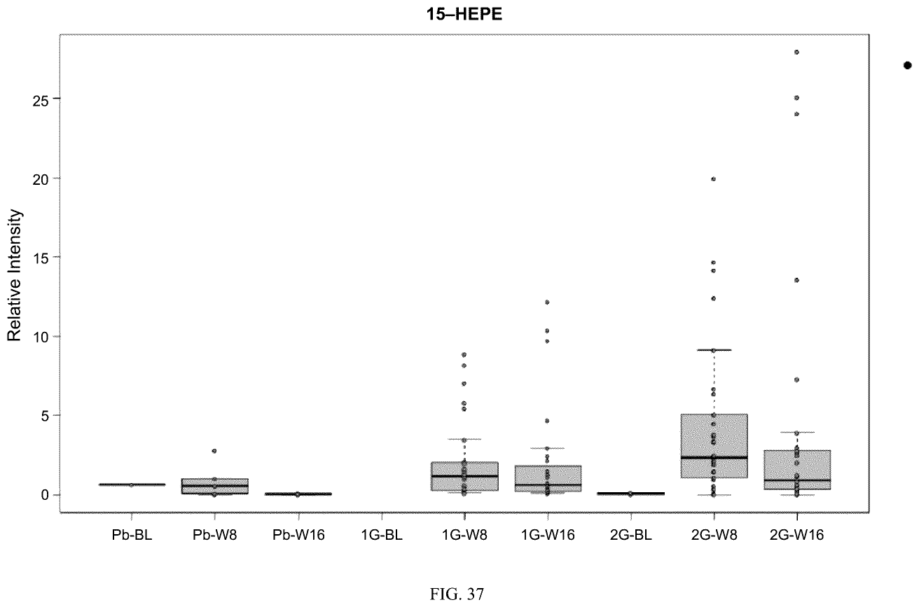

FIG. 37 is a boxplot of 15-HEPE ethyl ester (EE) trough plasma relative concentrations, which indicates that there was a higher systematic exposure in the DS102 treatment group at Weeks 8 and 16 as expected.

### 4.12 Summary

Overall, these results indicate that DS102 targets multiple stages of NASH pathology by significantly reducing metabolic load and improving insulin sensitivity. The administration DS102 also improved patient's lipid profiles by reversing the lipid accumulation levels associated with NASH. Specifically, those patients administered DS102 exhibited a reduction the accumulation of the hepatotoxic lipid levels to include total cholesterol, non-HDL cholesterol, RLP cholesterol, triglycerides, diglycerides, and VLDL-C as well as an increase of glycerophospholipid levels. These effects are significant as patients diagnosed with NASH are characterized by high total cholesterol, triglyceride, diglyceride, and VLDL-C levels and low glycerophospholipid and omega-3 PUFA levels.

Additionally, changes to multiple lipids are expected to confer a reduction of cardiovascular risk and improvement of multiple aspects of metabolic syndrome.

The results also suggested that there is a dose dependent effect in favor of administration 2 g or higher of DS102 with large and statistically significant improvements as compared to placebo.

In conclusion, DS102 is well suited as either monotherapy or part of combination therapy for treating NASH and is contemplated to reduce cardiovascular risk, including in patients diagnosed with NASH or cardiometabolic diseases including metabolic syndrome.

### Example 5: A 26 Week Toxicity Study of 15(S)-HEPE EE by Oral Gavage in Rats with a 4

### Week Recovery Period

The objectives of this study were to determine (1) the potential toxicity of 15(S)-HEPE EE, an omega-3 fatty acid used in the treatment of hepatic and pulmonary diseases, when given by oral gavage for 26 weeks to rats and to evaluate the potential reversibility of any findings; and (2) the toxicokinetic characteristics of 15(S)-HEPE EE.

### 5.1 Study Design

The test and control items were administered to the appropriate animals by once daily oral gavage 7 days a week for a minimum of 26 weeks. The volume for each animal was based on the most recent body weight measurements. The doses were given using a syringe with attached gavage cannula. The first day of dosing was designated as Day 1. The dosing formulations were removed from the refrigerator and stirred for at least 30 minutes before dosing. The dosing formulations were also stirred continuously during dosing.

Due to the onset of clinical signs, including hunched posture, erect fur and abnormal gait and body weight loss, animals 3206M, 3515F and 3703F were given a dosing holiday from Day 45 and remaining Group 3 animals were given a dosing holiday from Day 48. Animals will start dosing at the lower dose level (3000 mg/kg/day) from Day 64. The following tables illustrate the treatments (Table 22) and animal groupings (Table 23) that were used in the study.

TABLE 22
Treatment Chart

| | | | Dose | Dose | Adjusted Dose | Number of Animals | | | | | |
|---|---|---|---|---|---|---|---|---|---|---|---|
| Group | | Dose Level | Volume | Concentration | Concentration | Main Study | | Recovery | | Toxicokinetic | |
| No. | Treatment | (mg/kg/day) | (mL/kg) | (mg/mL) | (mg/mL) | M | F | M | F | M | F |
| 1 | Control | 0 | 10 | 0 | 0 | 15 | 15 | 5 | 5 | 3 | 3 |
| 2 | 15(S)-HEPE EE | 2000 | 10 | 200 | 206^{b}/204^{c} | 15 | 15 | 5 | 5 | 9 | 9 |
| 3 | 15(S)-HEPE EE | 6000^{a} | 10 | 600 | 618^{b} | 15 | 15 | 5 | 5 | 9 | 9 |
| 3 | 15(S)-HEPE EE | 3000^{a} | 10 | 300 | 309^{b}/306^{c} | 15 | 15 | 5 | 5 | 9 | 9 |

^{a}Due to the onset of clinical signs, including hunched posture, erect fur and abnormal gait and body weight loss, animals were given a dosing holiday from Day 48. Animals will start dosing at the lower dose level (3000 mg/kg/day) from Day 64.
^{b}Adjusted concentration for batches 2540M-1801 and 2540M-1802.
^{c}Adjusted concentration for batches 2540M/1901 and 2540M/1902.

TABLE 23
Animal Groupings

| | Animal Numbers | | | | | |
|---|---|---|---|---|---|---|
| Group | Main Study | | Recovery | | Toxicokinetic | |
| No. | Males | Females | Males | Females | Males | Females |
| 1 | 1001-1015 | 1501-1515 | 1101-1105 | 1601-1605 | 1201-1203 | 1701-1703 |
| 2 | 2001-2015 | 2501-2515 | 2101-2105 | 2601-2605 | 2201-2209 | 2701-2709 |
| 3 | 3001-3015 | 3501-3515 | 3101-3105 | 3601-3605 | 3201-3209 | 3701-3709 |

Spare animals will be numbered 4001M, 4002M and 4501F, 4502F.

Recovery Period: On completion of the dosing period, the designated recovery animals were retained for a minimum 4 week recovery period.

Justification of Route and Dosage Levels: The oral route of administration was selected for this study as this route is the intended route of human dosing. A previous 26-week oral toxicity in rats dosed once daily showed no adverse effects at the highest dose tested (1 mg/kg/day). See Warren, H (2017). A 26 Week Study of 15(S)-HEPE EE by Oral (Gavage) in Rats with a 4 Week Recovery Period. Charles River Study No. 529123. Given that no dose-limiting toxicity was seen on that study, an additional 28-day dose ranging study determined an oral maximum tolerated dose (MTD) of 6 g/kg/day. See Murie, E (ongoing). A Pilot 28-Day Study of DS102 by Oral Gavage in Rats. Charles River Study No. 506611. This current study was conducted to evaluate the chronic toxicity potential of 15(S)-HEPE EE (DS102) at this MTD to comply with ICH M3 (R2). See ICH Harmonised Tripartite Guideline M3 (R2). Nonclinical Safety Studies for the Conduct of Human Clinical Trials and Marketing Authorisation for Pharmaceuticals.

### 5.2 In-Life Procedures, Observations, and Measurements

The in-life procedures, observations, and measurements were performed for all main study and recovery animals. Toxicokinetic animals were weighed and had clinical observations recorded at the same timepoints as the main study animals.

Mortality/moribundity checks were made daily. Animals were observed for general health/mortality and moribundity. Animals were not removed from the cage during observation, unless necessary for identification or confirmation of possible findings.

Cage side observations were made daily. Animals were not removed from the cage during observation, unless for identification or confirmation of possible findings.

Detailed clinical observations were made weekly. Animals were removed from the cage for examination.

Post-dose observations were made regularly throughout the day. All animals were examined for reaction to treatment. The onset, intensity, and duration of these signs were recorded; particular attention was paid to the animals during and for the first hour after dosing.

Body weight measurements were made twice during the pretreatment period; once daily during the dosing period; and twice weekly during the recovery period. Animals were individually weighed. A weight was recorded on the first day of scheduled necropsy (for main and recovery animals only).

Food consumption was quantitatively measure weekly, beginning at week 1 through the dosing and recovery periods.

Water consumption was made regularly throughout the study by visual inspection of the water bottles.

Ophthalmic examinations were performed using an indirect ophthalmoscope after the application of a mydriatic agent (1% Tropicamide, Mydriacy). During pretreatment, all main study and recovery animals were examined. During weeks 13 and 26, main study animals (control and high dose) were examined. During week 30, all recovery animals were examined.

### 5.3 Laboratory Evaluations

Sample Collection: Blood was collected via the tail vein unless immediately prior to necropsy, when blood was collected from the orbital sinus under non-recoverable isoflurane anesthesia. Blood was collected in ascending animal order unless samples were collected prior to necropsy where the order of euthanasia was followed. Additional blood samples were obtained (e.g., due to clotting of non-serum samples) if permissible sampling frequency and blood volume were not exceeded. After collection, samples were transferred to the appropriate laboratory for processing. Samples were collected according to the following table.

TABLE 24
Samples for Clinical Pathology Evaluation

| | | Haema- | Coagu- | Clinical | Urinal- |
|---|---|---|---|---|---|
| Group Nos. | Time Point | tology | lation | Chemistry | ysis |
| Main Study and | Week 26 | — | — | — | X |
| Recovery animals | | | | | |
| Recovery | Day 183 | X | X | X | — |
| Main Study | Immediately | X | X | X | — |
| | Prior to | | | | |
| | Necropsy | | | | |
| Recovery animals | Week 30 | — | — | — | X |
| | Immediately | X | X | X | — |
| | Prior to | | | | |
| | Necropsy | | | | |
| Unscheduled | Before | X | X | X | — |
| euthanasia | euthanasia | | | | |
| (when possible) | | | | | |

X = sample to be collected; — = not applicable.

Hematology: A 0.5 mL blood sample was collected using the anticoagulant K₂EDTA. Blood smears were labelled, stained, and stored. The blood smears were analyzed according to the following parameters.

TABLE 25
Hematology Parameters

| | |
|---|---|
| Red blood cell count | White blood cell count |
| Haemoglobin concentration | Neutrophil count (absolute) |
| Haematocrit | Lymphocyte count (absolute) |
| Mean corpuscular volume | Monocyte count (absolute) |
| Red Blood Cell Distribution Width | Eosinophil count (absolute) |
| Mean corpuscular haemoglobin | Basophil count (absolute) |
| concentration | |
| Mean corpuscular haemoglobin | Large unstained cells (absolute) |
| Reticulocyte count (absolute) | |
| Platelet count | |

Coagulation: A 0.5 mL blood sample was collected using the anticoagulant 3.8% (w/v) trisodium citrate for processing to plasma. To assess coagulation the following coagulation parameters were measured.

TABLE 26
Coagulation Parameters

| | |
|---|---|
| Activated partial thromboplastin time | Prothrombin time |
| Fibrinogen | Sample Quality |

Clinical Chemistry: A 0.7 mL blood sample was collected using the anticoagulant lithium heparin for processing to plasma. To assess the clinical chemistry the following parameters were measured:

TABLE 27
Clinical Chemistry Parameters

| | |
|---|---|
| Alanine aminotransferase | Total protein |
| Aspartate aminotransferase | Albumin |
| Alkaline phosphatase | Globulin |
| Gamma-glutamyltransferase | Albumin/globulin ratio |
| Creatine Kinase | Glucose |
| Total bilirubin^{a} | Cholesterol |
| Urea | Triglycerides |
| Creatinine | Sodium |
| Calcium | Potassium |
| Phosphate | Chloride |
| | Sample Quality |

^{a}When total bilirubin is >8.55 μmol/L, indirect and direct bilirubin will also be measured.

Urinalysis: Urine was collected from animals over a period of 6 hours (+/−30 min) with the absence of food and the presence of water. After collection, samples were transferred to the appropriate laboratory for processing. To assess the urinalysis, the following parameters were measured:

TABLE 28
Urinalysis Parameters

| | |
|---|---|
| Colour | Protein |
| Appearance/Clarity | Glucose |
| Specific gravity | Bilirubin |
| Volume | Ketones |
| pH | Blood |

Bone Marrow Smear Analysis: Bone marrow was collected from all animals and preserved in 10% neutral buffered formalin.

Bioanalytic Sample Collection: A 0.5 mL blood sample was collected using the anticoagulant K₂EDTA via the jugular vein (or the tail vein if the jugular was not assessable) using sterile needles and disposable syringes. At completion of the blood collection schedule, the toxicokinetic animals were euthanized, and the carcasses were discarded without examination. Samples were collected according to the following table.

TABLE 29
Toxicokinetic Evaluation Sample Collection Schedule

| | | | Sample Collection Time Points | | | | | |
|---|---|---|---|---|---|---|---|---|
| Group | Animal | No. of | (Time Postdose) on Day 1 and Week 26 | | | | | |
| No. | Numbers | animals | 0 h^{a} | 1 h | 2 h | 4 h | 8 h | 24 h |
| 1 | 1201-1203 | 3M/3F | — | — | — | X | — | — |
| | 1701-1703 | | | | | | | |
| 2 | 2201-2203 | | X | — | — | X | — | — |
| | 2701-2703 | | | | | | | |
| | 2204-2206 | | — | X | — | — | X | — |
| | 2704-2706 | | | | | | | |
| | 2207-2209 | | — | — | X | — | — | X |
| | 2707-2709 | | | | | | | |
| 3 | 3201-3203 | | X | — | — | X | — | — |
| | 3701-3703^{b} | | | | | | | |
| | 3204-3206 | | — | X | — | — | X | — |
| | 3704-3706^{b} | | | | | | | |
| | 3207-3209 | | — | — | X | — | — | X |
| | 3707-3709^{b} | | | | | | | |

X = sample to be collected, — = not applicable.,
^{a}Sample will be collected before dosing
^{b}Animal 3702F had Day 1 samples collected, however on Day 44 this animal was euthanised for humane reasons. Extra samples will be collected at Week 26 from animals 3707F (0 h^{a} collection) and 3704F (4 h collection) to cover the missing timepoints for animal 3702F.

Bioanalytical Sample Processing: Samples were mixed gently and kept on crushed wet ice until centrifugation within 60 minutes of blood withdrawal. The samples were centrifuged at 1500 g for 10 minutes at 4° C. The resultant plasma was separated, transferred to uniquely labelled clear polypropylene tubes, and frozen as soon as possible over dry ice or in a freezer set to maintain −20° C.

Bioanalytical Sample Analysis: Plasma samples were analyzed for concentration of unesterified and total HEPE using validated analytical procedures. The following was determined:
a) Unesterified (HEPE occurring in plasma as the free fatty acid or bound to plasma albumin).
b) Total (quantifies unesterified HEPE and esterified HEPE (e.g., as triglycerides, cholesterol esters)) concentration at each sample time.

Incurred sample reanalysis (ISR) for unesterified HEPE was performed for this study as per the appropriate Standard Operating Procedure(s) of the bioanalytical laboratory. ISR has been successfully performed for the determination of total HEPE in rat plasma samples, using the analytical procedure referenced above, and is not required to be repeated in this study.

Any residual/retained bioanalytical samples were maintained for a minimum of 6 months following issue of the Draft Report after which samples were discarded. Alternatively, residual/retained samples were discarded prior to the 6 month period should the issue of the Final Report occur prior to the end of the 6 month retention period. An earlier discard of these residual/retained samples may also be requested and authorized by the Study Director.

Toxicokinetic Evaluation: Toxicokinetic parameters were estimated using Phoenix pharmacokinetic software. A non-compartmental approach consistent with the oral route of administration was used for parameter estimation. All parameters were generated from total free acid and unbound free acid of 15(S)-HEPE EE composition concentrations in plasma from Day 1 and Week 26 whenever practical. The following parameters were estimated.

TABLE 30
Parameters to be Estimated

| Parameter | Description of Parameter |
|---|---|
| Tmax | The time after dosing at which the maximum |
| | concentration was observed. |
| Cmax | The maximum observed concentration measured |
| | after dosing. |
| Cmax/D | The Cmax divided by the dose administered. |
| AUC(0-t) | The area under the concentration versus time curve |
| | from the start of dose administration to the last |
| | observed quantifiable concentration using the linear |
| | or linear/log trapezoidal method. |
| AUC(0-t)/D | The AUC(0-t) divided by the dose administered. |

Partial AUCs (between any given sample times) may be derived and reported to aid interpretation. Descriptive statistics (standard error for Cmax and AUC(0-t)) for appropriate grouping and sorting variables were generated using Phoenix. TK table and graphs were also generated.

### 5.4 Terminal Procedures

The following table summarizes the terminal procedures used in the study.

TABLE 31
Terminal Procedures for Main Study Animals

| | | | Scheduled | Necropsy Procedures | | | | |
|---|---|---|---|---|---|---|---|---|
| Group | Number | | Euthanasia | | Tissue | Organ | | |
| Number | M | F | Day | Necropsy | Collection^{a} | Weights^{a} | Histology^{a} | Histopathology^{a} |
| 1 | 15 | 15 | 183/184 | X | X | X | Full Tissue | Full Tissue |
| 2 | 15 | 15 | | | | | Gross Lesions | Gross Lesions |
| | | | | | | | Target Tissues^{b} | Target Tissues^{b} |
| 3 | 15 | 15 | | | | | Full Tissue | Full Tissue |
| 1 | 5 | 5 | 211 | | | | Gross Lesions | Gross Lesions |
| | | | | | | | Target Tissues^{b} | Target Tissues^{b} |
| 2 | 5 | 5 | | | | | Gross Lesions | Gross Lesions |
| | | | | | | | Target Tissues^{b} | Target Tissues^{b} |
| 3 | 5 | 5 | | | | | Gross Lesions | Gross Lesions |
| | | | | | | | Target Tissues^{b} | Target Tissues^{b} |

| | | | | | |
|---|---|---|---|---|---|
| Unscheduled Deaths | X | X | — | Full Tissue | Full Tissue |

X = procedure to be conducted; — = not applicable
^{a}See Tissue Collection and Preservation table for listing of tissues.
^{b}Target tissues are liver (male and female), thyroid (male and female), kidney (female only), bone marrow sternum (male only).

Unscheduled Deaths: If a main study or recovery animal died during the study, a necropsy was performed and specified tissues were saved. If necessary, the animals were refrigerated to minimize autolysis. Main study or recovery animals were euthanized for humane reasons. The body weight was recorded and samples for evaluation of clinical pathology parameters, were obtained if possible. These animals were necropsied, and specified tissues were retained. If necessary, the animals were refrigerated to minimize autolysis. Toxicokinetic animals that died during the study or were euthanized for humane reasons were subjected to a limited necropsy examination. The examination consisted of an evaluation of the organs and tissues in the thoracic, abdominal, and pelvic cavities, with no tissues retained. During the limited necropsy examination, special attention was directed to evidence of possible gavage trauma. The body weight was recorded and samples for evaluation of toxicokinetic analysis were collected.

Scheduled Euthanasia: Main study and recovery animals that survived until scheduled euthanasia were euthanized by exposing the animal to a rising level of carbon dioxide; a terminal body weight was recorded and the animal was subsequently exsanguinated. When possible, the animals were euthanized in a rotating order across dose groups such that similar numbers of animals from each group, including controls were necropsied throughout the day. Animals were not fasted before their scheduled necropsy. Toxicokinetic animals that survived until scheduled euthanasia were euthanized by approved methods. No necropsies were performed and no tissues were collected.

Necropsy: Main study and recovery animals were subjected to a complete necropsy examination, which included evaluation of the carcass and musculoskeletal system; all external surfaces and orifices; cranial cavity and external surfaces of the brain; and thoracic, abdominal, and pelvic cavities with their associated organs and tissues. Necropsy procedures were performed by qualified personnel with appropriate training and experience in animal anatomy and gross pathology.

Organ Weights: The organs identified for weighing in the Tissues Collection and Preservation table were weighed at necropsy (unless otherwise specified) for all scheduled euthanasia animals. Organ weights were not recorded for animals found dead or euthanized in poor condition or in extremis. Paired organs were weighed together. In the event of gross abnormalities, in addition to the combined weight, the weight of each organ of a pair was taken and entered as a tissue comment. Terminal body and brain weights were used for organ weight analysis.

Tissue Collection and Preservation: Tissues collected according to the table below were preserved in 10% neutral buffered formalin.

TABLE 32
Tissue Collection and Preservation Table

| | | Macroscopic | | |
|---|---|---|---|---|
| | | Evaluation and | Histology | Microscopic |
| Organ | Weigh | Collection | Processing | Evaluation |
| Animal ID | — | X | — | — |
| Artery, aorta | — | X | X | X |
| Body cavity, | — | X | — | — |
| nasal | | | | |
| Bone marrow, | — | X | X | X |
| sternum | | | | |
| Bone marrow | — | X^{a} | — | — |
| smear | | | | |
| Bone, femur | — | X (1) | X (1) | X (1) |
| Bone, sternum | — | X | X | X |
| Brain | X | X | X | X |
| Cervix | — | X | X | X |
| Epididymis | X (2) | X (2)^{b} | X (2) | X (2) |
| Esophagus | — | X | X | X |
| Eye | — | X (2)^{b} | X (2) | X (2) |
| Gland, adrenal | X (2) | X (2) | X (2) | X (2) |
| Gland, clitoral | — | X (2) | — | — |
| Gland, lacrimal | — | X (2, | — | — |
| | | extra-orbital) | | |
| Gland, | — | X (2) | X (1) | X (1) |
| Harderian | | | | |
| Gland, | — | X | X | X |
| mammary | | | | |
| Gland, | — | X (2) | X (2) | X (2) |
| parathyroid | | | | |
| Gland, pituitary | X | X | X | X |
| Gland, preputial | — | X (2) | — | — |
| Gland, prostate | X | X | X | X |
| Gland, salivary, | — | X (2) | X (1) | X (1) |
| submandibular | | | | |
| Gland, salivary, | — | X (2) | — | — |
| sublingual | | | | |
| Gland salivary, | — | X (2) | — | — |
| parotid | | | | |
| Gland, seminal | — | X (2) | X (2) | X (2) |
| vesicle | | | | |
| Gland, thyroid/ | X (2)^{d} | — | — | — |
| Gland parathyroid | | | | |
| Gland, thyroid | — | X (2) | X (2) | X (2) |
| Gland, Zymbal's | — | X (2) | — | — |
| Gut-associated | — | X | X | X |
| lymphoid tissue^{c} | | | | |
| Heart | X | X | X | X |
| Joint, | — | X (1) | X (1) | X (1) |
| femorotibial | | | | |
| Kidney | X (2) | X (2) | X (2) | X (2) |
| Large intestine, | — | X | X | X |
| cecum | | | | |
| Large intestine, | — | X | X | X |
| colon | | | | |
| Large intestine, | — | X | X | X |
| rectum | | | | |
| Larynx | — | X | — | — |
| Liver | X | X | X | X |
| Lung | — | X | X | X |
| Lymph node, | — | X (2) | X (1) | X (1) |
| mandibular | | | | |
| Lymph node, | — | X | X | X |
| mesenteric | | | | |
| Muscle, skeletal | — | X | X | X |
| Nasopharynx | — | X | — | — |
| Nerve, optic | — | X (2)^{b} | X (2) | X (2) |
| Nerve, sciatic | — | X (2) | X (1) | X (1) |
| Ovary | X (2) | X (2) | X (2) | X (2) |
| Oviduct | — | X (2) | — | — |
| Pancreas | — | X | X | X |
| Skin | — | X | X | X |
| Small intestine, | — | X | X | X |
| duodenum | | | | |
| Small intestine, | — | X | X | X |
| ileum | | | | |
| Small intestine, | — | X | X | X |
| jejunum | | | | |
| Spinal cord | — | X | X | X |
| Spleen | X | X | X | X |
| Stomach | — | X | X | X |
| Testis | X (2) | X (2)^{b} | X (2) | X (2) |
| Thymus | X | X | X | X |
| Tongue | — | X | X | X |
| Trachea | — | X | X | X |
| Ureter | — | X (2) | — | — |
| Urinary bladder | — | X | X | X |
| Uterus/Cervix | X | — | — | — |
| Uterus | — | X | X | X |
| Vagina | — | X | X | X |

X = Procedure to be conducted. — = Not applicable. (1) = one side. (2) = both sides.
Macroscopic abnormalities in the organs listed and in other organs will be sampled at necropsy, processed for histology and examined microscopically.
^{a}Two bone marrow smears will be collected from the femur at scheduled and unscheduled necropsies (for possible examination). Smears will not be collected from animals that are found dead or from animals that were euthanised moribund and then stored in the refrigerator prior to necropsy. Bone marrow smears are allowed to air dry and are not fixed in formalin.
^{b}Eyes and optic nerves are preserved in Davidson's fixative. Testes and epididymides are preserved in modified Davidson's fixative.
^{c}From small intestine: Peyer's patch or solitary lymphoid follicle.
^{d}Weighed after fixation.

### 5.5 Dose Formulation and Analysis

Control Item: The control item for this study was 0.5% HPMC in Milli-Q water. The control item was prepared as required and stored in a refrigerator set to maintain 4° C. when not in use. The prepared control item was removed from the refrigerator and stirred for at least 30 minutes before dosing. The control item was also stirred continuously during dosing.

Test Item: The test item for this study was DS102 or 15(S)-HEPE EE. Test item dosing formulations were prepared based on a method established at appropriate concentrations to meet dosage level requirements. The dosing formulations were prepared weekly and stored in a refrigerator set to maintain 4° C. and dispensed daily. The dosing formulations were removed from the refrigerator and stirred for at least 30 minutes before dosing. The dosing formulation were also be stirred continuously during dosing.

The analyzed samples were submitted within the established stability period. All analyzed samples were transferred at ambient temperature to the analytical laboratory. Any residual/retained analytical samples were maintained for a minimum of 6 months following issue of the draft report after which samples will be discarded. Alternatively, residual/retained samples were discarded prior to the 6 month period in the situation that the issue of the final report occurred prior to the end of the 6 month retention period. An earlier discard of these residual/retained samples may also be authorized by the study director after consultation with the sponsor. Does formulation samples were collected for analysis as indicated in the following table. Additional samples may be collected and analyzed at the discretion of the study director.

TABLE 33
Dose Formulation Sample Collection Schedule

| Interval | Concentration | Homogeneity | Stability |
|---|---|---|---|
| Day 1 | All groups | Groups 2-3^{a} | N/A |
| Week 2 | All groups | Groups 2-3^{a} | N/A |
| Week 10 | Group 3 | Group 3 | N/A |
| Week 13 | All groups | N/A | N/A |
| Week 14 | All groups | All groups^{a} | N/A |
| Week 26 | All groups | N/A | N/A |

N/A = not applicable.
^{a}The homogeneity results obtained from the top, middle and bottom for the Group 2 to 3 preparation will be averaged and utilized as the concentration results.

The analysis described below was performed by UPLC with UV detection using a validated analytical procedure.

### Concentration Analysis:

Sample for Analysis: Duplicate top, middle and bottom samples (duplicate middle only for control) were sent for analysis. Where concentration only is being assessed duplicate middle only samples were collected.
Backup Samples: Triplicate top, middle and bottom samples (triplicate middle only for control). Backup samples may be analyzed at the discretion of the study director. Where concentration only is being assessed triplicate middle only samples will be collected.
Sampling Containers: Appropriately sized volumetric flasks.
Sample Volume:
   Group 1: 0.5 mL into a 50 mL volumetric flask.
   Group 2: 0.25 mL into a 50 mL volumetric flask.
   Group 3: 0.25 mL into a 50 mL volumetric flask.
   The weight of all samples was recorded.
Storage Conditions: Kept in a refrigerator set to maintain 4° C.
Acceptance Criteria: The criteria for acceptability was mean sample concentration results within or equal to +10% of theoretical concentration. For homogeneity, the criteria for acceptability was a relative standard deviation (RSD) of concentrations of ≤10% for each group.

Stability Analysis: Stability analyses performed previously demonstrated that the test item is stable in the vehicle when prepared and stored under the same conditions at concentrations bracketing those used in the present study. See Rogers, E (2018). Validation of an Ultra High-Performance Liquid Chromatographic Method for the Determination of 15(S) HEPE EE in oral (Gavage) Dosing Formulations. Id.

### 5.6 Test System

Animals: The study used male and female Sprague Dawley rats purchased from Charles River UK Limited (Margate, Kent, UK). The target age at the initial dosing was 7-8 weeks. The target weight at the initial dosing was 175-300 g (males) and 120-250 g (females). Each animal was identified using a subcutaneously implanted electronic cylindrical, “glass-sealed” TROVAN microchip.

### 5.7 Husbandry

Housing: The animals were allowed to acclimate to the test facility rodent toxicology accommodation for a period of up to 3 weeks before the commencement of dosing. Animals were randomly assigned to groups. Males and females were randomized separately. Animals in poor health were not assigned to groups. Animals were housed 2 or 3 per cage by sex in appropriately sized suspended polycarbonate/polypropylene cages with stainless steel grid tops and solid bottoms. Sterilized white wood shavings were used as bedding material.

Environmental Conditions: The targeted conditions for animal room environment were as follows: temperature 19-23° C.; humidity 40-70%; ventilation a minimum of 10 air changes per hour; light cycle 12 hour light and 12 hour dark. Control of light, temperature, and humidity was automatically controlled and was continuously monitored and recorded.

Diet: SDS rat and mouse (modified) no. 1 diet SQC expanded was provided ad libitum. Water from a public supply was provided ad libitum from a water bottle. The water used by the test facility was analyzed at regular intervals for dissolved materials, heavy metals, pesticide residues, pH, nitrates and nitrites. Microbiological screening was also conducted.

Animal Enrichment: Animals were socially housed for psychological/environmental enrichment and were provided with items such as a device for hiding in and an object for chewing, except when interrupted by study procedures/activities.

Veterinary Care: Veterinary care was available throughout the course of the study and animals were examined by the veterinary staff as warranted by clinical signs or other changes. All veterinary examinations and recommended therapeutic treatments, if any, were documented in the study records.

### 5.8 Histology and Histopathology

Histology: Tissues identified in the Tissue Collection and Preservation table were embedded in paraffin, sectioned, mounted on glass slides, and stained with hematoxylin and eosin.

Histopathology: Histopathological evaluations were performed by a board-certified veterinary pathologist or a veterinary pathologist with training and experience in laboratory animal pathology. Microscopic examination was carried out for tissues from main study control and high dose animals, unscheduled deaths and gross lesions (including from the main study low dose group and recovery animals). If treatment effects were present in the main study high dose group, microscopic examination of target tissues from the main study low dose group and recovery animals were carried out after agreement with the Sponsor and Study Director by protocol amendment.

### 5.9 Computerized Systems

Computerized Systems: The follow table of computerized systems were available for use in the study:

TABLE 34
Critical Computerized Systems

| System Name | Description of Data Collected and/or Analysed |
|---|---|
| Dispense | Test item receipt, accountability and/or |
| | formulation activities. |
| Provantis | Applicable In-life, clinical pathology and |
| | postmortem |
| Empower 3 | Chromatography Data Collection and Processing |
| | (Formulation Analysis) |
| Analyst | Chromatography Data Collection (Bioanalysis) |
| Watson | Chromatography Processing (Bioanalysis) |
| Phoenix | Non-compartmental analysis and descriptive |
| | statistics (Toxicokinetic Evaluation) |
| In-house reporting | Applicable In-life, clinical pathology and |
| software Nevis 2012 | postmortem |
| (using SAS) | |
| Deviation Information | Deviations |
| Library | |
| Trend 963 Secure | Continuous temperature and humidity recording. |
| | Alarms for animal rooms, refrigerators, freezers |
| | and incubators. |
| Share Document | Reporting |
| Management System | |
| Docusign | Collection of Part 11 compliant signature |

### 5.10 Statistical Analysis

Data collected during the pre-dose period was tabulated, summarized and statistically analyzed. All statistical analysis was performed within the respective study phase, unless otherwise noted. Numerical data collected on scheduled occasions (with the exception of data from the toxicokinetic animals) was summarized and statistically analyzed as indicated below according to sex and occasion.

Constructed Variables: Body weight changes were calculated between each scheduled interval. Organ weight relative to body weight was calculated against the terminal body weight for scheduled intervals. Organ weight relative to brain weight was calculated against the brain weight for scheduled intervals.

Descriptive Statistical Analysis: Means, standard deviations (or % coefficient of variation or standard error, when deemed appropriate), percentages, numbers, and/or incidences will be reported as appropriate by dataset.

Inferential Statistical Analysis: All statistical analysis was conducted at the 5% significance level. All pairwise comparisons were conducted using two sided tests and were reported at the 1% and 5% levels, unless otherwise noted. The pairwise comparisons of interest are listed below:
Group 2 vs. Group 1
Group 3 vs. Group 1

Analysis were performed according to the matrix below when possible, but excluded any group with less than 3 observations.

TABLE 35
Statistical Matrix

| | Statistical Method |
|---|---|
| | Parametric/Non- |
| Variables for Inferential Analysis | parametric |
| Body Weight | X |
| Body Weight Gains | X |
| Haematology Variables | X |
| Coagulation Variables | X |
| Clinical Chemistry Variables | X |
| Urinalysis Variables | X |
| Organ Weights | X |
| Organ Weight relative to Body Weight | X |
| Organ Weight relative to Brain Weight | X |

Parametric/Non-parametric: Levene's Test was used to assess the homogeneity of group variances. The groups were compared using an overall one-way ANOVA F-test if Levene's test is not significant or the Kruskal-Wallis test was not significant. If the overall F-test or Kruskal-Wallis test was found to be significant, then pairwise comparisons were conducted using Dunnett's or Dunn's test, respectively.

### 5.11 Results

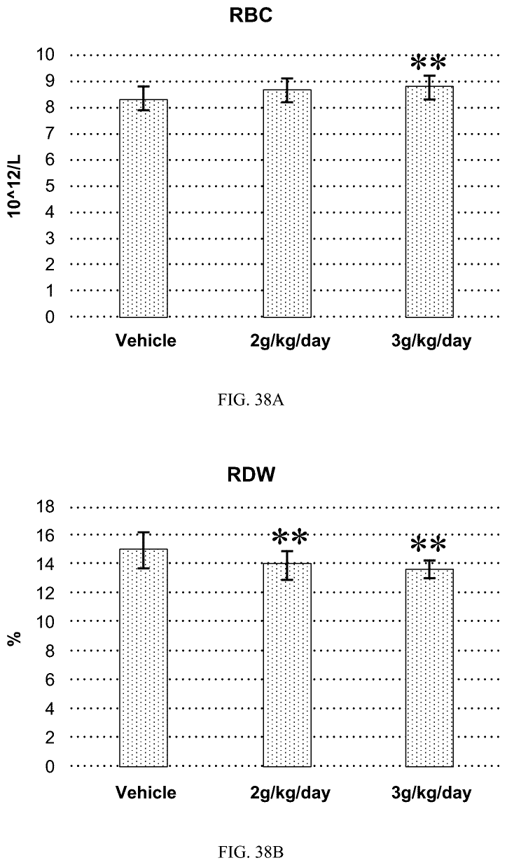

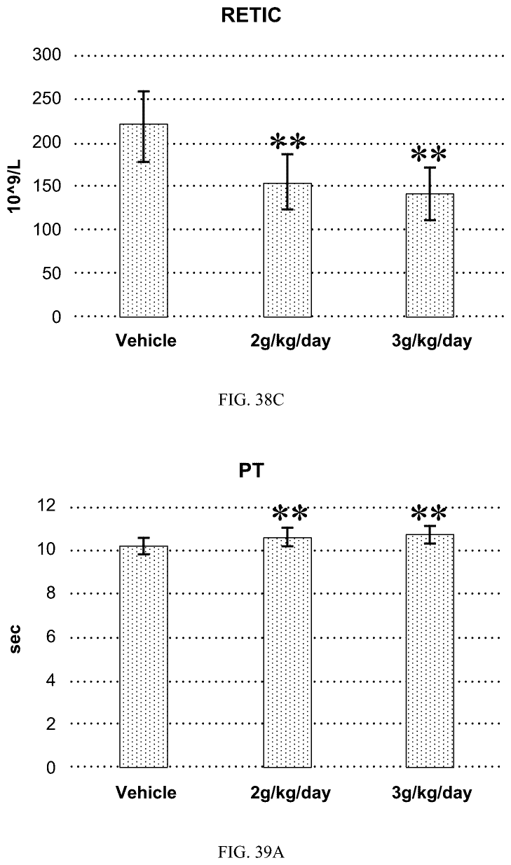

Change in red blood cell count, red blood cell distribution width, and reticulocyte count: FIG. 38A-38C shows change in red blood cell count (RBC), red blood cell distribution width (RDW), and reticulocyte count (RETIC) for rats administered 15(s)-HEPE EE or placebo. 15(S)-HEPE EE and placebo were administered once daily by oral gavage 7 days a week for a minimum of 26 weeks. The volume for each animal was based on the most recent body weight measurements. As noted in Table 36, the highest dose of 6 g/kg/day was not well tolerated and was replaced with a lower 3 g/kg/day.

TABLE 36
Change in RBC, RDW and RETIC in
Rats administered 15(S)-HEPE EE

| | STUDY | | |
|---|---|---|---|
| | 26 week rat study (CRL509554) | | |
| | 15 (S)-HEPE EE Dose (mg/kg/day) | | |
| | 0 (vehicle) | 2000 | 6000/3000 |
| Red blood cell count | | | |
| (RBC) (10\hat{ }12/L) | | | |
| Mean | 8.38 | 8.67 | 8.81** |
| (SD) | 0.42 | 0.41 | 0.42 |
| Red Cell Distribution | | | |
| Width (RDW) (%) | | | |
| Mean | 14.98 | 13.91** | 13.60** |
| (SD) | 1.23 | 0.96 | 0.57 |
| Reticulocyte count | | | |
| (RETIC) (10\hat{ }9/L) | | | |
| Mean | 219.51 | 154.60* | 141.46** |
| (SD) | 40.41 | 32.31 | 30.57 |

Anova & Dunnett:
*= p ≤ 0.05;
**= p ≤ 0.01
All presented values were observed in males
Highest dose of 6 g/kg/day was not well tolerated and was replaced with lower 3 g/kg/day dose

Change in RBC: FIG. 38A and Table 36 show that administration of the 3 g/kg/day dose resulted in a significant increase in RBC compared to control.

Change in RDW: FIG. 38B and Table 36 show that administration of both the 2 g/kg/day and 3 g/kg/day doses resulted in a significant decrease in RDW compared to control.

Change in RETIC: FIG. 38C and Table 36 show that administration of both the 2 g/kg/day and 3 g/kg/day doses resulted in a significant decrease in RETIC compared to control.

These results suggest that 15-HEPE has therapeutic potential for a number of conditions that affect hemoglobin and red blood cells.

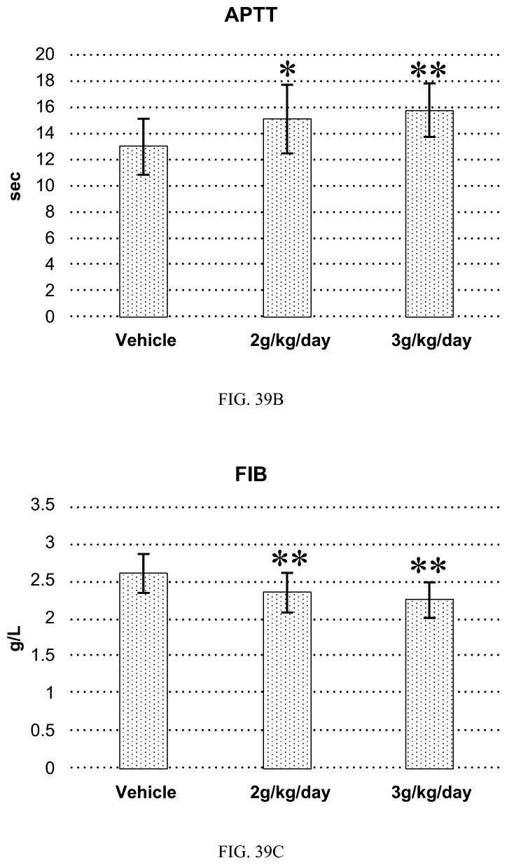

Change in prothrombin time, activated partial thromboplastin time, and fibrinogen concentration: FIG. 39A-39C shows change in prothrombin time (PT), activated partial thromboplastin time (APTT), and fibrinogen (FIB) concentration for rats administered 15(s)-HEPE EE or placebo. 15(S)-HEPE EE and placebo were administered once daily by oral gavage 7 days a week for a minimum of 26 weeks. The volume for each animal was based on the most recent body weight measurements. As noted in Table 37, the highest dose of 6 g/kg/day was not well tolerated and was replaced with a lower 3 g/kg/day.

TABLE 37
Change in PT, APTT, and FIB concentration
in Rats administered 15(S)-HEPE EE

| | STUDY | | |
|---|---|---|---|
| | 26 week rat study (CRL509554) | | |
| | 15 (S)-HEPE EE Dose (mg/kg/day) | | |
| | 0 (vehicle) | 2000 | 6000/3000 |
| Prothrombin Time (PT) (sec) | | | |
| Mean | 10.25 | 10.69** | 10.81** |
| (SD) | 0.34 | 0.41 | 0.47 |
| Activated Partial Thromboplastin | | | |
| Time (APTT) (sec) | | | |
| Mean | 12.92 | 15.03* | 15.73** |
| (SD) | 2.12 | 2.64 | 2.00 |
| Fibrinogen (FIB) (g/L) | | | |
| Mean | 2.61 | 2.35** | 2.26** |
| (SD) | 0.26 | 0.29 | 0.24 |

Anova & Dunnett:
*= p ≤ 0.05;
**= p ≤ 0.01
All presented values were observed in males
Highest dose of 6 g/kg/day was not well tolerated and was replaced with lower 3 g/kg/day dose

Change in PT: FIG. 39A and Table 37 show that administration of both the 2 g/kg/day and 3 g/kg/day dose resulted in a significant increase in PT compared to control.

Change in APTT: FIG. 39B and Table 37 show that administration of both the 2 g/kg/day and 3 g/kg/day doses resulted in a significant increase in APTT compared to control.

Change in FIB concentration: FIG. 39C and Table 37 show that administration of both the 2 g/kg/day and 3 g/kg/day doses resulted in a significant decrease in FIB concentration compared to control.

These results suggest that 15-HEPE has anticoagulant and antithrombotic effects.

### Example 6: Epeleuton, A Novel Synthetic ω-3 Fatty Acid, Diminishes Hypoxia/Reperfusion-induced Inflammatory Vasculopathy and Hemolysis in a Mouse Model of Sickle Cell Disease

Inflammatory vasculopathy is critical in sickle cell disease (SCD)-associated acute and chronic organ damage. An imbalance between pro-inflammatory and pro-resolving mechanisms in response to different triggers such as hypoxia/reoxygenation or infections has been proposed to contribute to SCD disease progression. Administration of specialized pro-resolving lipid mediators may provide an effective therapeutic strategy to target inflammatory vasculopathy and to modulate inflammatory response

Epeleuton (15-HEPE EE) is a novel orally administered second-generation ω-3 fatty acid with a favorable clinical safety profile. The studies of the present technology show that Epeleuton protects against systemic and local inflammatory response in target organs for SCD in humanized sickle cell mice. This was associated with the reprogramming of spleen macrophages towards a pro-resolving pattern and the improvement of red cell features, resulting in reduced hemolysis and sickling compared with vehicle treated SCD mice. In addition, Epeleuton prevents the hypoxia/reoxygenation induced activation of NF-kB with downregulation of NLRP3 inflammasome, and of vascular activation markers in SCD mice. Collectively this data support the potential therapeutic utility of Epeleuton and provide the rationale for the design of clinical trials to evaluate the efficacy of Epeleuton in patients with SCD.

SCD is a genetic disease in which there is a point mutation of the globin gene at the β7 position. This leads to the production of hemoglobin S (HbS), which has the unique property of forming polymers under low oxygen tensions, generating rigid and dense erythrocytes which have a reduced survival in the circulation, resulting in chronic hemolysis and anemia. SCD is also characterized by acute and chronic painful episodes, most often in the context of vaso-occlusive crisis (VOC), a definition based on the notion that occlusion of small vessels and/or capillaries by sickled cells is the triggering mechanism for the generation of inflammation and pain. This inflammatory vasculopathy plays a key role in the pathogenesis of both acute and chronic sickle cell-related complications, interfacing the whole blood compartment and being involved in cell-cell pro-adhesion phenomena. Regulatory approvals of new therapeutic tools for SCD such as crizanlizumab, an injectable monoclonal antibody against P-selectin, which interferes with erythrocyte-neutrophil-endothelial cells adhesion, and voxelotor, a HbS polymerization inhibitor, are important recent milestones. However, the persistence of VOCs, as well as chronic disease progression, still represent unmet therapeutic needs, which require additional therapeutic approaches.

Studies in different models of inflammatory vasculopathy such as atherosclerosis, diabetes, or ischemic cardiomyopathy, have highlighted the yin-yang role of inflammatory response when it is not balanced by pro-resolving mechanisms. Evidence from this laboratory demonstrates that the pathophysiology of SCD goes beyond the well-known role of polymerized Hb and includes imbalances in pro-inflammatory versus pro-resolving mechanisms. This imbalance in turn might promote disease progression and the severity of clinical manifestations. Consistent with this finding, multiple independent clinical studies profiling plasma, red cells, or platelet fatty acid composition in patients with SCD demonstrate that there is an underlying relative deficiency of omega-3 fatty acids and their pro-resolving metabolites in SCD under both basal and inflammatory conditions. The observed fatty acid decreases in SCD have also been shown to correlate with inflammation and hemoglobin in patients with SCD. An attempt to target inflammatory vasculopathy and to modulate inflammatory response through pro-resolving mechanisms has been made in other diseases such as in cardiovascular disease by administrating omega-3 polyunsaturated fatty acids (ω-3 PUFAs). Indeed, supplementation with omega-3 fatty acids might act as multimodal therapy by (i) affecting cell membrane lipid composition; (ii) modulating soluble and cellular inflammatory response and coagulation cascade; and (iii) favoring nitric oxide production.

As previously shown, the humanized mouse model for SCD, PUFA supplementation protected against acute sickle cell-related organ damage induced by hypoxia/reoxygenation stress as model for VOCs. These data were complemented by evidence from human studies that the administration of ω-3 PUFAs to SCD subjects reduced VOCs, pain episodes, and blood transfusions. However, the limited clinical and molecular data on PUFA administration for SCD patients still represent an obstacle to the routinely use of PUFA therapeutic approach for patients with SCD. In addition, the specific formulation of PUFA supplementation markedly affects their bioavailability and clinical outcomes, as demonstrated by the different effects of EPA and DHA formulations on cardiovascular outcomes. Notably, a phase II multicenter randomized double-blind placebo-controlled study in SCD patients reported that SC411, a novel docosahexaenoic acid (DHA) formulation, reduced pro-inflammatory markers and ameliorated home management of pain with a positive trend in decreasing pain episode in SCD subjects, although it did not reach statistical significance when compared to placebo (#NCT02973360).

Epeleuton is 15-hydroxy-eicosapentaenoic acid (15(S)-HEPE)ethyl ester, a second generation synthetic ω-3 fatty acid derivative of eicosapentaenoic acid (EPA). Epeleuton is a downstream 15-lipoxygenase metabolite of EPA, which is endogenously produced by hypoxic vascular endothelial cells and can be further transformed to produce lipoxin (LX) A5 and B5. As of February 2023, Epeleuton has completed an extensive toxicological evaluation and been administered to more than 340 subjects in phase 1 and phase 2 clinical studies, with a favorable safety and tolerability profile. Studies in animal models of atherosclerosis and in patients with non-alcoholic fatty liver disease show protective effects of Epeleuton against markers of inflammatory vasculopathy and systemic inflammation, suggesting its possible transferability to SCD. Notably, decreased concentrations of 15(S)-HEPE were observed in humanized SCD mice under both normoxic and hypoxic conditions compared to health controls, a finding which is consistent with previously reported decreased fatty acid concentrations in patients with SCD.

Using a humanized mouse model for SCD, the present technology shows the benefit of the oral administration of Epeleuton in preventing H/R induced stress in lung, liver, and kidney, which are target organs of SCD. Epeleuton reprograms the functional profile of spleen macrophages towards a pro-resolving signature, to prevent H/R induced vascular endothelial overactivation and to decrease intravascular hemolysis. Collectively this data support the potential therapeutic utility of Epeleuton and provide the rationale for the design of clinical trials to evaluate the efficacy of Epeleuton in patients with SCD.

### 6.1 Materials and Methods

Animals and design of the study: Experiments were performed on 4-5 months-old sex matched healthy control (Hba^{tm1(HBA) Tow} Hbb^{tm3(HBG1, HBB) Tow}) and SCD (Hba^{tm1(HBA)Tow} Hbb^{tm2(HBG1.HBB*)Tow)} mice. The study used 4-5 months-old mice. The sickle cell related organ damage is not so severe to generate confounding factors when the impact of H/R induced stress is evaluated. This is also corroborated by the observation of Kasztan M et al who show gender-related differences in kidney function of humanized SCD mice becoming significant after 24 weeks (6 months) of age. The size of the mouse groups was established based on studies with the same mouse model for SCD generated by us and other labs. Mice were randomly assigned to the different treatment groups and analysis. Healthy and SS mice were treated with ether vehicle (hydroxypropyl methyl cellulose, HPMC, 0.5%) or Epeleuton in HPMC 0.5% (1000 mg/kg/day) administrated by gavage for 6 weeks. The dosage of Epeleuton was based on previous pharmacokinetic studies in rodents, which yielded concentrations in plasma comparable to those observed in patients. When indicated, mice underwent a hypoxia/reoxygenation stress (H/R): 10 hours hypoxia (8% oxygen) followed by 3 hours reoxygenation (21% oxygen) to mimic acute vaso-occlusive crisis as previously described. Whole blood and organs were collected from each mouse under isoflurane anesthesia. Organs were immediately removed and divided into two portions which were either immediately frozen in liquid nitrogen or fixed in 10% formalin and embedded in paraffin for histology. Hematological parameters were evaluated with a Sysmex XN-1000 Hematology Analyzer and red cell morphology was evaluated as previously reported. Biochemical assays were performed using standard biochemical assays, as previously reported. Plasmatic Pentraxin 2 and CCL-2 and CXCL-2 were measured respectively using the Mouse Pentraxin 2/SAP Quantikine ELISA Kit and the next generation ELISA kit according to the manufacturer's instruction.

Lung, kidney and liver histological analysis: Paraffin-embedded tissue blocks were cut into 2-3 μm sections and mounted on adhesion microscope glass slides for Hematoxylin-Eosin (H&E), and Perls' staining for iron content. The analysis was performed on four different fields at a magnification 200×. Tissue pathological analysis, inflammatory cell infiltrate, the presence of thrombi and iron deposition were carried out by blinded pathologists as previously described.

Immunoblot analysis: Packed red cells were lysed in ice-cold phosphate lysis buffer (5 mM Na₂HPO₄, pH 8.0, containing protease inhibitor cocktail tablets, 3 mM benzamidine final concentration, 1 mM Na3VO4 final concentration) and centrifuged 10 min at 4° C. at 12,000 g. Red cell membrane (ghost) and cytosol fractions were obtained as previously reported. Frozen lung, kidney and aorta from each studied group were homogenized and lysed with iced lyses buffer (150 mM NaCl, 25 mM bicine, 0.1% SDS, 2% Triton X-100, 1 mM EDTA, protease inhibitor cocktail tablets, 1 mM Na3VO4 final concentration) then centrifuged 30 min at 4° C. at 12,000 g. Specific antibodies used are: anti Phospho-Tyrosine (clone PY99 from SCBT, Santa Cruz, CA (dilution 1:3000, 75 μg loaded) and clone 4G10 from Merck KGaA, Darmstadt, Germany (dilution 1:1600, 75 μgr loaded)); anti Phospho (Ser536) NF-kB p65 (dilution 1:1000, 75 μg loaded) and anti NF-kB p65 (clone C22B4) (dilution 1:1000, 75 μgr loaded) from Cell Signaling Technology (Danvers, MA, USA); anti-NLRP3/NALP3 from Vinci-Biochem Srl, Vinci, Italy (dilution 1:1000, 75 μgr loaded); anti Endothelin-1 (ET-1) form AbCam, Cambridge, UK (dilution 1:1000, 75 μgr loaded); anti VCAM-1 (R and D Systems, Minneapolis, MN, USA (dilution 1:1000, 40 μgr loaded)); anti ICAM-1 (clone EP1442Y, dilution 1:1000, 75 μgr loaded, AbCam, Cambridge, UK); anti TXAS-1 (Cayman, Ann Arbor, MI, USA); anti Nrf2-phospho-S40 (Clone EP1809Y, dilution 1:1000, 75 μgr loaded, AbCam, Cambridge, UK); anti-Nrf2 (dilution 1:1000, 75 μgr loaded, AbCam, Cambridge, UK); anti Nqo1 from Santa Cruz Biotechnology, Inc, CA, USA (clone C-19; dilution 1:1000, 75 μgr/μl loaded); anti-heme oxygenase 1(HO-1) form SCBT (Santa Cruz, CA, USA (dilution 1:1000, 50 μgr loaded); E-selectin (H-300) (dilution 1:1000, 75 μgr loaded); anti GAPDH from SCBT (Santa Cruz, CA, USA (dilution 1:5000, 50 μgr loaded)); anti Actin from SCBT (Santa Cruz, CA, USA (clone 2A3, dilution 1:1000, 50 μgr/loaded)). Secondary donkey anti-rabbit lgG (dilution 1:10000) and anti-mouse lgG (dilution 1:5000) HRP conjugated were from GE Healthcare Life Sciences (Little Chalfont, UK); secondary donkey anti goat lgG (dilution 1:10000) HRP conjugated was from SCBT, secondary donkey anti rat IgG (dilution 1:5000) HRP conjugated was from AbCam. Blots were developed with Luminata Forte Chemiluminescent HRP Substrate from Merk Millipore (Burlington, MA, USA), and images were acquired with the Alliance Q9 Advanced imaging system (Uvitec, UK). Densitometric analyses were performed with the Nine Alliance software (Uvitec, UK). Oxidized proteins were monitored by using the Oxyblot Protein Oxidation Detection Kit (EMD Millipore) following the manufacturer instructions.

Analysis of circulating and spleen neutrophils and kidney leukocyte: Circulating and splenic total leukocytes and neutrophils were identified in freshly collected blood and spleen by flow cytometry as CD45 positive cells and CD45/Ly6G double positive cells. See Supplementary Methods for details.

Analysis of Efferocytosis and Macrophage Receptors: Spleen and lungs were gently dissociated into single cells using GentleMACS dissociator and stained with anti-CD45 PE-Cy5.5, F4/80 PE, CD11b BV510 (Biolegend, CA, United States). Following staining cells were fixed, permeabilized, and counter-labeled with Ter-119 FITC to measure macrophage intracellular fluorescence associated with phagocytosed neutrophils and red cells respectively. Cells stained as above without permeabilization served as negative controls of intracellular staining, as previously reported (1). CD68 APC, and CD68 PE-Cy7 (Biolegend) were used to determine surface expression of phagocytic receptors on spleen and lungs macrophages identified using an anti-F4/80-APC-Cy7 antibody. Flow cytometry was carried out on a BD FACS Canto II and results were analyzed.

Statistical analysis: Normality was assessed with Shapiro-Wilk test. Two tailed unpaired Student t-test or two-way analysis of variance with Tukey's multiple comparisons were used for data analyses. Whenever indicated, the analyses were performed using unpaired Student t-test with Bonferroni correction. Data show values from individual mice and are presented with mean±SEM. Differences with p<0.05 were considered significant.

Flow-cytometric analysis of total leukocytes and neutrophils: 10 μl of heparinized blood was collected from anesthetized mice, RBCs were lysed with lysis solution (155 mM NH4Cl, 10 mM, KHCO3, 5% EDTA pH 7.4) for 10 min at RT, centrifuged at 300 g for 5 min and washed twice with PBS BSA 1%. Freshly harvested spleens were mechanically disaggregated using the Gentle MACS dissociator in 4 ml of PBS, BSA 1%, EDTA 2 mM, filtered through a 70 μm cell strainer to obtain single cell suspension, stratified on the Lymphoprep™ Density gradient medium, centrifuged at 800 g 20 min. The middle ring was collected and washed twice with PBS BSA 1% EDTA 2 mM. Cells from both blood and spleens were stained with CD16/CD32 blocking agent, CD45-eFluor450, Ly6G APC-eFluor780, B220 PE-Cy7, CD3 FITC, CD4 PE, CD8 PerCP-Cy5.5 for 30 min at RT. After 1 wash with PBS BSA 1%, samples were resuspended in 200 μl of PBS BSA 1% added of the CountBright Absolute Counting Beads and acquired with the Fortessa X-20 flow cytometer. The biparametric scatter plots were analyzed.

### 6.2 Results

### In SCD Mice, Epeleuton Reduces Neutrophil Counts and Modulates Inflammatory Response

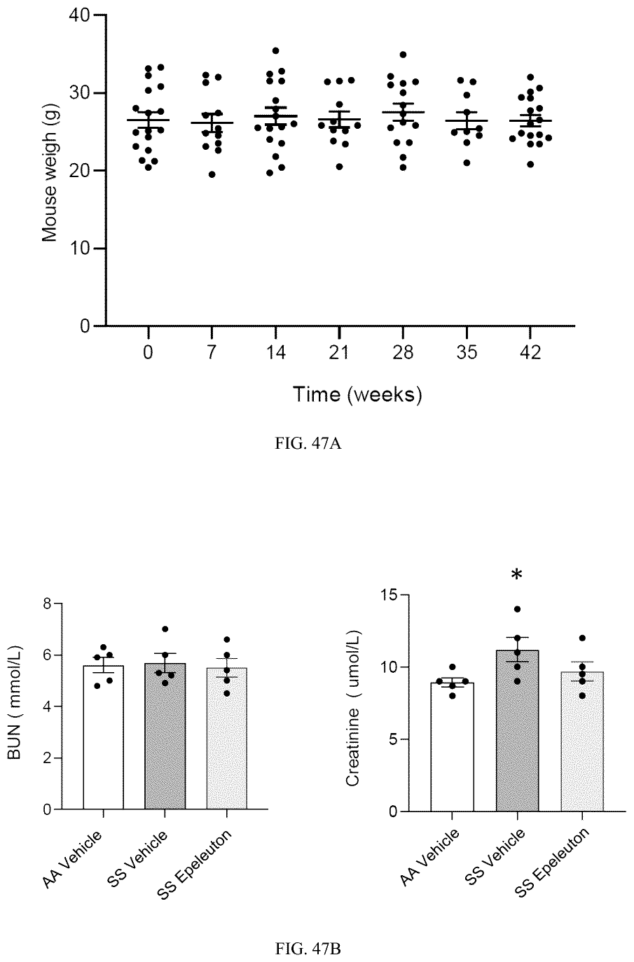

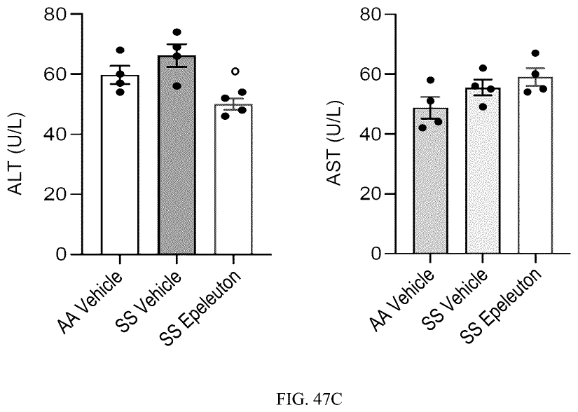

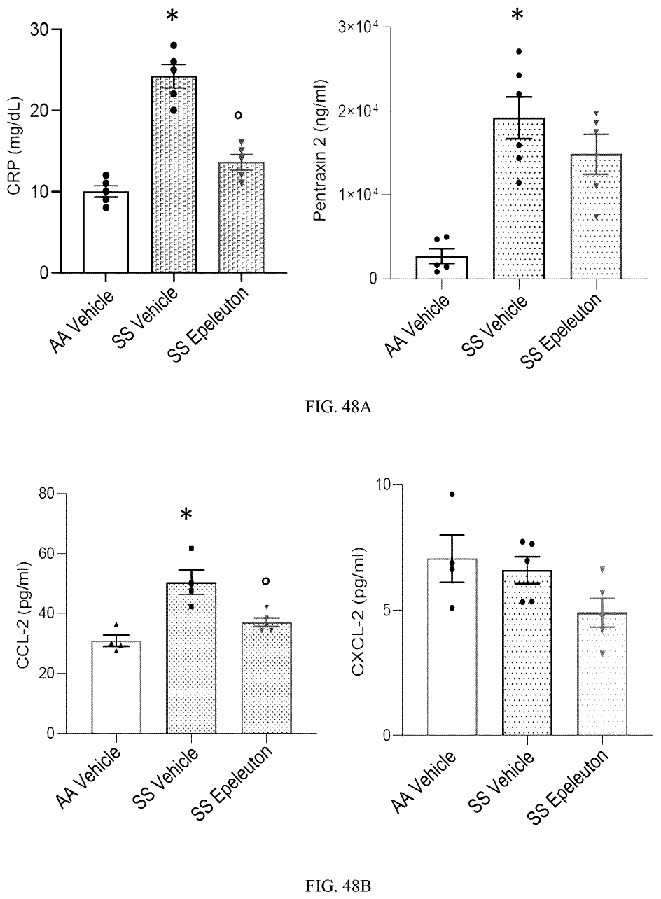

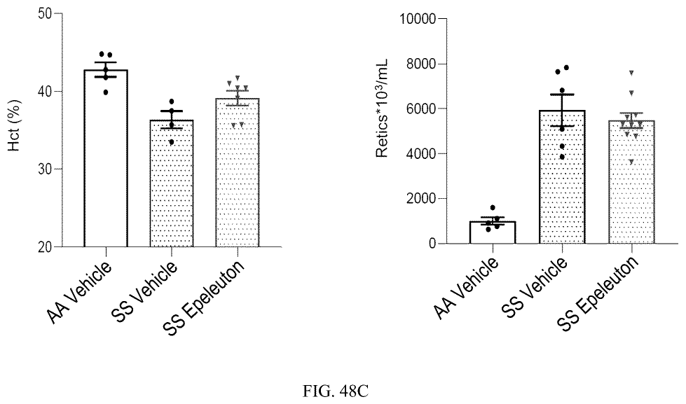

Epeleuton was well tolerated without major changes in body weight (FIG. 47A). A trend toward a reduction of serum creatinine in SS mice treated with Epeleuton compared to vehicle-treated animals was observed, with no change in BUN (FIG. 47B). A significant reduction in ALT was observed in SS mice treated with Epeleuton when compared to either healthy or SS mice, with no change in AST (FIG. 47C). A significant reduction in CRP was observed, and a trend toward a reduction of pentraxin-2, two markers of systemic inflammation (FIG. 48A), In Epeleuton-treated SS mice. These changes were accompanied by a lower plasma CCL2/MCP1, monocyte chemoattract protein 1, that mediates neutrophil recruitment and function (FIG. 48B). These data indicate a systemic anti-inflammatory effect of Epeleuton in humanized SCD mice.

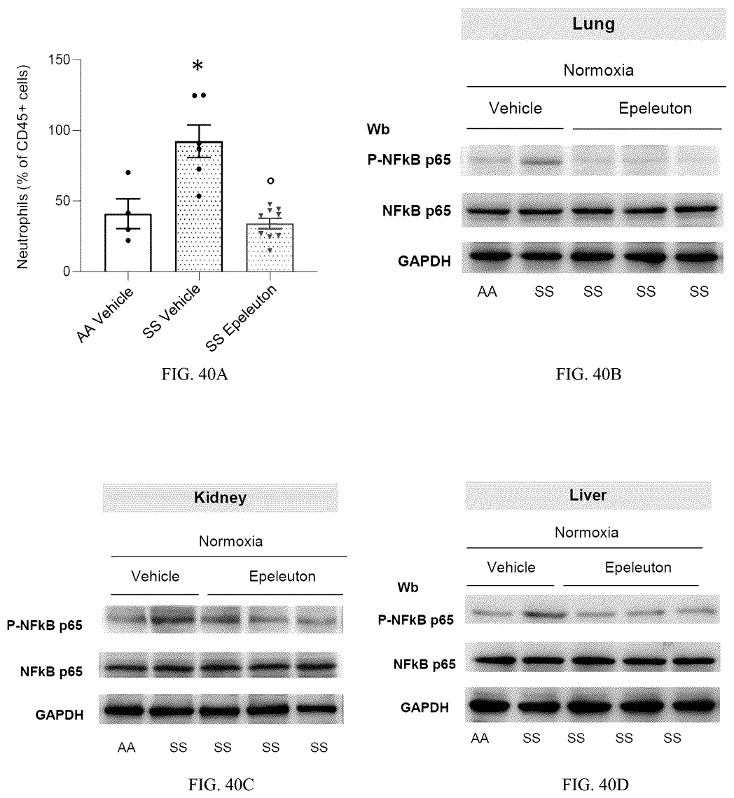

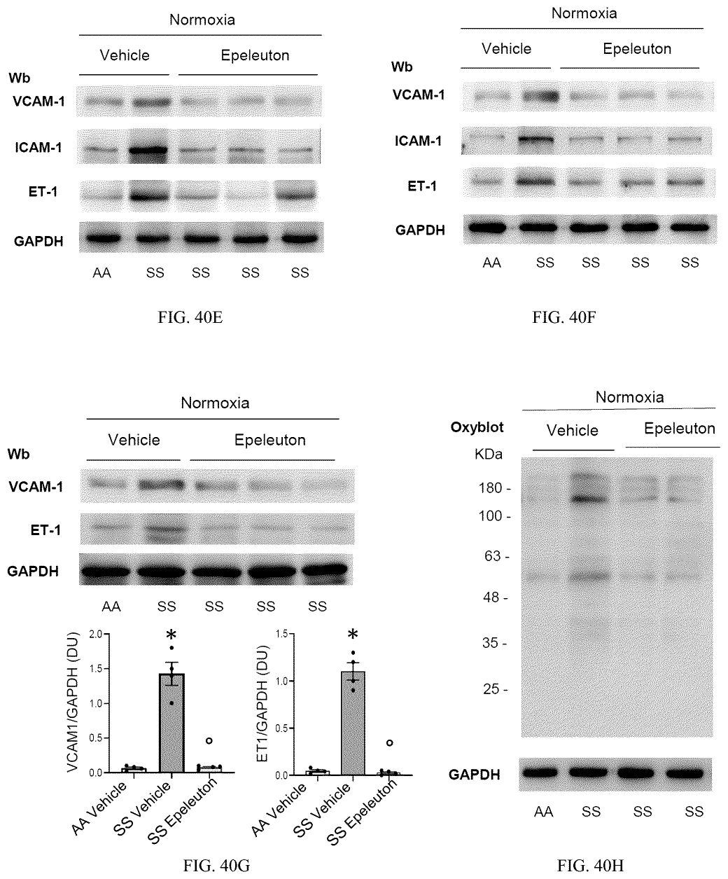

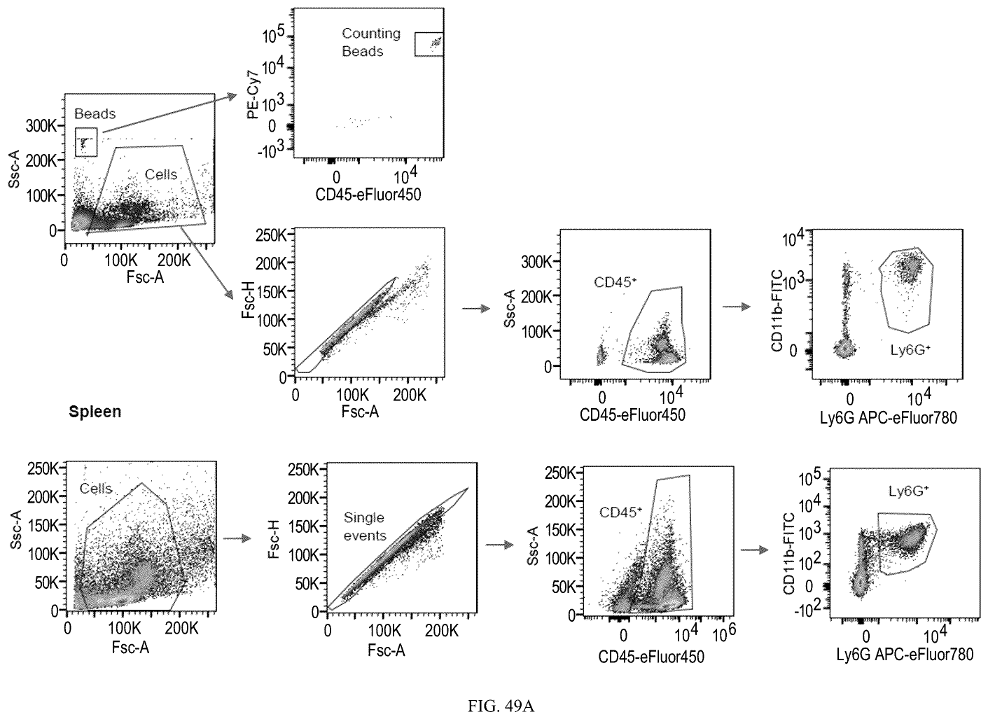

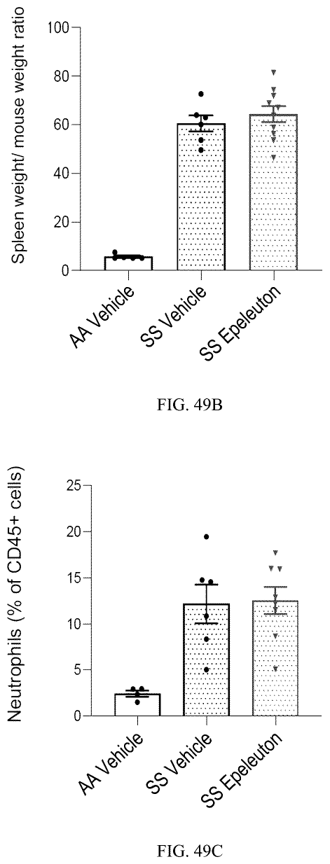

In Epeleuton-treated SS mice, there was no significant changes in either hematocrit or reticulocyte counts under normoxic conditions (FIGS. 48A-48C), whereas there was a significant reduction in circulating neutrophils (FIG. 40A, FIG. 49A). Since the spleen might act as reservoir of inflammatory cells but also contribute to their clearance, spleen-associated neutrophils were evaluated: No major change in spleen weight/mouse weight ratio and in spleen neutrophils in Epeleuton treated SS mice compared vehicle treated SS animals were observed (FIG. 49B).

### In SCD Mice, Epeleuton Prevents the Activation of NF-kB and Downregulates Markers of Inflammatory Vasculopathy

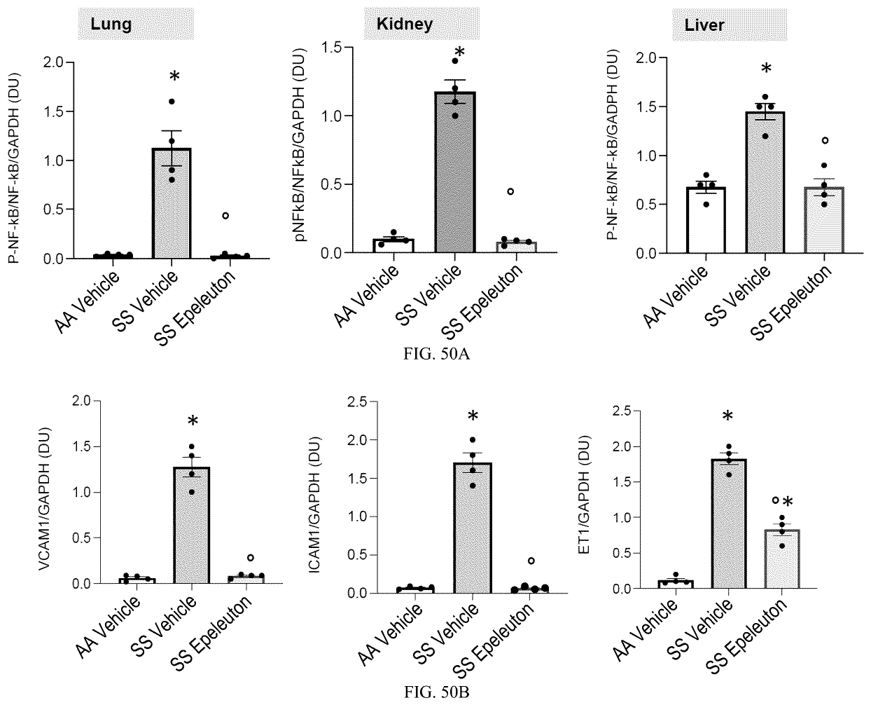

The transcriptional factor NF-kB is a key regulator of inflammation, and of the expression of genes involved in initiation of inflammation with cell-specific actions such as the modulation of vascular pro-adhesion molecules (e.g., VCAM-1 or ICAM-1). As previously reported in SCD mice that ω-3 fatty acid administration prevented the activation of NF-KB p65 in target organs for SCD such as lung, kidney, and liver. As shown in FIGS. 40B-40D, the activation of NF-kB was significantly reduced in lungs, kidneys, and livers from humanized SCD mice treated with Epeleuton when compared to vehicle treated SS animals (FIG. 50A), displaying a similar profile to healthy AA control animals.

Consistent with these observations, in lungs, kidneys, livers from Epeleuton treated SS mice there was a significantly reduction in the expression of (i) VCAM-1 and ICAM1, markers of inflammatory vasculopathy and endothelial activation; and (ii) endothelin-1 (ET-1) a potent vaso- and broncho-constrictive cytokine involved in sickle cell related organ damage (FIGS. 40E, 40F, 50B, 51A). Similar decreases were also observed in livers from Epeleuton treated mice, which also displayed a marked reduction in liver protein oxidation reaching values similar to those from livers of healthy mice (FIGS. 40G, 40H, 51B).

Collectively this data indicate that Epeleuton diminishes local sickle cell related inflammatory response, diminishing vascular endothelial activation in target organs for SCD.

### In SCD Mice, Epeleuton Protects Against Hypoxia/Reoxygenation Induced Hemolysis

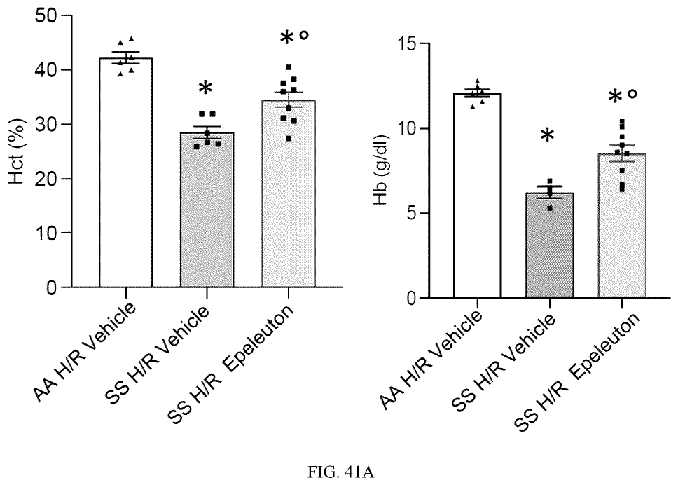

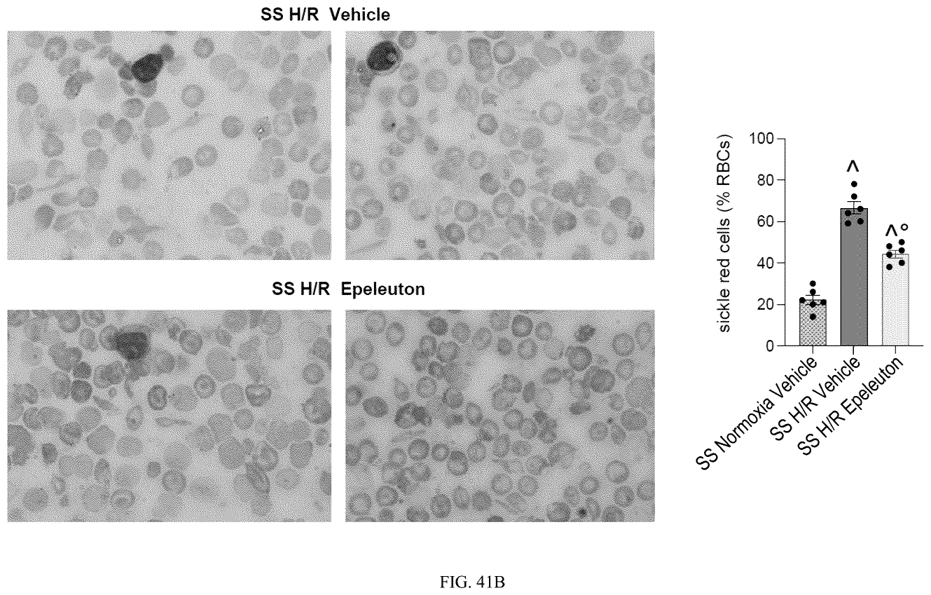

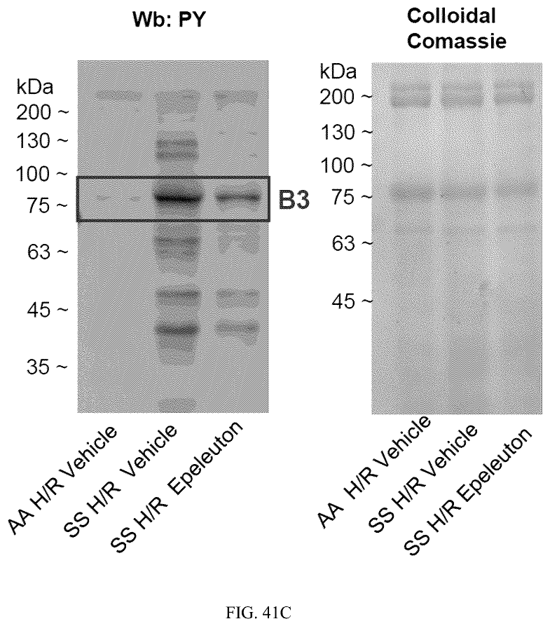

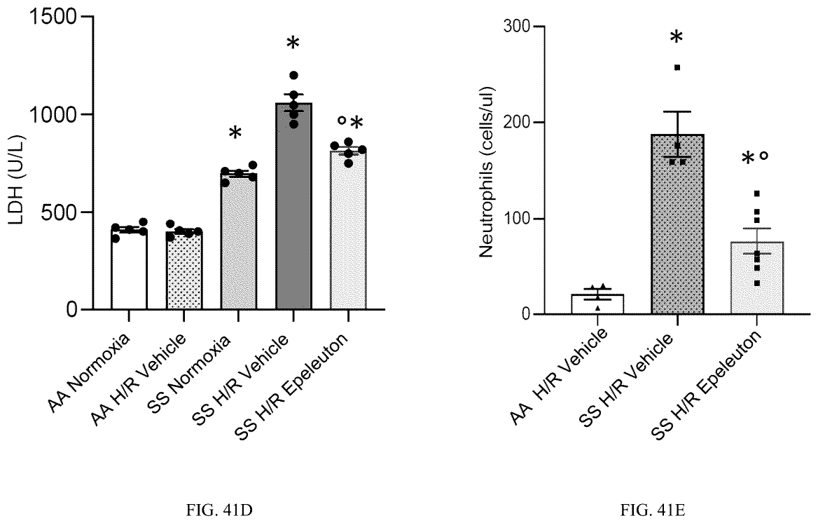

These experiments tested Epeleuton in sickle cell mice exposed to hypoxia/reoxygenation (H/R) stress, which is an experimental model of sickle cell-related VOCs. As shown in FIG. 41A, in SCD mice Epeleuton prevented the H/R induced reduction in hematocrit (Hct) and hemoglobin (Hb) and decreased the amount of sickled red blood cells (FIG. 41B). This indicates that in SCD mice Epeleuton protects red blood cells against the H/R induced stress. The improvement of red cell membrane features was also supported by the reduction in tyrosine phosphorylation state of band 3, an integral red cell membrane protein, involved in membrane stability and release of erythroid microparticles (FIG. 41C) and the reduction in lactate dehydrogenase (LDH), a known marker of hemolysis (FIG. 41D).

### Epeleuton Modulates Inflammatory Response to H/R and Re-programs Spleen Macrophages Towards a Pro-resolving Pattern

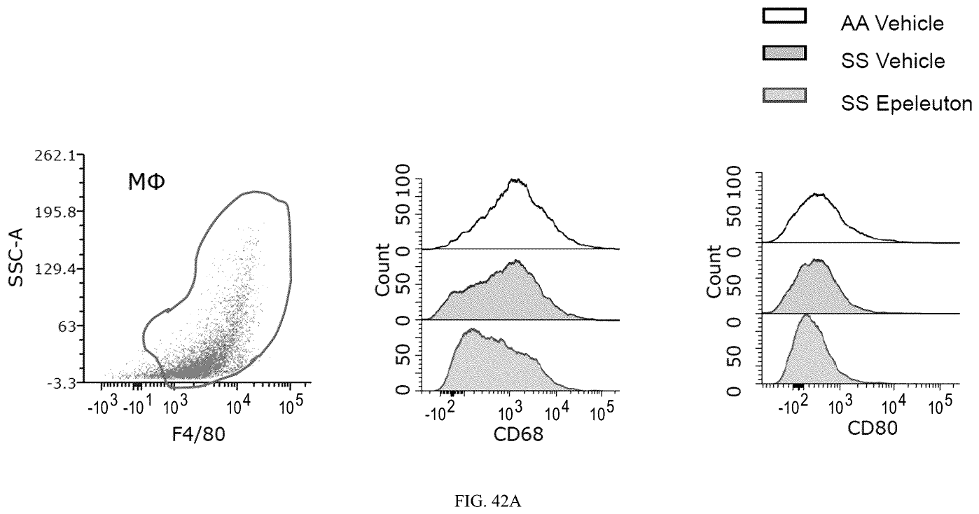

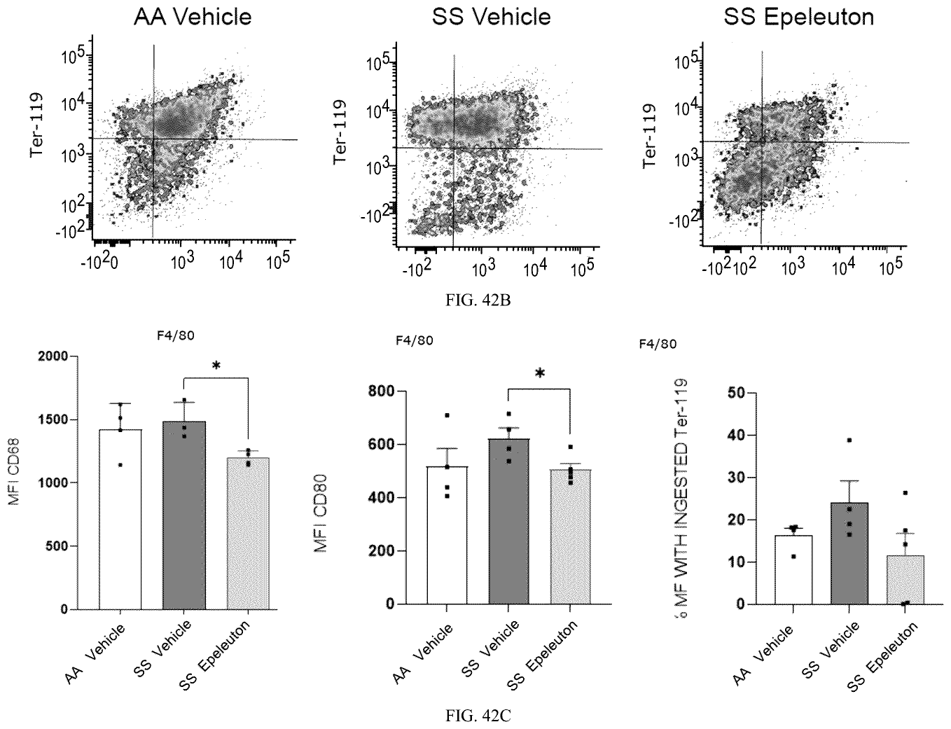

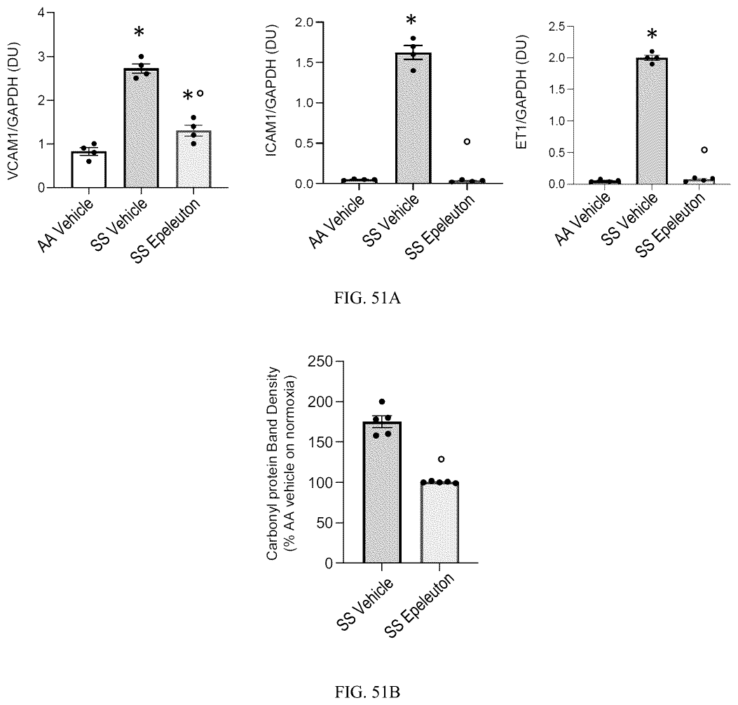

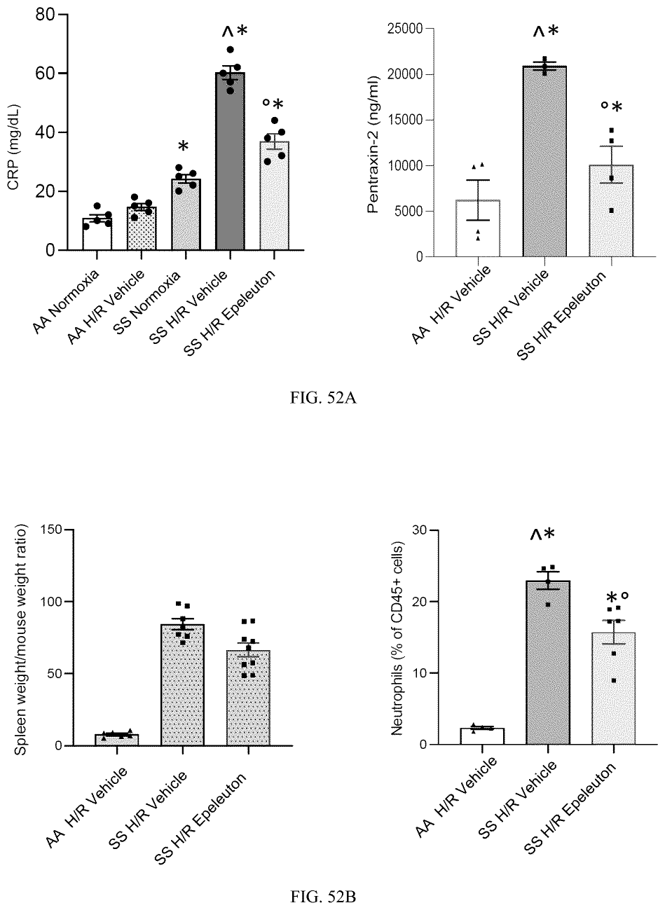

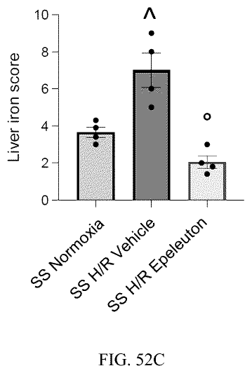

In SS mice Epeleuton significantly reduced the H/R-induced increase in neutrophil counts (FIG. 41E) while it decreased plasma C reactive protein (CRP) and pentraxin-2 (FIG. 52A), suggesting pro-resolving effects. There was a significant decrease of spleen neutrophils in Epeleuton treated SS mice exposed to H/R when compared to vehicle treated SS animals (FIG. 52B). This was associated with a significant reduction in spleen macrophage classic inflammatory markers (CD80, CD68) and a trend towards reduction in the clearance of damaged RBCs in SS mice treated with Epeleuton when compared with vehicle treated animals (FIGS. 42A-42C). It is noteworthy, that the effects of Epeleuton on neutrophils and macrophage re-programing in SCD mice are consistent with those previously reported in the same mouse model treated with exogenous 17R-RvD1, another specialized pro-resolving lipid mediator.

Taken together, these data indicate Epeleuton protects against H/R induced stress, attenuating inflammation, and leading to early initiation of resolution events.

### In SCD Mice, Epeleuton Reduces Lung Injury and Prevents the Overactivation of NF-kB and Hypoxia-induced Lung Inflammatory Vasculopathy

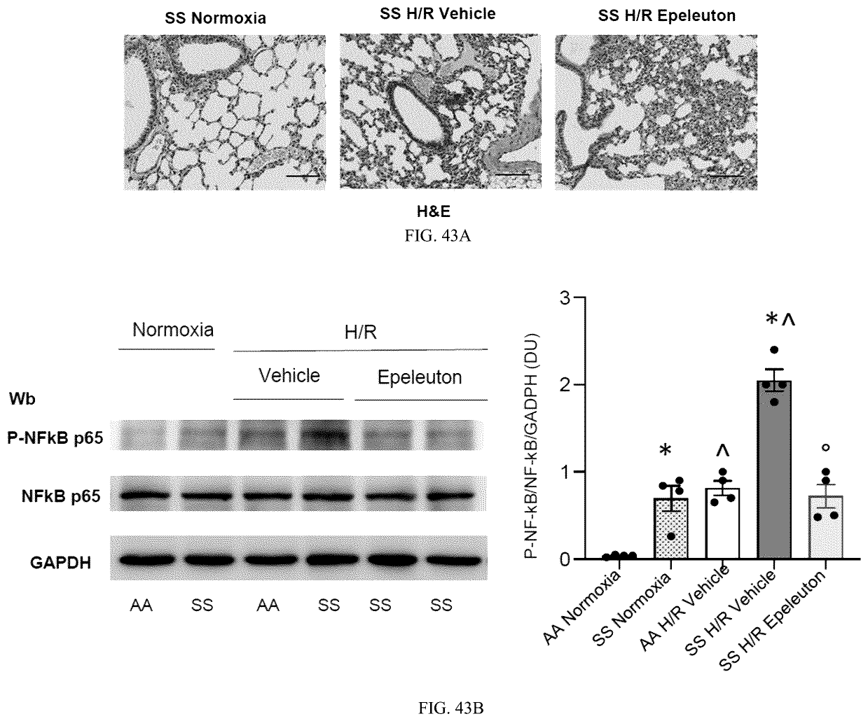

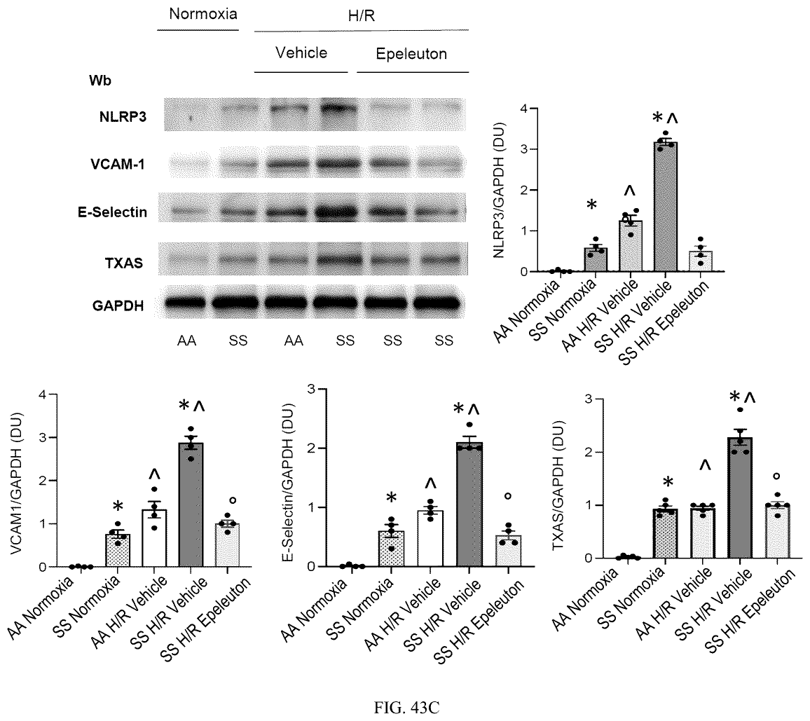

In SCD mice treated with Epeleuton, there was a trend towards a reduction in lung inflammatory cell infiltrates in lugs from mice treated with Epeleuton compared to vehicle treated SS animals (Table 38, FIG. 43A). Consistent with these observations, there was a significant decrease in the active forms of NF-kB p65 induced by H/R in SS mice treated with Epeleuton (FIG. 43B). To assess whether Epeleuton was protective against H/R induced amplified inflammatory response, and vascular dysfunction, lung expression of (i) NLRP3 inflammasome, a key player in sterile inflammation; (ii) vascular endothelial activation, and (iii) neutrophil vascular recruitment was evaluated. As shown in FIG. 43C, in lung tissue from Epeleuton treated SS mice exposed to H/R, there was downregulation of NLRP3 inflammasome, which has been reported to participate in inflammatory activation in models of acute lung injury such as acute respiratory disease syndrome (ARDS) or asthma. In addition, Epeleuton reduced the expression of VCAM-1, E-selectin, and thromboxane synthase (TXAS1) (FIG. 43C). TXAS1 is controlled by the NF-kB p65 signaling pathway, and it has been linked to activation of both vascular endothelial cells and platelets in other models of ischemia-reperfusion damage.

TABLE 38
Effects of Epeleuton on lung, kidney, and liver pathology
of sickle cell (SS) mice under normoxia and exposed
to hypoxia/reoxygenation (H/R) stress

| | Normoxia | H/R Vehicle | H/R Epeleuton |
|---|---|---|---|
| Lung | (n = 6) | (n = 4) | (n = 4) |
| Inflammatory | 8.8 ± 0.7 | 96.8 ± 8.2* | 87.3 ± 1.4* |
| cell infiltrates | | | |
| Thrombi | 0 | 0 | 0 |
| Kidney | (n = 6) | (n = 4) | (n = 4) |
| Inflammatory | 0 | 3.55 ± 0.46* | 4.2 ± 0.01* |
| cell infiltrates | | | |
| Thrombi | 0 | 0 | 0 |
| Liver | (n = 6) | (n = 4) | (n = 4) |
| Inflammatory | 34.7 ± 4.5 | 38.8 ± 6.1 | 28.5 ± 1.9 |
| cell infiltrates | | | |
| Thrombi | +(4/6) | +(4/4) | 0 |

H/R: hypoxia reoxygenation stress

### Lungs

Score for inflammatory cell infiltrate. Quantification of inflammatory cell infiltrates was expressed as the mean of cells per field at magnification of X250, as resulting by the analysis of at least 10 different fields on each hematoxylin-eosin-stained whole lung section. Data are expressed as means±SEM; * p<0.05 compared to normoxia.

Thrombi: 0: no thrombi; +: presence of thrombi per field at the magnification ×250.

### Kidney

Score for inflammatory cell infiltrate. Quantification of inflammatory cell infiltration in renal cortex of kidney was determined in hematoxylin-eosin-stained sections using a 0 to 4 scale based on the percentage of cell infiltrates occupied area in each filed. 0 (no sign of infiltration); 1 (1-10% of the area with cell infiltration); 2 (11-25%); 3 (26-50%); 4 (50%). The mean of 15 randomly selected field were analyzed at magnification ×400. Data are expressed as means±SEM; * p<0.05 compared to normoxia; ºp<0.05 compared to vehicle. Thrombi: 0: no thrombi; +: presence of thrombi presence of thrombi per field at the magnification ×250.

### Liver

Score for inflammatory cell infiltrate. Quantification of inflammatory cell infiltrates was expressed as the mean of cells per field at magnification of ×250, as resulting by the analysis of at least 10 different fields on each hematoxylin-eosin-stained whole liver section. Data are expressed as means±SEM; * p<0.05 compared to normoxia.

Thrombi: 0: no thrombi; +: presence of thrombi presence of thrombi per field at the magnification ×250.

Statistical analysis: Non-parametric Pairwise Wilcoxon Rank Sum Tests was used.

These data provide the evidence that Epeleuton protects SS mice against H/R induced lung injury by modulation of NF-kB and key mediators of vascular damage.

### In SCD Mice, Epeleuton Diminishes H/R Induced Kidney Damage and Markers of Vascular Dysfunction

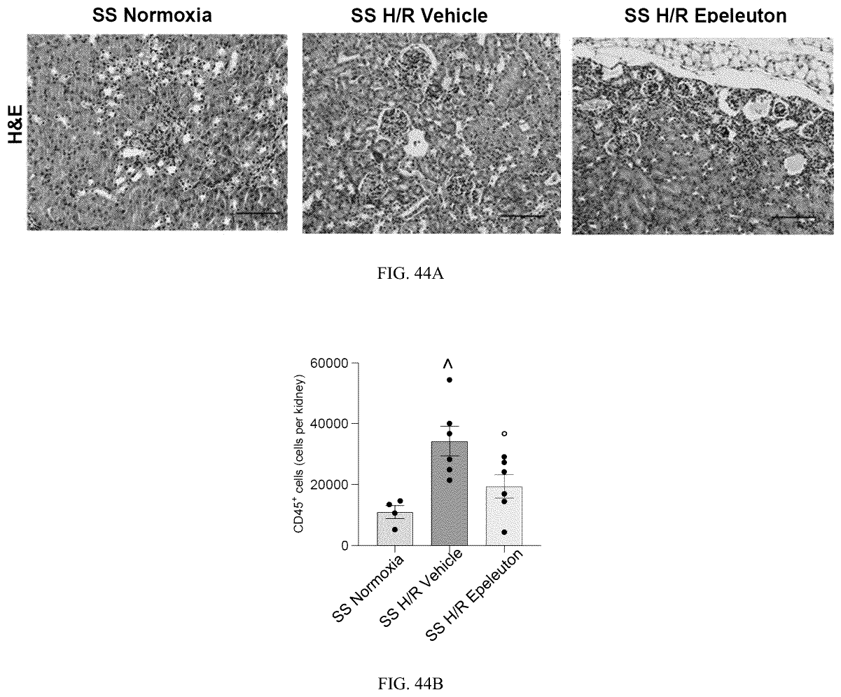

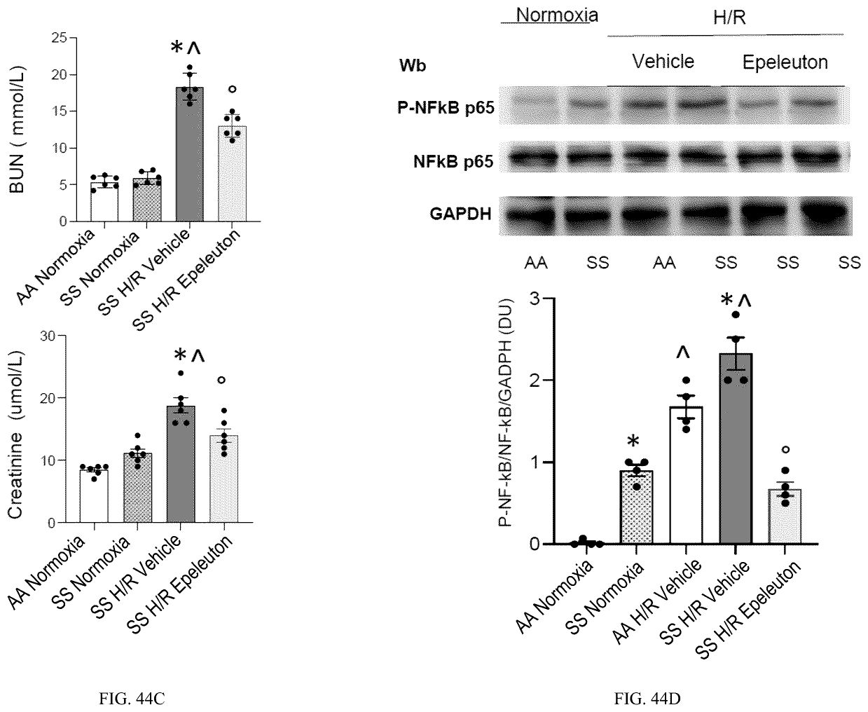

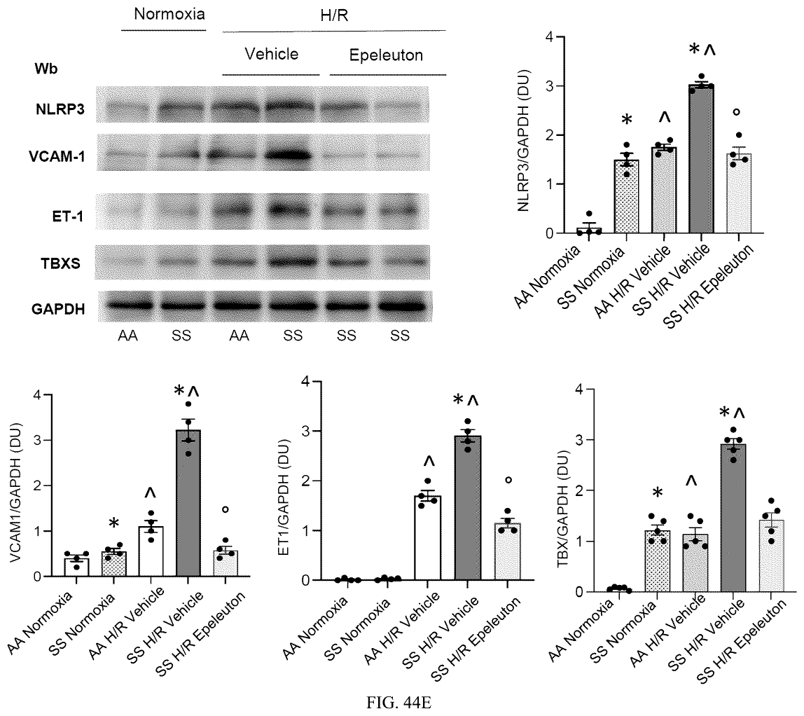

Histopathologic analysis of kidney from SS mice exposed to H/R and treated with or without Epeleuton revealed no major effect of Epeleuton in kidney inflammatory cell infiltrates (FIG. 44A, Table 38). Whereas a significant reduction in leukocyte infiltrates was found in kidneys from Epeleuton-treated SS mice compared to vehicle-treated animals

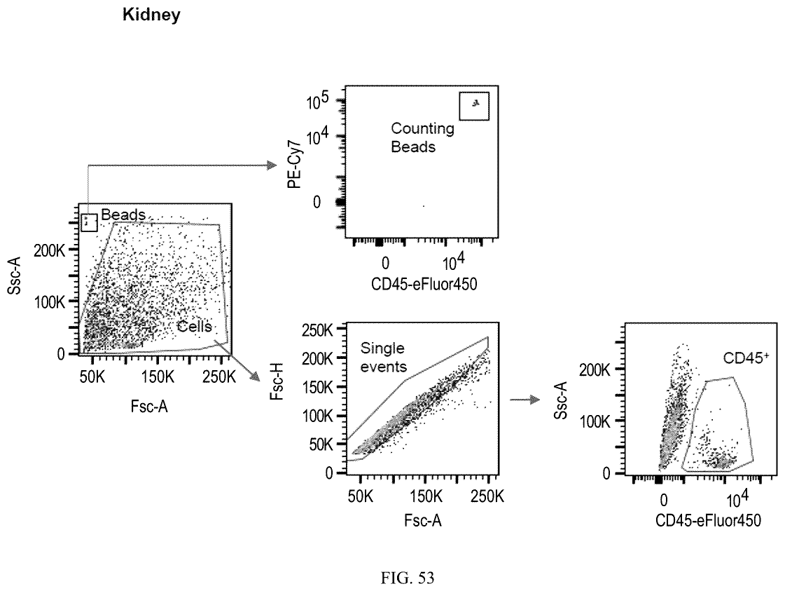

(FIG. 44B, FIG. 53). There also was a reduction of H/R-induced increases in creatinine and BUN plasma values compared to vehicle treated SS animals (FIG. 44C). This is consistent with the decreased H/R induced activation of NF-kB p65 in kidneys from Epeleuton-treated SS mice compared to vehicle-treated animals (FIG. 44D). The beneficial effects of Epeleuton in SS mice were further supported by the reduction in H/R induced increase in NLRP3, VCAM-1, ET-1 and TXAS-1 expression, which are involved in vascular activation, reduction of kidney vascular tone, and inflammation (FIG. 44E).

Taken together these data indicate that Epeleuton mitigates the H/R induced acute kidney damage and modulates the related amplified inflammation and vascular dysfunction.

### In SCD Mice Exposed to H/R Stress, Epeleuton Reduces Liver Injury and Prevents the Overactivation of Inflammatory and Redox Related Pathways

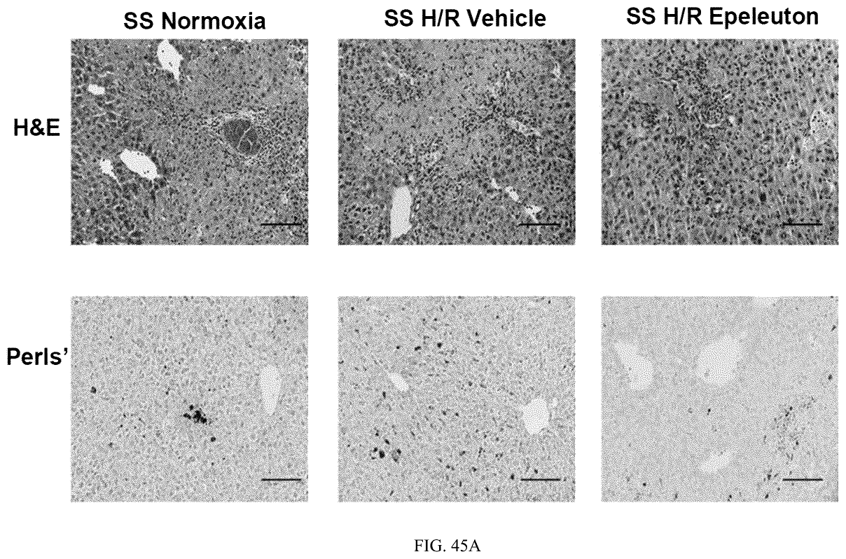

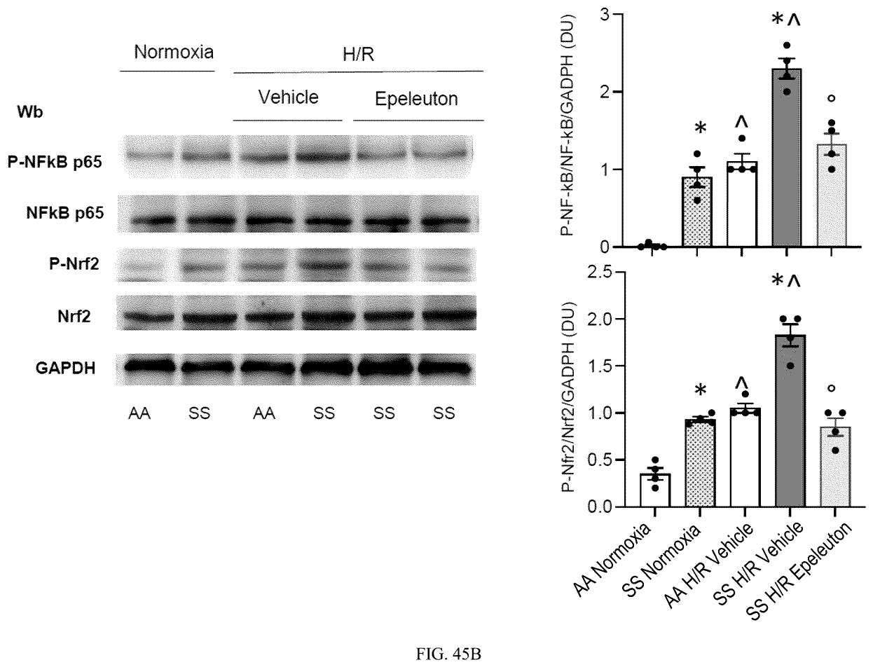

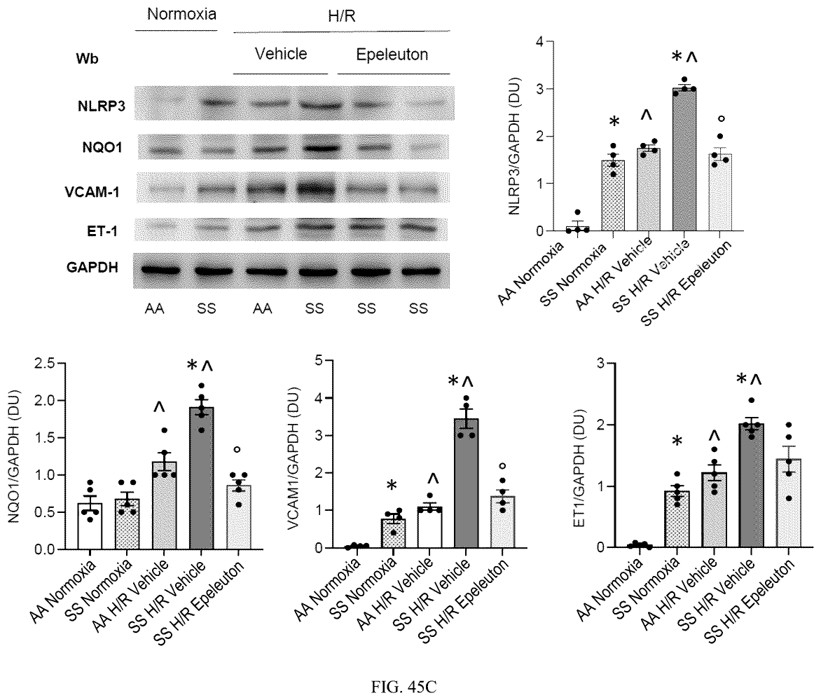

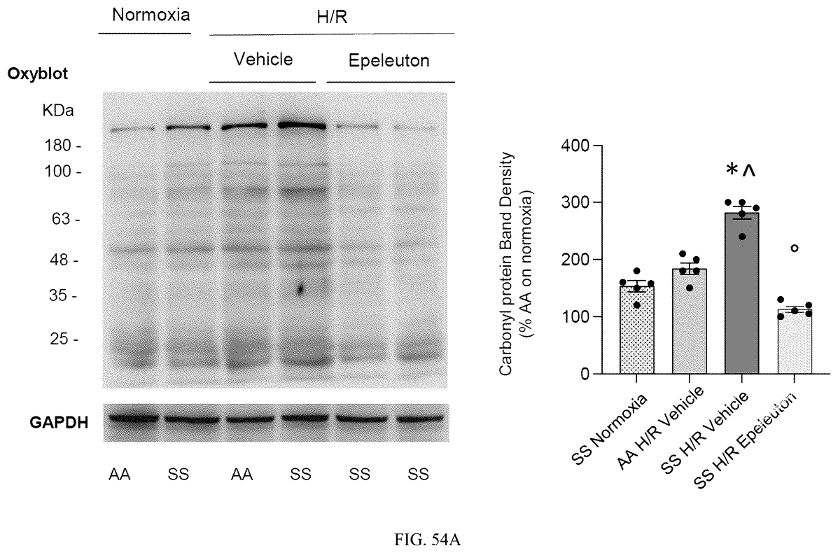

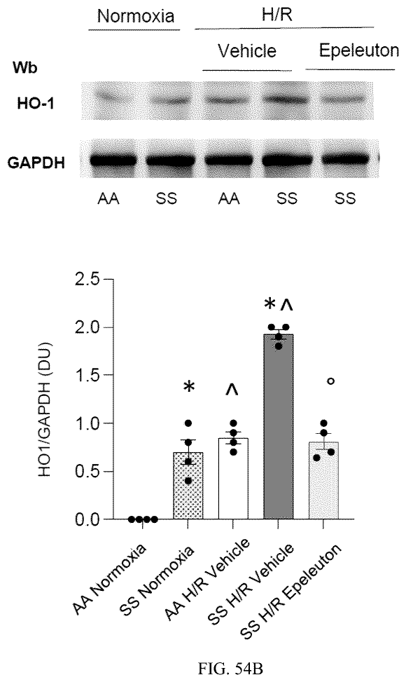

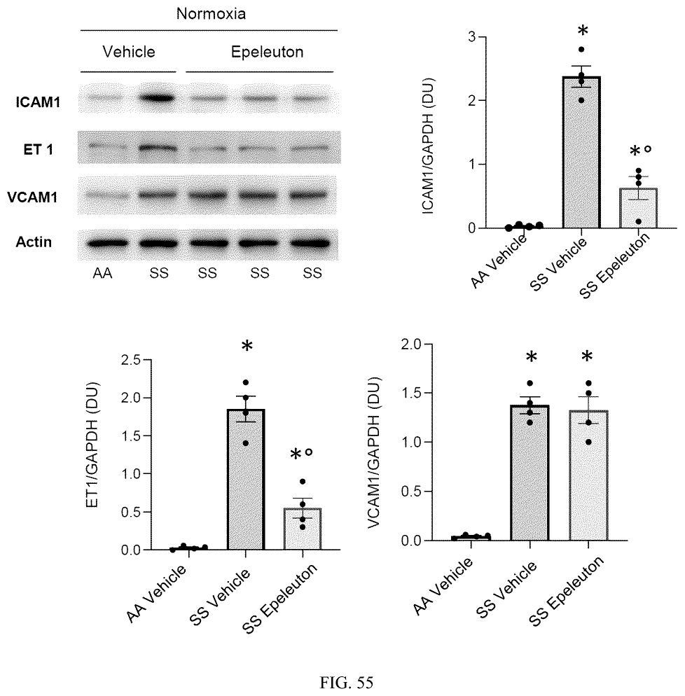

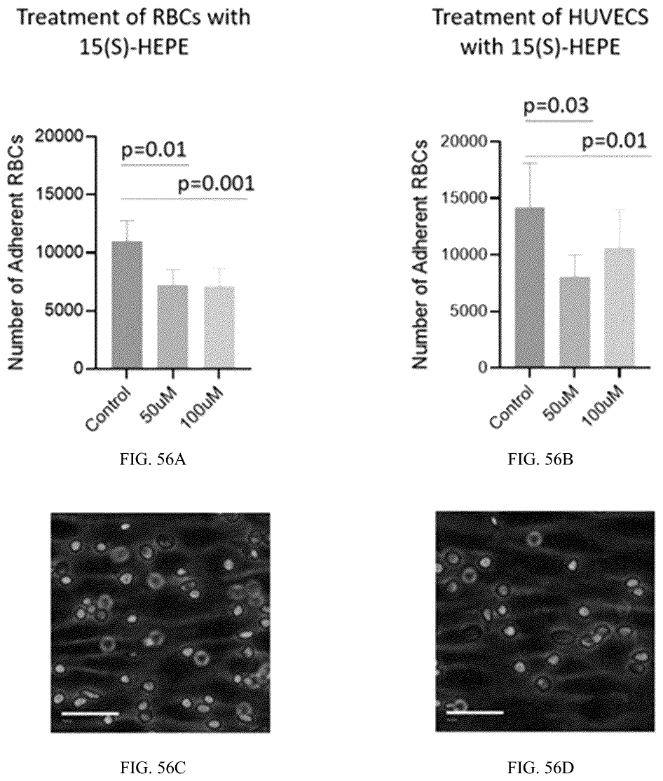

As shown in FIG. 45A, a trend in reduction of inflammatory cell infiltrates and a significant reduction in thrombi formation was observed in SS mice treated with Epeleuton exposed to H/R stress than in vehicle treated animals (FIG. 45A). In addition, there was a significant reduction in liver iron accumulation, mainly characterized by a decrease iron accumulation in hepatocytes of SS mice treated with Epeleuton exposed to H/R stress than in vehicle treated animals (FIG. 45A, lower panel, Perls' staining). In agreement with modulation of local inflammatory response and oxidation, there was reduced activation of both acute phase inflammatory and redox related transcriptional factor NF-kB p65 and Nrf2 in Epeleuton-treated SS mice exposed to H/R (FIG. 45B). This was associated with a marked reduction in liver protein oxidation (FIG. 54A) and a consistent downregulation of the antioxidants NQO1 and HO-1, which are regulated by Nfr2 (FIG. 45C, FIG. 54B). Next, the effects of Epeleuton on markers of inflammatory response and vascular activation were tested. As shown in FIG. 45C, there was lower expression of NLRP3 in livers from Epeleuton treated SS mice exposed to H/R (FIG. 45C). Similar observations of increased NLRP3 expression have been reported in models of acute on chronic liver diseases such as chronic hepatitis B/C or non-alcoholic steatohepatitis (NASH). In addition, Epeleuton prevented the H/R induced up-regulation of VCAM-1 in livers from SS mice exposed to H/R stress, while there was a trend towards reduction of ET-1 in the same group when compared to vehicle treated animals (FIG. 45C).

### Epeleuton Protects Against Progression of Inflammatory Vasculopathy Related to Acute Hypoxia/Reoxygenation Stress in SCD Mice

Given the beneficial effects of Epeleuton on vascular dysfunction in SCD target organs and of fatty acid supplementation on different models of vascular dysfunction and inflammatory vasculopathy, the impact of Epeleuton treatment on sickle cell related inflammatory vasculopathy was evaluated.

In isolated aortas from SCD mice under normoxia, Epeleuton significantly down-regulated the expression of ICAM-1 and ET-1 but not of VCAM-1 when compared to vehicle treated SS mice (FIGS. 54A and 54B).

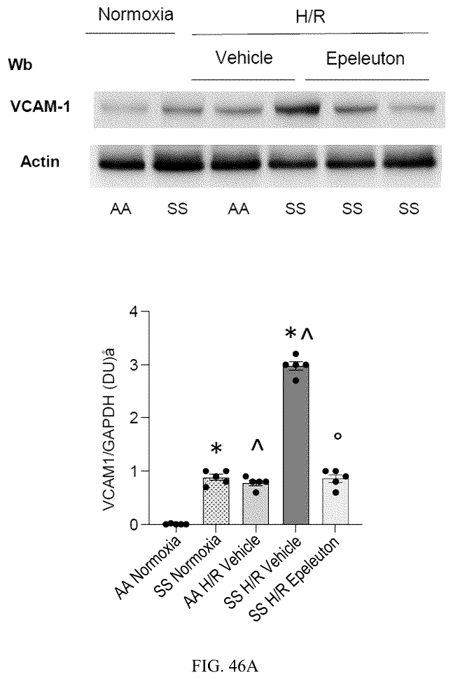

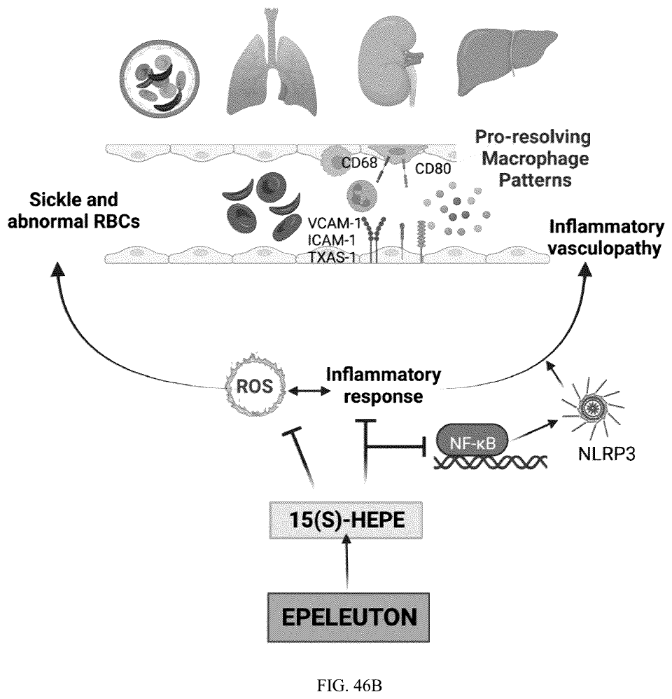

As shown in FIGS. 46A and 46B, there was a significant reduction in H/R induced expression of VCAM-1 compared to vehicle treated SS mouse group. These data indicate that treatment with Epeleuton may prevent worsening of vascular dysfunction induced by H/R stress with a potential to delay disease progression.

### 6.3 Discussion

The present technology shows that Epeleuton, a novel synthetic ω-3 fatty acid compound, protects against sickle cell related stress and supports the early initiation of resolution phase. The observed effects may limit disease progression, and the initiation of acute VOCs which negatively impact patients' quality of life.

Previous studies have shown the beneficial effects of ω-3 fatty acid supplementation on SCD phenotype. However, the formulation, the route of administration and bioavailability of ω-3 fatty acids still affect their use in clinical practice for patients with SCD. Epeleuton shows an advantageous functional profile compared to other formulations of ω-3 fatty acids tested in SCD such purified ω-3 formulations like Lovaza™ or SCA411 and fish oils. Lovaza™ is a prescription esterified ω-3-acid mixture (˜55.1% EPA, ˜ 44.9% DHA), while SC411 is DHA ethyl ester formulation with minimal food effect. Epeleuton as a second generation ω-3 enzymatic derivative that is metabolically downstream of EPA, provides advantages as a therapeutic for SCD. From a biochemical and pharmacological perspective, downstream purified ω-3 derivative therapeutics are likely to be more rapidly and potently bioactive per se or upon further enzymatic reactions. The advantage conferred in SCD from bypassing the initial stages of metabolism of fatty acids such as EPA and DHA is highlighted by the previous finding that humanized SCD mice were not as readily able to synthesize downstream fatty acid metabolites as healthy control mice. Notably, 15(S)-HEPE which has also been identified in resolving exudates from mice treated with ω-3 fatty acids, directly reduces polymorphonuclear leukocyte (PMN) trans-endothelial migration, as does its metabolite LXA5 at a higher potency. In SCD mice, Epeleuton might attenuate inflammatory response by favoring pro-resolving mechanisms mediated by 15(S)-HEPE. In agreement, there was a reduction in systemic inflammation, modulation of CCL2 chemotactic cytokine and a decrease in neutrophil counts in SS mice treated with Epeleuton. This is of particular interest since Epeleuton reduces splenic neutrophils and reprograms spleen macrophages towards a pro-resolution pattern.

In SCD mice, the dual anti-inflammatory and pro-resolution effects of Epeleuton are highlighted by the reduction in NF-kB p65 activation in target organs for SCD such as lung, kidney, and liver both under normoxia and H/R conditions (FIGS. 40A-40H, 42A-42C, 43A-43C, 44A-44E). This is further corroborated by the decreased expression of NLRP3 inflammasome, which activates inflammatory reactions in response to disrupted cell homeostasis in lung, kidney, and liver of SCD mice exposed to H/R stress. Growing evidence in both patients with SCD and cell/animal-based models of SCD support the importance of NLRP3 inflammasome activation in the severity of SCD clinical manifestation as well as in monocyte and platelet function/activation. Although the mechanism of activation of NLRP3 in SCD is still under investigation, oxidation and free heme/hemolysis have been suggested to trigger the NLRP3 inflammasome in SCD. In this model, Epeleuton reduces both oxidation and hemolysis in SS mice exposed to H/R stress. This prevents the worsening of liver iron overload and the activation of the redox related transcription factor Nrf2. Of note, previous studies have shown that NLRP3 inflammasome might favor Nrf2 degradation, affecting total cell anti-oxidant power, supporting the importance of limiting reactive-oxygen species induced activation of NRLP3 inflammasome. Here, Epeleuton protects against NLRP3 inflammasome by (i) preventing the activation of NF-kB p65; (ii) decreasing oxidation and the related activation of Nrf2; and (iii) reducing hemolysis. Indeed, studies in different models of acute or acute on chronic disease of lung, kidney or liver have previously shown that the inhibition of NLRP3 inflammasome ameliorates the diseases' outcomes. In addition, the observations that ω-3 fatty acids and specialized pro-resolving mediators prevent NLRP3 inflammasome activation in different models of inflammatory and degenerative disorders, further support the beneficial effects of Epeleuton on sickle cell related organ damage. In agreement, Epeleuton prevented the expression of markers of inflammatory vasculopathy in target organs for SCD as well as in isolated aorta, supporting the rationale to consider Epeleuton as new therapeutic tool for SCD.

Epeleuton is orally administered with a favorable safety and tolerability profile. It has recently received orphan drug designations from FDA and EMA based on the present results. The present findings show that in humanized SCD mice Epeleuton acts as a multimodal agent targeting hemolysis, inflammatory response, and vascular dysfunction. this data supports the potential therapeutic utility of Epeleuton and provide the rationale for the design of clinical trials to evaluate the efficacy of Epeleuton in patients with SCD. The next step is the conduct of clinical trials of Epeleuton in patients with SCD.

### Example 7: Epeleuton, A Novel Synthetic ω-3 Fatty Acid, Reduces Endothelial Adhesion of Red Blood Cells from Sickle Cell Disease Patients Under Physiological Flow Conditions

Acute vaso-occlusive crises (VOCs) are the leading cause of hospitalization for patients with sickle cell disease (SCD). VOCs are characterized by microvascular occlusion that is initiated and progressed by adhesion of sickle red blood cells (RBCs), neutrophils, and platelets to the vascular endothelium. Epeleuton is 15-hydroxy-eicosapentaenoic acid (15(S)-HEPE)ethyl ester, a novel synthetic second generation omega-3 fatty acid which targets key drivers of multicellular adhesion. In Townes humanized SCD mice, Epeleuton acts as a disease-modifying agent, decreasing (a) systemic and local inflammatory activation, (b) vascular dysfunction including adhesion molecule expression, (c) organ damage, and (d) intravascular hemolysis and sickling.

The aim of the present study is to evaluate the effect of 15(S)-HEPE, the active moiety of Epeleuton, on SCD patient RBCs' adhesion to heme-activated endothelium using a novel endothelialized microfluidic system.

### 7.1 Methods

Whole blood samples were collected from 7 SCD subjects with HbSS genotype at University Hospitals Cleveland Medical Center, Cleveland, OH, USA. RBCs were isolated after centrifugation of whole blood collected in EDTA and were resuspended in basal cell culture medium (EBM; Lonza, Morristown, NJ, USA) with 10 mM of HEPES to reach 20% hematocrit. Human umbilical vein endothelial cells (HUVECs; Lonza, Morristown, NJ, USA) were seeded within the microfluidic channels and cultured for at least 72 hours at 15 dyne/cm2 prior to experiments. Tested concentrations of 15(S)-HEPE were selected based on clinical pharmacokinetics for 15(S)-HEPE at 2 g/day and 4 g/day Epeleuton doses. Incubation of RBCs or HUVECs with 15(S)-HEPE was conducted at 37° C. for 6 hours. In one experiment 15(S)-HEPE-treated tor vehicle-treated RBCs flowed on untreated HUVECs and in the second experiment untreated RBCs flowed on 15(S)-HEPE-treated or vehicle-treated RBCs. In both cases a short-term acute activation was conducted where RBCs were supplemented with 40 μM heme and injected through the microfluidic channels for 15 minutes. Non-adherent RBCs were washed with basal cell culture medium. Phase-contrast images of the remaining RBCs were recorded using inverted microscope (DMi8 Leica Microsystems Inc. Deerfield, IL, USA) and the images were analyzed based on previous published methods. Statistical significance was assessed using paired t-test

### 7.2 Results

In a standardized endothelium-on-a-chip microfluidic platform treatment of both RBCs and endothelium with 15(S)-HEPE decreased RBC adhesion to heme-activated endothelium under physiological flow conditions. Treatment of heme-activated HUVECs with 15(S)-HEPE significantly decreased adhesion of SCD patient RBCs to endothelium at both 50 μM (p=0.03) and 100 μM concentrations (p=0.01). Similarly, treatment of RBCs with 15(S)-HEPE significantly decreased their adhesion to heme-activated endothelium at both 50 UM (p=0.01) and 100 UM concentrations (p=0.001).

Under physiological flow conditions, 15(S)-HEPE decreased adhesion of SCD patient RBCs at clinically relevant concentrations by affecting both the endothelium and RBCs independently. These findings support the clinical development of Epeleuton as a potential novel therapeutic option for patients with SCD.

### REFERENCES

1. Brugnara C, Armsby C C, De Franceschi L, Crest M, Euclaire M F, and Alper S L. Ca(2+)-activated K+ channels of human and rabbit erythrocytes display distinctive patterns of inhibition by venom peptide toxins. J Membr Biol. 1995; 147(1):71-82.
2. De Franceschi L, Brugnara C, Rouyer-Fessard P, Jouault H, and Beuzard Y. Formation of dense erythrocytes in SAD mice exposed to chronic hypoxia: evaluation of different therapeutic regimens and of a combination of oral clotrimazole and magnesium therapies. Blood. 1999; 94(12):4307-13.
3. Bunn H F. Pathogenesis and treatment of sickle cell disease. New England Journal of Medicine. 1997; 337(11):762-9.
4. Matte A, Recchiuti A, Federti E, Koehl B, Mintz T, El Nemer W, et al. Resolution of sickle cell disease-associated inflammation and tissue damage with 17 R-resolvin D1 Blood. 2019; 133:252-65.
5. De Franceschi L, Cappellini M D, and Olivieri O. Thrombosis and sickle cell disease. Semin Thromb Hemost. 2011; 37(3):226-36.
6. Matte A, Cappellini M D, lolascon A, Enrica F, and De Franceschi L. Emerging drugs in randomized controlled trials for sickle cell disease: are we on the brink of a new era in research and treatment? Expert Opin Investig Drugs. 2020; 29(1):23-31.
7. Telen M J, Malik P, and Vercellotti G M. Therapeutic strategies for sickle cell disease: towards a multi-agent approach. Nat Rev Drug Discov. 2019; 18(2):139-58.
8. Rossato P, Federti E, Matte A, Glantschnig H, Canneva F, Schuster M, et al. Evidence of protective effects of recombinant ADAMTS13 in a humanized model of sickle cell disease. Haematologica. 2022; 107(11):2650-60.
9. Hebbel R P. The systems biology-based argument for taking a bold step in chemoprophylaxis of sickle vasculopathy. Am J Hematol. 2009; 84(9):543-5.
10. Ataga K I, Kutlar A, Kanter J, Liles D, Cancado R, Friedrisch J, et al. Crizanlizumab for the Prevention of Pain Crises in Sickle Cell Disease. The New England journal of medicine. 2017; 376(5):429-39.
11. Nicholls S J, Lincoff A M, Garcia M, Bash D, Ballantyne C M, Barter P J, et al. Effect of High-Dose Omega-3 Fatty Acids vs Corn Oil on Major Adverse Cardiovascular Events in Patients at High Cardiovascular Risk: The STRENGTH Randomized Clinical Trial. Jama. 2020; 324(22):2268-80.
12. Massaro M, Scoditti E, Carluccio M A, and De Caterina R. Basic mechanisms behind the effects of n-3 fatty acids on cardiovascular disease. Prostaglandins Leukot Essent Fatty Acids. 2008;79(3-5):109-15.
13. Hasturk H, Abdallah R, Kantarci A, Nguyen D, Giordano N, Hamilton J, et al. Resolvin E1 (RvE1) Attenuates Atherosclerotic Plaque Formation in Diet and Inflammation-Induced Atherogenesis. Arterioscler Thromb Vasc Biol. 2015; 35(5):1123-33.

14 Fredman G, Hellmann J, Proto J D, Kuriakose G, Colas R A, Dorweiler B, et al. An imbalance between specialized pro-resolving lipid mediators and pro-inflammatory leukotrienes promotes instability of atherosclerotic plaques. Nat Commun. 2016; 7:12859.
15. Serhan C N, Jain A, Marleau S, Clish C, Kantarci A, Behbehani B, et al. Reduced inflammation and tissue damage in transgenic rabbits overexpressing 15-lipoxygenase and endogenous anti-inflammatory lipid mediators. J Immunol. 2003; 171(12):6856-65.
16. Freedman G, Williams K D, and Beer J S. Softening the Blow of Social Exclusion: The Responsive Theory of Social Exclusion. Front Psychol. 2016; 7:1570.
17. Gonzalez-Periz A, Horrillo R, Ferre N, Gronert K, Dong B, Moran-Salvador E, et al. Obesity-induced insulin resistance and hepatic steatosis are alleviated by omega-3 fatty acids: a role for resolvins and protectins. FASEB J. 2009; 23(6):1946-57.
18. Matte A, Recchiuti A, Federti E, Koehl B, Mintz T, El Nemer W, et al. Resolution of sickle cell disease-associated inflammation and tissue damage with 17R-resolvin D1. Blood. 2019; 133(3):252-65.
19. Setty BNY, Betal S G, Miller R E, Brown D S, Meier M, Cahill M, et al. Relationship of Omega-3 fatty acids DHA and EPA with the inflammatory biomarker hs-CRP in children with sickle cell anemia. Prostaglandins Leukot Essent Fatty Acids. 2019; 146:11-8.
20. Ren H, Okpala I, Ghebremeskel K, Ugochukwu C C, Ibegbulam O, and Crawford M. Blood mononuclear cells and platelets have abnormal fatty acid composition in homozygous sickle cell disease. Ann Hematol. 2005; 84(9):578-83.
21. Ren H, Obike I, Okpala I, Ghebremeskel K, Ugochukwu C, and Crawford M. Steady-state haemoglobin level in sickle cell anaemia increases with an increase in erythrocyte membrane n-3 fatty acids. Prostaglandins Leukot Essent Fatty Acids. 2005; 72(6):415-21.
22. Ren H, Ghebremeskel K, Okpala I, Ugochukwu C C, Crawford M, and Ibegbulam O. Abnormality of erythrocyte membrane n-3 long chain polyunsaturated fatty acids in sickle cell haemoglobin C (HbSC) disease is not as remarkable as in sickle cell anaemia (HbSS). Prostaglandins Leukot Essent Fatty Acids. 2006; 74(1):1-6.
23. Enomoto™, Isichei C, VanderJagt D J, Fry D E, and Glew R H. Decreased polyunsaturated fatty acids in sickle cell anaemia. J Trop Pediatr. 1998; 44(1):28-34.
24. VanderJagt D J, Trujillo M R, Bode-Thomas F, Huang Y S, Chuang L T, and Glew R H. Phase angle correlates with n-3 fatty acids and cholesterol in red cells of Nigerian children with sickle cell disease. Lipids Health Dis. 2003; 2:2.
25. Calder P C. Omega-3 fatty acids and inflammatory processes. Nutrients. 2010; 2(3):355-74.
26. Rangel-Huerta O D, Aguilera C M, Mesa M D, and Gil A. Omega-3 long-chain polyunsaturated fatty acids supplementation on inflammatory biomakers: a systematic review of randomised clinical trials. Br J Nutr. 2012; 107 Suppl 2:S159-70.
27. Russo C, Olivieri O, Girelli D, Azzini M, Stanzial A M, Guarini P, et al. Omega-3 polyunsaturated fatty acid supplements and ambulatory blood pressure monitoring parameters in patients with mild essential hypertension. J Hypertens. 1995;13(12 Pt 2):1823-6.
28. Arnardottir H, Pawelzik S C, Sarajlic P, Quaranta A, Kolmert J, Religa D, et al. Immunomodulation by intravenous omega-3 fatty acid treatment in older subjects hospitalized for COVID-19: A single-blind randomized controlled trial. Clin Transl Med. 2022;12(9):e895.
29. Kalish B T, Matte A, Andolfo I, lolascon A, Weinberg O, Ghigo A, et al. Dietary omega-3 fatty acids protect against vasculopathy in a transgenic mouse model of sickle cell disease. Haematologica. 2015; 100(7):870-80.
30. Okpala I, Ibegbulam O, Duru A, Ocheni S, Emodi I, Ikefuna A, et al. Pilot study of omega-3 fatty acid supplements in sickle cell disease. APMIS: acta pathologica, microbiologica, et immunologica Scandinavica. 2011; 119(7):442-8.
31. Daak A A, Ghebremeskel K, Hassan Z, Attallah B, Azan H H, Elbashir M I, et al. Effect of omega-3 (n-3) fatty acid supplementation in patients with sickle cell anemia: randomized, double-blind, placebo-controlled trial. The American journal of clinical nutrition. 2013; 97(1):37-44.
32. Tomer A, Kasey S, Connor W E, Clark S, Harker L A, and Eckman J R. Reduction of pain episodes and prothrombotic activity in sickle cell disease by dietary n-3 fatty acids. Thrombosis and haemostasis. 2001; 85(6):966-74.
33. Daak A, Rabinowicz A, and Ghebremeskel K. Omega-3 fatty acids are a potential therapy for patients with sickle cell disease. Nat Rev Dis Primers. 2018;4(1): 15.
34. Sherratt SCR, Libby P, Bhatt D L, and Mason R P. A biological rationale for the disparate effects of omega-3 fatty acids on cardiovascular disease outcomes. Prostaglandins Leukot Essent Fatty Acids. 2022; 182:102450.
35. Wu CYC, Lopez-Toledano M A, Daak A A, Clemons G A, Citadin C T, Sancilio F D, et al. SC411 treatment can enhance survival in a mouse model of sickle cell disease. Prostaglandins Leukot Essent Fatty Acids. 2020; 158:102110.
36. Daak A A, Dampier C D, Fuh B, Kanter J, Alvarez O A, Black L V, et al. Double-blind, randomized, multicenter phase 2 study of SC411 in children with sickle cell disease (SCOT trial). Blood Adv. 2018; 2(15):1969-79.
37. Climax J, Newsome P N, Hamza M, Weissbach M, Coughlan D, Sattar N, et al. Effects of Epeleuton, a Novel Synthetic Second-Generation n-3 Fatty Acid, on Non-Alcoholic Fatty Liver Disease, Triglycerides, Glycemic Control, and Cardiometabolic and Inflammatory Markers. J Am Heart Assoc. 2020;9(16):e016334.
38. Sanak M, Levy B D, Clish C B, Chiang N, Gronert K, Mastalerz L, et al. Aspirin-tolerant asthmatics generate more lipoxins than aspirin-intolerant asthmatics. Eur Respir J. 2000; 16(1):44-9.
39. Wong P Y, Hughes R, and Lam B. Lipoxene: a new group of trihydroxy pentaenes of eicosapentaenoic acid derived from porcine leukocytes. Biochemical and biophysical research communications. 1985; 126(2):763-72.
40. Reichel C A, Rehberg M, Lerchenberger M, Berberich N, Bihari P, Khandoga A G, et al. Ccl2 and Ccl3 mediate neutrophil recruitment via induction of protein synthesis and generation of lipid mediators. Arterioscler Thromb Vasc Biol. 2009; 29(11):1787-93.
41. Halade G V, Norris P C, Kain V, Serhan C N, and Ingle K A. Splenic leukocytes define the resolution of inflammation in heart failure. Sci Signal. 2018;11(520).
42. Kain V, Ingle K A, Colas R A, Dalli J, Prabhu S D, Serhan C N, et al. Resolvin D1 activates the inflammation resolving response at splenic and ventricular site following myocardial infarction leading to improved ventricular function. J Mol Cell Cardiol. 2015; 84:24-35.
43. Cerutti A, Cols M, and Puga I. Activation of B cells by non-canonical helper signals. EMBO Rep. 2012; 13(9):798-810.
44 Baker R G, Hayden M S, and Ghosh S. N F-kappaB, inflammation, and metabolic disease. Cell Metab. 2011; 13(1):11-22.
45. Liu T, Zhang L, Joo D, and Sun S C. N F-kappaB signaling in inflammation. Signal Transduct Target Ther. 2017; 2:17023-.
46. Sabaa N, de Franceschi L, Bonnin P, Castier Y, Malpeli G, Debbabi H, et al. Endothelin receptor antagonism prevents hypoxia-induced mortality and morbidity in a mouse model of sickle-cell disease. J Clin Invest. 2008; 118(5):1924-33.
47. Noomuna P, Risinger M, Zhou S, Seu K, Man Y, An R, et al. Inhibition of Band 3 tyrosine phosphorylation: a new mechanism for treatment of sickle cell disease. Br J Haematol. 2020; 190(4):599-609.
48. McVey M J, Steinberg B E, and Goldenberg N M. Inflammasome activation in acute lung injury. Am J Physiol Lung Cell Mol Physiol. 2021;320(2):L165-L78.
49. Amano H, Nakamura M, Ito Y, Kakutani H, Eshima K, Kitasato H, et al. Thromboxane A synthase enhances blood flow recovery from hindlimb ischemia. J Surg Res. 2016; 204(1):153-63.
50. Amano H, Ito Y, Eshima K, Kato S, Ogawa F, Hosono K, et al. Thromboxane A2 induces blood flow recovery via platelet adhesion to ischaemic regions. Cardiovasc Res. 2015; 107(4):509-21.
51. De Franceschi L, Bertoldi M, Matte A, Santos Franco S, Pantaleo A, Ferru E, et al. Oxidative stress and beta-thalassemic erythroid cells behind the molecular defect. Oxid Med Cell Longev. 2013; 2013:985210.
52. Wu X, Dong L, Lin X, and Li J. Relevance of the NLRP3 Inflammasome in the Pathogenesis of Chronic Liver Disease. Front Immunol. 2017; 8:1728.
53. Li Z, and Jiang J. The NLRP3 inflammasome mediates liver failure by activating procaspase-1 and pro-IL-1 beta and regulating downstream CD40-CD40L signaling. J Int Med Res. 2021;49(9):3000605211036845.
54. Osunkwo I, Andemariam B, Minniti C P, Inusa BPD, El Rassi F, Francis-Gibson B, et al. Impact of sickle cell disease on patients' daily lives, symptoms reported, and disease management strategies: Results from the international Sickle Cell World Assessment Survey (SWAY). Am J Hematol. 2021; 96(4):404-17.
55. Ugwu A, Iloanusi N, Ugwu N, Chukwu B, Ezenwosu O, Modebe E, et al. Pilot assessment of omega-3 fatty acids and potassium thiocyanate in sickle cell anemia patients with conditional peak systolic cerebral artery blood velocity. Blood Cells Mol Dis. 2021; 89:102564.
56. Serhan C N, Clish C B, Brannon J, Colgan S P, Chiang N, and Gronert K. Novel functional sets of lipid-derived mediators with antiinflammatory actions generated from omega-3 fatty acids via cyclooxygenase 2-nonsteroidal antiinflammatory drugs and transcellular processing. J Exp Med. 2000; 192(8):1197-204.
57. de Freitas Dutra V, Leal VNC, Fernandes F P, Souza CRL, Figueiredo M S, and Pontillo A. Genetic contribution and functional impairment of inflammasome in sickle cell disease. Cytokine. 2022; 149:155717.
58. Vogel S, Arora T, Wang X, Mendelsohn L, Nichols J, Allen D, et al. The platelet NLRP3 inflammasome is upregulated in sickle cell disease via HMGB1/TLR4 and Bruton tyrosine kinase. Blood Adv. 2018; 2(20):2672-80.
59. Pitanga T N, Santana S S, Zanette D L, Guarda C C, Santiago R P, Maffili V V, et al. Effect of lysed and non-lysed sickle red cells on the activation of NLRP3 inflammasome and LTB4 production by mononuclear cells. Inflamm Res. 2021; 70(7):823-34.
60. Erdei J, Toth A, Balogh E, Nyakundi B B, Banyai E, Ryffel B, et al. Induction of NLRP3 Inflammasome Activation by Heme in Human Endothelial Cells. Oxid Med Cell Longev. 2018; 2018:4310816.
61. Jin C, and Flavell R A. Molecular mechanism of NLRP3 inflammasome activation. J Clin Immunol. 2010; 30(5):628-31.
62. Garstkiewicz M, Strittmatter G E, Grossi S, Sand J, Fenini G, Werner S, et al. Opposing effects of Nrf2 and Nrf2-activating compounds on the NLRP3 inflammasome independent of Nrf2-mediated gene expression. Eur J Immunol. 2017; 47(5):806-17.
63. Liu X, Zhang X, Ding Y, Zhou W, Tao L, Lu P, et al. Nuclear Factor E2-Related Factor-2 Negatively Regulates NLRP3 Inflammasome Activity by Inhibiting Reactive Oxygen Species-Induced NLRP3 Priming. Antioxid Redox Signal. 2017; 26(1):28-43.
64. Heneka M T, Kummer M P, Stutz A, Delekate A, Schwartz S, Vieira-Saecker A, et al. NLRP3 is activated in Alzheimer's disease and contributes to pathology in APP/PS1 mice. Nature. 2013; 493(7434):674-8.
65. Yan Y, Jiang W, Liu L, Wang X, Ding C, Tian Z, et al. Dopamine controls systemic inflammation through inhibition of NLRP3 inflammasome. Cell. 2015;160(1-2):62-73.
66. Wang Z, Hu W, Lu C, Ma Z, Jiang S, Gu C, et al. Targeting NLRP3 (Nucleotide-Binding Domain, Leucine-Rich-Containing Family, Pyrin Domain-Containing-3) Inflammasome in Cardiovascular Disorders. Arterioscler Thromb Vasc Biol. 2018; 38(12):2765-79.
67. Yan Y, Jiang W, Spinetti T, Tardivel A, Castillo R, Bourquin C, et al. Omega-3 fatty acids prevent inflammation and metabolic disorder through inhibition of NLRP3 inflammasome activation. Immunity. 2013; 38(6):1154-63.
68. Lin C, Chao H, Li Z, Xu X, Liu Y, Bao Z, et al. Omega-3 fatty acids regulate NLRP3 inflammasome activation and prevent behavior deficits after traumatic brain injury. Exp Neurol. 2017; 290:115-22.
69. Zhu P, Zhang J J, Cen Y, Yang Y, Wang F, Gu K P, et al. High Endogenously Synthesized N-3 Polyunsaturated Fatty Acids in Fat-1 Mice Attenuate High-Fat Diet-Induced Insulin Resistance by Inhibiting NLRP3 Inflammasome Activation via Akt/GSK-3beta/TXNIP Pathway. Molecules. 2022;27(19).
70. Wang Y H, Gao X, Tang Y R, Chen F Q, Yu Y, Sun M J, et al. Resolvin D1 Alleviates Mechanical Allodynia via ALX/FPR2 Receptor Targeted Nod-like Receptor Protein 3/Extracellular Signal-Related Kinase Signaling in a Neuropathic Pain Model. Neuroscience. 2022; 494:12-24.
71. Cao L, Wang Y, Wang Y, Lv F, Liu L, and Li Z. Resolvin D2 suppresses NLRP3 inflammasome by promoting autophagy in macrophages. Exp Ther Med. 2021;22(5):1222.
72. Lopategi A, Flores-Costa R, Rius B, Lopez-Vicario C, Alcaraz-Quiles J, Titos E, et al. Frontline Science: Specialized proresolving lipid mediators inhibit the priming and activation of the macrophage NLRP3 inflammasome. J Leukoc Biol. 2019;105(1):25-36.
73. Kasztan M, Fox B M, Lebensburger J D, Hyndman K A, Speed J S, Pollock J S, et al. Hyperfiltration predicts long-term renal outcomes in humanized sickle cell mice. Blood Adv. 2019; 3(9):1460-75.
74. Beckman J D, Abdullah F, Chen C, Kirchner R, Rivera-Rodriguez D, Kiser Z M, et al. Endothelial TLR4 Expression Mediates Vaso-Occlusive Crisis in Sickle Cell Disease. Front Immunol. 2020; 11:613278.
75. Ansari J, Senchenkova E Y, Vital S A, Al-Yafeai Z, Kaur G, Sparkenbaugh E M, et al. Targeting the AnxA1/Fpr2/ALX pathway regulates neutrophil function, promoting thromboinflammation resolution in sickle cell disease. Blood. 2021; 137(11):1538-49.
76. Kalish B T, Matte A, Andolfo I, lolascon A, Weinberg O, Ghigo A, et al. Dietary ω-3 fatty acids protect against vasculopathy in a transgenic mouse model of sickle cell disease. Haematologica. 2015; 100(7):870-80.
77. Matte A, Federti E, Winter M, Koerner A, Harmeier A, Mazer N, et al. Bitopertin, a selective oral GLYT1 inhibitor, improves anemia in a mouse model of β-thalassemia. JCI Insight. 2019;4(22).
78. Matte A, Low P S, Turrini F, Bertoldi M, Campanella M E, Spano D, et al. Peroxiredoxin-2 expression is increased in beta-thalassemic mouse red cells but is displaced from the membrane as a marker of oxidative stress. Free radical biology & medicine. 2010; 49(3):457-66.
79. Matte A, Federti E, Kung C, Kosinski P A, Narayanaswamy R, Russo R, et al. The pyruvate kinase activator mitapivat reduces hemolysis and improves anemia in a beta-thalassemia mouse model. The Journal of clinical investigation. 2021;131 (10).
80. Matte A, De Falco L, lolascon A, Mohandas N, An X, Siciliano A, et al. The Interplay Between Peroxiredoxin-2 and Nuclear Factor-Erythroid 2 Is Important in Limiting Oxidative Mediated Dysfunction in beta-Thalassemic Erythropoiesis. Antioxidants & redox signaling. 2015; 23(16):1284-97.
81. Brugnara C, and de Franceschi L. Effect of cell age and phenylhydrazine on the cation transport properties of rabbit erythrocytes. J Cell Physiol. 1993; 154(2):271-80.
82. de Franceschi L, Turrini F, Honczarenko M, Ayi K, Rivera A, Fleming M D, et al. In vivo reduction of erythrocyte oxidant stress in a murine model of beta-thalassemia. Haematologica. 2004; 89(11):1287-98.
83. Cossarizza A, Chang H D, Radbruch A, Acs A, Adam D, Adam-Klages S, et al. Guidelines for the use of flow cytometry and cell sorting in immunological studies (second edition). Eur J Immunol. 2019; 49(10):1457-973.
84. Matté A, Pantaleo A, Ferru E, Turrini F, Bertoldi M, Lupo F, et al. The novel role of peroxiredoxin-2 in red cell membrane protein homeostasis and senescence. Free Radic Biol Med. 2014; 76:80-8.
85. Matte A, Low P S, Turrini F, Bertoldi M, Campanella M E, Spano D, et al. Peroxiredoxin-2 expression is increased in β-thalassemic mouse red cells but is displaced from the membrane as a marker of oxidative stress. Free Radic Biol Med. 2010; 49(3):457-66.
86. Federti E, Matté A, Ghigo A, Andolfo I, Cimino J, Siciliano A, et al. Peroxiredoxin-2 plays a pivotal role as multimodal cytoprotector in the early phase of pulmonary hypertension. Free Radical Biology and Medicine 2017; 112:376-86.
87. Kalish B T, Matte A, Andolfo I, lolascon A, Weinberg O, Ghigo A, et al. Dietary ω-3 fatty acids protect against vasculopathy in a transgenic mouse model of sickle cell disease. Haematologica. 2015; 100(7):870-80.
88. Matte A, Recchiuti A, Federti E, Koehl B, Mintz T, El Nemer W, et al. Resolution of sickle cell disease-associated inflammation and tissue damage with 17 R-resolvin D1 Blood. 2019; 133:252-65.
89. Matte A, Recchiuti A, Federti E, Koehl B, Mintz T, El Nemer W, et al. Resolution of sickle cell disease-associated inflammation and tissue damage with 17R-resolvin D1. Blood. 2019; 133(3):252-65.
90. Federti E, Matte A, Ghigo A, Andolfo I, James C, Siciliano A, et al. Peroxiredoxin-2 plays a pivotal role as multimodal cytoprotector in the early phase of pulmonary hypertension. Free Radic Biol Med. 2017; 112:376-86.
91. Cossarizza A, Chang H D, Radbruch A, Acs A, Adam D, Adam-Klages S, et al. Guidelines for the use of flow cytometry and cell sorting in immunological studies (second edition). Eur J Immunol. 2019; 49(10):1457-973.

### Additional Embodiments

1. A method of treating and/or preventing a hematologic disorder in a subject in need thereof, comprising administering to the subject a composition comprising 15-hydroxyeicosapentaenoic acid (15-HEPE) in free acid form or a pharmaceutically acceptable ester, conjugate, or salt thereof.

2. The method of embodiment 1, wherein the hematologic disorder is selected from the group consisting of a red blood cell disorder, a hemoglobin disorder, a bleeding disorder, and a blood cancer.

3. The method of embodiment 2, wherein the red blood cell disorder is selected from the group consisting of anemia, hemolytic anemia, iron deficiency anemia, hereditary spherocytosis, chronic hemolysis, pyruvate kinase deficiency, and glucose-6-phosphate dehydrogenase (G6PD) deficiency.

4. The method of embodiment 2, wherein the hemoglobin disorder is selected from the group consisting of sickle cell disease, alpha thalassemia, and beta thalassemia.

5. The method of embodiment 2, wherein the bleeding disorder is selected from the group consisting of hemophilia, thrombophilia, thrombocytopenia, idiopathic thrombocytopenia purpura (ITP), heparin-induced thrombocytopenia (HIT), venous thromboembolism, arterial thrombosis, and embolism.

6. The method of embodiment 2, wherein the blood cancer is selected from the group consisting of myelodysplastic syndrome (MDS), lymphoma, leukemia, and myeloma.

7. A method of treating and/or preventing sickle cell disease and/or symptoms associated thereof in a subject in need thereof, comprising administering to the subject a composition comprising 15-hydroxyeicosapentaenoic acid (15-HEPE) in free acid form or a pharmaceutically acceptable ester, conjugate, or salt thereof.

8. The method of embodiment 7, wherein the symptoms associated thereof comprise inflammatory vasculopathy, vasoconstriction, bronchoconstriction, iron overload, hemoglobinopathy, chromic hemolysis, sickling, anemia, inflammation, pain, acute and/or chronic damage to an organ, and/or oxidative stress.

9. The method of embodiment 8, wherein the organ is lung, kidney, liver, or spleen.

10. A method of treating and/or preventing heparin-induced thrombocytopenia (HIT) in a subject in need thereof, comprising administering to the subject a composition comprising 15-hydroxyeicosapentaenoic acid (15-HEPE) in free acid form or a pharmaceutically acceptable ester, conjugate, or salt thereof.

11. The method of any one of embodiments 1-10, wherein the subject has no change or a reduction in serum creatine levels after receiving the composition comprising 15-HEPE relative to baseline or to a control subject who is not administered the composition comprising 15-HEPE or who is administered placebo.

12. The method of any one of embodiments 1-10, wherein the subject has no change or a reduction in C-reactive protein (CRP) levels after receiving the composition comprising 15-HEPE relative to baseline or to a control subject who is not administered the composition comprising 15-HEPE or who is administered placebo.

13. The method of any one of embodiments 1-10, wherein the subject has no change or a reduction in pentraxin-2 levels after receiving the composition comprising 15-HEPE relative to baseline or to a control subject who is not administered the composition comprising 15-HEPE or who is administered placebo.

14. The method of any one of embodiments 1-10, wherein the subject has no change or a reduction in monocyte chemoattractant protein-1 (MCP-1/CCL2) levels after receiving the composition comprising 15-HEPE relative to baseline or to a control subject who is not administered the composition comprising 15-HEPE or who is administered placebo.

15. The method of any one of embodiments 1-10, wherein the subject has no change or a reduction in circulating neutrophils after receiving the composition comprising 15-HEPE relative to baseline or to a control subject who is not administered the composition comprising 15-HEPE or who is administered placebo.

16. The method of any one of embodiments 1-10, wherein the subject has no change or a reduction in vascular cell adhesion molecule 1 (VCAM-1) levels after receiving the composition comprising 15-HEPE relative to baseline or to a control subject who is not administered the composition comprising 15-HEPE or who is administered placebo.

17. The method of any one of embodiments 1-10, wherein the subject has no change or a reduction in intercellular adhesion molecule 1 (ICAM-1) levels after receiving the composition comprising 15-HEPE relative to baseline or to a control subject who is not administered the composition comprising 15-HEPE or who is administered placebo.

18. The method of any one of embodiments 1-10, wherein the subject has no change or a reduction in endothelin-1 (ET-1) levels after receiving the composition comprising 15-HEPE relative to baseline or to a control subject who is not administered the composition comprising 15-HEPE or who is administered placebo.

19. The method of any one of embodiments 1-10, wherein the subject has no change or an increase in hematocrit levels after receiving the composition comprising 15-HEPE relative to baseline or to a control subject who is not administered the composition comprising 15-HEPE or who is administered placebo.

20. The method of any one of embodiments 1-10, wherein the subject has no change or an increase in hemoglobin levels after receiving the composition comprising 15-HEPE relative to baseline or to a control subject who is not administered the composition comprising 15-HEPE or who is administered placebo.

21. The method of any one of embodiments 1-10, wherein the subject has no change or a reduction in sickled red blood cell numbers after receiving the composition comprising 15-HEPE relative to baseline or to a control subject who is not administered the composition comprising 15-HEPE or who is administered placebo.

22. The method of any one of embodiments 1-10, wherein the subject has no change or a reduction in CD68 levels after receiving the composition comprising 15-HEPE relative to baseline or to a control subject who is not administered the composition comprising 15-HEPE or who is administered placebo.

23. The method of any one of embodiments 1-10, wherein the subject has no change or a reduction in CD80 levels after receiving the composition comprising 15-HEPE relative to baseline or to a control subject who is not administered the composition comprising 15-HEPE or who is administered placebo.

24. The method of any one of embodiments 1-10, wherein the subject has no change or a reduction in NF-kB p65 activation after receiving the composition comprising 15-HEPE relative to baseline or to a control subject who is not administered the composition comprising 15-HEPE or who is administered placebo.

25. The method of any one of embodiments 1-10, wherein the subject has no change or a reduction in NLRP3 inflammasome activation after receiving the composition comprising 15-HEPE relative to baseline or to a control subject who is not administered the composition comprising 15-HEPE or who is administered placebo.

26. The method of any one of embodiments 1-10, wherein the subject has no change or a reduction in vascular endothelial activation after receiving the composition comprising 15-HEPE relative to baseline or to a control subject who is not administered the composition comprising 15-HEPE or who is administered placebo.

27. The method of any one of embodiments 1-10, wherein the subject has no change or a reduction in in neutrophil vascular recruitment after receiving the composition comprising 15-HEPE relative to baseline or to a control subject who is not administered the composition comprising 15-HEPE or who is administered placebo.

28. The method of any one of embodiments 1-10, wherein the subject has no change or a reduction in inflammatory cell infiltration after receiving the composition comprising 15-HEPE relative to baseline or to a control subject who is not administered the composition comprising 15-HEPE or who is administered placebo.

29. The method of any one of embodiments 1-10, wherein the subject has no change or a reduction in thrombi formation after receiving the composition comprising 15-HEPE relative to baseline or to a control subject who is not administered the composition comprising 15-HEPE or who is administered placebo.

30. The method of any one of embodiments 1-10, wherein the subject has no change or a reduction in iron overload and/or liver iron accumulation after receiving the composition comprising 15-HEPE relative to baseline or to a control subject who is not administered the composition comprising 15-HEPE or who is administered placebo.

31. The method of any one of embodiments 1-10, wherein the subject has no change or a reduction in oxidative stress after receiving the composition comprising 15-HEPE relative to baseline or to a control subject who is not administered the composition comprising 15-HEPE or who is administered placebo.

32. The method of any one of embodiments 1-10, wherein the subject has no change or a reduction in nuclear factor erythroid 2-related factor 2 (Nrf2) levels after receiving the composition comprising 15-HEPE relative to baseline or to a control subject who is not administered the composition comprising 15-HEPE or who is administered placebo.

33. The method of any one of embodiments 1-10, wherein the subject has no change or a reduction in oxidation and/or heme oxygenase-1 (HO-1) levels after receiving the composition comprising 15-HEPE relative to baseline or to a control subject who is not administered the composition comprising 15-HEPE or who is administered placebo.

34. The method of any one of embodiments 1-10, wherein the subject has no change or a reduction in E-selectin levels after receiving the composition comprising 15-HEPE relative to baseline or to a control subject who is not administered the composition comprising 15-HEPE or who is administered placebo.

35. The method of any one of embodiments 1-10, wherein the subject has no change or a reduction in red blood cell adhesion after receiving the composition comprising 15-HEPE relative to baseline or to a control subject who is not administered the composition comprising 15-HEPE or who is administered placebo.

36. The method of any one of embodiments 1-35, wherein the 15-HEPE is in free acid form.

37. The method of any one of embodiments 1-35, wherein the 15-HEPE is in esterified form.

38. The method of embodiment 37, wherein the esterified form is a C1-C5 alkyl ester.

39. The method of embodiment 38, wherein the esterified form is an ethyl ester.

40. The method of embodiment 37, wherein the esterified form is a triglyceride.

41. The method of any one of embodiments 1-35, wherein the 15-HEPE is in salt form.

42. The method of any one of embodiments 1-41, wherein the 15-HEPE is in 15(S)-HEPE enantiomeric form and/or 15(R)-HEPE enantiomeric form.

43. The method of any one of embodiments 1-42, wherein the composition comprises one or more additional active agent(s) selected from the group consisting of eicosapentaenoic acid (EPA), 5-hydroxy-eicosapentaenoic acid (5-HEPE), 12-hydroxy-eicosapentaenoic acid (12-HEPE), 18-hydroxy-eicosapentaenoic acid (18-HEPE), 15-hydroxy-eicosatrienoic acid (15-HETrE), 15-hydroxy-eicosatetraenoic acid (15-HETE), 14-hydroxy-docosahexaenoic acid (14-HDoHE), 17-hydroxy-docosahexaenoic acid (17-HDoHE), resolvin D1 (RvD1 or 17(S)-RvD1), and aspirin-triggered resolvin D1 (AT-RvD1 or 17(R)-RvD1).

44. The method of any one of embodiments 1-43, wherein 15-HEPE represents at least 80% wt. of the composition.

45. The method of any one of embodiments 1-43, wherein 15-HEPE represents at least 80% wt. of all fatty acids present in the composition.

46. The method of any one of embodiments 1-43, wherein the composition comprises no more than 10% wt. of omega-3 fatty acids other than 15-HEPE.

47. The method of any one of embodiments 1-43, wherein omega-3 fatty acids other than 15-HEPE represents no more than 10% wt. of all fatty acids present in the composition.

48. The method of any one of embodiments 1-47, wherein the composition is formulated in one or more orally deliverable dosage units.

49. The method of embodiment 48, wherein the dosage unit is a capsule.

50. The method of embodiment 49, wherein about 500 mg to about 1000 mg of 15-HEPE is present in each capsule.

51. The method of any one of embodiments 1-50, wherein the composition further comprises a pharmaceutically acceptable excipient.

52. The method of any one of embodiments 1-51, wherein the composition is administered to the subject to provide a daily dose of about 1 g to about 8 g of 15-HEPE.

53. The method of embodiment 52, wherein the composition is administered to the subject to provide a daily dose of about 8 g of 15-HEPE.

54. The method of embodiment 52 or claim 53, wherein the composition is administered in 1 to 8 capsules per day.

1. A method of treating and/or preventing a hematologic disorder in a subject in need thereof, comprising administering to the subject a composition comprising 15-hydroxyeicosapentaenoic acid (15-HEPE) in free acid form or a pharmaceutically acceptable ester, conjugate, or salt thereof.

2. The method of claim 1, wherein the hematologic disorder is selected from the group consisting of a red blood cell disorder, a hemoglobin disorder, a bleeding disorder, and a blood cancer.

3. The method of claim 2, wherein:
(a) the red blood cell disorder is selected from the group consisting of anemia, hemolytic anemia, iron deficiency anemia, hereditary spherocytosis, chronic hemolysis, pyruvate kinase deficiency, and glucose-6-phosphate dehydrogenase (G6PD) deficiency;
(b) the hemoglobin disorder is selected from the group consisting of sickle cell disease, alpha thalassemia, and beta thalassemia;
(c) the bleeding disorder is selected from the group consisting of hemophilia, thrombophilia, thrombocytopenia, idiopathic_thrombocytopenia purpura (ITP), heparin-induced thrombocytopenia (HIT), venous thromboembolism, arterial thrombosis, and embolism; and
(d) the blood cancer is selected from the group consisting of myelodysplastic syndrome (MDS), lymphoma, leukemia, and myeloma.

4-6. (canceled)

7. A method of treating and/or preventing sickle cell disease and/or symptoms associated thereof in a subject in need thereof, comprising administering to the subject a composition comprising 15-hydroxyeicosapentaenoic acid (15-HEPE) in free acid form or a pharmaceutically acceptable ester, conjugate, or salt thereof.

8. The method of claim 7, wherein the symptoms associated thereof comprise inflammatory vasculopathy, vasoconstriction, bronchoconstriction, iron overload, hemoglobinopathy, chromic hemolysis, sickling, anemia, inflammation, pain, acute and/or chronic damage to an organ, and/or oxidative stress.

9. The method of claim 8, wherein the organ is lung, kidney, liver, or spleen.

10. A method of treating and/or preventing heparin-induced thrombocytopenia (HIT) in a subject in need thereof, comprising administering to the subject a composition comprising 15-hydroxyeicosapentaenoic acid (15-HEPE) in free acid form or a pharmaceutically acceptable ester, conjugate, or salt thereof.

11. The method of claim 1, wherein the subject has no change or a reduction in serum creatine levels, C-reactive protein (CRP) levels, pentraxin-2 levels, monocyte chemoattractant protein-1 (MCP-1/CCL2) levels, circulating neutrophils, vascular cell adhesion molecule 1 (VCAM-1) levels, intercellular adhesion molecule 1 (ICAM-1) levels, endothelin-1 (ET-1) levels, CD68 levels, CD80 levels, NF-kB p65 activation, NLRP3 inflammasome activation, vascular endothelial activation, neutrophil vascular recruitment, inflammatory cell infiltration, thrombi formation, iron overload and/or liver iron accumulation, oxidative stress, nuclear factor erythroid 2-related factor 2 (Nrf2) levels, oxidation and/or heme oxygenase-1 (HO-1) levels, E-selectin levels, or red blood cell adhesion-after receiving the composition comprising 15-HEPE relative to baseline or to a control subject who is not administered the composition comprising 15-HEPE or who is administered placebo.

12-18. (canceled)

19. The method of claim 1, wherein the subject has no change or an increase in hematocrit levels or hemoglobin levels after receiving the composition comprising 15-HEPE relative to baseline or to a control subject who is not administered the composition comprising 15-HEPE or who is administered placebo.

20. (canceled)

21. The method of claim 1, wherein the subject has no change or a reduction in sickled red blood cell numbers after receiving the composition comprising 15-HEPE relative to baseline or to a control subject who is not administered the composition comprising 15-HEPE or who is administered placebo.

22-35. (canceled)

36. The method of claim 1, wherein the 15-HEPE is in free acid form, esterified form, or salt form, and wherein the esterified form is a C1-C5 alkyl ester, an ethyl ester, or a triglyceride.

37-41. (canceled)

42. The method of claim 1, wherein the 15-HEPE is in 15(S)-HEPE enantiomeric form and/or 15(R)-HEPE enantiomeric form.

43. The method of claim 1, wherein the composition comprises one or more additional active agent(s) selected from the group consisting of eicosapentaenoic acid (EPA), 5-hydroxy-eicosapentaenoic acid (5-HEPE), 12-hydroxy-eicosapentaenoic acid (12-HEPE), 18-hydroxy-eicosapentaenoic acid (18-HEPE), 15-hydroxy-eicosatrienoic acid (15-HETrE), 15-hydroxy-eicosatetraenoic acid (15-HETE), 14-hydroxy-docosahexaenoic acid (14-HDoHE), 17-hydroxy-docosahexaenoic acid (17-HDoHE), resolvin D1 (RvD1 or 17(S)-RvD1), and aspirin-triggered resolvin D1 (AT-RvD1 or 17(R)-RvD1).

44. The method of claim 1, wherein 15-HEPE represents at least 80% wt. of the composition, or at least 80% wt. of all fatty acids present in the composition.

45. (canceled)

46. The method of claim 1, wherein the composition comprises no more than 10% wt. of omega-3 fatty acids other than 15-HEPE or omega-3 fatty acids other than 15-HEPE represents no more than 10% wt. of all fatty acids present in the composition.

47. (canceled)

48. The method of claim 1, wherein the composition is formulated in one or more orally deliverable dosage units.

49. (canceled)

50. The method of claim 4948, wherein about 500 mg to about 1000 mg of 15-HEPE is present in each a capsule.

51. (canceled)

52. The method of claim 1, wherein the composition is administered to the subject to provide a daily dose of about 1 g to about 8 g of 15-HEPE.

53. The method of claim 52, wherein the composition is administered to the subject to provide a daily dose of about 8 g of 15-HEPE.

54. The method of claim 52, wherein the composition is administered in 1 to 8 capsules per day.


SEQUENCE LISTING (10 sequences)

SEQ ID NO: 1 - DNA, 19 bp, synthetic construct
  misc_feature 1..19: note = 36B4 MA057856 forward primer
  source 1..19: mol_type = other DNA
ttccaggctttgggcatca

SEQ ID NO: 2 - DNA, 24 bp, synthetic construct
  misc_feature 1..24: note = 36B4 MA057856 reverse primer
  source 1..24: mol_type = other DNA
atgttcagcatgttcagcagtgtg

SEQ ID NO: 3 - DNA, 22 bp, synthetic construct
  misc_feature 1..22: note = Alpha-SMA MA057911 forward primer
  source 1..22: mol_type = other DNA
aagagcatccgacactgctgac

SEQ ID NO: 4 - DNA, 24 bp, synthetic construct
  misc_feature 1..24: note = Alpha-SMA MA057911 reverse primer
  source 1..24: mol_type = other DNA
agcacagcctgaatagccacatac

SEQ ID NO: 5 - DNA, 20 bp, synthetic construct
  misc_feature 1..20: note = TIMP-1 MA098519 forward primer
  source 1..20: mol_type = other DNA
tgagccctgctcagcaaaga

SEQ ID NO: 6 - DNA, 21 bp, synthetic construct
  misc_feature 1..21: note = TIMP-1 MA098519 reverse primer
  source 1..21: mol_type = other DNA
gaggacctgatccgtccacaa

SEQ ID NO: 7 - DNA, 25 bp, synthetic construct
  misc_feature 1..25: note = TGF-beta MA030397 forward primer
  source 1..25: mol_type = other DNA
gtgtggagcaacatgtggaactcta

SEQ ID NO: 8 - DNA, 20 bp, synthetic construct
  misc_feature 1..20: note = TGF-beta MA030397 reverse primer
  source 1..20: mol_type = other DNA
ttggttcagccactgccgta

SEQ ID NO: 9 - DNA, 24 bp, synthetic construct
  misc_feature 1..24: note = Collagen Type 1 MA075477 forward primer
  source 1..24: mol_type = other DNA
ccaacaagcatgtctggttaggag

SEQ ID NO: 10 - DNA, 23 bp, synthetic construct
  misc_feature 1..23: note = Collagen Type 1 MA075477 reverse primer
  source 1..23: mol_type = other DNA
gcaatgctgttcttgcagtggta